SEQUENCE LISTING (104 sequences)

SEQ ID NO: 1 - DNA, 1611 bp, Corynebacterium diphtheriae
atgggtgcagatgacgtggttgacagctctaaatcttttgtgatggaaaacttcagttcctatcatggcaccaaaccgggctacgttgatagcattcagaaaggcatccaaaaaccgaaatctggcacgcagggtaactacgatgacgattggaaagaattttactctaccgacaacaaatacgatgcggccggttactcagtcgacaacgaaaatccgctgagcggtaaagcgggcggtgtcgtgaaagtgacgtatccgggtctgaccaaagttctggccctgaaagtcgataatgcagaaaccatcaaaaaagaactgggtctgagtctgacggaaccgctgatggaacaggttggcaccgaagaatttatcaaacgcttcggcgatggtgccagtcgtgttgtcctgtccctgccgttcgcagaaggctcatcgagcgttgaatatattaacaattgggaacaagcgaaagccctgagcgtcgaactggaaatcaactttgaaacccgcggcaaacgtggtcaggatgccatgtatgaatacatggcacaggcgtgcgccggtaatcgtgtgcgtcgcagcgttggctctagtctgtcttgtatcaacctggactgggatgttatccgtgataaaaccaaaacgaaaatcgaaagtctgaaagaacacggtccgatcaaaaacaaaatgtcagaatcgccgaataaaacggtgtccgaagaaaaagctaaacagtatctggaagaatttcaccaaaccgcactggaacatccggaactgtcagaactgaaaaccgttacgggcaccaacccggtctttgccggcgcaaattacgcagcttgggctgtcaacgtggcgcaagtgattgactcggaaacggcggataatctggaaaaaaccacggcggccctgagtattctgccgggcatcggttccgtcatgggtattgccgatggcgcagtgcatcacaacaccgaagaaattgttgcccagagtatcgcactgtcctcactgatggttgctcaagcgattccgctggtcggtgaactggtggatattggctttgcagcttataatttcgtggaatccattatcaacctgtttcaggtggttcataactcatataatcgcccggcgtactcgccgggtcacaaaacccaaccgttcctgcatgacggctacgccgtgagctggaatacggttgaagattctattatccgtaccggctttcagggtgaaagcggccacgacattaaaatcacggctgaaaacaccccgctgccgattgcgggtgttctgctgccgaccatcccgggtaaactggatgtcaataaatcaaaaacccatatctcggtgaacggtcgcaaaattcgtatgcgctgccgtgccatcgacggcgatgtgaccttctgtcgtccgaaaagcccggtttatgtcggcaacggtgtgcatgctaatctgcacgttgcgtttcatcgtagcagcagcgaaaaaattcacagtaatgaaatcagttccgactccattggtgtgctgggctaccagaaaacggtcgatcataccaaagtgaacagcaaactgtctctgtttttcgaaattaaatcttaa

SEQ ID NO: 2 - protein, 536 aa, Corynebacterium diphtheriae
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 3 - DNA, 34 bp, unknown
  Primer
cccatatgggtgcagatgacgtggttgacagctc

SEQ ID NO: 4 - DNA, 39 bp, Unknown
  Primer
ccggatccagatttaatttcgaaaaacagagacagtttg

SEQ ID NO: 5 - DNA, 42 bp, Artificial
  Primer
ccggatccttaagatttaatttcgaaaaacagagacagtttg

SEQ ID NO: 6 - protein, 381 aa, Mycobacterium tuberculosis
FSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 7 - protein, 456 aa, Mycobacterium tuberculosis
FSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGMIAGVDQALAATGQASQRAAGASGGVTVGVGVGTEQRNLSVVAPSQFTFSSRSPDFVDETAGQSWCAILGLNQFH

SEQ ID NO: 8 - protein, 13 aa, Streptococcus pyogenes
AHIVMVDAYKPTK

SEQ ID NO: 9 - protein, 16 aa, Streptococcus pyogenes
TDKDMTITFTNKKDAE

SEQ ID NO: 10 - protein, 12 aa, Streptococcus pneumoniae
KLGDIEFIKVNK

SEQ ID NO: 11 - protein, 12 aa, Streptococcus pneumoniae
KLGSIEFIKVNK

SEQ ID NO: 12 - protein, 23 aa, Streptococcus pneumoniae
DIPATYEFTDGKHYITNEPIPPK

SEQ ID NO: 13 - protein, 13 aa, Streptococcus pneumoniae
DPIVMIDNDKPIT

SEQ ID NO: 14 - protein, 16 aa, artificial
  Ionic peptide
LELELKLKLELELKLK

SEQ ID NO: 15 - DNA, 1143 bp, Mycobacterium tuberculosis
ttcagccgtccgggtctgccggtcgaatacctgcaagttccgtcgccgagcatgggtcgtgacattaaagttcagttccaaagcggtggtaacaatagcccggccgtctatctgctggacggtctgcgtgcacaggatgactacaatggctgggatattaacaccccggcgtttgaatggtattaccagtcaggcctgtcgatcgtgatgccggttggcggtcaaagctctttctatagcgattggtactctccggcgtgcggtaaagccggctgtcagacctataaatgggaaacctttctgacgagtgaactgccgcagtggctgtccgcaaatcgtgcagttaaaccgacgggttcagcggccattggcctgtcgatggcaggtagttccgctatgattctggcagcttatcatccgcagcaattcatctacgcaggtagtctgtccgctctgctggacccgagccagggtatgggtccgtctctgatcggtctggcaatgggtgatgccggcggttataaagcggccgatatgtggggtccgtcatcggacccggcatgggaacgtaacgatccgacccagcaaattccgaaactggtcgccaacaatacccgcctgtgggtgtactgcggcaacggtacgccgaatgaactgggcggtgcaaatatcccggctgaatttctggaaaatttcgttcgtagctctaacctgaaatttcaggatgcgtataacgcagctggcggtcataacgccgtctttaatttcccgccgaacggcacccacagttgggaatactggggtgcgcaactgaatgccatgaaaggtgacctgcagagttccctgggtgcaggcatgtctcaaattatgtataactacccggcaatgctgggtcacgcaggtgatatggcaggttatgctggcacgctgcagagcctgggtgcggaaattgccgtggaacaggcggccctgcaatctgcgtggcagggtgacaccggcatcacgtatcaagcatggcaggctcaatggaatcaggccatggaagatctggttcgtgcgtaccatgccatgtcatcgacccacgaagcaaacacgatggcaatgatggctcgcgacaccgccgaagcagctaaatggggcggt

SEQ ID NO: 16 - DNA, 1368 bp, Mycobacterium tuberculosis
ttcagccgtccgggtctgccggtcgaatacctgcaagttccgtcgccgagcatgggtcgtgacattaaagttcagttccaaagcggtggtaacaatagcccggccgtctatctgctggacggtctgcgtgcacaggatgactacaatggctgggatattaacaccccggcgtttgaatggtattaccagtcaggcctgtcgatcgtgatgccggttggcggtcaaagctctttctatagcgattggtactctccggcgtgcggtaaagccggctgtcagacctataaatgggaaacctttctgacgagtgaactgccgcagtggctgtccgcaaatcgtgcagttaaaccgacgggttcagcggccattggcctgtcgatggcaggtagttccgctatgattctggcagcttatcatccgcagcaattcatctacgcaggtagtctgtccgctctgctggacccgagccagggtatgggtccgtctctgatcggtctggcaatgggtgatgccggcggttataaagcggccgatatgtggggtccgtcatcggacccggcatgggaacgtaacgatccgacccagcaaattccgaaactggtcgccaacaatacccgcctgtgggtgtactgcggcaacggtacgccgaatgaactgggcggtgcaaatatcccggctgaatttctggaaaatttcgttcgtagctctaacctgaaatttcaggatgcgtataacgcagctggcggtcataacgccgtctttaatttcccgccgaacggcacccacagttgggaatactggggtgcgcaactgaatgccatgaaaggtgacctgcagagttccctgggtgcaggcatgtctcaaattatgtataactacccggcaatgctgggtcacgcaggtgatatggcaggttatgctggcacgctgcagagcctgggtgcggaaattgccgtggaacaggcggccctgcaatctgcgtggcagggtgacaccggcatcacgtatcaagcatggcaggctcaatggaatcaggccatggaagatctggttcgtgcgtaccatgccatgtcatcgacccacgaagcaaacacgatggcaatgatggctcgcgacaccgccgaagcagctaaatggggcggtatgatcgcaggcgtggatcaggctctggcagcaacgggtcaggcatcacaacgtgcagctggtgcatcgggcggtgtcaccgtgggcgttggtgtcggcacggaacagcgtaatctgagtgtggttgcgccgtcccaatttaccttcagctctcgcagcccggatttcgttgacgaaacggcgggccaaagctggtgtgccattctgggtctgaaccaattccac

SEQ ID NO: 17 - protein, 20 aa, Streptococcus pyogenes
LRRDLDASREAKNQVERALE

SEQ ID NO: 18 - protein, 20 aa, Streptococcus pyogenes
NSDNIKENQFEDFDEDWENF

SEQ ID NO: 19 - protein, 919 aa, Artificial
  Chimera
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKSGSFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 20 - protein, 994 aa, artificial
  Chimera
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKSGSFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGMIAGVDQALAATGQASQRAAGASGGVTVGVGVGTEQRNLSVVAPSQFTFSSRSPDFVDETAGQSWCAILGLNQFH

SEQ ID NO: 21 - protein, 388 aa, Artificial
  Chimera
MHHHHHHFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 22 - protein, 463 aa, Artificial
  Chimera
MHHHHHHFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGMIAGVDQALAATGQASQRAAGASGGVTVGVGVGTEQRNLSVVAPSQFTFSSRSPDFVDETAGQSWCAILGLNQFH

SEQ ID NO: 23 - protein, 560 aa, Corynebacterium diphtheriae
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGDVVKVTYPGLTKVLALKVDNAETIKKELGLSLTEPLMEQVGTEEFIKRFGDGASRVVLSLPFAEGSSSVEYINNWEQAKALSVKLEINFETRGKRGQDAMYEYMAQACAGNRVRRSVGGSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVEDSIIRTGFQGESGHDIKITAENTPLPIASVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 24 - protein, 560 aa, Corynebacterium diphtheriae
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFDDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 25 - protein, 560 aa, Corynebacterium diphtheriae
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFYRPKSPVYVGNGVHANLHVAFHRSSSEKIHSNEISSDSIGVLGYQKTVDHTKVNSKLSLFFEIKS

SEQ ID NO: 26 - protein, 411 aa, Corynebacterium diphtheriae
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKT

SEQ ID NO: 27 - protein, 560 aa, Corynebacterium diphtheriae
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 28 - protein, 50 aa, Hepatitis C virus
MSTNPKPQRKTKRNTNRRPQDVKFPGGGQIVGGVYLLPRRGPRLGVRATR

SEQ ID NO: 29 - protein, 204 aa, Hepatitis C virus
APTGSGKSTKVPAAYAAQGYKVLVLNPSVAATLGFGAYMSKAHGIDPNIRTGVRTITTGAPVTYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSGLGINAVAYYRGLDVSVIPTIGDVV

SEQ ID NO: 30 - protein, 137 aa, Hepatitis C virus
SAYEVRNASGVYHVTNDCSNSSIVYEADDMIMHTPGCVPCVREDNTSRCWVALTPTLAARNASVPTTTIRRHVDLLVGAAALCSAMYVGDLCGSVFLVSQLFTFSPRRHETAQDCNCSIYPGHVSGHRMAWDMMMNW

SEQ ID NO: 31 - protein, 212 aa, Hepatitis C virus
SQKIQLVNTNGSWHINRTALNCNDSLQTGFLAALFYVHRFNSSGCSDRMASCRPIDTFDQGWGPITYAEPRSLDQRPYCWHYAPQPCGIVPAAEVCGPVYCFTPSPVVVGTTDRSGVPTYNWGENETDVLLLNNTRPPLGNWFGCTWMNSTGFTKTCGGPPYNIGGVGNNTLTCPTDCFRKHPEATYTKCGLGPWLTPRCLVDYPYRLWHYP

SEQ ID NO: 32 - protein, 149 aa, Mycobacterium tuberculosis
MATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTNGGGGG

SEQ ID NO: 33 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 34 - protein, 100 aa, Mycobacterium tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 35 - protein, 294 aa, Mycobacterium tuberculosis
MVFALSNNLNRVNACMDGFLARIRSHVDAHAPELRSLFDTMAAEARFARDWLSEDLARLPVGAALLEVGGGVLLLSCQLAAEGFDITAIEPTGEGFGKFRQLGDIVLELAAARPTIAPCKAEDFISEKRFDFAFSLNVMEHIDLPDEAVRRVSEVLKPGASYHFLCPNYVFPYEPHFNIPTFFTKELTCRVMRHRIEGNTGMDDPKGVWRSLNWITVPKVKRFAAKDATLTLRFHRAMLVWMLERALTDKEFAGRRAQWMVAAIRSAVKLRVHHLAGYVPATLQPIMDVRLTKR

SEQ ID NO: 36 - protein, 119 aa, Mycobacterium tuberculosis
ADAVKYVVMCNCDDEPGALIIAWIDDERPAGGHIQMRSNTRFTETQWGRHIEWKLECRACRKYAPISEMTAAAILDGFGAKLHELRTSTIPDADDPSIAEARHVIPFSALCLRLSQLGG

SEQ ID NO: 37 - protein, 599 aa, Mycobacterium tuberculosis
VIPVMSARFTGFPLLPVALRHGITSGRGCGFILDVGAQRPFGNDVLLSVATRKIRSRLPGDRVGNHGALLPFRAEPRRIQMKRPPEVLRGAVTASRERLWAIGSQSERTLMLGTILLASVISAATAYALSQWYAVDVFSTLLVVPGDCWLDWGMNIGRHCFSDYAMVAAAGIQPNPADYLISLPADYQPTAVAAWAPARIPYAIFGLPSHWLGAPRLGLICYLVALTMAVISPAIWAARGARGLERVVIFVTLGAAAIPAWGVIDRGNSTGFVVPIALAYFVALSRQRWGLATITVILAVLVKPQFVVLGVVLLAARQWRWAGIGITGVVVSNIAAFLLWPRGFPGTIAQSIHGIIKFNSSFGGLRDPRNVSFGKALLLIPDSIKNYQSGKIPEGFLTGPRTQIGFAVLVIVVVAVLALGRRIPPVMVGIVLLATATFSPADVAFYYLVFVLPIAALVARDPNGPPGAGIFDQLAAHGDRRRAVGVCVSLAVALSIVNVAVPGQPFYVPLYGQLGAKGVVGTTPLVFTTVTWAPFLWLVTCVVIIVSYARKPARPHDSHNGPTRESDQDTAASTTSCLPNPVEESSPRGPGPICQNYTP

SEQ ID NO: 38 - protein, 81 aa, Mycobacterium tuberculosis
MSGHALAARTLLAAADELVGGPPVEASAAALAGDAAGAWRTAAVELARALVRAVAESHGVAAVLFAATAAAAAAVDRGDPP

SEQ ID NO: 39 - protein, 104 aa, Mycobacterium tuberculosis
PMTENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFT

SEQ ID NO: 40 - protein, 97 aa, Mycobacterium tuberculosis
MSLLDAHIPQLIASHTAFAAKAGLMRHTIGQAEQQAMSAQAFHQGESAAAFQGAHARFVAAAAKVNTLLDIAQANLGEAAGTYVAADAAAASSYTGF

SEQ ID NO: 41 - protein, 141 aa, Dengue virus
RLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLG

SEQ ID NO: 42 - protein, 99 aa, Dengue virus
NNQRKKARSTPFNMLKRERNRVSTVQQLTKRFSLGMLQGRGPLKLFMALVAFLRFLTIPPTAGILKRWGTIKKSKAINVLRGFRKEIGRMLNILNRRRR

SEQ ID NO: 43 - protein, 191 aa, Hepatitis C virus
MSTNPKPQRKTKRNTNRRPQDVKFPGGGQIVGGVYLLPRRGPRLGVRATRKTSERSQPRGRRQPILRDRRSTGKSWGKPGYPWPLYGNEGCGWAGWLLSPRGSRPTWGPTDPRHRSRNLGKVIDTITCSLADLMGYIPVIGAPVGGVARALAHGVRVLEDGVNYATGNLPGCSFSIFLLALLSCITVPVSA

SEQ ID NO: 44 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYAFYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRQAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 45 - protein, 412 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPSWGPTDPRRRSRNLGKVIDTLTCGFADLMGHIPLVGAPLGGAARALAHGVRVLEDGVNYATGNLPGCSFSIFLLALLSCLTIPASAYEVRNASGVYHVTNDCSNSSIVYETADMIMHTPGCVPCVREDNSSRCWVALTPTLAARNASIPTTTIRRHVDLLVGAAAFCSAMYVGDLCGSVFLVSQLFTFSPRRHETVQDCNCSIYPGHVSGHRMAWDMMMNWSPTAALMVSQLLRIPQAVVDMVAGAHWGVLAGLAYYSMAGNWAKVLIVMLLFAGVDGQTTVMGGVAGRTTFGFAALFNPGPSQKIQ

SEQ ID NO: 46 - protein, 650 aa, Hepatitis C virus
MSTNPKPQRKTKRNTNRRPQDVKFPGGGQIVGGVYLLPRRGPRLGVRATRKTSGRSQPRGRRQPIPKARRPEGRSWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSLFLLALLSCLTIPVSAYEVRNASGVYHVTNDCSNSSIVYEADDMIMHTPGCVPCVREDNTSRCWVALTPTLAARNASVPTTTIRRHVDLLVGAAALCSAMYVGDLCGSVFLVSQLFTFSPRRHETVQDCNCSIYPGHVSGHRMAWDMMMNWSPSTALVVSQLLRIPQAVVDMVAGAHWGVLAGLAYYSMVGNWAKVLIVMLLFAGVDGTGTYVTGGTAARGVSQFTGLFTSGPSQKIQLVNTNGSWHINRTALNCNDSLQTGFLAALFYVHRFNSSGCSDRMASCRPIDTFDQGWGPITYAEPRSLDQRPYCWHYAPQPCGIVPAAEVCGPVYCFTPSPVVVGTTDRSGVPTYNWGENETDVLLLNNTRPPLGNWFGCTWMNSTGFTKTCGGPPYNIGGVGNNTLTCPTDCFRKHPEATYTKCGLGPWLTPRCLVDYPYRLWHYPCTVNFTIFKVRMYVGGVEHRLTAACNWTRG

SEQ ID NO: 47 - protein, 495 aa, Dengue virus
MRCIGISNRDFVEGVSGGSWVDIVLEHGSCVTTMAKNKPTLDFELIKTEAKQPATLRKYCIEARLTNTTTESRCPTQGEPSLKEEQDKRFVCKHSIVDRGWGNGCGLFGKGGIVTCAMFTCKKNMEGKIVQPENLEYTIVITPHSGEEHAVGNDTGKHGKEIKITPQSSITEAELTGYGTITMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGAMVQA

SEQ ID NO: 48 - protein, 774 aa, Dengue virus
NNQRKKARSTPFNMLKRERNRVSTVQQLTKRFSLGMLQGRGPLKLFMALVAFLRFLTIPPTAGILKRWGTIKKSKAINVLRGFRKEIGRMLNILNRRRRTAGVIVMLIPTAMAFHLTTRNGEPHMIVGRQEKGKSLLFKTEDGVNMCTLMAIDLGELCEDTITYKCPLLRQNEPEDIDCWCNSTSTWVTYGTCTTTGEHRREKRSVALVPHVGMGLETRTETWMSSEGAWKHVQRIETWILRHPGFTIMAAILAYTIGTTHFQRALIFILLTAVAPSMTMRCIGISNRDFVEGVSGGSWVDIVLEHGSCVTTMAKNKPTLDFELIKTEAKQPATLRKYCIEAKLTNTTTESRCPTQGEPSLNEEQDKRFICKHSMVDRGWGNGCGLFGKGGIVTCAMFTCKKNMEGKVVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEITDLEKRHVLGRLITVNPIITEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGAMVQA

SEQ ID NO: 49 - protein, 560 aa, Corynebacterium diphtheriae
MSRKLFASILIGALLGIGAPPSAHAGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 50 - protein, 535 aa, Corynebacterium diphtheriae
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 51 - protein, 535 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGDVVKVTYPGLTKVLALKVDNAETIKKELGLSLTEPLMEQVGTEEFIKRFGDGASRVVLSLPFAEGSSSVEYINNWEQAKALSVKLEINFETRGKRGQDAMYEYMAQACAGNRVRRSVGGSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVEDSIIRTGFQGESGHDIKITAENTPLPIASVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 52 - protein, 535 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFDDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 53 - protein, 535 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFYRPKSPVYVGNGVHANLHVAFHRSSSEKIHSNEISSDSIGVLGYQKTVDHTKVNSKLSLFFEIKS

SEQ ID NO: 54 - protein, 386 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKT

SEQ ID NO: 55 - protein, 535 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 56 - protein, 418 aa, Coronavirus SARS-CoV-2
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 57 - protein, 211 aa, Coronavirus SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 58 - protein, 680 aa, Coronavirus SARS-CoV-2
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 59 - protein, 368 aa, Coxiella burnetii
MASFQNYLNDYGPGPGVTLVEFFDYGPGPGHYLVNHPEVLVEASQGPGPGDVNYGYNAATGEYGDGPGPGFDSSYKRGQPATFPLGPGPGGKLGVAYTYNRANAGGPGPGVAMIWSVAAVAQTVGGPGPGPVSASITQFGPVGELGPGPGLLTKKQYDKAQASFQGPGPGTPTFVIGNKALTKFGFGPGPGKIGVIKAIRTITGLGLKEAGPGPGMMEHLQNITNLVSTGRQGAGPGPGKIPVKIIKPPFVRRGGPGPGRLGFMSFFTKAVVEALKRFGPGPGVAKLRGDLSSIIHKLGPGPGLSSIIHKLTSFSKTEAGPGPGSPAVLSAAKKIFGDGAGPGPGLRPVRYFTGVPSPVKTPEGPGPG

SEQ ID NO: 60 - protein, 252 aa, Coxiella burnetii
MKNRLTALFLAGTLTAGVAIAAPSQFSFSPQQVKDIQSIVHHYLVNHPEVLVEASQALQKKTEAQQEEHAQQAIKENAKKLFNDPASPVAGNPHGNVTLVEFFDYQCGHCKAMNSVIQAIVKQNKNLRVVFKELPIFGGQSQYAAKVSLAAAKQGKYYAFHDALLSVDGQLSEQITLQTAEKVGLNVAQLKKDMDNPAIQKQLRDNFQLAQSLQLAGTPTFVIGNKALTKFGFIPGATSQQNLQKEIDRVEK

SEQ ID NO: 61 - protein, 224 aa, artificial
  Predicted T and B epitopes from OmpH
APQIKDINTRLEKQFSGGGGGMSKVNGAVKRVAERENGGGGGLSAICLSVAMIWSVAAGGGGGLRKEIQNDESTLRQQQGGGGGTRLEKQFSPQREKMTKGGGGGRVAERENLDLVLPKDTGGGGGYAKNSKDITSNGGGGGQNKAMSDGGGGGLYAKNSKDITSNGGGGGGKKEAENLRKEIQNDEGGGGGTLRQQQQQFQQEGGGGGQELFVAQNKAMSDFM

SEQ ID NO: 62 - protein, 305 aa, Coxiella burnetii
MRLIKMKIKTLCVSSALAALMLSAPLTWADAPVEDISAQPQPTKTTVSPSETPETAIPTAPVSLPTTQTDLTVTHRLARLEQQLNNIINMNLPQQISDLQQRLAQVRGQLQVQERNLELLNNQQRSFYRDLDQRITQLKNLNSNNSDSSNDNSASSSQKPSSGDTSNTNNIQLQDSNTYRQALDLLTKKQYDKAQASFQNYLNDYPNGSYVANAHYWLGEIYLQQKDRKNAAHEFQTVRDKFPKSEKVLDAKLKLAIIDAEDGKIKQAKEELTEIKKQHPESTAAQLANIRLQQLEEVDSATTTP

SEQ ID NO: 63 - protein, 257 aa, Artificial
  predicted B and T cell epitopes derived from Q fever peptide antigen - GroEL
TEVEMKEKKARVEDALGGGGGGYLSPYFIGGGGGVEEGVVPGGGVGGGGGKKISNIRGGGGGVTKDDTTIIDGSGDAGDIKNGGGGGIKNRVEQIRKEIENSSSDYDKEKLQERLGGGGGTEAPKKKEESMPGGGGGASRTSDDAGDGTTTATGGGGGSKPCKDQKAGGGGGSANSDKSIGDGGGGGEKVGKEGGGGGGNNQQNMSGGGGGDSVEVENEDQRVGGGGGGATGEYGDGGGGGSHEVLHAMSRGVEVLA

SEQ ID NO: 64 - protein, 1273 aa, Coronavirus SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 65 - protein, 611 aa, Artificial
  Chimera
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKSGPGPGLRRDLDASREAKNQVERALEGPGPGLRRDLDASREAKNQVERALEGPGPGLRRDLDASREAKNQVERALE

SEQ ID NO: 66 - protein, 611 aa, Artificial
  Chimera
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKSGPGPGNSDNIKENQFEDFDEDWENFGPGPGNSDNIKENQFEDFDEDWENFGPGPGNSDNIKENQFEDFDEDWENF

SEQ ID NO: 67 - protein, 686 aa, Artificial
  Chimera
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKSGPGPGLRRDLDASREAKNQVERALEGPGPGLRRDLDASREAKNQVERALEGPGPGLRRDLDASREAKNQVERALEGPGPGNSDNIKENQFEDFDEDWENFGPGPGNSDNIKENQFEDFDEDWENFGPGPGNSDNIKENQFEDFDEDWENF

SEQ ID NO: 68 - protein, 686 aa, Artificial; identical to SEQ ID NO: 67
  Chimera

SEQ ID NO: 69 - protein, 645 aa, Hepatitis C virus
GSVVIVGRIILSGSGSITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQSFQVAHLHAPTGSGKSTKVPAAYAAQGYKVLVLNPSVAATLGFGAYMSKAHGIDPNIRTGVRTITTGAPVTYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSGLGINAVAYYRGLDVSVIPTIGDVVVVATDALMTGYTGDFDSVIDCNTCVTQTVDFSLDPTFTIETTTVPQDAVSRSQRRGRTGRGRRGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVT

SEQ ID NO: 70 - protein, 32 aa, Hepatitis C virus
SAYEVRNASGVYHVTNDCSNSSIVYEADDMIM

SEQ ID NO: 71 - skipped

SEQ ID NO: 72 - protein, 165 aa, Coxiella burnetii
MIKRLLSAICLSVAMIWSVAAVAQTVGLVDMRQIFQTAPQIKDINTRLEKQFSPQREKMTKLTQSLQQNLQKLKRDEAVMGKKEAENLRKEIQNDESTLRQQQQQFQQELFVAQNKAMSDFMSKVNGAVKRVAERENLDLVLPKDTVLYAKNSKDITSNVVSALK

SEQ ID NO: 73 - protein, 552 aa, Coxiella burnetii
MAAKVLKFSHEVLHAMSRGVEVLANAVKVTLGPKGRNVVLDKSFGAPTITKDGVSVAKEIELEDKFENMGAQMVKEVASRTSDDAGDGTTTATVLAQAILVEGIKAVIAGMNPMDLKRGIDKAVTAAVAELKKISKPCKDQKAIAQVGTISANSDKSIGDIIAEAMEKVGKEGVITVEDGSGLENALEVVEGMQFDRGYLSPYFINNQQNMSAELENPFILLVDKKISNIRELIPLLENVAKSGRPLLVIAEDIEGEALATLVVNNIRGVVKVAAVKAPGFGDRRKAMLQDIAVLTGGKVISEEVGLSLEAASLDDLGSAKRVVVTKDDTTIIDGSGDAGDIKNRVEQIRKEIENSSSDYDKEKLQERLAKLAGGVAVIKVGAATEVEMKEKKARVEDALHATRAAVEEGVVPGGGVALIRVLKSLDSVEVENEDQRVGVEIARRAMAYPLSQIVKNTGVQAAVVADKVLNHKDVNYGYNAATGEYGDMIEMGILDPTKVTRTALQNAASIAGLMITTECMVTEAPKKKEESMPGGGDMGGMGGMGGMGGMM

SEQ ID NO: 74 - protein, 16 aa, Coxiella burnetii
APQIKDINTRLEKQFS

SEQ ID NO: 75 - protein, 16 aa, Coxiella burnetii
MSKVNGAVKRVAEREN

SEQ ID NO: 76 - protein, 16 aa, Coxiella burnetii
LSAICLSVAMIWSVAA

SEQ ID NO: 77 - protein, 16 aa, Coxiella burnetii
LRKEIQNDESTLRQQQ

SEQ ID NO: 78 - protein, 16 aa, Coxiella burnetii
TRLEKQFSPQREKMTK

SEQ ID NO: 79 - protein, 16 aa, Coxiella burnetii
RVAERENLDLVLPKDT

SEQ ID NO: 80 - protein, 11 aa, Coxiella burnetii
YAKNSKDITSN

SEQ ID NO: 81 - protein, 7 aa, Coxiella burnetii
QNKAMSD

SEQ ID NO: 82 - protein, 12 aa, Coxiella burnetii
LYAKNSKDITSN

SEQ ID NO: 83 - protein, 16 aa, Coxiella burnetii
GKKEAENLRKEIQNDE

SEQ ID NO: 84 - protein, 12 aa, Coxiella burnetii
TLRQQQQQFQQE

SEQ ID NO: 85 - protein, 15 aa, Coxiella burnetii
QELFVAQNKAMSDFM

SEQ ID NO: 86 - protein, 16 aa, Coxiella burnetii
TEVEMKEKKARVEDAL

SEQ ID NO: 87 - protein, 8 aa, Coxiella burnetii
GYLSPYFI

SEQ ID NO: 88 - protein, 11 aa, Coxiella burnetii
VEEGVVPGGGV

SEQ ID NO: 89 - protein, 7 aa, Coxiella burnetii
KKISNIR

SEQ ID NO: 90 - protein, 20 aa, Coxiella burnetii
VTKDDTTIIDGSGDAGDIKN

SEQ ID NO: 91 - protein, 28 aa, Coxiella burnetii
IKNRVEQIRKEIENSSSDYDKEKLQERL

SEQ ID NO: 92 - protein, 12 aa, Coxiella burnetii
TEAPKKKEESMP

SEQ ID NO: 93 - protein, 16 aa, Coxiella burnetii
ASRTSDDAGDGTTTAT

SEQ ID NO: 94 - protein, 9 aa, Coxiella burnetii
SKPCKDQKA

SEQ ID NO: 95 - protein, 10 aa, Coxiella burnetii
SANSDKSIGD

SEQ ID NO: 96 - protein, 7 aa, Coxiella burnetii
EKVGKEG

SEQ ID NO: 97 - protein, 7 aa, Coxiella burnetii
NNQQNMS

SEQ ID NO: 98 - protein, 13 aa, Coxiella burnetii
DSVEVENEDQRVG

SEQ ID NO: 99 - protein, 7 aa, Coxiella burnetii
ATGEYGD

SEQ ID NO: 100 - protein, 16 aa, Coxiella burnetii
SHEVLHAMSRGVEVLA

SEQ ID NO: 101 - protein, 696 aa, Coronavirus SARS-CoV-2
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTM

SEQ ID NO: 102 - protein, 4 aa, Coronavirus SARS-CoV-2
RRAR

SEQ ID NO: 103 - protein, 12 aa, Coxiella burnetii
SVASQSIIAYTM

SEQ ID NO: 104 - protein, 153 aa, Hepatitis C virus
SQKIQLVNTNGSWHINRTALNCNDSLQTGFLAALFYVHRFNSSGCSDRMASCRPIDTFDQGWGPITYAEPRSLDQRPYCWHYAPQPCGIVPAAEVCGPVYCFTPSPVVVGTTDRSGVPTYNWGENETDVLLLNNTRPPLGNWFGCTWMNSTGF

(19) United States
(12) Patent Application Publication
(10) Pub. No.: US 2022/0378894 A1
(43) Pub. Date: Dec. 1, 2022

(54) PROTEIN PARTICLES COMPRISING A DIPHTHERIA TOXIN CROSS REACTING MATERIAL (CRM) AMINO ACID SEQUENCE AND USES THEREOF

(71) Applicant: GRIFFITH UNIVERSITY, Nathan, QLD (AU)

(72) Inventors: Bernd Helmut Adam REHM, Nathan (AU); Shuxiong CHEN, Nathan (AU)

(21) Appl. No.: 17/761,744

(22) PCT Filed: Sep. 21, 2020
(86) PCT No.: PCT/AU2020/000107

§ 371 Date: Mar. 18, 2022

(30) Foreign Application Priority Data
Sep. 20, 2019 (NL) 2023863

(51) Int. Cl. A61K 39/04; A61K 39/385; A61K 39/39; A61K 39/02; A61P 31/06; A61P 31/14

(52) U.S. Cl. CPC A61K 39/04; A61K 39/385; A61K 39/39; A61K 39/0208; A61P 31/06; A61P 31/14; A61K 2039/55516

(57) ABSTRACT

Methods of eliciting and/or modulating immune responses, therapeutic methods, and antigen delivery methods that include the step of administering a protein particle derived from a cell, the protein particle comprising a diphtheria toxin Cross Reacting Material (CRM) amino acid sequence are disclosed. Included are diagnostic methods using the protein particle derived from a cell, the protein particle comprising a diphtheria toxin CRM amino acid sequence. The methods disclosed herein may be useful as an antigen carrier delivery system.

### CROSS-REFERENCE TO RELATED APPLICATIONS

This application is a U.S. National Stage application of International Patent Application No. PCT/AU2020/000107, filed on Sep. 21, 2020, which claims the benefit of Dutch Patent Application No. 2023863, filed on Sep. 20, 2019.

### SEQUENCE LISTING

The instant application contains a Sequence Listing which has been submitted electronically in ASCII format and is hereby incorporated by reference in its entirety. Said ASCII copy, created on Mar. 30, 2022, is named 898_4UTIL_SL.txt and is 251,832 bytes in size.

### FIELD

This invention relates generally to particulate antigen carrier systems. More particularly, this invention relates to a non-toxic mutant form of diphtheria toxin, CRM, in methods of eliciting an immune response, therapeutic methods, delivery methods, detection methods and/or compositions.

### BACKGROUND

Development of suitable antigen carrier and delivery systems remains an evolving process due, partly, to an unmet need for vaccines against major pathogens and emerging diseases, particularly those that require a rapid public health response such as during a pandemic. Diphtheria toxin (DTx or DT), an extracellular toxin, is a secreted molecule of about 58.35 kDa produced by Corynebacterium diphtheriae (C. diphtheriae), the causative agent of diphtheria [1, 2]. Uchida et al. [12] described in 1973 five diphtheria toxin-related proteins obtained by mutation with nitrosoguanidine of corynephage β DNA containing the gene tox for diphtheria toxin. Following infection and lysogenisation of Corynebacterium diphtheriae, a number of mutated tox genes were expressed by the host bacterium and purified from culture supernatants. These products were given the general name ‘CRM’. The isolation of various non-toxic and partially toxic immunologically cross-reacting forms of diphtheria toxins (CRMs or cross reacting materials) resulted in discovery of CRM197 (Uchida et al., Journal of Biological Chemistry 248, 3845-3850, 1973; see also Giannini et al. Nucleic Acids Res. 1984 May 25; 12(10):4063-9). Other forms of CRMs are also known, for example CRM45.

CRM197 (“cross-reacting material 197”) is an enzymatically inactive and non-toxic form of diphtheria toxin with an approximate molecular weight of 58.415 kDa. CRM197 carries a single amino acid substitution of glycine to glutamate at residue 52 in the catalytic domain of DTx [3]. Although this substitution eliminates toxic activity of DTx, the overall structure of DTx and its mutated non-toxic derivative CRM197 are almost identical [3]. Moreover, the naturally nontoxic soluble form of CRM197 has been licensed for human use in conjugate vaccines as a carrier protein for a few capsular polysaccharide antigens wherein soluble CRM197 and polysaccharide antigens are covalently linked [4, 5]. The soluble active form CRM197 is also used as a potential vaccine candidate and potential alternative to conventional diphtheria toxoid vaccines, especially as a boosting antigen [3-5]. Despite offering an alternative to conventional diphtheria toxoid vaccines [3-5] and as an antigen carrier for other vaccine applications, use of soluble CRM197 at an industrial level has been hampered by low yields of the soluble form of the protein in expression systems, and the high costs and reliability issues associated with obtaining soluble CRM197.

There exists a continued need for development of an alternative antigen carrier and/or delivery system, which may be cost effective to manufacture.

### SUMMARY

In broad aspects, the present invention is directed, in part, to methods of eliciting an immune response in subject, modulating an immune response in a subject, therapeutic methods, and/or antigen delivery methods that include administering a particulate protein molecule derived from a cell, the protein particle comprising a diphtheria toxin Cross Reacting Material (CRM) amino acid sequence, and optionally one or more other immunogens. Suitably, the diphtheria toxin CRM amino acid sequence may be derived from Corynebacterium diphtheriae or corresponds to, or is, a diphtheria toxin CRM of Corynebacterium diphtheriae. The present invention includes compositions and/or diagnostic methods using said protein particles.

In a first aspect, the invention provides a method of eliciting in a subject an immune response to an agent, the method including the step of administering to the subject an effective amount of a protein particle comprising a diphtheria toxin CRM amino acid sequence, wherein the protein particle comprising the diphtheria toxin CRM amino acid sequence is derived from a cell, to thereby elicit in the subject the immune response against the agent.

In a second aspect, the invention provides a method of immunising a subject against a disease, disorder, or condition, the method including the step of administering to the subject an effective amount of a protein particle comprising a diphtheria toxin CRM amino acid sequence, wherein the protein particle comprising the diphtheria toxin CRM amino acid sequence is derived from a cell, to thereby immunise the subject against the disease, disorder, or condition.

In a third aspect, the invention provides a method of treating or preventing a disease, disorder, or condition in a subject, the method including the step of administering to the subject an effective amount of a protein particle comprising a diphtheria toxin CRM amino acid sequence, wherein the protein particle comprising the diphtheria toxin CRM amino acid sequence is derived from a cell, to thereby treat or prevent the disease, disorder, or condition, in the subject.

In a fourth aspect, the invention provides a method of modulating an immune response in a subject, the method including the step of administering to the subject an effective amount of a protein particle comprising a diphtheria toxin CRM amino acid sequence, wherein the protein particle comprising the diphtheria toxin CRM amino acid sequence is derived from a cell, to thereby modulate the immune response in the subject.

In a fifth aspect, the invention provides a method of delivering to a subject a protein particle comprising a diphtheria toxin CRM amino acid sequence, wherein the protein particle comprising the diphtheria toxin CRM amino acid sequence is derived from a cell, the method including the step of administering to the subject the protein particle comprising a diphtheria toxin CRM amino acid sequence, wherein the protein particle comprising the diphtheria toxin CRM amino acid sequence is derived from a cell, to thereby deliver the protein particle to the subject.

In a sixth aspect, the invention provides a method of detecting a target in a sample, the method including the step of contacting the sample with a protein particle comprising a diphtheria toxin Cross Reacting Material (CRM) amino acid sequence, wherein the protein particle comprising the diphtheria toxin CRM amino acid sequence is derived from a cell, to thereby detect the target in the sample.

In a seventh aspect, the invention provides a composition comprising a protein particle comprising a diphtheria toxin Cross Reacting Material (CRM) amino acid sequence, wherein the protein particle comprising the diphtheria toxin CRM amino acid sequence is derived from a cell, and a pharmaceutically-acceptable diluent, carrier, or excipient.

In an eighth aspect, the invention provides use of a protein particle comprising a diphtheria toxin Cross Reacting Material (CRM) amino acid sequence, wherein the protein particle comprising the diphtheria toxin CRM amino acid sequence is derived from a cell, or a composition according to the seventh aspect, in the manufacture of a medicament to (i) elicit in a subject an immune response to an agent; or (ii) immunise a subject against a disease, disorder, or condition; or (iii) treat or prevent a disease, disorder, or condition in a subject; or (iv) modulate an immune response in a subject; or (v) deliver the protein particle to a subject.

In a ninth aspect, the invention provides a kit comprising a protein particle comprising a diphtheria toxin CRM amino acid sequence, wherein the protein particle comprising the diphtheria toxin CRM amino acid sequence is derived from a cell as herein described.

In some embodiments, the protein particle comprising a diphtheria toxin CRM amino acid sequence wherein the protein particle is derived from a cell, may be formed, substantially formed, assembled, or produced from the diphtheria toxin CRM amino acid sequence. In other embodiments, the protein particle comprising a diphtheria toxin CRM amino acid sequence may be formed or substantially formed from the diphtheria toxin CRM amino acid sequence when the diphtheria toxin CRM amino acid sequence is produced or expressed in the cell.

In some embodiments, the diphtheria toxin CRM amino acid sequence is not derived from a diphtheria toxin CRM protein (or a fragment, variant, or derivative of a diphtheria toxin CRM protein) that has been subjected to a protein refolding treatment. In certain embodiments, the diphtheria toxin CRM protein when the diphtheria toxin CRM amino acid sequence is not derived from a diphtheria toxin CRM protein, or a fragment, variant, or derivative thereof, may be selected from the group consisting of a CRM197 protein, a CRM45 protein, a CRM1001 protein, a CRM228 protein, a CRM176 protein, and a CRM30 protein, or a fragment, variant, or derivative thereof, and any combination thereof. Suitably, the diphtheria toxin CRM protein when the diphtheria toxin CRM amino acid sequence is not derived from a diphtheria toxin CRM protein, or a fragment, variant, or derivative thereof, may be a CRM197 protein, or a fragment, variant, or derivative thereof.

In some embodiments, the protein particle comprising a diphtheria toxin CRM amino acid sequence may be a substantially insoluble protein particle. In some embodiments, the protein particle and/or substantially insoluble protein particle may be derived from an insoluble component of the cell. In some embodiments, the insoluble component of the cell may not have been subjected to a protein refolding treatment. According to some embodiments, the insoluble component may be an inclusion body formed in the cell. In some embodiments, an inclusion body may be an inclusion body formed when the CRM amino acid sequence is expressed or produced in the cell.

In some embodiments, the diphtheria toxin CRM amino acid sequence may comprise, consist of, consist essentially of, or may be, an amino acid sequence derived from, or corresponding to, a CRM protein selected from the group consisting of a CRM197 protein, a CRM45 protein, a CRM1001 protein, a CRM228 protein, a CRM176 protein, and a CRM30 protein, or a fragment, variant, or derivative of any one of the aforementioned CRM proteins, and any combination thereof.

In certain embodiments, the diphtheria toxin CRM amino acid sequence may be derived from, or corresponds to, an amino acid sequence of, or from, a CRM197 protein, or a fragment, variant, or derivative thereof. In certain embodiments, the amino acid sequence of, or from, a CRM197 protein may comprise, consist of, consist essentially of, or is, an amino acid sequence as set forth in any one of SEQ ID NO:2, SEQ ID NO:49, and/or SEQ ID NO:50, or a fragment, variant or derivatives of any one of the aforementioned sequences. In some embodiments, the amino acid sequence of, or from, a CRM197 protein may comprise, consist of, consist essentially of, or is, an amino acid sequence as set forth in SEQ ID NO:50, or a fragment, variant, or derivative thereof.

In further embodiments, the cell may be a host cell suitable for use in recombinant technology. In some embodiments, the cell may be of prokaryotic origin or eukaryotic origin. In some embodiments, the prokaryotic cell may be selected from a Pseudomonas species, an E. coli, a Lactococcus, and/or a Bacillus. In some embodiments, the Pseudomonas may be a Pseudomonas fluorescens. In some embodiments, the Bacillus may be a Bacillus subtilis or a Bacillus megaterium. In some embodiments, the Lactococcus may be a Lactococcus lactis. In other embodiments, the eukaryotic cell may be a yeast cell. Suitably, the yeast cell may be a Saccharomyces or a Pichia. In some embodiments, the Saccharomyces may be a Saccharomyces cerevisiae. In some embodiments, the Pichia may be a Pichia pastoris.

In some embodiments, the protein particle comprising a diphtheria toxin CRM amino acid sequence wherein the protein particle is derived from a cell may be produced by recombinant technology. In certain embodiments, production may be by recombinant DNA technology.

In yet further embodiments, the protein particle comprising a diphtheria toxin CRM amino acid sequence wherein the protein particle is derived from a cell, may further comprise one or more immunogens other than a diphtheria toxin CRM amino acid sequence. In some embodiments, the, or each, immunogen may be derived from a pathogen. In some embodiments, the protein particle may comprise one or a plurality of immunogens of, or from, the same agent, source, or molecule. In some embodiments, the protein particle may comprise one or a plurality of immunogens of, or from, each of a plurality of different agents, sources, or molecules.

In some embodiments, the, or each, immunogen other than a diphtheria toxin CRM amino acid sequence may comprise, consist essentially of, or consist of, or is, an immunogenic amino acid sequence. In certain embodiments, the immunogenic amino acid sequence may be derived from, or corresponds to, at least one of: a pathogen; a protein derived from or of a pathogen; a cancer antigen; an autoantigen; a transplantation antigen; and an allergen (or a fragment, a variant, or a derivative of any one of the aforementioned), and any combination thereof.

In some embodiments, the pathogen may be selected from the group consisting of a virus, a bacterium, a parasite, and a fungus, and any combination thereof.

In certain embodiments, the pathogen may be a virus.

In some embodiments, the immunogenic amino acid sequence is derived from, or corresponds to, a viral protein selected from the group consisting of a capsid protein, an envelope protein, a nucleocapsid protein, a non-structural protein, a structural protein, a fusion protein, and a surface protein, or a fragment, variant, or derivative of any one of the aforementioned viral proteins, and any combination thereof.

In some embodiments, the virus may be selected from the group consisting of a Hepadnaviridae virus, a Flaviviridae virus, a Coronaviridae virus, an influenza virus, and a human immunodeficiency virus (HIV), and any combination thereof.

In some embodiments, the Flaviviridae virus may be a hepatitis C virus (HCV). According to some embodiments that relate to an HCV, an immunogenic amino acid sequence may be derived from, or correspond to, an HCV protein selected from the group consisting of a core protein, a NS3 protein, an E1, and an E2 protein, or a fragment, variant, or derivative thereof, and any combination thereof.

In some embodiments, that relate to an HCV, an immunogenic amino acid sequence may be derived from, or correspond to, an amino acid sequence as set forth in SEQ ID NO:44, or a fragment, variant, or derivative thereof.

In certain embodiments, an HCV core protein immunogenic amino acid sequence may comprise, consist essentially of, consist of, or may be, an amino acid sequence selected from the group consisting of an amino acid sequence as set forth in SEQ ID NO:28 and/or SEQ ID NO:43, or a fragment, variant, or derivative thereof.

In some embodiments, an HCV NS3 protein immunogenic amino acid sequence may comprise, consist essentially of, consist of, or may be, an amino acid sequence as set forth in SEQ ID NO:29 and/or SEQ ID NO:69, or a fragment, variant, or derivative thereof.

In yet other embodiments, an HCV E1 protein immunogenic amino acid sequence may comprise, consist essentially of, consist of, or may be, an amino acid sequence selected from the group consisting of an amino acid sequence as set forth in SEQ ID NO:30, SEQ ID NO:45, and SEQ ID NO:70, or a fragment, variant, or derivative thereof, and any combination thereof.

In some embodiments, an HCV E2 protein immunogenic amino acid sequence may comprise, consist essentially of, consist of, or may be, an amino acid sequence selected from the group consisting of an amino acid sequence as set forth in SEQ ID NO:31, SEQ ID NO:46, SEQ ID NO:71, and SEQ ID NO:104, or a fragment, variant, or derivative thereof, and any combination thereof.

In some embodiments, the immunogenic amino acid sequence derived from, or corresponding to, an HCV protein comprises, consists of, consists essentially of, or is, an amino acid sequence selected from the group consisting of an amino acid sequence as set forth in SEQ ID NO:28, SEQ ID NO:29, SEQ ID NO:30, SEQ ID NO:31, SEQ ID NO:43, SEQ ID NO:44, SEQ ID NO:45, SEQ ID NO:46, SEQ ID NO:69, SEQ ID NO:70, SEQ ID NO:71, and SEQ ID NO:104, or a fragment, variant, or derivative of any one of the aforementioned sequences, and any combination thereof.

In some embodiments, the Flaviviridae virus may be a Dengue virus. In some embodiments, the Dengue virus may be selected from a Dengue virus Type 1, a Dengue virus Type 2, a Dengue virus Type 3, and a Dengue virus Type 4, and any combination thereof. In some embodiments, the immunogenic amino acid sequence is derived from, or corresponds to, a Dengue virus protein that may be selected from an envelope protein, or a fragment, variant, or derivative thereof, and/or a capsid protein, or a fragment, variant, or derivative thereof. In some embodiments, the immunogenic amino acid sequences derived from, or corresponding to, a Dengue virus protein may comprise an amino acid sequence selected from the group consisting of an amino acid sequence as set forth in SEQ ID NO:41, SEQ ID NO:42, SEQ ID NO:47, and SEQ ID NO:48, or a fragment, variant or derivative of any one of the aforementioned sequences, and any combination thereof.

In some embodiments, the Coronaviridae virus may be a coronavirus. In some embodiments, the coronavirus may be a severe acute respiratory syndrome (SARS) coronavirus. In some further embodiments, the SARS coronavirus may be a SARS coronavirus 1 (SARS-CoV-1) and/or a SARS coronavirus 2 (SARS-CoV-2). In certain embodiments, the SARS coronavirus may be a SARS-CoV-2.

In some embodiments, the viral protein of a Coronaviridae virus may be a structural protein, or a fragment, variant, or derivative thereof. In some embodiments, the Coronaviridae virus structural protein may be selected from the group consisting of a spike (S) protein, an envelope (E) protein, a membrane (M) protein, and a nucleocapsid (N) protein, or a fragment, variant, or derivative thereof, and any combination thereof. In further embodiments, the Coronaviridae virus structural protein may be a N protein, or a fragment, variant, or derivative thereof, and/or a S protein, or a fragment, variant, or derivative thereof. In some embodiments, the immunogenic amino acid sequence derived from, or corresponding to, a coronavirus protein may comprise, consist of, consist essentially or, or is, an amino acid sequence selected from the group consisting of an amino acid sequence as set forth in SEQ ID 56; SEQ ID NO:57; SEQ ID NO:58, SEQ ID NO:64, SEQ ID NO:101, SEQ ID NO: 102, and SEQ ID NO:103, or a fragment, variant, or derivative of any one of the aforementioned sequences, and any combination thereof.

In some embodiments, the pathogen may be a parasite. Suitably, the parasite may be a schistosome and/or a malaria parasite. In some further embodiments, the schistosome may be selected from a Schistosoma mansoni, a Schistosoma japonicum, and a Schistosoma haematobium, and any combination thereof. In some embodiments, the malaria parasite may be at least one Plasmodium spp selected from the group consisting of Plasmodium falciparum, Plasmodium vivax, Plasmodium malariae, and Plasmodium ovale, and any combination thereof.

In some embodiments, the pathogen may be a bacterium. In some embodiments, the bacterium may be selected from a Streptococcus species, a Mycobacterium species, and/or a Coxiella species, and any combination thereof.

Suitably, the Streptococcus species may be a Streptococcus pyogenes. In certain embodiments, the immunogenic amino acid sequence derived from, or corresponding to, a Streptococcus pyogenes may be of a virulence factor, a neutrophil inhibitor, a peptidase, and/or a fibronectin-binding protein, or a fragment, variant, or derivative thereof. In some embodiments, the virulence factor may be an M-protein, a fragment, variant, or derivative thereof. In certain embodiments, the M-protein derived immunogenic fragment may comprise, consist of, consist essentially of, or is the amino acid sequence LRRDLDASREAKNQVERALE (SEQ ID NO:17). In other embodiments, the neutrophil factor may be a protease, or a fragment, variant, or derivative thereof. The protease may be an IL-8 protease. In certain embodiments, the IL-8 protease may be a SpyCEP protein, or a fragment, variant, or derivative thereof, and may preferably be a linear B-cell epitope of a SpyCEP protein. In certain embodiments, the SpyCEP protein fragment may comprise, consist of, consist essentially of, or is the amino acid sequence NSDNIKENQFEDFDEDWENF (SEQ ID NO:18). In some embodiments, the peptidase may be a C5a peptidase (ScpA), or a fragment, variant, or derivative thereof.

In some embodiments, a plurality of GAS-derived immunogenic fragments or amino acid sequences derived from the same or different GAS proteins, may be used. In some embodiments, the GAS-derived immunogen may comprise, consist of, consist essentially of, or is, an amino acid sequence as set forth in SEQ ID NO: 17 and/or SEQ ID NO:18, or a fragment, variant, or derivative thereof.

In some embodiments, the Mycobacterium species may be a Mycobacterium tuberculosis, and/or a Mycobacterium bovis. In certain embodiments, the immunogenic amino acid sequences derived from, or corresponding to, a Mycobacterium tuberculosis and/or a Mycobacterium bovis may be derived from, or correspond to, a Mycobacterium protein. In some embodiments, the Mycobacterium protein is an early stage antigen, or a fragment, variant, or derivative thereof. In some embodiments, the early stage antigen may be selected from an Ag85B antigen and/or an TB10.4 antigen, or a fragment, variant, or derivative thereof. In some embodiments, the Mycobacterium protein may be derived from, or correspond to, a latency-associated antigen, or a fragment, variant, or derivative thereof. In some embodiments, the latency-associated antigen may be a rv2660c protein, or a fragment, variant, or derivative thereof. Some further embodiments may include an amino acid sequence derived from, or corresponding to, one or more early stage antigens, optionally in combination with an amino acid sequence from a latency-associated antigen. In some embodiments relating to Mycobacterium, the immunogenic amino acid sequence may comprise an amino acid sequence derived from, or corresponding to, an Ag85B antigen and/or an TB10.4 antigen, optionally may further comprise an amino acid sequence derived from, or corresponding to, a rv2660c protein.

In certain embodiments, the immunogenic amino acid sequence relating to a Mycobacterium and suitably a Mycobacterium tuberculosis and/or a Mycobacterium bovis, and/or a Mycobacterium protein, may comprise, consists essentially of, consists of, or may be, an amino acid sequence selected from the group consisting of an amino acid sequence as set forth in SEQ ID NO:6, SEQ ID NO:7, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40, or a fragment, variant, or derivative of any one of these aforementioned sequences, and any combination thereof.

In some embodiments, the Coxiella species may be a Coxiella burnetti. In some embodiments, an immunogenic amino acid sequence may be derived from, or correspond to, a Coxiella protein selected from the group consisting of a Com1 protein, an OmpH protein, a YbgF protein, a COX protein, and a GroEK protein, or a fragment, variant, or derivative of any one of the aforementioned Coxiella proteins, and any combination thereof.

According to some embodiments that relate to a Coxiella, an immunogenic amino acid sequence may be derived from, or correspond to, comprise, consist of, consist essentially of, or is, an amino acid sequence selected from the group consisting of an amino acid sequence as set forth in SEQ ID NO:59, SEQ ID NO:60, SEQ ID NO:61, SEQ ID NO:62, SEQ ID NO:63, SEQ ID NO:72, SEQ ID NO:73, and SEQ ID NOs:74-100, or a fragment, variant, or derivative of any one of the aforementioned sequences, and any combination thereof.

In certain embodiments, a diphtheria toxin CRM amino acid sequence and an immunogenic amino acid sequence derived from, or corresponding to, one or more immunogens other than the diphtheria toxin amino acid sequence may be a chimera or a chimeric molecule. In some embodiments, the chimera or chimeric molecule may form, produce, code for, or correspond to, a chimeric protein. Suitably, a chimera, chimeric molecule, or chimeric protein is produced, generated, formed, or expressed in a recombinant expression system.

In yet further embodiments, the protein particle comprising a diphtheria toxin CRM amino acid sequence as herein described may be substantially formed from, or from expression of, a chimeric protein as herein described.

In some embodiments, the protein particle comprising a diphtheria toxin CRM197 amino acid sequence, wherein the protein particle is derived from a cell, may be formed by self-assembly and in some embodiments, may be formed from self-assembly in a cell. In some embodiments, the cell may be derived from, or suitable for, recombinant protein expression.

In some embodiments, the protein particle comprising a diphtheria toxin CRM197 amino acid sequence, wherein the protein particle is derived from a cell, may form, be produced, assemble, or aggregate into a suitable particle when expressed in a suitable host microorganism as herein described.

In certain embodiments of any one of the aforementioned aspects, the agent or the disease, disorder, or condition, may be associated with a cancer and/or may be caused by a pathogen. Accordingly, the pathogen may be selected from the group consisting of a virus, a bacterium, a parasite, and a fungus, and combinations thereof. Suitably, the cancer may be selected from the group consisting of a prostate cancer, a breast cancer, a liver cancer, a colorectal cancer, a renal cancer, and a melanoma.

In some embodiments, the composition may be a pharmaceutical composition. In some embodiments, the composition or pharmaceutical composition may be an immunogenic composition. In certain embodiments, the immunogenic composition may be an immunotherapeutic composition. In further embodiments, the immunogenic composition and/or immunotherapeutic composition may be a vaccine. The composition as described may be suitable for administration to a subject. The composition may be suitable for administration to a subject. In some embodiments, the composition may further comprise an adjuvant. In some embodiments, the adjuvant may be an alum and/or dimethyl dioctadecyl ammonium bromide.

It is contemplated that in some embodiments, the composition of the seventh aspect may be for use according to a method of any one of the aforementioned aspects.

In some embodiments, a method of the sixth aspect may be a method to detect an immune response, or one or more elements of an immune response. Accordingly, in some embodiments, the immune response may be against, or associated with, one or more of an agent, a pathogen, a protein of or from a pathogen, a cancer antigen, an autoantigen, a transplantation antigen, and an allergen, or a fragment, variant, or derivative of any one of the aforementioned, and any combination thereof. In some embodiments, the pathogen may be selected from a virus, a bacterium, a parasite, and a fungus, and any combination thereof. According to some methods of the sixth aspect, a protein particle as described herein may further comprise an amino acid sequence for suitable for use in detection or diagnosis of a pathogen.

In some embodiments of the sixth aspect, the method may detect a Mycobacterium infection and/or a Mycobacterium specific immune response in a sample. In some embodiments, the Mycobacterium may be a Mycobacterium tuberculosis and/or a Mycobacterium bovis. According to some embodiments relating to Mycobacterium, the sample may be a skin portion and/or a blood sample. In some embodiments that relate to detection of a Mycobacterium infection and/or a Mycobacterium specific immune response, the protein particle may further comprise an amino acid sequence as set forth in any one of SEQ ID NOS:32 to 40, and any combination thereof.

In some embodiments of the sixth aspect, the sample may be derived from sputum, blood, skin, an epithelial tissue, an intranasal tissue or cell, an oropharyngeal tissue or cell, or a component thereof. In some embodiments, the sample may be derived from a subject.

In some embodiments, wherein the method of the sixth aspect may be performed in vitro.

In other aspects, the invention provides a kit comprising a protein particle comprising a diphtheria toxin CRM amino acid sequence, wherein the protein particle comprising the diphtheria toxin CRM amino acid sequence is derived from a cell as herein described. The kit may be used in any one of the methods of the present invention, particularly as set out in any one of the first to sixth aspects, suitably in the sixth aspect. In some embodiments, a kit may comprise a composition comprising a protein particle as herein described. In some embodiments, the kit may detect an immune response, or one or more elements of an immune response. The immune response may be to, against or in response to an agent, or component thereof (e.g, a pathogen) as described herein. In some embodiments, the kit may be an immunodiagnostic kit.

In some embodiments of any one of the aforementioned aspects, the subject may be a mammal. Preferably, the mammal may be a human.

In certain embodiments, a method, composition, use, or kit of any one of aforementioned aspects may elicit, is, detect, or comprise, a protective immune response.

In some embodiments, the agent or the disease, disorder, or condition may be associated with a cancer and/or may be caused by a pathogen. In some embodiments, the pathogen may be selected from the group consisting of a virus, a bacterium, a parasite, and a fungus, and any combination thereof. In some embodiments, the cancer may be selected from the group consisting of a prostate cancer, a breast cancer, a liver cancer, a colorectal cancer, a renal cancer, and a melanoma, and any combination thereof.

In related aspects, the invention provides an isolated protein comprising a diphtheria toxin CRM amino acid sequence and an immunogenic amino acid sequence derived from a Mycobacterium species. The invention further provides an isolated nucleic acid encoding said isolated protein comprising a diphtheria toxin CRM amino acid sequence and an immunogenic amino acid sequence derived from a Mycobacterium species, a genetic construct comprising said isolated nucleic acid and a host cell comprising said genetic construct. In other related broad aspects, the invention provides a protein particle derived from or comprising said isolated protein, and pharmaceutical compositions comprising said isolated protein and/or protein particles. Therapeutic methods and methods of eliciting an immune response, or immunising a subject are also provided.

The articles “a” and “an” are used herein to refer to one or to more than one (i.e., to at least one) of the grammatical object of the article and should not be taken as meaning or defining “one” or a “single” element or feature. By way of example, “an element” means one element or more than one element. As used herein, the use of the singular includes the plural (and vice versa) unless specifically stated otherwise.

Throughout this specification, unless otherwise indicated, “comprise”, “comprises”, and “comprising”, (and variants thereof) or related terms such as “includes”, “including”, (and variants thereof), are used inclusively rather than exclusively, so that a stated integer or group of integers may include one or more other non-stated integers or groups of integers.

By “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. In some embodiments, the phrase “consisting essentially of” in the context of a recited subunit sequence (e.g., amino acid sequence or nucleic acid sequence) indicates that the sequence may comprise at least one additional upstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits; e.g., amino acids or nucleotides) and/or at least one additional downstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits; e.g., amino acids or nucleotides), wherein the number of upstream subunits and the number of downstream subunits are independently selectable.

The term “and/or”, e.g., “A and/or B” shall be understood to mean either “A and B” or “A or B” and shall be taken to provide explicit support for both meanings or for either meaning.

### BRIEF DESCRIPTION OF THE DRAWINGS

By way of example only, embodiments of the invention are described more fully hereinafter with reference to the accompanying drawings, wherein:

FIG. 1: Plasmid construction for formation of CRM197 only particles and CRM197 particles displaying the tuberculosis H4 or H28 antigens. The CRM197 gene fragment was isolated from pUC57-CRM197 by DNA digestion using NdeI. BamHI restriction site was introduced to the 3′ end of CRM197 using PCR. The resulting CRM197 was ligated to the pET-14b vector, generated by restriction enzyme digestion with NdeI and BamHI, using T4 DNA ligase to form the final plasmid pET-14b CRM197. Gene fragment H4 or H28, prepared from the plasmid pUC57 H4 or pUC57 H28 using BamHI enzyme digestion, was ligated to linearized pET-14b CRM197, digested with BamHI, to generate the final plasmids, pET-14b CRM197-H4 and pET-14b CRM197-H28.

FIG. 2: Solubility analysis of CRM197 produced in ClearColi cell. (A) Protein profile of whole cell lysate containing CRM197 was analyzed with 10% Bis-Tris Gel. (B) The protein profile of supernatant fraction of crude cell lysate without 8 M urea treatment was analyzed after sonication and centrifugation. (C) The protein profile of supernatant fraction of crude cell lysate treated with 8 M urea was analyzed after sonication and centrifugation. Lane 1, molecular weight marker (Mark 12; Invitrogen); lane 2, CRM197 (58.544 kDa).

FIG. 3: Protein profile of CRM197 particles purified by (A) 0.5× lysis buffer, (B) by 0.5× lysis buffer and wash buffer containing 2 M urea, and (C) by 0.5× lysis buffer and wash buffer containing 2 M urea and 5% Triton X-100 after cell disruption using microfluidizer. Lane 1, molecular weight marker (Mark 12; Invitrogen); lane 2, CRM197 (58.544 kDa).

FIG. 4: Analysis of purified CRM197 protein particles and soluble antigen controls using 10% Bis-Tris gel. (A) Protein profile of purified CRM197 particles. Lane 1, molecular weight marker (Mark 12; Invitrogen); lane 2, CRM197 (58.544 kDa); lane 3, CRM197-H4 (99.705 kDa); lane 4, CRM197-H28 (107.269 kDa). (B) Analysis of soluble His6-tagged H4 and H28 mycobacterial peptides. Lane 1, molecular weight marker (Mark 12; Invitrogen); lane 2, His6-H4 (41.988 kDa); lane 3, His6-H28 (49.553 kDa).

FIG. 5: Scanning electron microscopy (SEM) images of ClearColi BL21 (DE3) cells harbouring various plasmids (A-C) and of purified CRM197 protein particles displaying immunogenic TB fragments (A1-C1). (A and A1), pET-14b CRM197; (B and B1), pET-14b CRM197-H4; (C and C1), pET-14b CRM197-H28.

FIG. 6: Transmission electron microscopy (TEM) images of Escherichia coli (ClearColi strain) cells harboring various plasmids (A-C) and of purified CRM197 protein particles displaying H4 and/or H28 immunogens (A1-C1). (A and A1), pET-14b CRM197; (B and B1), pET-14b CRM197-H4; (C and C1), pET-14b CRM197-H28.

FIG. 7: Zeta potential of CRM197 particle samples before and after emulsification in DDA. The Zeta potential of each sample was measured three times by Zetasizer Nano ZS. Each data point stands for the mean+the standard error of the mean.

FIG. 8: Particle size of CRM197 particle samples before and after emulsification in DDA. Particle samples were treated with sonication before the size distribution measurement. Particle size was consecutively measured three times by Mastersizer 3000 and the standard deviation was less than 0.01.

FIG. 9: Measurement of CRM197 particle samples (A) and soluble His6-tagged H4 and H28 antigen concentrations (B) using densitometry analysis of protein profile on Bis-Tris Gel. Different amounts (50 ng, 100 ng, 300 ng, and 500 ng) of BSA standard were loaded on Bis-Tris gel to generate a standard curve, used to determine the antigen concentrations. The image was taken by the Gel Doc system (BioRad Laboratories, Hercules, Calif.), and analyzed with the image Lab software (BioRad Laboratories, Hercules, Calif.).

FIG. 10: Specific recognition of CRM197 protein displaying tuberculosis (TB) antigens by pooled sera from mice immunized with various TB antigens. (A) Protein profile of ClearColi cells producing CRM197 particle displaying H4/H28 antigens, purified CRM197 particle displaying H4/H28 antigens, and soluble H4/H28 antigens. (B) Immunogenicity analysis of CRM197 particle platform by using pooled sera of mice immunized with purified CRM197 particles (C particles). The amount of protein loaded in FIG. 10B is 50 times less than the protein loaded on SDS-PAGE shown in FIG. 10A. (C) Western blot analysis of various antigens using pooled sera from mice immunized with CRM197-H4 particles (C—H4 particles). The amount of protein loaded in FIG. 10C is 10-50 times less than the protein loaded on SDS-PAGE shown in FIG. 10A. (D) Western blot analysis of various samples using pooled sera from mice immunized with CRM197-H28 particles (C—H28 particles). The amount of protein loaded in FIG. 10E is 50 times less than the protein loaded on SDS-PAGE shown in FIG. 10A. (E) Western blot analysis of various antigens using pooled sera from mice immunized with H4. The amount of protein loaded in FIG. 10E is 50 times less than the protein loaded on SDS-PAGE shown in FIG. 10A. (F) Western blot analysis of various antigens using pooled sera from mice immunized with H28. The amount of protein loaded in FIG. 10F is 50 times less than the protein loaded on SDS-PAGE shown in FIG. 10A. Lane 1, molecular weight marker (GangNam-Stain prestained protein ladder; iNtRon); lane 2, ClearColi (DE3)/pET-14b; lane 3, ClearColi (DE3)/pET-14b CRM197, 58.544 kDa; lane 4, ClearColi (DE3)/pET-14b CRM197-H4, 99.705 kDa; lane 5, ClearColi (DE3)/pET-14b CRM197-H28, 107.269 kDa; lane 6, CRM197 (58.544 kDa); lane 7, CRM197-H4 (99.705 kDa); lane 8, CRM197-H28 (107.269 kDa); lane 9, soluble His6-H4 (41.988 kDa); lane 10, soluble His6-H28 (49.553 kDa).

FIG. 11: Antibody response in mice immunized with different antigens presented as the EC50 in response to soluble His6-H4 (A) and soluble His6-H28 (B). Levels of specific antibodies of the IgG1 and the IgG2c isotype were measured by ELISA. Each data point represents results from six mice ± the standard error of the mean (Minitab 17).

FIG. 12: Cytokine release by murine splenocytes following 24 hours (h) the stimulation with soluble His6-H4 and soluble His6-H28. Three weeks after final inoculations, splenocytes were cultured for 24 h with soluble His6-H4 and soluble His6-H28. Release of cytokines was measured by cytometric bead array. Each data point represents the mean for 6 mice ± the standard error of the mean (Minitab 17).

FIG. 13: Cytokine release by murine splenocytes following 60 h the stimulation with soluble His6-H4 and soluble His6-H28. Three weeks after final inoculations, splenocytes were cultured for 24 h with soluble His6-H4 and soluble His6-H28. Release of cytokines was measured by cytometric bead array. Each data point represents the mean for 6 mice ± the standard error of the mean (Minitab 17).

FIG. 14: TEM images of ClearColi, SHuffle, and Origami cells harbouring pET-14b CRM197 (A-C, scale bar=500 nm) and of purified CRM197 protein particles (A1-C1, scale bar=200 nm).

FIG. 15: Alignment of CRM protein amino acid sequences and diphtheria toxin. FIG. 15A is the alignment of the sequences with a signal peptide of CRM; the boxed sequences in FIG. 15A delineates the signal peptide sequence. FIG. 15B is the alignment of the sequences without the signal peptide. In FIG. 15A, the sequences are identified as follows: a CRM197 protein (SEQ ID NO:49); a CRM228 protein (SEQ ID NO:23); a CRM176 protein (SEQ ID NO:24); a CRM1001 protein (SEQ ID NO:25); a CRM45 protein (SEQ ID NO:26); and a diphtheria toxin protein derived from Corynebacterium diphtheriae (SEQ ID NO:27). In FIG. 15B, the sequences are identified as follows: a CRM197 protein (SEQ ID NO:50); a CRM228 protein (SEQ ID NO:51); a CRM176 protein (SEQ ID NO:52); a CRM1001 protein (SEQ ID NO:53); a CRM45 protein (SEQ ID NO:54); and a diphtheria toxin protein derived from Corynebacterium diphtheriae (SEQ ID NO:55).

FIG. 16: Effect of DDA adjuvant on immune responses induced by CRM197-TB particle. a T cell proliferation in response to H4 or C—H4 (CRM197 protein fused to H4) at various concentrations ranging from 0.1-100 μg/mL. b IFNγ ELISpot assay of splenocytes from mice tested with and/or without DDA adjuvant in response to H4 stimulation. c, d, e, f, g, h CD4+ T cell intracellular cytokine staining (ICS) assays. CD4+ T cells from various inoculated mice were stimulated with H4. CD4+ T cells producing intracellular cytokines (IFNγ, IL-2, IL-17, and TNF) were detected by ICS and flow cytometry. i, j, k, 1, m, n CD8+ T cell intracellular cytokine staining assay in response to H4 stimulation. CD8+ T cells producing intracellular cytokines (IFNγ, IL-2, IL-17, and TNF) were detected by ICS and flow cytometry. o Multiple intracellular cytokine staining (ICS) assays of CD4+ T cells. CD4+ T cells producing multiple intracellular cytokines (IFNγ, IL-2, and TNF) were detected by ICS and flow cytometry. Each data point stands for the results from 4 mice ±SEM. Statistical significance was calculated by 1-way ANOVA, with pairwise comparison of multi-grouped data sets achieved using Tukey's or Dunnet's post hoc test (Prism). C-WT=CRM197 particle; C-WT/DDA=CRM197 particle emulsified in DDA adjuvant; C—H4=CRM197-H4 particle; C—H4/DDA=CRM197-H4 particle emulsified in DDA adjuvant; BCG=live, attenuated Mycobacterium bovis known as Bacille Calmette-Guérin.

FIG. 17: IFNγ ELISpot assay of splenocytes from mice injected with various CRM197-TB antigen particles in response to H4, H28, CFP, ConA, or media stimulation. Each data point stands for the results from 4 mice ±SEM. Statistical significance was calculated by 1-way ANOVA, with pairwise comparison of multi-grouped data sets achieved using Tukey's (Prism). C-WT=CRM197 particle; C—H4=CRM197-H4 particle; C—H28=CRM197-H28 particle; CFP=culture filtrate proteins from Mycobacterium tuberculosis; BCG=live, attenuated Mycobacterium bovis known as Bacille Calmette-Guérin.

FIG. 18: CD4+ and CD8+ T cells intracellular cytokine staining (ICS) assays upon H4 stimulation. a, b, c, d, e, f CD4+ T cells from various inoculated mice were stimulated with H4. CD4+ T cells producing intracellular cytokines (IFNγ, IL-2, IL-17, and TNF) were detected by ICS and flow cytometry. g, h, i, j, k, 1 CD8+ T cells producing intracellular cytokines (IFNγ, IL-2, IL-17, and TNF) were detected by ICS and flow cytometry. Each data point stands for the results from 4 mice ±SEM. Statistical significance was calculated by 1-way ANOVA, with pairwise comparison of multi-grouped data sets achieved using Tukey's or Dunnet's post hoc test (Prism). CFP—culture filtrate proteins from Mycobacterium tuberculosis; C-WT=CRM197 particle; C—H4=CRM197-H4 particle; C—H28=CRM197-H28 particle; BCG=live, attenuated Mycobacterium bovis known as Bacille Calmette-Guérin.

FIG. 19: CD4+ and CD8+ T cells intracellular cytokine staining (ICS) assays upon H28 stimulation. a, b, c, d, e, f CD4+ T cells from various test mice were stimulated with H28. CD4+ T cells producing intracellular cytokines (IFNγ, IL-2, IL-17, and TNF) were detected by ICS and flow cytometry. g, h, i, j, k, 1 CD8+ T cells producing intracellular cytokines (IFNγ, IL-2, IL-17, and TNF) were detected by ICS and flow cytometry. Each data point stands for the results from 4 mice ±SEM. Statistical significance was calculated by 1-way ANOVA, with pairwise comparison of multi-grouped data sets achieved using Tukey's or Dunnet's post hoc test (Prism). CFP=culture filtrate proteins from Mycobacterium tuberculosis; C-WT=CRM197 particle; C—H4=CRM197-H4 particle; C—H28=CRM197-H28 particle; BCG=live, attenuated Mycobacterium bovis known as Bacille Calmette-Guérin.

FIG. 20: CD4+ and CD8+ T cells intracellular cytokine staining (ICS) assays upon TB10.4 stimulation. a, b, c, d, e, f CD4+ T cells from various inoculated mice were stimulated with TB10.4. CD4+ T cells producing intracellular cytokines (IFNγ, IL-2, IL-17, and TNF) were detected by ICS and flow cytometry. g, h, i, j, k, 1 CD8+ T cells producing intracellular cytokines (IFNγ, IL-2, IL-17, and TNF) were detected by ICS and flow cytometry. Each data point stands for the results from 4 mice ±SEM. Statistical significance was calculated by 1-way ANOVA, with pairwise comparison of multi-grouped data sets achieved using Tukey's or Dunnet's post hoc test (Prism). CFP=culture filtrate proteins from Mycobacterium tuberculosis; C-WT=CRM197 particle; C—H4=CRM197-H4 particle; C—H28=CRM197-H28 particle; BCG=live, attenuated Mycobacterium bovis known as Bacille Calmette-Guérin.

FIG. 21: Analysis of antibody responses using ELISA. IgG1 and IgG2c in response to different test samples were analyzed by ELISA. Each data point stands for the results from 4 mice ±SEM. Statistical significance was calculated by 1-way ANOVA, with pairwise comparison of multi-grouped data sets achieved using Tukey's (Prism). CFP=culture filtrate proteins from Mycobacterium tuberculosis; C-WT=CRM197 particle; C—H4=CRM197-H4 particle; C—H28=CRM197-H28 particle; BCG=live, attenuated Mycobacterium bovis known as Bacille Calmette-Guérin.

FIG. 22: Lung (a) and spleen CFU (b) of mice administered with DDA adjuvant, BCG, CFP, and adjuvanted soluble or particulate TB test samples produced in ClearColi BL21 (DE3). Mice were infected with M. tuberculosis H37Rv six weeks after the final administration with the test sample. Infection with M. tuberculosis was via the aerosol route using a Middlebrook airborne infection apparatus (Glas-Col) with an infective dose of approximately 100 viable bacilli. Postmortem was followed four weeks after aerosol M. tuberculosis infection. Each data point stands for the results from 8 mice ±SEM. Statistical significance was calculated by 1-way ANOVA, with pairwise comparison of multi-grouped data sets achieved using Tukey's or Dunnet's post hoc test (Prism). Asterisk indicates significantly different from BCG, CFP, C-WT, H4, C—H4, and H28 test group. “ns” refers to “not significant”. CFP=culture filtrate proteins from Mycobacterium tuberculosis, C-WT=CRM197 particle; C—H4=CRM197-H4 particle; C—H28=CRM197-H28 particle; BCG=live, attenuated Mycobacterium bovis known as Bacille Calmette-Guérin.

FIG. 23: Plasmid construction of (A) pET14b_CRM-P*17, (B) pET14b_CRM-S2 and (C) pET14b_CRM-P*17-S2 for particle production in E. coli strain ClearColi™ BL21 (DE3). The DNA fragments encoding the P*17/S2/P*17-S2 genes were isolated from pUC57_P*17/pUC57_S2/pUC57_P*17-S2 by DNA hydrolysis with XhoI and BamHI. The resulting individual genes were ligated into the linearized pET14b_CRM vector (generated by XhoI and BamHI enzyme digestion) using T4 DNA ligase to generate the final plasmids: pET14b_CRM-P*17/pET14b_CRM-S2/pET14b_CRM-P*17-S2. Reference to CRM is reference to CRM197. CRM=CRM197; CRM-P*17=CRM197-P*17; CRM-S2=CRM197-S2; CRM-P*17-S2=CRM197-P*17-S2.

FIG. 24: A schematic representation of hybrid genes encoding fusion proteins to produce CRM, CRM-P*17, CRM-S2 and CRM-P*17-S2 particles in recombinant E. coli ClearColi BL21™ (DE3) strain. CRM=CRM197; CRM-P*17=CRM197-P*17; CRM-S2=CRM197-S2; CRM-P*17-S2=CRM197-P*17-S2.

FIG. 25: Protein profile analysis of the whole-cell lysate and the isolated CRM197 (‘CRM’) containing particles separated by SDS-PAGE and gel stained with Coomassie Blue. CRM (58.5 kDa), CRM-P*17 (66.7 kDa), CRM-S2 (66.7 kDa) and CRM-P*17-S2 (74.9 kDa) fusion proteins were isolated from E. coli strain ClearColi™ BL21 (DE3) containing the respective plasmids. Fusion proteins were confirmed by mass spectrometry. CRM=CRM197; CRM-P*17=CRM197-P*17; CRM-S2=CRM197-S2; CRM-P*17-S2=CRM197-P*17-S2.

FIG. 26: Particle size and Zeta potential of the formulated CRM197 (‘CRM’) containing particles. (A) Size of CRM particle before and after mixture with Alum adjuvant. (B) Zeta potential of various CRM particles before and after mixture with Alum adjuvant. The particle size and zeta potential of each CRM particle preparation was measured three times using Zetasizer Nano ZS. Each data point of measurement represents the mean± the standard error of the mean. CRM particles=CRM197 particles; CRM-P*17 particles=CRM197-P*17 particles; CRM-S2 particles=CRM197-S2 particles; CRM-P*17-S2 particles=CRM197-P*17-S2 particles.

FIG. 27: Experimental plan of StrepA particles study in mice. (1) Various StrepA particles, CRM197 particles, CRM197-P*17 particles, CRM197-S2 particles, and CRM197-P*17-S2 particles were extracted from an endotoxin free E. coli strain ClearColi™ BL21 (DE3). (2) Mice were administered with sterilized CRM197 particle preparations with Alum for immunogenicity study for antibody analysis. (3) Two weeks later after the final immunization with CRM197 particles carrying StrepA antigens emulsified in Alum adjuvant, mice were infected intranasally with Streptococcus pyogenes with an infected dose of approximately 5×10⁸ cfu/ml in the volume of 10 μL. Primary immunization (PI). Submandibular bleeding (SB). Intramuscular (IM). CRM particles=CRM197 particles; CRM-P*17 particles=CRM197-P*17 particles; CRM-S2 particles=CRM197-S2 particles; CRM-P*17-S2 particles=CRM197-P*17-S2 particles.

FIG. 28: Antigen specific antibody response in mice to vaccination with CRM197-containing particle preparations. (A) Total IgG titers in response to P*17 and K4S2 soluble proteins analyzed by ELISA for each group. (B) IgG subtypes, IgG1, IgG2a, IgG2b and IgG3 titers. The sera analysis was done 42 days after PI. Each data point represents results from 5 mice ± the standard error of the mean. Statistical analysis was done by one-way ANOVA with statistical significance (p<0.05) indicated by letter-based representation of pairwise comparisons between groups using Tukey's post-hoc test. CRM particles=CRM197 particles; CRM-P*17 particles=CRM197-P*17 particles; CRM-S2 particles=CRM197-S2 particles; CRM-P*17-S2 particles=CRM197-P*17-S2 particles.

FIG. 29: Antigen specific recognition of induced antibodies assessed by Western blot analysis using the pooled sera from mice administered with CRM197-containing particles. Particles used for mice in this study are indicated above the blot, Alum as negative control and P*17-DT+K4S2-DT as positive control. The corresponding SDS-PAGE is shown on the left. CRM particles=CRM197 particles; CRM-P*17 particles=CRM197-P*17 particles; CRM-S2 particles=CRM197-S2 particles; CRM-P*17-S2 particles=CRM197-P*17-S2 particles.

FIG. 30: Three time points of antigen specific antibody response to mice injected with CRM197-containing particles. Total IgG titers in response to (A) P*17 and (B) K4S2 soluble proteins analyzed by ELISA for each group. First bleed was done 20 days after primary immunization (PI); second bleed was done 27 days after PI; and third bleed was done 35 days after PI. Each data point represents results from 5 mice ± the standard error of the mean. Statistical analysis was done by one-way ANOVA with statistical significance (p<0.05) indicated by letter-based representation of pairwise comparisons between groups using Tukey's post-hoc test. CRM particles=CRM197 particles; CRM-P*17 particles=CRM197-P*17 particles; CRM-S2 particles=CRM197-S2 particles; CRM-P*17-S2 particles=CRM197-P*17-S2 particles.

FIG. 31: Schematic overview of recombinant gene encoding fusion proteins for production of CRM197 particles comprising HCV antigens.

FIG. 32: Protein profile of purified CRM197-HCV antigen particles. Lane 1, molecular weight marker (GangNam-Stain prestained protein ladder; iNtRon); lane 2, CRM197, 58.5 kDa; lane 3, CRM197-chimeric protein, 109.3 kDa; lane 4, CRM197-E1-E2-NS3, 118.89 kDa; lane 5, CRM197-HepC, 80.04 kDa.

FIG. 33: Schematic overview of recombinant gene encoding fusion proteins for production of CRM197 particles incorporating TB diagnostic antigens.

FIG. 34: Solubility analysis of CRM197 TB diagnostic reagents. Protein profile of ClearColi BL21(DE3) cells harbouring (A) pET-14b CRM197, 58.5 kDa, (B) pET-14b CRM197-TB7.7-ESAT6-CFP10, 87.6 kDa, (C) pET-14b CRM197-HspX-ESAT6-CFP10, 96.4 kDa, or (D) pET-14b CRM197-TB7.7-HspX-ESAT6-CFP10, 104.1 kDa. kDa, molecular weight marker (GangNam-Stain prestained protein ladder; iNtRon); lane 1, ClearColi BL21(DE3) cells producing CRM197 TB diagnostic reagents; lane 2, supernatant fraction of the cell suspension without 8 M urea treatment after sonication and centrifugation; land 3, supernatant fraction of the cell suspension with 8 M urea treatment after sonication and centrifugation.

FIG. 35: Protein profile of purified CRM197 particle-based TB diagnostic reagents. kDa, molecular weight marker (GangNam-Stain prestained protein ladder; iNtRon); lane 1, CRM197, 58.5 kDa; lane 2, CRM197-TB7.7-ESAT6-CFP10, 87.6 kDa; lane 3, CRM197-HspX-ESAT6-CFP10, 96.4 kDa; lane 4, CRM197-TB7.7-HspX-ESAT6-CFP10, 104.1 kDa.

FIG. 36: Immunogenicity analysis of particulate CRM197-SARS-CoV-2 antigen particle produced from ClearColi BL21(DE3). a Schematic overview of hybrid genes encoding fusion proteins for production of CRM197-SARS-CoV-2 antigen particle (particulate CRM197-RBD and particulate CRM197-N protein). b Protein profile of various purified CRM197-SARS-CoV-2 antigen particles. kDa, molecular weight marker (GangNam-Stain prestained protein ladder; iNtRon); lane 1, CRM197, 58.5 kDa; lane 2, CRM197-RBD, 82.2 kDa; lane 3, CRM197-N protein, 104.6 kDa. c,d Antibody response of mice tested with various CRM197-SARS-CoV-2 antigen particle 1 week after the first boost. e,f Antibody response of mice tested with various CRM197-SARS-CoV-2 antigen particle 2 weeks after the second boost. CRM particles=CRM197 particles; CRM197-N pro particles=CRM197-N protein particles; CRM-RBD particles=CRM197-RBD particles.

FIG. 37: Determination of SARS-CoV-2 antigen functional conformation when incorporated into CRM197 particles. The particles were produced in ClearColi BL21(DE3) harbouring pMCS69E and structural conformation of S1 or RBD in CRM197 particles was assessed by analysing ACE2 binding. High-binding plates (Greiner Bio-One, Germany) were coated overnight at 4° C. with 100 μL of 5 μg mL⁻¹ purified CRM197-SARS-CoV-2 antigen particles diluted in phosphate-buffered saline containing 0.05% (v/v) Tween 20, pH7.5 (PBST). CRM197 particles and CRM197-N protein particles are negative controls. Glycosylated soluble S1 (University of Queensland, Australia) is used as a positive control. Plate was incubated with Angiotensin-Converting Enzyme (ACE2)(Human) Fc fusion (HEK293) (Aviscera Bioscience Inc, USA) diluted with PBST at the concentration of 1/1000 for 1 h at 25° C. After three times wash with PBST, plate was incubated with protein A-HRP for 1 h at 25° C. o-phenylenediamine substrate (Abbott Diagnostics, IL, USA) was added on plate for signal development. The result was measured at 490 nm with on an ELx808iu ultramicrotiter plate reader (Bio-Tek Instruments Inc., USA). b Evaluation of CRM197-SARS-CoV-2 antigen particle performance by diagnosing infected human serum samples using ELISA. This experiment is done as a single blind study. H, CRM197 particles; I, CRM197-RBD particles; J, CRM197-N protein particles; K, CRM197-S1 particles. S1-RBD is a positive control. Briefly, high-binding plates were coated with 100 μL of 1 g mL-1 of antigens in carbonate coating buffer pH9.6 at 4° C. overnight. Plates were blocked with 5% skim milk in PBST for 90 mins at 37° C. before adding the primary antibody (infected and noninfected human plasma samples) at the concentration of 1/2,000 for 90 mins at 37° C. After washings, plates were then incubated with the secondary IgG at the concentration of 1/3,000 and OPD was used as the substrate for signal development. The results were measured at 492 nm.

FIG. 38: Immunogenicity analysis of CRM197-SARS-CoV-2 antigen particles produced from ClearColi BL21(DE3) harbouring pMCS69. A: Schematic overview of hybrid genes encoding fusion proteins for production of CRM197 particles incorporating SARS-Co-V-2 antigen particles. a.1: Protein schematic structure mediating the production of CRM197 particles carrying SARS-Co-V-2 antigens. B: Protein profile of purified CRM197-SARS-CoV-2 antigen particles. kDa, molecular weight marker (GangNam-Stain prestained protein ladder; iNtRon); lane 1, CRM197, 58.5 kDa; lane 2, CRM197-N protein, 104.6 kDa; lane 3, CRM197-S1, 136.9 kDa. c,d Antibody response of mice inoculated with various CRM197-SARS-CoV-2 antigen particles 1 week after the first boost. CRM197-N pro=CRM197-N protein particles; CRM-S1=CRM197-S1 particles.

FIG. 39: Protein profile of CRM197 particle-based Q fever particles. Lane 1: Purified CRM197-COX particles (101.1 kDa); Lane 2: Purified wild type CRM197 (58.5 kDa); Lane 3: Whole cell CRM197-COX particles; Lane 4: Whole cell wild type CRM197.

FIG. 40: Protein profile of CRM197 based Q fever diagnostic reagents. Lane 1: Whole cell CRM197-COM1 (86.4 kDa); Lane 2: Whole cell CRM97-GroEL (83.1 kDa); Lane 3: Whole cell CRM197-OmpH (81.5 kDa); Lane 4: Whole cell CRM197-YbgF (93.0 kDa); Lane 5: Purified CRM197-COM1 particle; Lane 6: Purified CRM197-GroEL particle; Lane 7: Purified CRM197-OmpH particle; Lane 8: Purified CRM197-YbgF particle.

FIG. 41: Induction of neutralizing antibodies using CRM197-SARS-CoV-2 antigen particle formulations. Pooled sera were analyzed using the SARS-CoV-2 plaque reduction assay. CRM=CRM197 particles; CRM-N=CRM197-N protein particles; CRM-RBD=CRM197-RBD particles.

FIG. 42: Schematic diagram of full-length SARS-CoV-2 S protein. S1, receptor-binding subunit; RBD, receptor-binding domain; S2, membrane fusion subunit.

FIG. 43: Antibody responses of mice tested with various CRM197-SARS-CoV-2 antigen particles 2 weeks after the second boost. CRM197-SARS-CoV-2 antigen particles are CRM197 particles carrying S1 and CRM197 particles carrying N protein. The antigen particles were administered in the 3 formulations, alum adjuvant only without CRM197-SARS-CoV-2 antigen particle, two separate CRM197 particles carrying each antigen mixed and formulated in alum, and CRM197 particle carrying S1 formulated in alum. CRM-N pro=CRM197-N protein particles; CRM-S1=CRM197-S1 particles.

Some figures may contain colour representations or entities. Colour illustrations are available from the Applicant upon request or from an appropriate Patent Office. A fee may be imposed if obtained from the Patent Office.

### BRIEF DESCRIPTION OF THE SEQUENCES

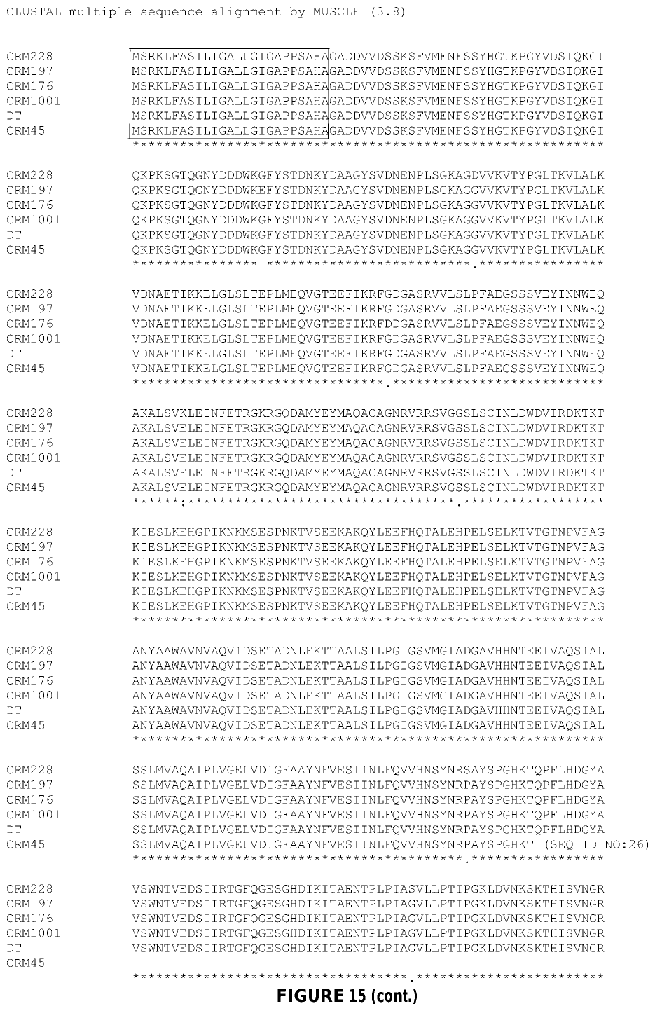

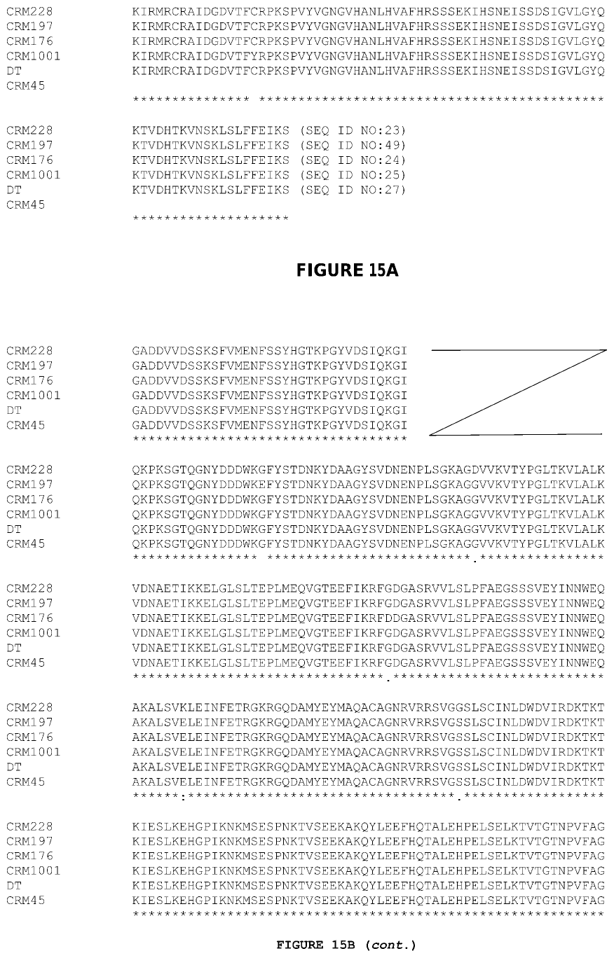

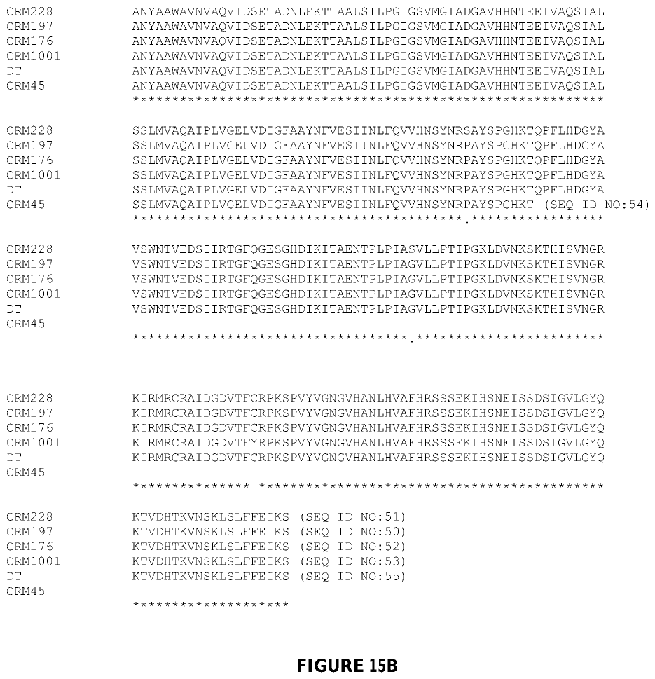

SEQ ID NO:1 A nucleic acid sequence of a CRM197 coding sequence
SEQ ID NO:2 An amino acid sequence of a CRM197 protein as set out in Table 2
SEQ ID NO:3 CRM197_NdeI_Fwd oligonucleotide sequence
SEQ ID NO:4 CRM197_BamHI_Rev oligonucleotide sequence
SEQ ID NO:5 CRM197stop_BamHI_Rev oligonucleotide sequence
SEQ ID NO:6 An amino acid sequence of a tuberculosis H4 antigen (AG85B-TB10.4) as set out in Example 1
SEQ ID NO:7 An amino acid sequence of a tuberculosis H28 antigen (AG85B-TB10.4-rv2660c), as described in Example 1
SEQ ID NO:8 An amino acid sequence of a SpyTag peptide
SEQ ID NO:9 An amino acid sequence of an Isopeptag peptide
SEQ ID NO:10 An amino acid sequence of a SnoopTag peptide
SEQ ID NO:11 An amino acid sequence of a SnoopTagJr peptide
SEQ ID NO:12 An amino acid sequence of a DogTag peptide
SEQ ID NO:13 An amino acid sequence of a SdyTag peptide
SEQ ID NO:14 An amino acid sequence of an ELK16 peptide
SEQ ID NO:15 A nucleic acid sequence of a tuberculosis H4 antigen (AG85B-TB10.4)
SEQ ID NO:16 A nucleic acid sequence of a tuberculosis H28 antigen (AG85B-TB10.4-rv2660c)
SEQ ID NO:17 An amino acid sequence of a peptide fragment (referred to as “P*17 peptide”) derived from an M-protein of Streptococcus pyogenes
SEQ ID NO:18 An amino acid sequence of a peptide fragment (referred to as “S2 peptide”) derived from a SpyCEP protein of Streptococcus pyogenes
SEQ ID NO:19 An amino acid sequence of a CRM197-Ag85B-TB10.4 (CRM197-H4) chimeric protein as set out in Table 2
SEQ ID NO:20 An amino acid sequence of a CRM197-Ag85B-TB10.4-Rv2660c (CRM197-H28) chimeric protein as set out in Table 2
SEQ ID NO:21 An amino acid sequence of His6-Ag85B-TB10.4 (His6-H4) as set out in Table 3
SEQ ID NO:22 An amino acid sequence of His6-Ag85B-TB10.4-Rv2660c (His6-H28) as set out in Table 3
SEQ ID NO:23 An amino acid sequence of a CRM228 protein as set out in FIG. 15A
SEQ ID NO:24 An amino acid sequence of a CRM176 protein as set out in FIG. 15A
SEQ ID NO:25 An amino acid sequence of a CRM1001 protein as set out in FIG. 15A
SEQ ID NO:26 An amino acid sequence of a CRM45 protein as set out in FIG. 15A
SEQ ID NO:27 An amino acid sequence of a diphtheria toxin protein derived from Corynebacterium diphtheriae as set out in FIG. 15A
SEQ ID NO:28 An amino acid sequence of an HCV core protein fragment, as described in Example 5
SEQ ID NO:29 An amino acid sequence of an HCV NS3 protein fragment, as described in Example 5
SEQ ID NO:30 An amino acid sequence of an HCV E1 protein fragment, as described in Example 5
SEQ ID NO:31 An amino acid sequence of an HCV E2 protein fragment, as described in Example 5
SEQ ID NO:32 An amino acid sequence of an α-crystallin (HspX) polypeptide from Mycobacterium tuberculosis as set out in Table 4
SEQ ID NO:33 An amino acid sequence of an early secreted antigenic target 6 kDA (ESAT6) polypeptide from Mycobacterium tuberculosis as set out in Table 4
SEQ ID NO:34 An amino acid sequence of a culture filtrate protein 10 kDa (CFP10) from Mycobacterium tuberculosis as set out in Table 4
SEQ ID NO:35 An amino acid sequence of a Rv1509 polypeptide from Mycobacterium tuberculosis as set out in Table 4
SEQ ID NO:36 An amino acid sequence of a Rv2658c polypeptide from Mycobacterium tuberculosis as set out in Table 4
SEQ ID NO:37 An amino acid sequence of a Rv1508c polypeptide from Mycobacterium tuberculosis as set out in Table 4
SEQ ID NO:38 An amino acid sequence of a TB7.7 polypeptide from Mycobacterium tuberculosis as set out in Table 4
SEQ ID NO:39 An amino acid sequence of a Rv3615c polypeptide from Mycobacterium tuberculosis as set out in Table 4
SEQ ID NO:40 An amino acid sequence of a Rv3020c polypeptide from Mycobacterium tuberculosis as set out in Table 4
SEQ ID NO:41 An amino acid sequence of a Dengue virus envelope fragment, as described in Example 7
SEQ ID NO:42 An amino acid sequence of a Dengue virus capsid protein fragment, as described in Example 7
SEQ ID NO:43 An amino acid sequence of an HCV core protein fragment, as described in Example 5
SEQ ID NO:44 An amino acid sequence of a polyprotein from an HCV genome, as described in Example 5
SEQ ID NO:45 An amino acid sequence of an HCV E1 protein, as described in Example 5
SEQ ID NO:46 An amino acid sequence of an HCV E1/E2 polyprotein, as described in Example 5
SEQ ID NO:47 An amino acid sequence of a Dengue virus envelope protein, as described in Example 7
SEQ ID NO:48 An amino acid sequence of a Dengue virus capsid protein, as described in Example 7
SEQ ID NO:49 An amino acid sequence of a CRM197 protein as set out in FIG. 15A
SEQ ID NO:50 An amino acid sequence of a CRM197 protein as set out in FIG. 15B
SEQ ID NO:51 An amino acid sequence of a CRM228 protein as set out in FIG. 15B
SEQ ID NO:52 An amino acid sequence of a CRM176 protein as set out in FIG. 15B
SEQ ID NO:53 An amino acid sequence of a CRM1001 protein as set out in FIG. 15B
SEQ ID NO:54 An amino acid sequence of a CRM45 protein as set out in FIG. 15B
SEQ ID NO:55 An amino acid sequence of a diphtheria toxin protein derived from Corynebacterium diphtheriae, as set out in FIG. 15B
SEQ ID NO:56 An amino acid sequence of a SARS-CoV-2 N (nucleocapsid) polypeptide, as described in Example 9
SEQ ID NO:57 An amino acid sequence of a receptor binding domain (RBD) domain of a SARS-CoV-2 S protein, as described in Example 9
SEQ ID NO:58 An amino acid sequence of an S1 domain of a SARS-CoV-2 S protein, as described in Example 10
SEQ ID NO:59 An amino acid sequence of a Q fever antigen—COX, as described in Example 11
SEQ ID NO:60 An amino acid sequence of a Q fever antigen—Com1, as described in Example 12
SEQ ID NO:61 An amino acid sequence of predicted B and T cell epitopes derived from a Q fever antigen—OmpH, as a fusion protein, as described in Example 12
SEQ ID NO:62 An amino acid sequence of a Q fever antigen—YbgF, as described in Example 12
SEQ ID NO:63 An amino acid sequence of predicted B and T cell epitopes derived from Q fever peptide antigen—GroEL, as a fusion protein as described in Example 12
SEQ ID NO:64 An amino acid sequence of a SARS-CoV-2 spike protein, as described in Example 9
SEQ ID NO:65 An amino acid sequence of a CRM197-P*17 peptide fusion protein, as set out in Table 6
SEQ ID NO:66 An amino acid sequence of a CRM197-S2 peptide fusion protein as set out in Table 6
SEQ ID NO:67 An amino acid sequence of a CRM197-P*17-S2 peptide fusion protein as set out in Table 6
SEQ ID NO:68 An amino acid sequence of a CRM197-P*17-S2 peptide fusion protein as set out in Table 6
SEQ ID NO:69 An amino acid sequence of an HCV NS3 protein, as described in Example 5
SEQ ID NO:70 An amino acid sequence of a peptide derived from an HCV E1 protein, as described in Example 5
SEQ ID NO:71 An amino acid sequence of a peptide derived from an HCV E2 protein, as described in Example 5
SEQ ID NO:72 An amino acid sequence of a wild-type Q fever antigen—OmpH, as described in Example 12
SEQ ID NO:73 An amino acid sequence of a wild-type Q fever antigen—GroEL, as described in Example 12
SEQ ID NO:74 An amino acid sequence of a B cell epitope from OmpH, as set out in Table 7
SEQ ID NO:75 An amino acid sequence of a B cell epitope from OmpH, as set out in Table 7
SEQ ID NO:76 An amino acid sequence of a B cell epitope from OmpH, as set out in Table 7
SEQ ID NO:77 An amino acid sequence of a B cell epitope from OmpH, as set out in Table 7
SEQ ID NO:78 An amino acid sequence of a B cell epitope from OmpH, as set out in Table 7
SEQ ID NO:79 An amino acid sequence of a B cell epitope from OmpH, as set out in Table 7
SEQ ID NO:80 An amino acid sequence of a B cell epitope from OmpH, as set out in Table 7
SEQ ID NO:81 An amino acid sequence of a B cell epitope from OmpH, as set out in Table 7
SEQ ID NO:82 An amino acid sequence of a B cell epitope from OmpH, as set out in Table 7
SEQ ID NO:83 An amino acid sequence of a B cell epitope from OmpH, as set out in Table 7
SEQ ID NO:84 An amino acid sequence of a B cell epitope from OmpH, as set out in Table 7
SEQ ID NO:85 An amino acid sequence of a T cell epitope from OmpH, as set out in Table 7
SEQ ID NO:86 An amino acid sequence of a B cell epitope from GroEL, as set out in Table 7
SEQ ID NO:87 An amino acid sequence of a B cell epitope from GroEL, as set out in Table 7
SEQ ID NO:88 An amino acid sequence of a B cell epitope from GroEL, as set out in Table 7
SEQ ID NO:89 An amino acid sequence of a B cell epitope from GroEL, as set out in Table 7
SEQ ID NO:90 An amino acid sequence of a B cell epitope from GroEL, as set out in Table 7
SEQ ID NO:91 An amino acid sequence of a B cell epitope from GroEL, as set out in Table 7
SEQ ID NO:92 An amino acid sequence of a B cell epitope from GroEL, as set out in Table 7
SEQ ID NO:93 An amino acid sequence of a B cell epitope from GroEL, as set out in Table 7
SEQ ID NO:94 An amino acid sequence of a B cell epitope from GroEL, as set out in Table 7
SEQ ID NO:95 An amino acid sequence of a B cell epitope from GroEL, as set out in Table 7
SEQ ID NO:96 An amino acid sequence of a B cell epitope from GroEL, as set out in Table 7
SEQ ID NO:97 An amino acid sequence of a B cell epitope from GroEL, as set out in Table 7
SEQ ID NO:98 An amino acid sequence of a B cell epitope from GroEL, as set out in Table 7
SEQ ID NO:99 An amino acid sequence of a B cell epitope from GroEL, as set out in Table 7
SEQ ID NO:100 An amino acid sequence of a T cell epitope from GroEL, as set out in Table 7
SEQ ID NO:101 A selected S protein amino acid sequence of a SARS-CoV-2 spike protein, as described in Example 10
SEQ ID NO:102 An amino acid sequence of S1/S2 furin cleavage site of a SARS-CoV-2 spike protein, as described in Example 10
SEQ ID NO:103 An amino acid sequence of the N terminus of an S2 domain of a SARS-CoV-2 spike protein, as described in Example 10
SEQ ID NO:104 An amino acid sequence of a peptide from an HCV E2 protein, as set out in Example 5

### DETAILED DESCRIPTION

The present invention is predicated, as least in part, by an unexpected finding that a material that was otherwise thought of a biological waste during recombinant production of a CRM protein, particularly CRM197, may be used as an immunogenic agent and more particularly, an immunogen carrier/delivery system.

Typically, the soluble form of CRM197 (also referred to herein as “soluble CRM197”) has been the desired or target agent for use in applications such as a carrier protein and/or immunological agent where use of a CRM197 protein is required (e.g., conjugate immunogenic compositions, such as vaccines). Accordingly, production of CRM197 as an immunogen carrier system (noting that herein, the term “immunogen carrier system” is used interchangeably with “antigen carrier system”) in recombinant expression systems is focussed and driven towards production of soluble CRM197. As is known in the art, production of soluble CRM197 in a recombinant system may be achieved by expression of the protein in a soluble fraction or a soluble component or portion of a cell (such as the culture medium or periplasmic space, or recovered in the supernatant of a cell lysate after centrifugation), or alternatively by recovering, converting, or extracting soluble CRM197 from an insoluble cellular entity such as an inclusion body, although without limitation thereto. During recovery of soluble CRM197, the cellular portion or component which includes insoluble CRM197 (typically, an inclusion body) has been disregarded for further use and treated as biological waste. Moreover, improvements in CRM197 protein recovery from recombinant expression has focussed on increasing yields of soluble CRM197. Surprisingly, the present inventors have found that the normally discarded insoluble form or fraction of CRM197 produced during recombinant CRM197 expression can act as an immunogenic agent and/or immunogen carrier system. In particular, it has been found that an in vivo assembled protein particle comprising a CRM197 protein fused to a target immunogen when in the form of an isolated and purified CRM197:target immunogen chimera from an inclusion body produced in a recombinant cell, can act as an immunogenic agent and/or an immunogen carrier system, and an immunodiagnostic agent, although it will be appreciated that the present invention is not limited to this finding.

Additionally, the present inventors have found that in some forms, a protein particle comprising a CRM (in particular, CRM197) amino acid sequence and an immunogenic amino acid sequence, the protein particle being derived from an inclusion body of a cell, and in particular a cell of a recombinant expression system, retained conformation of the immunogenic protein in the particle such that the immunogenic protein (in particular, the spike protein of SARS-CoV-2) was able to bind its cognate human receptor. As such, the present invention may be useful where in some embodiments, it is desirable to retain a conformation or proper protein folding of an immunogenic protein, fragment, or epitope. It will be appreciated that the present invention may be generalizable for use with one or a plurality of immunogens from a wide variety of sources, agents, or molecules. The present inventors have also found that in some instances, the protein particles of the present invention can be produced at high density. It has also been observed the protein particles may be formulated into a highly concentrated solution for administration to a subject. It is also observed that in some instances, said highly concentrated formulation is a substantially clear solution and is easy to inject or administer to the subject. As such, high doses may be able to be administered if a large-scale immunisation program is desired. Therefore, the present invention may overcome one or more conventional barriers to production of soluble CRM197 such as, but not limited thereto, cost, yield, scalability, downstream processing (for example to remove soluble impurities) during manufacture of components of a composition such as, but not limited to, a vaccine composition.

In some embodiments, the present invention may offer a useful alternative to other immunogen carrier systems or immunogenic agents that may be hampered by a limitation to the size of the immunogen of interest and/or may be cumbersome or difficult to produce. Once such example are virus-like particles (VLPs), which are expensive to produce (e.g., often requiring production in dedicated cell culture lines that can fold the viral structural protein to assemble the VLP), rely on conformation of the backbone virus structural protein for presentation of the immunogen, and/or can only tolerate insertion of an immunogen of interest of a certain size in order to preserve the structural integrity of the VLP. The present inventors have found that the protein particles of the present invention may not be as sensitive to size limitations of a candidate immunogen, and as such may be able to produce multivalent protein particles in a rapid and cost-effective manner.

Therefore in broad aspects, the invention may relate to methods which include administration of a protein particle comprising a diphtheria toxin CRM amino acid sequence, wherein the protein particle comprising the diphtheria toxin CRM amino acid sequence is derived from a cell, compositions comprising said protein particle, isolated proteins comprising a diphtheria toxin CRM amino acid sequence and one or more immunogenic amino acid sequence derived from, corresponding to, or of one or more immunogens, and other uses for such protein particles such as in a detection method. Accordingly, the invention also relates to methods as described herein wherein a protein particle may further include one or more immunogens of interest.

A “a diphtheria toxin CRM amino acid sequence” as used herein refers to an amino acid sequence derived from, or corresponding to, an amino acid sequence of a Cross-Reacting Material (CRM) protein, being a mutant diphtheria toxin from Corynebacterium diphtheriae as herein described. The term “a diphtheria toxin CRM amino acid sequence” may be used interchangeably herein with “a CRM amino acid sequence”. In addition, the term “a diphtheria toxin CRM protein” may be used interchangeably with “a CRM protein”. It will be appreciated that “a diphtheria toxin CRM protein”, “a CRM protein”, or the variations as described herein, may be referred to as a toxoid. It will be understood that “a CRM amino acid sequence” is inclusive of an amino acid sequence of a fragment, variant, or derivative of a CRM protein. Exemplary amino acid sequences of one or more CRM proteins, and DT may be found in FIGS. 15A and 15B herein.

As hereinbefore described, a CRM protein of a diphtheria toxin relates to a substantially non-toxic mutant form of diphtheria toxin that is immunologically cross-reactive with a diphtheria toxin, and generally may share a sequence or structural similarity with diphtheria toxin but is distinct to diphtheria toxin as would be understood by a skilled addressee. A CRM protein may have one or more amino acid substitutions compared to a native or wild-type diphtheria toxin amino acid sequence. It is envisaged that a CRM protein may have a deletion of an amino acid sequence compared to a native or wild-type diphtheria toxin amino acid sequence. Typically, although not exclusively, a CRM protein may derived be from a mutated tox gene of Corynebacterium diphtheriae. A CRM protein may have a chain termination mutation. A CRM protein may have a missense mutation. Suitably, a CRM protein is a mutant form of diphtheria toxin. It will be appreciated that a CRM protein cross reacts with a diphtheria antitoxin due to one or more antigenic/immunogenic similarities to diphtheria toxin. Accordingly, a CRM protein may be a non-toxic, immunologically cross-reactive form of diphtheria toxin, or may be at least substantially non-toxic, immunologically cross-reactive form of diphtheria toxin. It is envisaged that a CRM protein and/or CRM amino acid sequence as described herein is suitable for use as an immunogenic agent and/or carrier protein, particularly for use in immunogenic compositions such as vaccine formulations, although without limitation thereto. It will be appreciated that in some embodiments, a suitable CRM protein will not substantially retain and/or display one or more toxic properties of a diphtheria toxin. Diphtheria toxin-related toxicity would be readily ascertainable or discernible by a skilled addressee and may include in vitro assays (e.g., cell-based or cell-free cytotoxicity assays) or in vivo assay (e.g., lethal dose assays, or LD50 assays in a suitable non-human animal model). In some embodiments, a CRM protein may have lost a portion, perhaps all, of an activity or a property (or a plurality thereof) normally found in a diphtheria toxin. Diphtheria toxin (DT) is a two-component exotoxin of Corynebacterium diphtheriae synthesized as a single polypeptide chain of 535 amino acids containing an A (active) domain and a B (binding) domain linked together by a disulphide bridge. Diphtheria toxin is encoded by the tox gene of Corynebacterium diphtheriae. Diphtheria toxin will be known to a person of skill in the art. An exemplary amino acid sequence of a diphtheria toxin may be found by reference to GenBank Accession No. AAV70486.1, although without limitation thereto. A further exemplary sequence of a diphtheria toxin may be found in an amino acid sequence as set forth in SEQ ID NO:27 or SEQ ID NO:55. The amino acid sequence of DT as set forth in SEQ ID NO:55 is the amino acid sequence of the mature, fully-processed form of DT without a signal peptide or a start methionine. Throughout this document, when reference is made to an amino acid position or numbering in a DT protein, such numbering is made with reference to the amino acid sequence as set forth in SEQ ID NO:55 with the first amino acid residue in SEQ ID NO:55 (a glycine residue) being position 1.

Reference is made to Holmes, R. (2000), The Journal of Infectious Diseases, 181: S156-S167 which provides a non-limiting description of DT and properties thereof, which is incorporated herein by reference. DT is an ADP-ribosylating enzyme comprising two fragments (A and B). Fragment A (amino acid residues 1 to 190 of DT) uses NAD as a substrate, catalyzing the cleavage of the N-glycosidic bond between the nicotinamide ring and the N-ribose and mediating the covalent transfer of the ADP-ribose (ADPRT activity) to the modified histidine 715 (diphthamide) of the elongation factor EF-2. This post-translational diphthamide modification inactivates EF-2, halting protein synthesis and resulting in cell death. The A fragment (also named C domain) carries the catalytic active site and is the only fragment of the toxin required for the final step of intoxication. The R domain (spanning amino acid residues 386 to 535 of DT), carried on the B fragment, mediates binding to receptors on the host cell surface and the T domain (spanning amino acid residues 201 to 384 of DT), also carried on the B fragment, promotes the pH-dependent transfer of fragment A to the cytoplasm. An arginine-rich disulfide-linked loop connects fragment A to fragment B (or domain C to domains TR). This inter-chain disulfide bond is the only covalent link between the two fragments after proteolytic cleavage of the chain at position 186. The diphtheria toxin binds to heparin-binding epidermal growth factor precursor. It will be appreciated that an activity or property may relate to fragment A-associated nuclease activity, translational inhibitory activity, or receptor binding activity, although without limitation thereto. By way of example only, a CRM protein as used herein may have a reduced ability to bind NAD, which may in turn at least partially reduce or possibly eliminate a toxic property of diphtheria toxin. A CRM protein may display structural similarity to a diphtheria toxin. Suitably, a CRM protein may retain the immunostimulatory activity of diphtheria toxin. It is envisaged that a CRM can be of any size and composition, and may contain at least a portion of DT.

Non-limiting examples of a CRM protein that is immunologically cross reactive with a diphtheria toxin, which can be used in the present invention includes, but is not limited to, a CRM197 (as described herein), a CRM45 (CRM45 lacks the last 149 C-terminal amino-acid residues of native diphtheria toxin; exemplary amino acid sequences of a CRM197 and a CRM45 may be found in Giannini et al., Nucleic Acids Res. (1984), 12(10): 4063, which is incorporated herein by reference), a CRM30, a CRM228 (a CRM228 comprises five residues substituted compared to native diphtheria toxin: G79D, E162K, S197G, P378S, and G431S, resulting in a CRM228 displaying about 15% to about 20% of the binding activity of native diphtheria toxin and no ADPRT activity), and a CRM176 (substitution of glycine at 128 to aspartic acid; an exemplary amino acid sequence and partial functional characterisation of a CRM176 is described in Maxwell et al., (1987) Mol Cell Biol. 7: 1576, which is incorporated herein by reference). A CRM1001, a functional and non-toxic mutant of DT, includes a single mutation, C471Y, as described by David M. Neville et al., (1986) Ann Rev Biochem. 55:195-224, which is incorporated herein by reference. The present invention also contemplates a diphtheria toxin CRM amino acid sequence comprising an amino acid sequence derived from, or corresponding to, a plurality of diphtheria toxin CRM proteins. Reference is made to “The Comprehensive Sourcebook of Bacterial Protein Toxins”, Eds Alouf et al., 2005, Third Edition, Academic Press, which provides an overview of diphtheria toxin CRMs, and is incorporated herein by reference. An amino acid sequence alignment of some CRM proteins is shown in FIGS. 15A and 15B. According to some embodiments, a diphtheria toxin CRM amino acid sequence may comprise, consist essentially of, consist of, or may be, an amino acid sequence derived from, or corresponding to, a CRM protein selected from the group consisting of a CRM197 protein, a CRM45 protein, a CRM1001 protein, a CRM228 protein, a CRM176 protein, and a CRM30 protein, and any combination thereof, and may be inclusive of fragments, variants and derivatives thereof. Accordingly, it is envisaged that in some embodiments, a diphtheria toxin CRM amino acid sequence may comprise an amino acid sequence derived from, or corresponding to, a plurality of different CRM proteins, inclusive of a fragment, variant, or derivative thereof. It is also envisaged that a diphtheria toxin CRM amino acid sequence may comprise an amino acid sequence derived from or corresponding to different portions or regions of the same CRM protein (inclusive of a fragment, variant, or derivative thereof).

In some preferred embodiments, a CRM protein may be a CRM197 protein, or a fragment, variant, or derivative thereof. As used herein, the term “CRM197” and “CRM197 protein” (and variations thereof) refers to a non-toxic mutant of diphtheria toxin, which differs from diphtheria toxin by an amino acid substitution within the catalytic domain of a glycine residue at position 52 of the wild-type diphtheria toxin to glutamate. Although not wishing to be bound by any particular theory, this mutation is considered responsible for the loss of ADP-ribosyltransferase activity in CRM197. CRM197 retains binding activity. Reference is made to Giannini et al (1984) Nucleic Acids Research, 12: 4063, which describes an amino acid sequence of an exemplary CRM197 protein. Reference is made to Bröker et al. (2011) Biologicals 39: 195, which describes exemplary properties of a CRM197 protein, which is incorporate herein by reference. In the context of the present invention, exemplary amino acid sequences of a CRM197 protein are set forth in any one of SEQ ID NOS:2, 49, and/or 50. In some embodiments, an amino acid sequence of, or from, a CRM197 protein comprises an amino acid sequence as set forth in SEQ ID NO:50. Particular reference is made to SEQ ID NO:50, being an amino acid sequence of a fully-processed CRM197 protein, namely without the leader or signal peptide sequence, or a start methionine. Throughout this document, when reference is made to an amino acid position or numbering in a CRM197 protein, such numbering is made with reference to the amino acid sequence as set forth in SEQ ID NO:50 with the first amino acid residue in SEQ ID NO:50 (a glycine residue) being position 1. A further exemplary amino acid sequence of a CRM197 protein is set forth in Giannini et al (1984; supra).

In the context of the present invention, an exemplary amino acid sequence of a CRM228 protein is set forth in SEQ ID NO:23 and/or SEQ ID NO:51. An exemplary amino acid sequence of a CRM176 protein is set forth in SEQ ID NO:24 and/or SEQ ID NO:52. It will be appreciated that an exemplary amino acid sequence of a CRM1001 protein is set forth in SEQ ID NO:25 and/or SEQ ID NO:53. In the context of the present invention, an exemplary amino acid sequence of a CRM45 protein is set forth in SEQ ID NO:26 and/or SEQ ID NO:54.

In some embodiments, a CRM amino acid sequence may comprise, consist essentially of, consist of, or is, an amino acid sequence selected from the group consisting of an amino acid sequence as set forth in SEQ ID NO:2, SEQ ID NO:23, SEQ ID NO:24, SEQ ID NO:25, SEQ ID NO:26, SEQ ID NO:49, SEQ ID NO:50, SEQ ID NO:51, SEQ ID NO:52, SEQ ID NO:53, and SEQ ID NO:54, or a fragment, variant, or derivative thereof, and any combination thereof. In certain embodiments, the CRM amino acid sequence may comprise, consist essentially of, consist of, or may be, an amino acid sequence as set forth in SEQ ID NO:50.

The present invention contemplates a CRM amino acid sequence derived from, or corresponding to, a full-length CRM protein, or fragments, variants, or derivatives thereof as herein described. It would be understood by a person of skill in the art that the invention also contemplates mutations or variations (including, but not limited to, substitution, deletion and/or addition) that may naturally occur in or are introduced artificially into a CRM amino acid sequence without affecting one or more biological and/or physical properties of a CRM protein. It will be appreciated that in context of the present invention, a CRM protein and/or a CRM amino acid sequence encompasses all such proteins, polypeptides, fragments, mutants, and variants, including a polypeptide as set forth in any one of SEQ ID NOS:2, SEQ ID NOS:23 to 26 and/or SEQ ID NOS:49 to 54, and its natural or artificial variants, wherein the variants retain one or more biological and/or physical properties of a CRM protein, e.g., no or reduced cytotoxicity compared to DT, immunogenicity (although without limitation thereto). In addition, fragments of a CRM protein include not only the fragments of a protein as described herein such as, but not limited to, sequences as set forth in any one of SEQ ID NO:2, SEQ ID NOS: 23 to 26 and/or SEQ ID NOS:49 to 54, but also the corresponding fragments of the natural or artificial variants of the protein.

In some preferred embodiments, a CRM amino acid sequence may be derived from, or corresponds to, an amino acid sequence of, or from, a CRM197 protein, or a fragment, variant, or derivative thereof. Preferably, the CRM sequence amino acid sequence and/or the amino acid sequence derived from, or corresponding to a CRM197 protein, may comprise, consist of, consist essentially of, or may be, an amino acid sequence as set forth in any one of SEQ ID NO: 2, SEQ ID NO:49, and/or SEQ ID NO:50. In some preferred embodiments, the CRM sequence and/or the amino acid sequence derived from, or corresponding to the CRM197 protein, is or comprises, consists of, consists essentially of, or may be, an amino acid sequence as set forth in SEQ ID NO:50. As will be understood, the invention contemplates fragments, variants, or derivatives of a CRM197 protein, and in some embodiments, a CRM197 protein as set out in SEQ ID NO:2, SEQ ID NO:49, and/or SEQ ID NO:50.

As generally used herein, “immunological”, and “immunogenic” refers to an ability or property of an agent (e.g., protein particle, protein, fragment, composition, etc) to elicit an immune response upon administration to a subject.

The terms “immunogen” and “immunogen of interest”, as used herein, refer to a molecule that is capable of eliciting an immune response, and more particularly a specific or desired immune response such as, but not limited to, a protective immune response, a cell-mediated response, an antibody (e.g., neutralising antibody) response, or a memory immune response. The terms “immunogen” and “immunogen of interest” may be used interchangeably herein with “antigen” or “antigenic”. An immunogen may be a proteinaceous molecule. It is also envisaged that an immunogen may be non-proteinaceous molecule such as, but not limited to, a polysaccharide and/or a glycan. The terms “immunogenic sequence”, “immunogenic protein”, “immunogenic fragment”, or “immunogenic amino acid sequence” typically relate to embodiments encompassing an immunogen derived from, or corresponding to, or of, a protein, or a fragment, variant, or derivative of said protein. The term “epitope” may be also used to describe an immunogenic protein, sequence, fragment, or an amino acid sequence. A protein-derived immunogen may comprise a continuous or discontinuous sequence amino acids of a protein, wherein the immunogen can be recognised or bound by an element of the immune system, such as an antibody or other antigen receptor. An immunogen or antigen may comprise one or more epitopes (e.g., either linear, conformational, or both) that elicit an immunological response, as described below and known to a person of skill in the art. Normally, a B-cell epitope derived from an immunogenic protein may include at least about 5 amino acids but can be as small as 3-4 amino acids. A T-cell epitope derived from an immunogenic protein, such as a cytotoxic T-cell (CTL) epitope, may include at least about 7-9 amino acids, and a helper T-cell epitope may include at least about 12-20 amino acids. Non-protein derived immunogens such as polysaccharides (but not limited to) may comprise one or more epitopes such as described herein. The present invention also contemplates use of a mimotope which mimics a structure of an epitope, as is known in the art. The present invention also contemplates “polytope” proteins that may comprise one or a plurality of immunogenic fragments or sequences from the same or different agents, molecules, or sources. For example, said sequences or fragments in a polytope may be present singly or as repeats, which also includes tandemly repeated fragments. A “polytope” may be useful when an amplified immune response is desired, or different types of immune responses are desired. A person of skill in the art will readily appreciate or derive an appropriate length of an epitope that may be suitable for an intended purpose. The term “immunogen” may denote both subunit immunogens, e.g., immunogens which are separate and discrete from a whole organism with which the immunogen is associated in nature, as well as killed, attenuated or inactivated bacteria, viruses, fungi, parasites or other microbes. An immunogen such as a polysaccharide may elicit a T-cell independent response. Antibodies such as anti-idiotype antibodies, or fragments thereof, and synthetic peptide mimotopes, which can mimic an immunogen or immunogenic determinant, are also envisaged. In light of the foregoing, it will be appreciated that the protein particle as described herein may be useful for use with a wide variety of target antigens.

In certain embodiments, a protein particle as described herein further comprises one or more immunogens other than a diphtheria toxin CRM amino acid sequence. It will be appreciated that the, or each, immunogen other than a diphtheria toxin CRM amino acid sequence as described herein may be any immunogen other than a diphtheria toxin CRM amino acid sequence (inclusive of a protein or polypeptide derived from the diphtheria toxin CRM amino acid sequence) that may elicit an immune response. It will be appreciated that in certain embodiments, the diphtheria toxin CRM amino acid sequence (inclusive of a protein or polypeptide derived from the diphtheria toxin CRM amino acid sequence) may itself elicit an immune response upon suitable administration to a subject.

In certain embodiments, the, or each, immunogen other than a diphtheria toxin CRM amino acid sequence comprises an immunogenic amino acid sequence. In some embodiments, the one or more immunogens may be one or more immunogens other than a CRM197 amino acid sequence.

It is contemplated that in some embodiments, an immunogenic amino acid sequence, immunogenic sequence, immunogenic protein, immunogenic fragment and the like, may be derived from, or correspond to, comprise, consist of, consist essentially of, or is, one or more amino acid sequences as set forth in one or more Examples, Tables, and/or Figures as described herein.

Throughout the specification, the terms “polypeptide,” “proteinaceous molecule,” “peptide,” and “protein” are used interchangeably herein to refer to a polymer of amino acid residues and to variants, fragments, derivatives, and synthetic analogues of the same. Thus, these terms may apply to amino acid polymers in which one or more amino acid residues is a synthetic non-naturally-occurring amino acid, such as a chemical analogue of a corresponding naturally-occurring amino acid, as well as to naturally-occurring amino acid polymers. The amino acid residues may also apply to D- or L-amino acids. These terms do not exclude modifications, for example, glycosylations, acetylations, phosphorylations, and the like.

By “corresponds to” or “corresponding to” in the context of the present invention, is meant an amino acid sequence or nucleic acid sequence which shares primary sequence characteristics of another amino acid sequence or nucleic acid sequence but is not necessarily derived or obtained from the same source as said another amino acid sequence or said nucleic acid sequence.

By “protein particle comprising a diphtheria toxin CRM amino acid sequence is derived from a cell” and the like, is meant a particulate structure comprising a diphtheria toxin CRM amino acid sequence, wherein the protein particle is derived, obtained, or otherwise prepared from a cell. The term “protein particle” may be used interchangeably herein with this expression. In some embodiments, the protein particle derived from a cell may be isolated, purified, and/or substantially purified from a cell as herein described. The protein particle may have a substantially particulate protein structure. It is envisaged that in some embodiments, the majority (for example, at least 40%, 50%, 60%, 70%, 80%, 90%, or more) but not necessarily all may have a particulate form. In some embodiments, a protein particle may be free, or substantially free, of material other than the protein molecule/s from which the particle is derived or formed. In some embodiments, the protein particle may be formed or assembled in a cell. In some embodiments, the protein particle may be expressed in a cell. In certain embodiments, a protein particle may be formed, substantially formed, derived, obtained, assembled, or otherwise produced from a diphtheria toxin CRM amino acid sequence, suitably in some embodiments when the diphtheria toxin CRM amino acid sequence is expressed in a cell. Suitably, the cell may be a host cell for recombinant expression. In some embodiments, the protein particle may be produced, formed, assembled, or expressed in a cell by recombinant technology, suitably may be recombinant DNA technology, as described herein. In some embodiments, the protein particle may be derived, obtained, assembled, prepared, produced or formed by recombinant expression of an amino acid sequence comprising a diphtheria toxin CRM amino acid sequence and optionally further comprising one or more immunogens other than a diphtheria toxin CRM amino acid sequence. According to some of these embodiments, the, or each, immunogen may comprise an immunogenic amino acid sequence. According to some preferred embodiments, a protein particle may be derived from, obtained from, isolated from, produced, assembled, or formed by or from, recombinant expression of an amino acid sequence comprising a diphtheria toxin CRM amino acid sequence and an immunogenic amino acid sequence of one or more immunogens other than a diphtheria toxin CRM amino acid sequence. In some embodiments, the protein particle may be obtained, isolated, or derived from an intracellular component or portion of a cell as described herein. It will be understood that one or more components of a protein particle may be linked to each other by any appropriate bond type e.g., non-covalent, covalent, or a mixture thereof.

The protein particle may be any suitable shape, and may be spherical, ellipsoidal, filamented, in sheets, discs, or any other shape. The protein particles may be of any size. It will be appreciated that in some embodiments, a protein particle or a composition or formulation comprising the same, may have substantially the same mean particle size (e.g., be a monodispersed). Alternatively, a heterogenous particle size may be desirable. A protein particle as described herein may have a size and/or shape that facilitates or promotes uptake by one or more cells of the immune system. By way of example, a protein particle (or a composition or formulation comprising the same) with a mean particle size range of between about 0.01 μm and about 10 μm may be suitable for uptake by an antigen presenting cell in some embodiments. Uptake of a protein particle by an antigen presenting cell may be by way of phagocytosis, although without limitation thereto. In another example, a protein particle may be taken up by antigen presenting cells by way of phagocytosis, and according to these examples, a protein particle with a mean particle size range of between about 0.5 μm and about 10 μm may be suitable. In yet another example, a protein particle may have an average size and/or shape that facilitates direct uptake or delivery into one or more components of the lymphatic system and thus elicit or stimulate an immune response. Accordingly, a protein particle with a mean particle size of less than, or equal to, about 50 nm may be suitable for uptake into one or more components of the lymphatic system. In some embodiments, a protein particle as described herein may have a mean particle size between about 1 nm and about 800 μm, may have a mean particle size between about 100 nm and about 600 μm, may have a mean particle size between about 300 nm and about 500 μm, may have a mean particle size between about 400 nm and about 400 μm, may have a mean particle size between about 800 nm and about 200 μm, or may have a mean particle size between about 1 μm and about 150 μm. In some embodiments, the protein particle may have a mean particle size less than, or equal to, about 100 μm. In some embodiments, a protein particle may have a mean particle size less than, or equal to, about 3 μm, less than, or equal to, about 5 μm, less than, or equal to, about 10 μm, less than, or equal to, about 20 μm, less than, or equal to, about 30 μm, less than, or equal to, about 50 μm, less than, or equal to, about 70 μm or less than, or equal to, about 90 μm. In other embodiments, a protein particle may have a mean particle size less than, or equal to, about 50 nm. In other embodiments, a protein particle may have a mean particle size between about 500 nm and about 10 μm.

A protein particle as described herein may be derived, assembled, or formed from, or comprise a single protein (e.g., chimeric protein). Alternatively, a protein particle may be derived from, assembled from, formed from, or comprise two or more different proteins (e.g., chimeric proteins). It is contemplated that the protein particle may partially or totally encapsulate an immunogen in the interior of the protein particle, or alternatively display an immunogen on the surface of the protein particle. It is also envisaged that a protein particle may have a combined morphology of encapsulating and displaying an immunogen.

It is envisaged that a protein particle as described herein may be a mixture of an unfolded, a misfolded, a partially folded, and/or a folded protein. The folded protein may be a substantially fully folded protein. The folded protein may be biologically active and/or may be properly folded (for example to present conformational epitopes). Alternatively, a protein particle may be substantially formed from an unfolded, a misfolded, folded protein and/or a partially folded protein. It is also envisaged that a protein particle may be substantially formed from misfolded proteins. The ratio or relative amounts of different states or forms of a protein may be dependent on factors such as expression levels, expression systems, the immunogen of interest etc, although without limitation thereto. In some embodiments, a protein particle may be formed by a structured assembly of a protein molecule and/or it may form an aggregate structure (e.g., such as known from inclusion bodies), although without limitation thereto.

In some embodiments, a protein particle as described herein may self-assemble as, or into, a higher order structure with or without a defined structural geometry. In some embodiments, a protein particle may self-assemble in an insoluble component of a cell, wherein in some embodiments, the insoluble component may be an inclusion body. In some embodiments, the insoluble component and/or inclusion body may be derived from, purified from, produced from, prepared from, isolated from, or obtained from, a cell. In some embodiments, the cell may be suitable for recombinant expression as herein described.

The term “self-assembly” (and variations thereof) refers to a process of spontaneous assembly of a higher order structure from a lower order structure (e.g., a single protein molecule, or a proteinaceous molecule comprising a plurality of protein molecules) that involves, at least in part, on natural attractions of the components of the higher order structure (e.g., protein molecules) for each other. Typically, although not exclusively and not wishing to be bound by any particular theory, self-assembly occurs through random movements of the molecules and formation of bonds based on size, shape, composition, and/or chemical properties. A self-assembling protein component of the particles according to the present invention may be a peptide/protein known to form inclusion bodies (IB) when expressed in a suitable manner in a suitable host, or it may be a specially designed sequence capable of forming an insoluble particle having the desired characteristics. In the context of the present invention, self-assembly may occur during and/or as a result of increased, high, or overexpression of a protein from which the particle is derived in a suitable manner in a suitable host. The present invention also encompasses self-assembly of one or a plurality of protein molecules into a higher order structure, and in particular, the higher order structure may be a protein particle comprising a CRM amino acid sequence, and optionally one or a plurality immunogens or immunogenic amino acid sequences other than a CRM amino acid sequence. In some embodiments, the protein particle as described herein may be assembled into, produced as, or formed into, a protein particle when expressed in an appropriate host organism. In some embodiments, the CRM amino acid sequence may be derived from, or corresponds to, an amino acid sequence from a CRM197 protein, or a fragment, variant, or derivative thereof.

In some embodiments, a protein particle may be assembled, expressed, produced, or formed from, or comprise, a CRM amino acid sequence, and preferably a CRM197 amino acid sequence. Suitably, a protein particle is formed from the CRM amino acid sequence when the CRM amino acid sequence is expressed in a host cell, and preferably the particle is formed from the CRM197 amino acid sequence.

The term “substantially” as used herein generally means the majority but not necessarily all.

In some embodiments, the protein particle as described herein may be formed, produced, or expressed in a cell. In other embodiments, the protein particle may be a substantially insoluble protein particle derived from, formed, produced, or expressed in a cell. Accordingly, the protein particle (e.g., derived from an expressed protein) may form, fold into, or aggregate into, a substantially insoluble protein particle, preferably when expressed in a cell. In some embodiments, a substantially insoluble protein particle as described herein may refer a substantially insoluble entity in the form of a particle comprising a protein as described herein. A substantially insoluble protein particle may comprise a portion of a soluble protein. In some embodiments, the portion of soluble protein may be less than 20%, less than 15%, 10%, 5%, 1% or essentially free, of a soluble protein.

In some embodiments, a protein particle and/or substantially insoluble protein particle may be derived from, obtained from, produced, prepared from, or otherwise isolated or removed from, or is, an insoluble (or a substantially insoluble) component, portion, or a fraction of a cell. In some embodiments, a protein particle and/or substantially insoluble protein particle may also be derived from an aggregate or an aggregate structure formed in a cell. An aggregate or an aggregate structure may be part of, or derived from, an insoluble component, portion, or a fraction of a cell. An insoluble component of a cell may be any area, portion, or fraction of a cell that displays one or more characteristics of being insoluble, typically as a result of expression of a protein of interest, suitably by recombinant technology. Typically, an insoluble component will include a protein or protein particle of interest, possibly exclusively include the protein of interest, in various conformational states (e.g., unfolded, misfolded, partially misfolded or unfolded, and correctly folded forms). A protein of interest or a protein particle forming part of an insoluble component may or may not be biologically active, or may be partially-biologically active. Generally, a protein of interest in an insoluble component is substantially unfolded or misfolded forms, or partially misfolded. An insoluble component may be substantially-free of material other than the protein of interest (e.g., other proteins, lipids, small-molecules, etc). It will be appreciated that an insoluble component comprising the protein of interest or the protein particle may be a dense electron-refractile area or particle, such as visualised by microscopy and other imaging methods. An insoluble component may be substantially resistant to protein solubilisation by techniques known to the skilled addressee. An insoluble component may also be characterised or determined by size characterisation using sedimentation field-flow fractionation, for example. An insoluble component may also be characterised by separation of a cell lysate into a supernatant and a cell pellet, where the cell pellet includes the insoluble component. Such separation methods typically include sedimentation by centrifugation, although without limitation thereto. Alternatively, an insoluble component may be characterised by solubilisation in denaturing agents (e.g. urea) followed by separation via gel electrophoresis, as is known in the art. An insoluble component, or substantially insoluble component, may be found in the nucleus, cytoplasm and/or periplasmic space of a cell. An insoluble component, or substantially insoluble component, may arise as a result of expression in a recombinant expression system, and in particular may result from high levels of recombinant expression, as will be known by the skilled addressee. An insoluble component, or substantially insoluble component, may be a discrete body, which may be surrounded by a membrane. In some embodiments, an insoluble component, or substantially insoluble component, may be an inclusion body. In some embodiments, the inclusion body may comprise a protein particle, an aggregate and/or an aggregate structure, wherein the aggregate and/or the aggregate structure is, comprises, consists, or consists essentially of a protein particle as described herein.

It will be appreciated that formation of insoluble components, aggregates, structured particles, inclusion bodies, and the like may be induced by linking a protein to an aggregation prone peptide, optionally by a suitable linker. Non-limiting examples of an aggregation prone peptide includes a self-assembling ionic peptide such as an ELK16 peptide (LELELKLKLELELKLK; SEQ ID NO: 14) or a surfactant like peptide such as that described in Zhou et al. (2012) Microb Cell Fact. 11: 10, which is incorporated herein by reference. Other suitable sequences to induce formation of a protein of interest into an insoluble component, suitably an inclusion body, will be known to a skilled addressee. In some embodiments, a CRM amino acid sequence may further comprise an aggregation prone peptide as described herein.

In some embodiments, protein particles as described herein may have a comparable size and/or shape to a pathogen (e.g., a virus, a bacterium, a parasite etc.), which in turn may assist or enhance uptake by antigen presenting cells and thus may increase immunogenicity. Although not wishing to be bound by any particular theory, it will also be appreciated that a protein particle size that is comparable to, or substantially corresponds to, a pathogen may stimulate an innate immune response, although without limitation thereto.

Several detection techniques may be used in order to confirm that a protein has taken on the conformation of a protein particle or assembled into a protein particle, or other particle characteristics such as size, charge distribution, and mechanical stability. Such techniques include microscopy including electron microscopy (e.g., SEM, TEM), X-ray crystallography, isothermal calorimetry, image flow cytometry, dynamic light scattering, X-ray scattering, zeta potential measurement, or indirect methods by HPLC analysis and the like. For example, cryoelectron microscopy can be performed on vitrified aqueous samples of the protein particle preparation in question, and images recorded under appropriate exposure conditions. Detection methods such as microscopy may also be suitable to ascertain size distribution. Size distribution studies or analysis may be conducted in the presence or absence of an agent to assist with homogenisation or dispersal of particles such as, but not limited to, a detergent. Stability studies will be known to the skilled addressee. By way of example, thermal or solvent stability studies may be utilised to ascertain or understand the stability of the protein particle. Mechanical stability may be understood using a single-molecule method based on atomic force microscopy, for example. In some embodiments, the protein particles as described herein have enhanced, improved, or an increased mechanical stability compared to comparable protein particles made by other methods. Non-limiting examples of suitable methods to characterise protein particles, particularly proteins particles for use in therapeutic applications, may be found in Probst et al. (2017) J Pharm Sci. 106(8):1952-1960; Analysis of Aggregates and Particles in Protein Pharmaceuticals, EDs: Hanns-Christian Mahler and Wim Jiskoot, John Wiley & Sons, Inc., both of which are incorporated herein by reference.

It is envisaged that a surface charge of a protein particle may be measured or analysed. As will be known by the skilled addressee, a surface charge of particles may affect cellular uptake by one or more cells of the immune system e.g., an antigen presenting cell. By way of example, uptake of a protein particle by a dendritic cell may be facilitated or promoted when a protein particle possesses a net positively charged surface. By way of further example, negatively charged particles may be efficiently taken up by an antigen presenting cell, possibly by opsonisation or adsorption of negatively charged particles at cationic sites in the cell membranes, although without limitation thereto. In some embodiments, a protein particle as described herein may have a net negative charge or a net positive charge. In some embodiments, a, or the, surface of a protein particle as described herein may have a net negative charge or a net positively charge. A suitable surface charge of a protein particle for use in the present invention will be known and/or readily ascertainable to a skilled addressee. It is contemplated that a zeta potential may be useful for determination of net charge measured using, for example, a laser Doppler Micro-electrophoresis technique to measure zeta potential (e.g., using a Zetasizer Nano ZS by Malvern Panalytical). In such a technique, an electric field is applied to a solution of molecules or a dispersion of particles, which then move with a velocity related to their zeta potential. This velocity may be measured using a suitable technique such as light scattering in order to calculate electrophoretic mobility, and from this the zeta potential and zeta potential distribution may be calculated. It is envisaged that in some embodiments, a zeta potential measurement of a protein particle may be in the range between about −100 mV and about 100 mV, between about −70 mV and about 70 mV, between about −50 mV and about 50 mV, between about −30 mV and about 30 mV, between about −20 mV and about 20 mV, between about −5 mV and about −50 mV, or may be between about −10 mV and about −30 mV. In some embodiments, a zeta potential may be less than, or equal to, about 100 mV, may be less than, or equal to, about 50 mV, may be less than, or equal to, about 20 mV, may be less than, or equal to, about 10 mV, may be less than, or equal to, about 5 mV, may be less than, or equal to, about 1 mV, may be less than, or equal to, about −1 mV, may be less than, or equal to, about −5 mV, may be less than, or equal to, about −10 mV, may be less than, or equal to, about −15 mV, or may be less, or equal to, than about −20 mV.

Formation of a protein particle as described herein, and particularly formation by a self-assembly process, may be preferentially or selectively facilitated, increased, or enhanced in some embodiments by modulation or modification of one or more parameters under which an amino acid sequence is expressed. By way of example, very high levels of protein expression (e.g., 50% (w/w) of total proteins in biomass, which equates to 50 g wet weight of total proteins per 100 g wet weight of total biomass) in a cell may overload one or more protein folding pathways of the cell, which in turn may trigger formation or assembly of a protein particle, and suitably an insoluble component of a cell. Although not wishing to be bound by any particular theory, this triggering event may be due to the very rapid speed of protein production, which leads to interprotein interactions. Alternatively, overexpression of a CRM amino acid sequence, and in particular a CRM197 amino acid sequence, inside a cell may result in partial folding of the protein, leading to an increase in hydrophobic regions on the surface of a resultant protein molecule. Interprotein assembly may then occur between the protein molecules via these hydrophobic regions, to come together to form a protein particle (and preferably a substantially insoluble protein particle), a supramolecular structure, and/or an insoluble component of a cell. Non-limiting examples of one or more parameters under which the protein is expressed that may be modified to increase a protein expression level and/or yield relative to an expression level and/or yield when the one or more parameters have not been modified includes a culture condition (e.g., culture temperature, expression host, induction temperature and duration, use of an additive to enhance biomass production (such as glucose (e.g., at about 1% w/v glucose)), expression in the absence of a purification tag (such as 6×HIS) or other fusion partner sequence may, at least partially, reduce soluble protein production, and inclusion of sequences to increase protein production such as codon-optimisation as described herein. Such parameters will be known or ascertainable to a person of skill in the art.

It will be appreciated that various methods may be employed to determine or ascertain whether an immunogen is suitably associated with, formed with, folded or present in, or on, a protein particle described herein. By way of example only, a receptor-binding assay may be useful. Alternatively, an antibody-binding assay may be useful, although without limitation thereto. A person of skill in the art will readily ascertain suitable methods to be employed according to these embodiments.

In some preferred embodiments, the protein particle as described herein may be derived from, obtained from, isolated from, produced in, prepared from, or otherwise removed from, or is, an insoluble component of a cell. In some embodiments, the insoluble component may be an inclusion body formed, produced, derived from, obtained from, removed from, isolated from, or expressed in a cell. In some embodiments, the protein particle may be derived from or formed from a CRM amino acid sequence that optionally may comprise an amino acid sequence of interest as described herein. Accordingly, the protein particle may be derived, obtained, or isolated from an insoluble component of a cell, and in some preferred embodiments, the insoluble component may be an inclusion body from a cell or an inclusion body preparation from a cell or obtained from a cell, where the protein particle may be formed from, or derived from, an amino acid sequence comprising a CRM amino acid sequence, and more particularly may be formed from or derived from expression of a CRM amino acid sequence in a cell. Suitably, the protein particle may be formed from or derived from a CRM amino acid sequence when the CRM amino acid sequences is expressed in a cell. According to some embodiments, an amino acid sequence comprising a CRM amino acid sequence may be expressed as a recombinant protein to form a protein particle in a cell, and preferably may be a substantially insoluble protein particle, where preferably the protein particle may be derived from, obtained from, isolated from, produced, prepared, or otherwise removed from, or is, an insoluble component of a cell. Suitably, the insoluble component may be an inclusion body. Preferably, the CRM amino acid sequence may be derived from, or corresponds to, an amino acid sequence from a CRM197 protein, inclusive or fragments, variants, or derivatives.

A non-limiting advantage of the present invention in some embodiments may include that the methods and compositions may at least partially circumvent or eliminate the need to use a highly purified soluble form of a CRM protein, and in particular a CRM197, as an immunogenic agent or carrier protein. As will be known to the skilled addressee, recovery of a soluble and active protein from an insoluble component, portion, or fraction of a cell (such as an inclusion body) generally requires solubilisation/denaturation followed by protein refolding, which typically are time consuming, expensive and/or laborious steps that in turn, require further downstream processing to remove agents such as detergents and/or refolding agents such as urea and guanidine hydrochloride. As such, a protein particle as described herein may minimise laborious and/or expensive downstream processing steps typically associated with production of a soluble CRM protein (e.g., a soluble CRM197 protein), although without limitation thereto, in recombinant systems for use as an immunogenic agent.

According to broad embodiments, the invention contemplates a protein particle as described herein wherein the diphtheria toxin CRM amino acid sequence has not been derived from a diphtheria toxin CRM protein, or a fragment, variant, or derivative of a diphtheria toxin CRM protein, that has been subjected to a protein refolding treatment. According to some embodiments, the invention contemplates a protein particle derived from a cell, the protein particle comprising a CRM197 amino acid sequence, wherein the CRM197 amino acid sequence has not been derived from a CRM197 protein, or a fragment, variant, or derivative thereof, that has been subjected to a protein refolding treatment.

The term “a protein refolding treatment” as used herein refers to one or more steps (executed either consecutively or not) wherein a soluble proteinaceous molecule is formed from, or obtained, isolated, or derived from, an insoluble proteinaceous molecule. As will be understood, an insoluble protein molecule may be formed in a cell by overexpression, misfolding, and other mechanisms by which insoluble protein formation is preferred over formation in or as a soluble component or fraction of a cell. Protein refolding processes to form or obtain a soluble protein from an insoluble proteinaceous entity will be understood by the skilled artisan. A protein refolding treatment may occur during expression in a cell (e.g., soluble expression into a cellular component), or during recovery of a protein particle from the cell once expression of a protein has been completed. For example, an insoluble proteinaceous molecule or insolubly formed proteinaceous molecule may be exposed to one or more solubilisation steps, possibly followed by a refolding step that may include removal of a denaturing agent (if used), to thereby obtain or produce a soluble form of the protein of interest. A solubilisation step may include exposing or contacting the insoluble form to one or more solubilising agents to at least partially denture the insoluble form. A solubilising agent may be denaturing, non-denaturing, or mildly solubilising. Non-limiting examples of solubilising agents include a detergent (e.g., a non-ionic detergent) or a chaotropic agent (e.g., guanidine hydrochloride or urea). Mild solubilising agents may preserve the native-like protein structures present in inclusion bodies and thus bypasses the refolding steps. A non-limiting example of a mild solubilising agent includes a low concentration of organic solvents like 5% n-propanol and DMSO and detergents like 0.2% N-lauroyl sarcosine. Combinations of solubilising agents and/or refolding methods are contemplated. Appropriate conditions (e.g., one or more parameters such as temperature, pH, and concentration of each agent, although without limitation thereto) will be understood and ascertainable to a skilled artisan. When a refolding step is contemplated after denaturation, a denaturing agent may be removed from the solubilised entity by any suitable technique such as dialysis, ultrafiltration, microfluidic chip technology, enzyme-mediated refolding (e.g., urease-mediated refolding), chromatography (e.g., on-column chromatography such as, but not limited to, gel filtration, liquid chromatography, affinity chromatography), dilution, and centrifugation, although without limitation thereto. Use of one or more additives to aid refolding is contemplated that may improve yield and/or at least partially inhibit aggregation. Non-limiting examples of a suitable additive include an amino acid, a sugar, a polyhydric alcohol, low concentrations of chaotropic agents, a sulfobetaine, a substituted pyridine or pyrrole, and an acid substituted aminocyclohexane. Singh et al (2015) Microb Cell Fact. 14: 41 discusses non-limiting examples of suitable protein refolding techniques and is incorporated herein by reference. In some preferred embodiments, a protein refolding treatment may be performed during recovery of a protein particle from the cell once expression has been completed.

In some embodiments, the protein particle has not been subjected to a protein refolding treatment. Preferably, a protein particle derived from an insoluble component (suitably an inclusion body) has not been subjected to a protein refolding treatment. According to some embodiments, the protein particle may be derived, isolated, removed, prepared, produced, obtained, or otherwise removed from a cell wherein the protein particle has not been subjected to a protein refolding treatment.

In certain embodiments, the protein particle as described herein is not formed, prepared, produced, or otherwise assembled from a solubly expressed, or a solubly derived, CRM amino acid sequence. In some embodiments, the protein particle may be substantially free, or free, of a soluble CRM protein, or a solubly-formed CRM protein, or a fragment, variant, or derivative thereof. In other embodiments, the protein particle is not formed or assembled by one or more CRM proteins or CRM amino acid sequences that have been subjected to a protein refolding treatment after synthesis or production of a protein particle, and preferably recombinant expression. Preferably, the CRM protein, or a CRM amino acid sequence may be an amino acid sequence derived from, or corresponding to, a CRM197 protein, or a fragment, variant, or derivative thereof. A solubly expressed protein may be a protein in a soluble component, portion, fraction of a cell and not an insoluble component of a cell (e.g., inclusion body). A solubly derived protein may be a protein subjected to a protein refolding treatment, and more particularly a protein refolding treatment after expression of the protein.

In some embodiments, the protein particle described herein is not obtained, prepared, subjected to, or otherwise produced by a protein refolding treatment performed after protein expression, and suitably, recombinant protein expression. In some embodiments, the protein particle derived from a cell as herein described may be produced, obtained or prepared without being subjected to a protein refolding treatment. In certain embodiments, a protein particle as described herein may prepared, isolated, produced, removed, derived, or otherwise obtained from, or is, an insoluble component of a cell or an insoluble component preparation of a cell that has not been subjected to or substantially subjected to a protein refolding treatment. In some embodiments, a protein particle as described herein may prepared, produced, isolated, removed, derived, or otherwise obtained from, or is, an inclusion body or an inclusion body preparation that has not been subjected to or substantially subjected to a protein refolding treatment. In some embodiments, the protein particle may be an inclusion body that has not been subjected to a protein refolding treatment.

It will be appreciated that a protein particle as described herein may be an isolated protein particle. It is envisaged that a protein as described herein may be an isolated protein. In some embodiments, the protein particle may be isolated from a cell, or a component thereof as described herein. The protein particle may be isolated and/or purified as will be known to a skilled addressee, and according to some embodiments as described herein.

By “isolated” is meant present in an environment removed from a natural state or otherwise subjected to human manipulation. Isolated material may be substantially or essentially free from components that normally accompany it in its natural state, or may be manipulated so as to be in an artificial state together with components that normally accompany it in its natural state. Isolated material may be in recombinant, chemical synthetic, enriched, purified, and/or partially purified form.

As used herein, by “synthetic” is meant not naturally occurring but made through human technical intervention. In the context of synthetic proteins and nucleic acids, this encompasses molecules produced by recombinant or chemical synthetic and combinatorial techniques as are well understood in the art.

It will be appreciated that according to some embodiments of the invention, a protein particle and/or an isolated protein as described herein may be purified, and in particular may be purified from a cell. A protein particle and/or isolated protein may be substantially pure or substantially purified, and in some embodiments, may be substantially purified from a cell. In certain embodiments, the cell may be a host cell for recombinant protein expression of the protein particle and/or isolated protein. A purified or substantially purified protein particle and/or isolated protein as described herein may be suitable for use in a composition according to methods of the present invention.

By “purify”, “purified”, and “purification”, particularly in the context of protein purification, is meant enrichment of a protein or a protein particle and preferably a recombinant protein or a recombinant protein particle so that the relative abundance and/or specific activity of said protein or protein particle and preferably recombinant protein or recombinant protein particle is increased compared to that before enrichment. In some embodiments, “purity” may relate to at least about 50%, 60%, 65%, 70%, 75%, 80%, 85% and more preferably 90%, 95%, 96%, 98%, 99% and about 100% purity of a desired molecule.

The terms “substantially pure” or “substantially purified” as used herein describes a substance (inclusive of a proteinaceous material such as, but not limited to, a protein particle or an isolated protein) that has been separated from components (including contaminating materials) that naturally or normally accompany it. Typically, a substance is substantially pure when at least about 60% or 65%, preferably least about 70% or 75%, more preferably at least about 80% or 85%, even more preferably at least about 90%, and most preferably at least 95%, 96%, 97%, 98% or even 99% of the total material (by volume, by specific activity, by wet or dry weight, or by mole percent or mole fraction) is the material of interest.

In some embodiments, the protein particle and/or substantially insoluble protein particle as described herein is obtained, isolated, produced, derived, purified, or substantially purified, from an insoluble component, and/or substantially insoluble component, of a cell. According to some of these embodiments, the insoluble component and/or substantially insoluble component, of a cell may be an inclusion body as described herein. It will be appreciated that in some embodiments, the insoluble component and/or substantially insoluble component is formed by or from recombinant expression in a cell, and suitable recombinant protein expression in a cell.

It will be appreciated that purity of a target protein or a target protein particle as described herein, for example a CRM protein or a protein particle may be expressed or determined as the concentration or level of the total protein in a purified protein particle fraction.

Purity of a substance can be determined or assessed by any applicable method as would be known to a skilled artisan. By way of example, densitometric methods may be utilised, and may be particularly advantageous for determining protein purity. Mass spectrometry is another suitable technique. Other spectrometric methods such as UV-Vis spectrophotometry or colourimetric assays such as a Bradford Assays may be suitable. Size analysis based on electrophoresis or chromatographic techniques are envisaged. HPLC fluidic analysis (e.g., microfluidic diffusional sizing) or dynamic light scattering are other techniques that may be employed. Use of combinations of methods to determine purity, and in particular protein purity, are also contemplated.

As will be appreciated in light of the foregoing, in general embodiments, a protein particle, an isolated protein particle, an isolated protein, or isolated nucleic acid as described herein may be prepared by recombinant techniques.

The term “recombinant” as used herein refers to a molecule resulting from manipulation into a form not normally found in nature.

The term “recombinant” may be used herein to describe a nucleic acid molecule and means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” includes a molecule (such as, but not limited to, a protein) when produced by a cell, or in a cell-free expression system, in an altered amount or at an altered rate compared to its native state. A recombinant protein also encompasses a protein expressed by artificial recombinant means when it is within a cell, tissue or subject, e.g., in which it is expressed. The term “recombinant” as used with respect to a protein (inclusive of fragments, derivatives, or variants thereof) includes a protein (inclusive of fragments, derivatives, or variants thereof) produced by expression in a recombinant system, and suitably by a recombinant polynucleotide. Typically, a recombinant molecule is produced by recombinant DNA technology.

A recombinant protein, or fragments, derivatives, or variants thereof may be conveniently prepared by a person skilled in the art using standard protocols as for example described in Sambrook et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-2009), incorporated herein by reference, in particular Chapters 10 and 16; and CURRENT PROTOCOLS IN PROTEIN SCIENCE Eds. Coligan et al., (John Wiley & Sons, Inc. 1995-2009) which is incorporated by reference herein, in particular Chapters 1, 5 and 6. In producing the protein particles, proteins, or fragments described herein, recombinant molecular biology techniques can be utilized to produce DNA encoding the desired molecule. Recombinant methodologies required to produce a DNA encoding a desired protein are well known and routinely practiced in the art.

It is readily contemplated that any recombinant protein expression system may be used for the present invention such as bacterial, yeast, plant, insect cells, mammalian cell lines such as lymphoblastoid cell lines and splenocytes isolated from transformed host organisms such as humans and mice, and insect-based expression systems but is not limited thereto. It will be appreciated that the recombinant protein expression system employed may be chosen on the basis of suitability for expression of certain characteristics, such as expression levels or yield of protein or post-translational modifications for suitability of downstream applications (e.g., eukaryotic cells for post-translation modifications), although without limitation thereto. In some general embodiments, a recombinant expression system that promotes, enhances, or otherwise augments self-assembly of a protein particle is desirable. Non-limiting examples of suitable expression strains and systems for use in the present invention may be found in Ferrer-Miralles and Villaverde (2013) Microb Cell Fact. 12:113, which is incorporated herein by reference.

In some embodiments, recombinant protein expression occurs in cells of prokaryotic origin. Suitable host cells for recombinant protein expression are bacterial cells such as Escherichia coli (E. coli) (BL21 and various derivative strains thereof which have been optimised for certain applications, such as Rosetta and DE3, for example; K-12 and various derivatives thereof have also been optimised for selected application such as Origami and SHuffle T7), a Pseudomonas (e.g., Pseudomonas fluorescens and various derivative strains, and a Bacillus strain (e.g., a Bacillus subtilis, a Bacillus megaterium) and various derivative strains, and Corynebacterium diphtheriae and various derivative strains, a Lactococcus strain (e.g., a Lactococcus lactis) and derivative strains, although without limitation thereto. Preferably, the host cell is an endotoxin-free strain of E. coli. More preferably, the endotoxin-free E. coli strain is BL21 ClearColi (DE3).

In other embodiments, recombinant expression occurs in insect cells which are suited to recombinant expression e.g., cell lines derived from Spodoptera frugiperda, Sf9 and Sf21.

In other preferred embodiments, recombinant expression may occur in yeast cells such as a Saccharomyces sp (e.g., Saccharomyces cerevisiae), Hansenula polymorpha, Yarrowia lipolytica, Arxula adeninivorans, Kluyveromyces lactis, Schizosaccharomyces pombe, or a Pichia sp (e.g., Pichia pastoris), although without limitation thereto. Yeast may be particularly suitable for expression of proteins with post-translation modifications, such as glycosylation, although without limitation thereto. Preferably, the yeast cell is Pichia pastoris. A non-limiting overview of yeast expression systems in provided in Baghban et al. (2019) Mol Biotechnol., 61(5):365-384, which is incorporated herein by reference.

Expression in a continuous cell culture line is also contemplated. Such cell lines may be derived from a mammalian host (e.g, HEK293 cells, CHO cells, VERO cells), may be primary cell lines (e.g., hepatocytes), or immortalised cell lines. A person of skill in the art will appreciate which cell line is suitable in certain applications (e.g., when post-translation modifications are desired).

It will be appreciated that certain methods and uses may require a protein particle or a protein where potential toxicity has been reduced or minimised, and/or other certain properties are either at least partially avoided or facilitated. For example, it may be desirable to avoid an endotoxic response in humans and as such, the inclusion of endotoxins (also referred to lipopolysaccharide) may be undesirable in certain contexts. In such instances, high yields of protein expression may also be desirable. Accordingly, expression in cells and/or strains that are endotoxin-free may be used. Non-limiting examples include ClearColi BL21(DE3) (Lucigen), which is a genetically modified E. coli strain that includes a genetically modified lipopolysaccharide that does not cause an endotoxic response in human cells and in particular, disables the endotoxin signal that is normally part of the lipopolysaccharide while still retaining competency and protein expression capability, but the advantage of high yield by expression in E. coli is retained. This was accomplished by blocking the production of LPS from the precursor lipid IVA through the incorporation of seven genetic deletions (ΔgutQ ΔkdsD ΔlpxL ΔlpxM ΔpagP ΔlpxP ΔeptA). One additional compensating mutation (msbA148) enables viability in the presence of lipid IVA. In other illustrative examples, a host cell that facilitates protein folding may be utilised and non-limiting examples are Shuffle T7 or Origami E. coli. Origami and SHuffle T7 are particularly advantageous in forming disulphide bonds and thus biologically active protein.

To facilitate recombinant protein expression, an expression-enhancing tag or purification tag amino acid sequence may be included with a protein or amino acid sequence. That is, a genetic construct of the present invention may also include a fusion partner (an expression-enhancing tag or purification tag amino acid sequence; typically provided by a vector or an expression vector) so that the recombinant protein of interest is expressed as a fusion protein with said fusion partner. An advantage of fusion partners is that may assist identification and/or purification of said fusion protein. However, it will also be appreciated that the choice of fusion partner may also assist with protein properties such as (but not limited to) stability, and yield. A fusion partner may be added to an N- or C-termini of a protein or amino acid sequence, or may be added within the interior of the protein or amino acid sequence. An addition at a terminus may be directly adjacent to the terminus or there may be a spacer between the fusion partner sequence and the start of the other amino acid sequence.

A “purification tag amino acid sequence” is any amino acid sequence that is specifically fused to or associated with a second amino acid sequence to assist with purification, and in particular chromatographic purification (and more suitably, affinity chromatography) of a protein, peptides etc. The term may also be referred to as a “purification tag molecule”. Non-limiting examples of a purification tag include Protein A, glutathione S-transferase (GST), green fluorescent protein (GFP) maltose-binding protein (MBP), hexahistidine (HISe) and epitope tags such as V5, FLAG, haemagluttinin and c-myc tags. Inclusive of such sequences are sequences which specifically allow cleavage of the fusion partner from the other partner sequence, as normal protein engineering approaches would tend to incorporate a method for removal of the purification tag. An “expression enhancing sequence” is any amino acid sequence which aids with the recombinant expression of proteins and includes SUMO protein or fragments thereof.

A fusion partner sequence may facilitate a fusion protein binding to an affinity matrix to enable protein purification and/or detection. For the purposes of fusion polypeptide purification by affinity chromatography, relevant matrices for affinity chromatography are antibody, protein A- or G-, glutathione-, amylose-, and nickel- or cobalt-conjugated resins respectively. Many such matrices are available in “kit” form, such as the QIAexpress™ system (Qiagen) useful with (HISe) fusion partners and the Pharmacia GST purification system. In many cases, the fusion partner can be cleaved by an appropriate protease or chemical reagent to release the protein of interest from the fusion partner.

In some embodiments, it may be desirable to force, drive, or promote expression of a protein into an insoluble component. A purification-tag such as a HIS6 (but not limited thereto) in a protein may facilitate solubility of the protein. The absence of a purification-tag or expression-enhancing tag may facilitate expression into an insoluble component of a cell. In certain embodiments, it may be desirable to decrease the solubility of a recombinantly produced protein, to thus increase the expression of the protein in an insoluble compartment (and more preferably an inclusion body) of a host cell. According to some embodiments, it may be desirable not to include a purification-tag and/or expression-enhancing tag to an amino acid sequence for expression of a protein as described herein. In certain embodiments, proteins as described herein are expressed in the absence of a fusion partner sequence, and preferably the absence of a purification-tag amino acid sequence and/or expression-enhancing tag amino acid sequence. Preferably, a CRM amino acid sequence, or fragments, variants, and derivatives thereof, does not comprise a purification-tag amino acid sequence and/or expression-enhancing tag amino acid sequence. More preferably, an amino acid sequence derived from, or corresponding to, a CRM197 protein, or fragments, variants, and derivatives thereof, does not comprise a purification-tag amino acid sequence and/or expression-enhancing tag amino acid sequence. It will be appreciated that in some embodiments, the absence of a fusion partner sequence in a protein particle may also be desirable for one or more other parameters, functions, or effects such as, but not limited to, protein size and/or molecular weight, downstream processing of a recombinant protein (e.g., avoiding requirement to remove a fusion partner sequence from an expressed protein), and/or potential immunogenicity issues arising from a fusion partner sequence (e.g., potential for a fusion partner sequence to elicit an unwanted immune response).

Protein expression may be augmented, improved, or increased by codon optimisation techniques as are known in the art. Codon optimisation may take into a consideration a variety of factors involved in different stages of protein expression, such as codon adaptability, mRNA structure, and various cis-elements in transcription and translation, although without limitation thereto. Codon optimisation may be employed where it is desirable to promote strong expression of a protein of interest. In such instances, codon optimisation and strong expression resulting therefrom may shift the balance towards inclusion body formation in a recombinant system. As such, the present invention also contemplates nucleic acids that have been modified such as by taking advantage of codon sequence redundancy. In a more particular example, codon usage may be modified to optimize expression of a nucleic acid in a particular organism or cell type. As an illustrative example, the nucleic acid sequence as set forth in SEQ ID NO:1 has been codon optimised for expression of a CRM197 protein in E. coli. Such methodology may employ a reference sequence, and in particular the reference sequence may be a wild-type or native sequence. The invention also contemplates use of modified purines (for example, inosine, methylinosine and methyladenosine) and modified pyrimidines (for example, thiouridine and methylcytosine) in nucleic acids of the invention.

The protein particle may be an isolated protein particle produced, formed, prepared, or expressed in a suitable manner, and preferably a recombinant system. In some embodiments, the protein particle may be an isolated recombinant protein particle derived from a cell. In other embodiments, the isolated recombinant protein particle, or the protein particle may be substantially purified or substantially pure.

In light of the foregoing it would be readily appreciated that the present invention contemplates isolated nucleic acids encoding isolated proteins, or fragments thereof.

The term “nucleic acid” as used herein designates single- or double-stranded mRNA, RNA, cRNA and DNA inclusive of cDNA, genomic DNA and DNA-RNA hybrids. Nucleic acids may also be conjugated with fluorochromes, enzymes and peptides as are well known in the art.

The term “gene” is used herein to describe a discrete nucleic acid locus, unit or region within a genome that may comprise one or more of introns, exons, splice sites, open reading frames and 5′ and/or 3′ non-coding regulatory sequences such as a polyadenylation sequence.

As used herein, “wild-type” or “native” or “naturally occurring” sequences, refers to nucleic acid sequences or polypeptide encoding sequences that are essentially as they are found in nature.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

It will be well appreciated by a person of skill in the art that the isolated nucleic acids of the invention can be conveniently prepared by a person of skill in the art using standard protocols such as those described in Chapter 2 and Chapter 3 of CURRENT PROTOCOLS IN MOLECULAR BIOLOGY (Eds. Ausubel et al. John Wiley & Sons NY, 1995-2008). Furthermore, codon optimisation techniques are also well known in the art, and may be undertaken by computer algorithms such as, but not limited to, OptimumGene by GenScript. These algorithms may incorporate computer modelling.

In one particular embodiment, an isolated nucleic acid of the present invention is operably-linked to one or more regulatory nucleotide sequences in a genetic construct. A person skilled in the art will appreciate that a genetic construct is a nucleic acid comprising any one of a number of nucleotide sequence elements, the function of which depends upon the desired use of the construct. Uses range from vectors for the general manipulation and propagation of recombinant DNA to more complicated applications such as prokaryotic or eukaryotic expression of the isolated nucleic acid. Typically, although not exclusively, genetic constructs are designed for more than one application. By way of example only, a genetic construct whose intended end use is recombinant protein expression in a eukaryotic system may have incorporated nucleotide sequences for such functions as cloning and propagation in prokaryotes in addition to sequences required for expression. A consideration when designing and preparing such genetic constructs are the required nucleotide sequences for the intended application. In view of the foregoing, it is evident to a person of skill in the art that genetic constructs are versatile tools that can be adapted for any one of a number of purposes. Methods for the generation of said genetic constructs are well known to those of skill in the art.

In a preferred embodiment, the genetic construct is an expression construct which is suitable for recombinant expression. Preferably, the expression construct comprises at least a promoter and in addition, one or more other regulatory nucleotide sequences which are required for manipulation, propagation and expression of recombinant DNA. In particular aspects, the invention contemplates an expression construct comprising an isolated nucleic acid, operably-linked to one or more regulatory nucleotide sequences in an expression vector. A person skilled in the art will appreciate that the isolated nucleic acid may be inserted into the expression vector by a variety of recombinant techniques using standard protocols as for example described in Sambrook et al., MOLECULAR CLONING, A Laboratory Manual (Cold Spring Harbor Press, 1989), which is incorporated herein by reference. An “expression vector” may be either a self-replicating extra-chromosomal vector such as a plasmid, or a vector that integrates into a host genome, inclusive of vectors of viral origin such as adenovirus, lentivirus, poxvirus and flavivirus vectors as are well known in the art. By “operably linked”” is meant that said regulatory nucleotide sequence(s) is/are positioned relative to the recombinant nucleic acid of the invention to initiate, control, regulate or otherwise direct transcription and/or other processes associated with expression of said nucleic acid. Preferable vectors include any of the well-known prokaryotic expression vectors, recombinant baculoviruses, COS cell specific vectors, vaccinia recombinants, or yeast-specific expression constructs. Among expression vectors preferred for use in cells of prokaryotic origin include pQE60 available from Qiagen, pGEX series of vectors available from GE Life Sciences and pET vector system available from Novagen.

Regulatory nucleotide sequences will generally be appropriate for the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences for secretion of a translated protein, ribosomal binding sites, transcriptional start and termination sequences, translational start and termination sequences, splice donor/acceptor sequences, enhancer or activator sequences and nucleic acid packaging signals. Preferably, said promoter is operable in a prokaryotic cell. Non-limiting examples include T7 promoter, tac promoter and T5 promoter. Inducible/repressible promoters (such as tet-repressible promoters and IPTG-, alcohol-, metallothionine- or ecdysone-inducible promoters) are well known in the art and are contemplated by the invention, as are tissue-specific promoters such as α-crystallin promoters. It will also be appreciated that promoters may be hybrid promoters that combine elements of more than one promoter (such as SRα promoter).

The expression construct may also include a fusion partner (typically provided by the expression vector) so that the protein (or fragment thereof) of the invention is expressed as a fusion protein with said fusion partner, as hereinafter described.

In some embodiments, the present invention contemplates a chimeric protein particle or a chimeric protein.

A “chimera” or a “chimera” gene, nucleic acid, protein, peptide or polypeptide is meant a gene, nucleic acid, protein, fragment or polypeptide that comprises two or more genes, nucleic acids, proteins, amino acid sequence, fragments or polypeptides not normally associated together. A “chimera” includes within its scope a fusion between fragments, and may be referred to herein a fusion partner. Typically, although not exclusively, the chimera is a fusion between unrelated sequences however it is readily contemplated that the sequences may be homologues. One or more preferred embodiments of the present invention relate to a chimeric protein comprising a CRM amino acid sequence and one or more immunogenic amino acid sequences derived from, or corresponding to, one or more immunogens or proteins of interest, and a protein particle including or formed from said chimeras. The, or each, immunogen in a chimera may be derived from the same or different agents, proteins, molecules, or sources. By way of example only, one or more immunogens in a chimera contemplated by the invention may be from the same pathogen or a plurality of different pathogens, and without limitation thereto. It will be appreciated that the chimeric proteins may include other amino acid sequences as herein described. In certain embodiments, a chimera (inclusive of a chimeric protein) as described herein is formed from or produced by recombinant DNA technology, and may suitably expressed in a host organism (e.g., E. coli but not limited thereto) suitable for recombinant expression.

A chimera or a fusion as described herein may be a protein comprising at least two sequences of interest that are encoded by separate genes that have been joined so that they are transcribed and translated as a single unit, producing a single polypeptide. Alternatively, expression may be in the form of a chimera in which each sequence of interest is expressed a distinct and single polypeptide.

It is envisaged that in some embodiments, a protein particle as described herein may be substantially formed, assembled, prepared from a chimeric protein, or formed, assembled, or prepared from a chimeric protein. In some embodiments, the protein particle as described herein that is substantially formed, or formed, from a chimeric protein may a substantially insoluble protein particle as described herein. In some embodiments, the protein particle comprising a diphtheria toxin CRM amino acid sequence and/or the substantially insoluble protein particle derived, obtained, or produced from a chimeric protein may be derived from an insoluble component of a cell, wherein the insoluble component of the cell has not been subjected to a protein refolding treatment. In certain embodiments, the insoluble component is an inclusion body formed in the cell.

Where additions at termini contemplated herein, such additions may be directly adjacent to the terminus (i.e., contiguous between the last nucleotide of the terminus sequence and the first nucleotide of the added sequence). Alternatively, there may be a spacer sequence between a first sequence (e.g., a CRM197 amino acid sequence) and a second sequence (e.g., an immunogenic sequence), such as a spacer sequence generated by a restriction enzyme site although without limitation thereto. It will be appreciated that it is envisaged that a third, fourth, fifth, or more sequence may be included in such an arrangement, and in particular two or more of said sequences can be derived from the same source or different sources.

It is envisaged that an amino acid sequence of an immunogen other a diphtheria toxin CRM amino acid sequence, or a fragment thereof, may be included in any relationship to the CRM amino acid sequence so as long as the resultant molecule/s is able to form a protein particle as described herein, and preferably induce a desired activity. Accordingly, the one or more immunogenic amino acid sequences may be positioned at, adjacent, or near, an N and/or C terminus of a CRM amino acid sequence. In certain preferred embodiments, an immunogenic amino acid sequence may be positioned at, adjacent, or near a C terminus of a CRM amino acid sequence. The amino acid sequence of an immunogen other than a CRM amino acid sequence may be within the CRM amino acid sequence if suitable to produce a suitable protein particle for use in the methods described herein.

In some embodiments that relate to a Mycobacterium, a chimera, fusion, or chimeric molecule may comprise, consist essentially of, or consist of, or is, an amino acid sequence as set forth in SEQ ID NOS:19 and/or 20, or a fragment, variant or derivative thereof.

In some embodiments that relate to a Streptococcus, a chimera, fusion, or chimeric molecule may comprise, consist essentially of, or consist of, or is an amino acid sequence as set forth in any one of in SEQ ID NO:65, SEQ ID NO:66, SEQ ID NO:67, and/or SEQ ID NO:68, or a fragment, variant or derivative thereof, and any combination thereof.

In some embodiment that relate to a coronavirus, a chimera, fusion, or chimeric molecule may comprise, consist essentially of, or consist of, or is an amino acid sequence as set forth in SEQ ID NO:101, or a fragment, variant or derivative thereof, and any combination thereof.

It is envisaged that in some embodiments, a protein particle may be derived from a plurality of chimeric proteins comprising one or a plurality of immunogens.

In light of the description herein, it will be appreciated that the present invention also contemplates isolated proteins and, protein particles comprising one or more immunogenic amino acid sequences (e.g., an entire protein sequence or a fragment sequence) wherein said sequences or fragments may be present singly or as repeats, which also includes tandemly repeated sequences or fragments. “Spacer” amino acids may also be included between one or the plurality of the immunogenic sequences or fragments. Such arrangement may be suitable to elicit, modulate, or augment an immune response, although without limitation thereto.

The term “foreign” or “exogenous” or “heterologous” refers to any molecule (e.g., a polynucleotide or polypeptide) which is introduced into a host by experimental manipulations and may include gene/nucleic acid sequences found in that host so long as the introduced gene contains some modification (e.g., a point mutation, the presence of a selectable marker gene, the presence of a recombination site, etc.) relative to the naturally-occurring gene.

Alternatively, protein particles according to the present invention can be produced by co-expressing two or more separate chimeric proteins one of which has a particle-forming component linked to a first sequence of interest and one which has a particle-forming component linked to a second sequence of interest, and a further chimeric protein which has a particle-forming component linked to a third sequence of interest etc. Suitably, the particle-forming component is a CRM amino acid sequence, and preferably, a CRM197 amino acid sequence.

As will be appreciated, the methods and compositions as described herein may use a protein particle as described herein which includes an immunogen wherein the immunogen is formed with the particle in a cell by, for example, recombinant expression. Alternatively, the immunogen is linked to the protein particle after the particle has been produced. For example, once a protein particle comprising a CRM197 amino acid sequence has been prepared from a suitable host, the protein particle can be conjugated to a target immunogen. The immunogen other than a CRM amino acid sequence may be conjugated to the protein particle by any of the several methods known in the art (see, e.g., Bioconjugate Techniques, Greg. T. Hermanson Ed., Academic Press, New York. 1996; Farkaš and Bystrický (2010) Chemical Papers. 64(6): 683-695; and Spicer and Davis (2014) Nature Communications 5: 4740, each of which is incorporated herein by reference). For example, protein-protein (i.e. protein particle-immunogen other than CRM197) conjugation could be carried by using sulfo-SMCC linkers (sulfosuccinimidyl esters) for conjugation using standard protocols.

The present invention encompasses protein ligation (may also referred to as “bioconjugation”) techniques for production or generation of a protein particle or a protein that includes an immunogenic amino acid sequence. Protein ligation techniques may be used to create covalently stabilised fusion molecules. As such, protein ligation techniques to couple at least one recombinant protein and a desired partner molecule are contemplated by the present invention. Protein ligation is particularly amenable for use with at least two recombinant proteins that would otherwise be restrictive or impossible with traditional direct genetic fusion between the two proteins, although without limitation thereto.

A protein ligation system that facilitates a spontaneous formation of an irreversible covalent link between at least two proteins, or at least one protein and another agent is contemplated. By way of example, systems which utilise the characteristics of bacterial pilins and adhesins in the form of an affinity or inherent formation of an intramolecular isopeptide bond, to form a peptide interaction. Reference is made to Veggiani et al., (2014) Trends Biotechnol. October; 32(10):506-12 which describes such systems generally and non-limiting examples thereof, and is incorporated herein by reference.

A non-limiting example of a suitable protein ligation system based on this technology is the SpyTag/SpyCatcher system, which uses a modified domain from a Streptococcus pyogenes surface protein (SpyCatcher) that naturally recognizes a cognate 13-amino-acid peptide (SpyTag; AHIVMVDAYKPTK; SEQ ID NO:8). A SpyCatcher protein and a SpyTag protein are derived from the CnaB2 domain of the fibronectin-binding protein FbaB from Streptococcus pyogenes. CnaB2 was initially split into peptide and protein partners, surface-exposed hydrophobic residues were removed, and interactions at the binding interface were enhanced. This process generated the optimized 13-residue SpyTag and 116-residue SpyCatcher binding partners. Upon recognition, a SpyTag and a SpyCatcher form a covalent isopeptide bond between the side chains of a lysine in SpyCatcher and an aspartate in SpyTag. By way of example in the context of the present invention, a first amino acid sequence (e.g., a CRM amino acid sequence or fragment thereof) can be engineered to include a SpyCatcher protein whilst a second amino acid sequence (e.g., an immunogen (such as, but not limited to, an immunogen derived from a virus)) that may be produced in a glycosylated form in a suitable expression such as yeast, although without limitation thereto) can be engineered to include a SpyTag. Upon exposure of the so modified first and second proteins, the proteins form an irreversible covalent linkage through a SpyCatcher and a SpyTag pairing to thus form a spontaneous protein ligation event. This example is illustrative only. Reddington and Howarth (2015) Current Opinion in Chemical Biology, 29: 94-99, and Hatlem et al (2019) Int. J. Mol. Sci., 20: 2129, International Publication Numbers WO2011/098772, WO 2016/193746, and WO/2018/197854 each provide a non-limiting description of a SpyCatcher/SpyTag system and a method therefor, each of which is incorporated herein by reference. Further non-limiting examples of a suitable protein ligation system include Isopeptag, a peptide (TDKDMTITFTNKKDAE; SEQ ID NO:9) which binds covalently to a pilin-C protein of Streptococcus pyogenes; SnoopTag-SnoopCatcher developed from a Streptococcus pneumoniae pilin wherein SnoopTag, a peptide (KLGDIEFIKVNK; SEQ ID NO:10) which binds covalently to a SnoopCatcher protein; SnoopTagJr (KLGSIEFIKVNK; SEQ ID NO:11) which to binds to either a SnoopCatcher protein or a DogTag protein (mediated by SnoopLigase); DogTag, a peptide (DIPATYEFTDGKHYITNEPIPPK; SEQ ID NO:12) which covalently binds to SnoopTagJr, mediated by SnoopLigase; and SdyTag, a peptide (DPIVMIDNDKPIT; SEQ ID NO:13) which binds covalently to a SdyCatcher protein. A person of skill in the art will understand that a plurality of protein ligation systems as herein described may be used to generate a protein of interest. Method to incorporate these sequences by recombinant DNA technology will be known and routine to a person of skill in the art.

In those embodiments which contemplate fragments, peptides, said fragments, peptides may be in the form of fragments, peptides prepared by chemical synthesis, inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2009). In this regard, reference is also made to International Publication WO 99/02550 and International Publication WO 97/45444.

Typically, a protein and/or an amino acid sequence (inclusive of fragments, variants, or derivatives) from which a protein particle is derived may be expressed (e.g., by recombinant expression) under conditions wherein the protein particles are otherwise formed, produced, assembled, or expressed in a host cell e.g., as an aggregate, as an inclusion body, or a structured assembly. It is also envisaged that a lower order protein particle may first be isolated from the host cell and incubated under conditions which permit self-assembly into higher order protein particles.

A further non-limiting advantage of the present invention may include formation in a cell of a protein particle as described herein which may act as an immunogenic agent and/or carrier agent, and due, at least in part, to formation of the particle in a cell, the particle can be recovered, isolated, or prepared using conventional techniques such as washing, centrifugation, chromatography, sedimentation and filtration (and combinations thereof), although without limitation thereto. As such, intracellular protein particle formation may avoid one or more downstream processing steps/parameters, such as, but not limited to, protein concentration, pH adjustment, temperature, ionic strength, and addition of specific solvent ingredients, particularly (although not exclusively) when compared to in vitro particle formation. In some embodiments, the CRM amino acid sequence is derived from, or corresponds to, a CRM197 protein, or a fragment, variant, or derivative thereof, or one or more other CRM proteins as herein described.

It is envisaged that isolation and/or purification of a protein particle as described herein, particularly recombinantly produced protein particles, or recombinant proteins, can be carried out by methods known in the art including, but not limited to, ion exchange chromatography, gel filtration, size-exclusion chromatography, size-fractionation, sedimentation (e.g., centrifugation), washing, and affinity and immunoaffinity chromatography. Combinations of methods are also contemplated.

By “chromatography” such as in the context of chromatographic steps of the invention, is meant any technique used for the separation of biomolecules (e.g., protein and/or nucleic acids) from complex mixtures that typically employs at least two phases: a stationary bed phase and a mobile phase that moves through the stationary bed. Molecules may be separated on the basis of a particular physicochemical property such as charge, size, affinity and hydrophobicity, or a combination thereof.

Chromatography may be performed by a person skilled in the art using standard protocols as for example described in CURRENT PROTOCOLS IN PROTEIN SCIENCE Eds. Coligan et al., (John Wiley & Sons, Inc. 1995-1999) which is incorporated by reference herein, in particular Chapters 8 and 9.

A person of skill in the art will be able to ascertain appropriate methodology for protein particle or protein isolation and/or purification. By way of example, intracellularly produced protein particles may be obtained, derived, removed, purified, or isolated from a prepared cell lysate. The cell lysate may be prepared by subjecting a host cell to disruption by a suitable technique (e.g., mechanical or homogenisation disruption to lyse the cells by sonication, or a high-pressure treatment). Some cells may require additional agents or treatments to disrupt the cell wall e.g., yeast cells, as will be known by the skilled addressee. The disrupted cells may be separated into a supernatant and a pellet, typically by centrifugation. According to this exemplary embodiment, the target molecules in the form of protein particles are present in the pellet. The supernatant includes soluble protein and may be removed, disregarded, or disposed of during protein particle preparation. The pelleted protein particles may then be washed in an appropriate buffer to remove contaminating material or impurities. A typical washing step may include resuspension of the pelleted protein particle in a solution (e.g., a buffer), followed by separation of the pellet and the supernatant by centrifugation. The pellet may further be subjected to a resuspension step in a wash buffer. The process may include a homogenisation treatment of the pelleted suspension after each washing step in order to disperse the protein particle in solution. A homogenisation treatment may include a means to disperse the protein particle in solution. The homogenisation means may be a physical treatment (e.g., sonication, high pressure homogenisation) or a chemical treatment (e.g., use of a dispersion agent such as a detergent), although without limitation thereto. Inclusion of the homogenisation treatment with washing may aid with obtaining a particle suspension, and in particular a homogenous particle suspension. Multiple washing steps are contemplated. After the final washing step (and optionally a homogenisation treatment), the pelleted material which includes the protein particle may then be resuspended in an appropriate solution or buffer. The resulting protein particle preparation may be subjected to further steps such as chromatography. In some embodiments, the protein particle preparation may include one or more washing steps.

In some embodiments, the protein particle as described herein may be derived, obtained, produced, prepared, or otherwise removed from, or is, an isolated and/or purified (or may be substantially purified) protein particle.

In some embodiments, the protein particle as described herein may be derived, obtained, produced, prepared, or otherwise removed from, or is, an isolated and/or purified (or may be substantially purified) insoluble component, portion, or fraction of a cell. Suitably, an isolated and/or purified (or substantially purified) insoluble component, portion, or fraction of a cell may be an inclusion body.

The present invention includes fragments, variants and derivatives of an isolated protein, an immunogenic amino acid sequence, an immunogen, a CRM protein, or a CRM amino acid sequence.

A “fragment” as used herein is a segment, domain, portion or region of a protein or peptide (such as the sequences set out in the Examples, Tables, and Figures, or other immunogens) which constitutes less than 100%, but at least 20%, 30%, 40%, 50%, 60%, 70%, 80% or 90%, 92%, 94%, 96%, 98%, or 99% of the entire protein or peptide. In an example, fragments of a CRM protein are contemplated, wherein the CRM protein may comprise, consist of, or consist essentially of, of an amino acid sequence as set forth in any one of SEQ ID NO:2, SEQ ID NOS: 23 to 26, and/or SEQ ID NOS:49-54, although without limitation thereto. The present invention encompasses fragments of any one of the sequences disclosed herein inclusive of an amino acid sequence as set forth in any one of SEQ ID Nos:6, 7, 17-22, 28-48, and 56-104, although without limitation thereto. It will be appreciated that the fragment may be a single fragment or may be repeated alone or with other fragments. As such, it will also be appreciated that larger peptides and isolated proteins comprising a plurality of the same or different fragments are contemplated. Suitably, the fragment is an immunogenic fragment.

In particular embodiments, a protein fragment may comprise, for example, at least 5, 10, 20, 30, 40, 50 60, 70, 80, 90, 100, 120, 140, 150, 200, 250, 300, 350, 400, 450, 500, 510, 520, or 530 contiguous amino acids of a protein.

In some embodiments, a fragment of a CRM protein may comprise, for example, a region corresponding to a Catalytic Domain C (amino acids 1-190 of Fragment A of diphtheria toxin as described herein, or as set out in SEQ ID NO:55), a Transmembrane Domain T (amino acids 201-384), and/or Receptor Binding domain R (amino acid 386-535) of the corresponding region in wild-type or native diphtheria toxin. For example, in some embodiments that relate to a CRM197 protein, a fragment of CRM197 may comprise a region corresponding to Fragment A only, or Fragment A and Transmembrane Domain T, although without limitation thereto. In some further exemplary embodiments, a fragment of a CRM197 protein may comprise, consist essentially of, consist of, or is, amino acid residues 1-190 of a CRM197 protein with reference to or as set forth in SEQ ID NO:50, and/or may comprise, consist essentially of, consist of, or is, amino acid residues 1-389 of a CRM197 protein with reference to or as set forth in SEQ ID NO:50.

Protein fragments may be obtained through the application of standard recombinant nucleic acid techniques or synthesized using conventional liquid or solid phase synthesis techniques, such as those described herein. Alternatively, peptides can be produced by digestion of an isolated protein of the invention with proteases such as endoLys-C, endoArg-C, endoGlu-C and staphylococcus V8-protease. The digested fragments can be purified by, for example, high performance liquid chromatographic (HPLC) techniques as are well known in the art.

The present invention encompasses variants of a protein, an amino acid sequence, or a protein fragment.

In the context of the specification, a protein “variant” is distinguished from a reference sequence by the deletion, or substitution of one or more amino acid residues. The reference amino acid sequence may be an amino acid sequence as set forth in any one of the Examples, Table, and/or Figures herein. In some embodiments, the reference amino acid sequence may be an amino acid sequence as set forth in any one of SEQ ID NO:2, SEQ ID NO:6, SEQ ID NO:7, and SEQ ID NO:17-104, for example, although without limitation thereto. The “variant” may have one or a plurality of amino acids of the reference amino acid sequence deleted or substituted by different amino acids. “Variants” include within their scope naturally-occurring variants such as allelic variants, orthologs and homologs and artificially created mutants, for example. The term “mutant” may also be used to describe a variant. It will be well understood by a person of skill in the art that some amino acids may be substituted or deleted without changing the activity of the protein, or a fragment thereof (conservative substitutions). In some embodiments, protein variants may share at least 50%, 55%, 60%, 65%, 70% or 75%, preferably at least 80% or 85% or more preferably at least 90%, 91%, 92%, 93%, 94%, 95%, 96%, 97%, 98%, or 99% sequence identity with a reference amino acid sequence.

A variant may include a substitution or deletion of one or more amino acid residues that alter or modulate one or more properties or activities of a reference polypeptide. By way of example, it may be desirous in certain embodiments to include a variant of an immunogen that corresponds to a pathogen-escape mutant that may be evolved to escape or evade a host cell immunity.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FAST A, and TFASTA in the Wisconsin Genetics Software Package Release 7.0, Genetics Computer Group, 575 Science Drive Madison, Wis., USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25: 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

As used herein, the term “conservative substitution” refers to amino acid substitutions which would not negatively affect or change the essential characteristics of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, with a residue similar to the corresponding amino acid residue physically or functionally (such as, having similar size, shape, charges, chemical properties including the capability of forming covalent bond or hydrogen bond, etc.). The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, an amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl Acad. Set USA 94: 412-417 (1997).

It will also be understood that non-conservative substitutions are contemplated by the present invention as would be required by the context of use. Generally, non-conservative substitutions which are likely to produce the greatest changes in protein structure and function are those in which (a) a hydrophilic residue (e.g. Ser or Thr) is substituted for, or by, a hydrophobic residue (e.g. Ala, Leu, Ile, Phe or Val); (b) a cysteine or proline is substituted for, or by, any other residue; (c) a residue having an electropositive side chain (e.g. Arg, His or Lys) is substituted for, or by, an electronegative residue (e.g Glu or Asp) or (d) a residue having a bulky hydrophobic or aromatic side chain (e.g. Val, Ile, Phe or Trp) is substituted for, or by, one having a smaller side chain (e.g. Ala, Ser) or no side chain (e.g Gly).

With regard to protein variants and in particular those which are artificially-created mutants, these can be created by mutagenising a protein or by mutagenising an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra which is incorporated herein by reference. Site-directed mutagenesis techniques are well known in the art. Non-limiting examples of suitable commercial kits include Phusion Site-Directed Mutagenesis Kit (ThermoFisher Scientific), QuikChange II (Agilent) and Q5 Site-Directed Mutagenesis Kit (New England Biolabs).

It will be appreciated by the skilled person that site-directed mutagenesis may be performed where knowledge of the amino acid residues that contribute to biological activity is available. In some cases, this information is not available, or can only be inferred by molecular modelling approximations, for example.

In such cases, random mutagenesis is contemplated. Random mutagenesis methods include chemical modification of proteins by hydroxylamine (Ruan et al., 1997, Gene 188: 35), incorporation of dNTP analogs into nucleic acids (Zaccofo et al., 1996, J. Mol. Biol. 255: 589) and PCR-based random mutagenesis such as described in Stemmer, 1994, Proc. Natl. Acad. Sci. USA 91 10747 or Shafikhani et al., 1997, Biotechniques 23 304, each of which references is incorporated herein. It is also noted that PCR-based random mutagenesis kits are commercially available, such as the Diversify™ kit (Clontech).

It will be appreciated that changes to a protein variant can arise either spontaneously or by manipulations by man, by chemical energy (e.g., X-ray), or by other forms of chemical mutagenesis as will be known in the art.

The invention contemplates derivatives of a proteinaceous molecule.

As used herein, “derivatives” are molecules such as proteins, fragments, or variants thereof that have been altered, for example by complexing with other chemical moieties, by post-translational modification (e.g. phosphorylation, acetylation and the like), modification of glycosylation (e.g. adding, removing or altering glycosylation), lipidation and/or inclusion of additional amino acid sequences as would be understood in the art.

Additional amino acid sequences may include fusion partner amino acid sequences which create a fusion protein as described herein. By way of example, fusion partner amino acid sequences may assist in detection and/or purification of the isolated fusion protein. Non-limiting examples include metal-binding (e.g., polyhistidine) fusion partners, maltose binding protein (MBP), Protein A, glutathione S-transferase (GST), fluorescent protein sequences (e.g. GFP), epitope tags such as myc, FLAG and haemagluttinin tags. Other derivatives contemplated by the invention include, but are not limited to, modification to side chains, incorporation of unnatural amino acids and/or their derivatives during peptide, or protein production, amino acid analogs having variant side chains with functional groups (such as, for example, canavanine, norleucine, homoserine, 3-phosphoserine, b-cyanoalanine, and 1- or 3-methylhistidine), and other methods which impose conformational constraints on the proteins, fragments and variants as described herein. Also contemplated are peptidomimetics of a protein, as would be understood in the art. In this regard, the skilled person is referred to Chapter 15 of CURRENT PROTOCOLS IN PROTEIN SCIENCE, Eds. Coligan et al. (John Wiley & Sons NY 1995-2008) for more extensive methodology relating to modification of proteins.

As would be appreciated by a person of skill in the art, fragments, variants, or derivatives may be produced with the aim of improving one or more properties of a protein of interest such as immunogenicity, side effects or toxicity profile, pharmacodynamics and/or pharmacokinetics, although without limitation thereto. Such methods will be readily understood by a person of skill in the art.

In general embodiments, the fragment, variant, or derivative is a “biologically-active” fragment, variant or derivative, which retains biological, structural and/or physical activity of a given protein, or an encoding nucleic acid. In some embodiments, the biologically-active fragment, variant or derivative has not less than 10%, preferably no less than 25%, more preferably no less than 50%, and even more preferably no less than 75%, 80%, 85%, 90%, or 95% of the desired activity of the parent molecule. It will be understood that such activity may be evaluated using standard testing methods and bioassays recognisable by the skilled artisan in the field as generally being useful for identifying such activity. In some embodiments a biologically-active fragment, variant, or derivative may have the capability or ability to form a protein particle inside a cell or self-assemble into a protein particle inside a host cell. A biologically-active fragment may be a fragment of a CRM protein that is capable of forming a protein particle in a cell or capable of self-assembly in a cell.

In other embodiments, the “biologically-active” fragment, variant, or derivative is an immunogenic fragment, variant, or derivative. In the context of the present invention, the term “immunogenic” as used herein indicates the ability or potential to generate or elicit an immune response to an agent (such as, but not limited to, a pathogen or molecular components thereof) upon administration of the immunogenic agent (such as a protein particle, protein, fragment, variant or derivative) to a subject. Accordingly, in some embodiments a fragment, variant, or derivative and in particular an immunogenic fragment, variant, or derivative, may comprise at least one T-cell epitope and/or at least one B-cell epitope. Preferably, the immune response elicited by the immunogenic fragment, variant, or derivative may be a protective immune response as described herein.

It will be appreciated that a protein particle as described herein may comprise a CRM amino acid sequence as a “backbone” or “scaffold” in combination with the following non-limiting examples: (a) a single immunogen derived from a single source, agent, or molecule; (b) one or a plurality of different immunogens derived from the same source, agent, or molecule; or (c) one or a plurality of immunogens of or from each of a plurality of different sources, agents, or molecules. The present invention is also readily amenable to production and/or administration of a mixed population of protein particles to thereby produce a multivalent therapeutic, immunogenic, immunotherapeutic, and/or antigen or delivery system or compositions, for example. By way of example only, a first protein particle may comprise a plurality of immunogenic amino acid sequences other than a CRM sequence derived from, or corresponding to, the same or different viral immunogen co-administered with a second, third, fourth, or more, protein particle comprising one or a plurality of immunogenic amino acid sequences derived immunogen derived from a parasite and/or a bacterium. By way of further example, a single protein particle may comprise a plurality of immunogenic amino acid sequences other than a CRM sequence wherein the, or each, immunogenic amino acid sequence may be from the same or different agents (e.g., different pathogens). The present invention also contemplates a monovalent antigen delivery system or composition. A monovalent protein particle may comprise one or more copies of an immunogenic amino acid sequence. The present invention also contemplates production of a mixed population of protein particles by means of introduction of one or more expression constructs into a cell. As such, the protein particles described herein may be used in methods to deliver, immunise etc. against a plurality of target or candidate immunogens from the same or different origins.

Accordingly, any one of the immunogens when formed with the protein particle as described herein may elicit an immune response when administered as a protein particle. Alternatively, the immune response to any of these immunogens may be enhanced when they are co-administered with the protein particle. As hereinbefore described, administration of a protein particle as described herein may or may not contain a second antigen of interest. It is envisaged that the one or more immunogens can be administered separately from the protein particle at the same or at different sites. As will be appreciated, the one or more immunogens may be one or more immunogens other than a CRM amino acid sequence, and more preferably, a CRM197 amino acid sequence.

It will be appreciated that a protein particle comprising a diphtheria toxin CRM amino acid sequence derived from a cell as described may elicit or induce an immune response (e.g., anti-CRM antibodies) against a diphtheria pathogen. In some embodiments, the immune response against a diphtheria pathogen may be protective. Therefore, the protein particles as described herein may elicit a diphtheria pathogen-associated immune response as well as a response to one or more candidate immunogens directed to a targeted disease, disorder, or condition. Thus, according to some embodiments where a protein particle includes one or more immunogens other than a CRM amino acid sequence, it is contemplated that the protein particles may be at least partially protective against diphtheria as well as the specifically targeted disease, disorder, or condition.

In broad aspects, the present invention encompasses use of the protein particles as described herein in methods of eliciting an immune response to an agent or modulating an immune response in a subject, methods of immunisation of a subject, therapeutic methods, and/or methods of delivering one or more immunogens to a subject. Without being bound by any particular theory or mode of action, it is proposed that delivery or administration of an immunogen with a CRM protein particle (and in some embodiments, a CRM197 protein particle) may induce enhanced cellular, antibody, and/or immune responses, preferably both (although without limitation thereto). Alternatively, or in addition, slow or sustained release of an immunogen from a protein particle as described herein may reduce the need for multiple administrations and/or generate higher titre/strength cellular or antibody responses. That is, it will be appreciated that in some embodiments, although without being bound by any particular mode or theory, the protein particles as described herein may assist with enhanced immunogenicity by serving as a depot for prolonged immunogen display due, at least in part, to slow or retarded degradation of the particles.

Some broad aspects may provide methods of eliciting an immune response in a subject, the immune response being to an agent, by administering a protein particle as described herein. An aspect of the invention may provide a method of eliciting in a subject an immune response to an agent, the method including the step of administering to the subject an effective amount of a protein particle comprising a diphtheria toxin CRM amino acid sequence, wherein the protein particle comprising a diphtheria toxin CRM amino acid sequence is derived from a cell as herein described, to thereby elicit in the subject the immune response against the agent. It will be appreciated that according to some embodiments of this aspect, the agent against which the immune response may be generated may or may not be present in the subject, or the subject may or may not have been exposed to the agent. By way of example, in some embodiments of methods of eliciting an immune response in a subject for a preventative or prophylactic purpose, the subject may not have been exposed to the agent. As such, according to some embodiments, the agent may not necessarily be present in the subject in order to elicit an immune response to the agent.

Another aspect of the present invention provides a method of immunising a subject against a disease, disorder, or condition, wherein the method includes the step of administering to the subject an effective amount of a protein particle comprising a diphtheria toxin CRM amino acid sequence, wherein the protein particle comprising a diphtheria toxin CRM amino acid sequence is derived from a cell as herein described, to thereby immunise the subject against the disease, disorder, or condition.

A further aspect of the invention provides a method of treating or preventing a disease, disorder, or condition in a subject, the method including the step of administering to the subject an effective amount of a protein particle comprising a diphtheria toxin CRM amino acid sequence, wherein the protein particle comprising a diphtheria toxin CRM amino acid sequence is derived from a cell as herein described, to thereby treat or prevent the disease, disorder, or condition, in the subject.

A yet further aspect of the present invention provides a method of modulating an immune response in a subject, the method including the step of administering to the subject an effective amount of a protein particle comprising a diphtheria toxin CRM amino acid sequence, wherein the protein particle comprising a diphtheria toxin CRM amino acid sequence is derived from a cell as herein described, to thereby modulate the immune response in the subject.

Another aspect of the invention provides a method of delivering to a subject a protein particle comprising a diphtheria toxin Cross Reacting Material (CRM) amino acid sequence, wherein the protein particle comprising the diphtheria toxin CRM amino acid sequence is derived from a cell, the method including the step of administering to the subject the protein particle comprising a diphtheria toxin CRM amino acid sequence, wherein the protein particle comprising the diphtheria toxin CRM amino acid sequence is derived from a cell, to thereby deliver the protein particle to the subject.

By “administration” or “administering” is meant the introduction of a specified agent or a composition (e.g., a composition comprising one or more protein particles derived from a cell as described herein) to a subject by a chosen route or vehicle. Routes of administration may include topical, parenteral, and enteral which includes oral, buccal, sub-lingual, nasal, anal, gastrointestinal, subcutaneous, intravenous, intranasal, intraperitoneal, intra-articular, transdermal, inhalational, intraocular, intracerebroventricular, intramuscular, and intradermal routes of administration, although without limitation thereto.

It will be understood that methods of the invention include administration of a plurality of protein particles with different target immunogens of interest. It is envisaged that protein particles, proteins, or compositions can be administered simultaneously (i.e., at substantially the same time, or desirably together in the same composition) or separately (i.e., administered at an interval, for example, an interval of hours, days to several weeks or months). The protein particles, isolated proteins, or compositions may be administered sequentially (i.e., administered in sequence, for example at an interval). The protein particles, isolated proteins, or compositions may be administered in any order. If appropriate, the protein particles may be administered in a regular repeating cycle.

It will be appreciated that the protein particles, proteins, or compositions can be co-administered to a subject prior or subsequent to, or concurrent with, a further agent and in particular a further immunogenic or therapeutic agent (such as an adjuvant, an analgesic agent, and/or a second antigen, but not limited thereto). It will be appreciated that methods of the invention may (but not necessarily) include one or more additional steps such as, but not limited to, a booster step or a priming step. The methods as described herein may include one or more steps to identify whether a subject is in need of the method. By way of example, methods described herein may further include identifying whether the subject has or is at risk of developing an infection, or a cancer, or immune related disease, disorder or condition, as required. Any one of the methods as described herein also contemplate one or more steps to administer additional agents that may be useful in the method. By way of example, an antibiotic, an anti-inflammatory compound, an antiviral compound, or a corticosteroid (although without limitation thereto) may be administered prior to, concurrent with, or after administration of the protein particle of the present invention. A skilled addressee will readily appreciate whether an additional agent is required.

The term “effective amount” as used herein is an amount or a concentration of a specified agent or molecule sufficient to effect beneficial or desired results. By way of example only and without limitation thereto, in the context of the present invention this can be an amount or concentration of a protein particle as described herein which include a pathogen-specific antigen necessary to elicit an immune response to the pathogen when administered. An effective amount can be administered in one or more administrations, or as part of a series or slow release system. The effective amount will vary depending upon the health and physical condition of the subject and the taxonomic group of individual to be treated, the formulation of the composition, the assessment of the medical situation, the manner of administration, and other relevant factors. Ideally, an effective amount of an agent is an amount sufficient to induce the desired result without causing a substantial adverse or unwanted effect in the subject. A suitable effective amount can be readily determined by a person of skill in the art. An “effective amount” may be a therapeutically effective amount or a prophylactically effective amount, although without limitation thereto.

The terms “subject”, “individual”, “patient”, or “host” used interchangeably herein, refer to any subject for whom the methods of the present invention can be applied, particularly a vertebrate subject, and preferably a mammalian subject. Accordingly, the methods, agents, protein particles, and compositions disclosed herein may have human and/or veterinary applications. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys, marine mammals (e.g., dolphins, whales) and etc., to name only a few illustrative examples. In a preferred form, the mammal herein may be a human. The terms “subject”, “individual”, “patient”, or “host” includes avians (e.g., chickens, turkeys, ducks, geese, companion birds such as canaries, budgerigars), marine mammals (e.g., dolphins, whales), reptiles (e.g., snakes, frogs, lizards), amphibians, and fish.

In some embodiments, a “subject” may refer to “a subject in need thereof”. “Subject in need thereof” means a subject identified as in need of a therapy, treatment, or immunisation, as required.

By “elicit an immune response” or “elicit an immunological response” is meant generate, upregulate, activate, enhance, or stimulate the production or activity of one or more elements of the immune system inclusive of the cellular immune system, the humoral immune system, and/or the native immune system. Suitably, the one or more elements of the immune system include B lymphocytes, T-lymphocytes, antibodies, neutrophils, dendritic cells (such as Langerhans cells, plasmacytoid cells, lymphoid dendritic cells, interstitial dendritic cells, dermal dendritic cells, inflammatory dendritic cells, and myeloid dendritic cells, although without limitation thereto), memory cells, cytokines and/or chemokines, although without limitation thereto. The immune response may be a mucosal immune response. It will be appreciated that the immune response may be mediated by one or a plurality of elements of the immune system. Inclusive is a specific immune response wherein antibodies or sensitized T lymphocytes can be formed in the immune system of a subject after stimulating the subject with an agent. In some embodiments, the immune response may be a protective immune response. In other embodiments, the immune response may be a protective immune response that may, in some embodiments, include the elicitation of immunological memory.

For purposes of the present invention, a “cellular immune response” is one mediated by T-lymphocytes and/or other white blood cells. An aspect of cellular immunity involves an antigen-specific response by cytolytic T-cells (also known as “cytotoxic T-cells”, or “CTLs”). CTLs have specificity for peptide antigens that are presented in association with proteins encoded by the major histocompatibility complex (MHC) and expressed on the surfaces of cells. CTLs help induce and promote the intracellular destruction of intracellular pathogens, or the lysis of cells infected with such pathogens. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” may also refer to the production of cytokines, chemokines, and other such molecules or agents produced by activated T-cells and/or other white blood cells, including those derived from CD4+ and CD8+ T-cells. Thus, an immune response as used herein may be one which stimulates the production of CTLs, and/or the production or activation of helper T-cells. In some embodiments, the immune response as described herein comprises, or is a T-cell mediated immune response.

The antigen or immunogen of interest may also elicit an antibody-mediated immune response. As will be understood, an immune response mediated by an antibody molecule may also referred to as a “humoral immune response”. Inclusive of an antibody-mediated immune response is a neutralising antibody response. In some embodiments, the immune response comprises an antibody-mediated response. In other embodiments, the antibody-mediated response is a neutralising antibody response. In some embodiments, an antibody response may include or be mediated by an immunoglobin class or subtype such as, but not limited to, IgG, IgM, IgA etc.

Assays for assessing an immune response are described in the art and in the Examples herein, and may comprise in vivo assays, such as assays to measure antibody responses, delayed type hypersensitivity responses, antibody dependent cell cytotoxicity, or assays to measure the ability of a particular immunogen to stimulate a cell-mediated immunological response may be determined by a number of assays, such as by (e.g., lymphoproliferation (lymphocyte activation) assays, CTL cytotoxic cell assays, or by assaying for T-lymphocytes specific for the immunogen in a sensitized subject, although without limitation thereto), cytokine release assays, and a neutralising antibody response (e.g, by way of ELISA). Such assays are well known to a person of skill in the art, see for example, although without limitation thereto, Erickson et al., J. Immunol. (1993) 151:4189-4199; Doe et al., Eur. J. Immunol. (1994) 24:2369-2376.

In preferred embodiments, the immune response is, or comprises, a protective immune response.

A protective immune response may be against an agent in the form of a pathogen or molecular component thereof, whereby subsequent infection by the pathogen is at least partly prevented or minimised. A protective immune response may include an immune response that is sufficient to prevent or at least reduce the severity of symptoms of a disease, disorder, or condition. A protective immune response may include protective immunity to a cancer antigen.

As generally used herein the terms “immunise”, “vaccinate”, and “vaccine” refer to methods and/or compositions that elicit or potentiate a protective immune response. Accordingly, it will be understood that by “vaccinate” or “immunise” is meant delivery of a protein particle of the present invention and/or compositions comprising said particles to a subject to thereby elicit or potentiate a protective immune response in the subject. As such, in some embodiments, the protein particle and composition comprising said protein particle, may be a vaccine. Methods of immunisation, or methods of eliciting an immune response, may include immunising against an agent, or a disease, condition, or a disorder, as described herein.

The present invention contemplates methods that include administration of the protein particles and compositions as described herein to modulate an immune response in a subject.

The terms “modulate”, “modulation”, or modulating” as used herein means to alter, modify, or change an immune response or immunity in a subject. In some embodiments, such an immune response can be directed to an agent, or an immunogen. In some embodiments, “modulating an immune response” means promote, enhance, or other augment an immune response in the subject. In other embodiments, “modulating an immune response” means at least partly suppressing, inhibiting, dampen, or prevent an immune response in the subject. By way of example, it may be desirous to at least partly dampen an immune response in a subject suffering from an inflammatory autoimmune disease such as, rheumatoid arthritis (although without limitation thereto) by administration to a subject of a protein particle that comprises a CRM197 amino acid sequence as described herein and an agent to target a TNFα-induced immune response such an anti-TNFα antibody or a fragment thereof, but without limitation thereto.

The present invention contemplates therapeutic methods to treat and/or prevent a disease, disorder, or condition by administering the protein particles as described herein.

As used herein, “treating”, “treat” or “treatment” refers to a therapeutic intervention that at least partly ameliorates, eliminates, or reduces a symptom or pathological sign of a disease, disorder or condition after it has begun to develop. The term “ameliorating”, with reference to a disease, disorder, or condition, refers to any observable beneficial effect of the treatment. Treatment need not be absolute to be beneficial to the subject. The beneficial effect can be determined using any methods or standards known to the ordinarily skilled artisan. Treatment may be effected prophylactically or therapeutically.

In certain embodiments, the immune response may be suitable for preventing or treating a subject.

As used herein, “preventing” (or “prevent” or “prevention”) refers to a course of action (such as administering a protein particle as described herein and a composition comprising the same) initiated prior to the onset of a symptom, aspect, or characteristic of a disease, disorder or condition, so as to prevent or reduce the symptom, aspect, or characteristic. It is to be understood that such preventing need not be absolute to be beneficial to a subject. A “prophylactic” treatment is a treatment administered to a subject who does not exhibit signs of a disease, disorder or condition, or exhibits only early signs for the purpose of decreasing the risk of developing a symptom or clinical characteristic or outcome of the disease, disorder or condition.

Also contemplated herein is the use of a protein particle as described herein in the manufacture of a medicament to treat or prevent a disease, disorder, or condition, or to elicit an immune response, or to modulate an immune response, or to immunise a subject.

The term “agent” can broadly refer to any molecule or component thereof, that may elicit or be a part of a pathological or disease response, and in particular an immune response. An agent may be a pathogen or non-pathogenic organism. An agent may be a disease-associated immunogen (e.g., a cancer immunogen). An agent may be an autoallergen or transplantation allergen, although without limitation thereto. A disease, disorder, or condition may be caused by an agent. A disease, disorder, or condition may be associated with an agent.

In certain preferred embodiments, an immunogen is derived from, or corresponds to, a protein of interest, a target immunogen, or a candidate immunogen.

In another broad embodiment, the disease, disorder, or condition may be associated with a protein of interest, a target immunogen, or a candidate immunogen.

As described herein, the invention provides methods and/or compositions for eliciting an immune response to a pathogen, inducing immunity against a pathogen, and/or preventing or treating a pathogen-associated disease, disorder, or condition in a subject, or preventing or treating an infection caused by a pathogens. In some broad embodiments, the disease, disorder, or condition may be caused by a pathogen. It is contemplated for the present invention that an immunogen, and in some embodiments an immunogenic amino acid sequence of an immunogen, may be derived from a pathogen such as, but not limited to, any of several known viruses, bacteria, parasites and fungi, as described more fully below. In some embodiments, an immunogen may be a proteinaceous and/or a non-proteinaceous component molecule of a pathogen (e.g., a surface protein, a cell surface protein, immunogenic peptide or other component thereof such as in a “subunit vaccine”, a polytope comprising multiple B- and/or T-epitopes, VLPs, capsids, or capsomeres), an inactivated pathogen (e.g., an inactivated virus, attenuated parasite-infected RBC, or attenuated bacterium) or any other molecule capable of eliciting an immune response to the pathogen. A non-limiting example of other molecules capable of eliciting an immune response includes carbohydrates on the surface of bacteria, and in particular carbohydrates in the form of capsular polysaccharides and/or lipopolysaccharides, and/or other virulence factors involved in the pathogenicity of a pathogen such as a bacteria. Therefore, the present invention contemplates in some embodiments one or a plurality of immunogens other than a diphtheria CRM amino acid sequence wherein the, or each, immunogen may be a proteinaceous and/or a non-proteinaceous immunogen for use in a protein particle as described herein. In some embodiments, the, or each, immunogen other than a diphtheria CRM amino acid sequence is of, derived from, or from a pathogen.

The term “pathogen” as used herein relates to any living or non-living organism that is capable of causing a disease, disorder, or condition in a subject, such as (but not limited to) a mammal. The pathogen may be a virus, a bacterium, a protozoan, a fungus, or a parasite (such as a parasitic worm).

The one or more immunogens may be derived from, or correspond to, a virus. The one or more immunogens may be derived from, or correspond to, a viral protein and/or a viral protein sequence.

Reference herein to a virus or a viral protein includes without limitation a virus or a protein therefrom from any virus family.

As used herein, a virus includes an enveloped virus and a non-enveloped virus. A non-enveloped virus may also be referred to as a naked virus. As will be appreciated, a non-enveloped virus refers to a virus having only a capsid that encapsulates a virus genome. An enveloped virus typically comprises a virus genome and a capsid coated by a viral envelope. Typically, the viral envelope may comprise host cell derived lipids and proteins, as well as one or more viral proteins (e.g., glycoproteins). A viral protein may refer to any protein present in or on, or incorporated into the virus or virion particle (e.g. on the surface of the particle or as part of the envelope and/or capsid), or coded for by the viral genome that is part of replication of a virus, or host cell interactions. The viral protein may be a capsid protein, an envelope protein, a nucleocapsid protein, a surface protein (e.g., a fusion protein present on virus particle surface to facilitate fusion with a cell), a structural protein, a regulatory protein, an accessory protein, and/or a non-structural protein (or combinations thereof). Typically, although not exclusively, a non-structural protein is involved in replication of a virus genome, and is expressed in an infected cell, and is generally not incorporated into a virus or virion particle. A polymerase protein is a non-limiting example of a non-structural protein. A structural protein is typically incorporated into the virus or virion particle as part of a structure that encapsulate the viral genome. A surface protein may bind to a host cell by way of a receptor.

In preferred embodiments that contemplate a virus and/or an immunogenic amino acid sequence or immunogen derived from, or corresponding to, a virus, a viral protein and/or or a viral protein sequence, the invention contemplates any member of the dsDNA Viruses group including (and without limitation thereto) any member of the family Adenoviridae inclusive of a mastadenovirus (e.g., a human adenovirus) and an aviadenovirus (e.g., a fowl adenovirus), although without limitation thereto; any member of the family Herpesviridae inclusive of an Alphaherpesvirinae such as, but not limited to, a simplexvirus (e.g., a human herpesvirus 1) and a varicellosus (e.g., a human herpesvirus 3); a Betaherpesvirinae such as, but not limited to, a cytomegalovirus (e.g., human herpesvirus 5), a muromegalovirus (e.g., a mouse cytomegalovirus 1), a roseolovirus (e.g., a human herpesvirus 6); a Gammaherpesvirinae such as, but not limited to, a lymphocryptovirus (e.g., a human herpesvirus 4), a rhadinovirus (e.g., an ateline herpesvirus 2); any member of the family Papillomaviridae inclusive of a papillomavirus, preferably human papillomavirus, and preferably subtypes 16, 18, 31, 33, 35, 39, 45, 51, 52, 56, 58, and 59, although without limitation thereto; any member of the family Iridoviridae inclusive of a ranavirus and such as, epizootic haematopoietic necrosis virus, but not limited to; any member of the family Polyomaviridae inclusive of a polyomavirus and preferably murine polymavirus; any member of the family Poxviridae inclusive of an orthopoxvirus (e.g., a vaccinia virus), a parapoxvirus (e.g., a orf virus), an avipoxvirus (e.g., a fowlpox virus), an capripoxvirus (e.g., a sheep pox virus), a leporipoxvirus (e.g., a myxoma virus) and a suipoxvirus (e.g., a swinepox virus). A virus of the invention further contemplates any member of the ssDNA Viruses group including (and without limitation thereto) any member of the family Parvoviridae inclusive of a parvovirus (e.g., Rheumatoid arthritis virus, B19).

In the preferred embodiments that contemplate a virus and/or an immunogenic amino acid sequence or immunogen derived from, or corresponding to, a virus, a viral protein and/or a viral protein sequence, the invention further contemplates any member of the dsRNA Viruses group including (and without limitation thereto) any member of the family Birnaviridae inclusive of an aquabirnavirus (e.g., an infectious pancreatic necrosis virus) and an avibirnavirus (e.g., infectious bursal disease virus); any member of the family Reoviridae inclusive of an orthoreovirus (e.g., a reovirus 3), a orbivirus (e.g., a bluetongue virus 1), a rotavirus, a coltivirus (e.g., a Colorado tick fever virus and an aquareovirus.

In the preferred embodiments that contemplate a virus and/or an immunogenic amino acid sequence or immunogen derived from, or corresponding to, a virus, a viral protein and/or a viral protein sequence, the invention contemplates any member of the (+) sense RNA Virus group including (and without limitation thereto) any member of the family Astroviridae inclusive of an astrovirus (e.g., a human astrovirus) and an arterivirus (e.g., an equine arteritis virus); any member of the family Caliciviridae inclusive of a Norwalk virus; any member of the family Hepeviridae inclusive of a Hepatitis E virus; any member of the family Coronaviridae inclusive of a coronavirus and a SARS coronavirus and a torovirus; any member of the family Flaviviridae inclusive of a flavivirus such as, but not limited to, a yellow fever virus, a dengue virus and a West Nile virus; a pestivirus (e.g., bovine diarrhoea virus) and hepatitis C-like viruses (e.g., a hepatitis C virus); any member of the family Picornaviridae inclusive of an enterovirus, a rhinovirus (e.g., a human rhinovirus 1A), a hepatovirus (e.g., a hepatitis A virus), a cardiovirus (e.g. an encephalomyocarditis virus) and an aphtovirus (e.g., foot-and-mouth disease virus); any member of the family Togaviridae inclusive of an alphavirus (e.g., a Sindbis virus, a Chikungunya virus) and a rubivirus (e.g., a rubella virus).

In embodiments that contemplate a Coronaviridae virus, the Coronaviridae virus may be a coronavirus (“CoV”). The coronavirus may infect humans. The coronavirus may be the causative agent of, or associated with, a severe acute respiratory syndrome in mammals, particularly humans. In some embodiments, the coronavirus may be a severe acute respiratory syndrome (SARS) coronavirus and/or Middle Eastern respiratory syndrome (MERS)-CoV. In some embodiments, the SARS coronavirus may be either SARS-CoV-1 and/or SARS CoV-2. It is understood that SARS-CoV-1 (also referred to as SARS-CoV) is associated with an outbreak in 2003. SARS-CoV-2 is a causative agent of, or associated with, COVID-19, being the disease term associated with an outbreak of SARS CoV-2 which first emerged in Hubei province, China, in December 2019. SARS-CoV-1 and SARS-CoV-2 are genetically related yet distinct viruses. In some embodiments, the SARS coronavirus may be a SARS-CoV-2.

In some embodiments relating to a coronavirus, the viral protein may be a structural protein, and preferably the structural protein may be a fusion protein located on virus envelope, or a capsid protein. In certain embodiments, the structural protein is selected from the group consisting of a spike (S) protein, an envelope (E) protein, a membrane (M) protein, and a nucleocapsid (N) protein, and any combination thereof. According to the aforementioned embodiments, fragments, variants, or derivatives of said viral protein are contemplated.

Some embodiments contemplate a coronavirus viral protein that may in the form of a spike (S) protein (also referred to as “spike glycoprotein”). Although not wishing to be bound by a particular theory, coronavirus spike glycoproteins (S protein) form a trimeric structure on the viral envelope and facilitate binding and viral entry. Typically, an S protein includes the S1 domain, which contains a receptor binding domain (‘RBD’) that binds to the receptor on the cell surface.

In particular, an RBD of an S protein typically contains the domain termed the “Critical Neutralizing Domain” (‘CND’) may be capable of inducing highly potent neutralizing antibody responses and cross-protection against divergent SARS-CoV strains. Coronavirus S proteins typically form a trimeric structure on the viral envelope and facilitate binding and viral entry. SARS-CoV-2 typically uses the same entry receptor as SARS-CoV, the Angiotensin Converting Enzyme 2 (ACE2). One or more amino acid residues involved in the binding of SARS-CoV to ACE2 are conserved in SARS-CoV-2. Previous work done on both SARS-CoV and MERS-CoV S proteins have indicated that neutralizing antibodies, as well as T cell responses are generated against the S protein in surviving patients or in vaccinated animals. This is likely similar for SARS-CoV-2. A SARS-CoV-2 S protein may be capable of inducing neutralizing antibodies, this protein may be a potential candidate antigen molecule for compositions and in particular, immunogenic compositions. The SARS-CoV-2 N (nucleocapsid) protein is a structural protein located to the virus core. Convalescent patients may show high antibody titers to this protein. The N protein likely may contain several T cell epitopes based on analysis of T cell responses (CD4+ and CD8+) in survivors. Although not wishing to be bound by any particular theory, several of these T cell epitopes are conserved in the various SARS-CoV variants. Hence in some embodiments, an N protein, or a fragment, variant, or derivative thereof, optionally in combination with a S protein (or a fragment, variant, or derivative thereof such as, but not limited to an S1 domain or the RBD) may constitute a suitable immunogen that may, in some embodiments, induce neutralising antibodies and/or T cell responses. Accordingly, in some embodiments, such an immunogen may protect against a coronavirus infection. Exemplary amino acid sequences for the S protein and N protein are set forth in SEQ ID NO:64 and SEQ ID NO:56, respectively.

In some embodiments, a coronavirus S protein immunogenic amino acid sequence is derived from, or corresponds to, a S1 domain of the S protein, or a fragment, derivative, or variant thereof. An S1 domain may span, or include, amino acid residues 1-681 of an S protein (e.g., an S protein sequence as set forth in SEQ ID NO:64). In certain embodiments, an immunogenic amino acid sequence of, or from, an S1 domain may comprise, consist essentially of, consist of, or is, an amino acid sequence as set forth in SEQ ID NO:58, or a fragment, variant, or derivative thereof.

In some embodiments, the fragment of an S1 domain of an S protein may include an RBD domain. An RBD domain may span amino acid residues 319 to 529 of an S protein (e.g., an S protein sequence as set forth in SEQ ID NO:64). In further embodiments, an RBD domain may comprise, consist essentially of, consist of, or is, an amino acid sequence as set forth in SEQ ID NO:57, or a fragment, variant, or derivative thereof.

In some embodiments, a coronavirus S protein immunogenic amino acid sequence is derived from, or corresponds to, a S2 domain of the S protein, or a fragment, derivative, or variant thereof. An S2 domain may span, or include, amino acid residues 686-1273 of an S protein (e.g., an S protein sequence as set forth in SEQ ID NO:64).

Some embodiments of the present invention contemplate a coronavirus protein that may be in the form of an N protein, or a fragment, variant, or derivative thereof. It will be appreciated that a coronavirus N protein may include several conserved T cell epitopes. In some embodiments, an immunogenic amino acid sequence derived from, or corresponding to, a coronavirus N protein may comprise, consist essentially of, consist of, or is, an amino acid sequence as set forth in SEQ ID NO:56, or a fragment, variant, or derivative thereof.

In certain embodiments, the coronavirus viral protein is a spike (S) protein, or a fragment, variant, or derivative thereof. In some embodiments, an immunogenic amino acid sequence derived from, or corresponding to, a spike protein may comprise, consist essentially of, consist of, or is, an amino acid sequence as set forth in SEQ ID NO:64.

In some embodiments, an S protein immunogenic amino acid sequence may be derived from a fragment, peptide, epitope, or a portion of, or is, a S protein having an amino acid sequence as set forth in SEQ ID NO:64. Suitably, the coronavirus S protein may be a SARS-CoV-2 viral protein.

In some embodiments that encompass an S protein, the immunogenic amino acid sequence may be derived from, or correspond to, a variant of the S protein which includes a substitution of an aspartic acid residue at position 640 to a glycine residue of an S protein (e.g., substitution of an S protein amino acid sequence as set forth in SEQ ID NO:64), or a fragment or derivative thereof.

In some embodiments, an immunogenic amino acid sequence derived from, or corresponding to, a coronavirus protein comprises, consists of, consists essentially of, or is, an amino acid sequence selected from the group consisting of an amino acid sequence as set forth in SEQ ID NO:56, SEQ ID NO:57, SEQ ID NO:58, SEQ ID NO:64, SEQ ID NO:101, SEQ ID NO:102, and SEQ ID NO: 103 (or a fragment, variant, or derivative thereof), and any combination thereof. In some embodiments, the immunogenic amino acid sequence derived from, or corresponding to, a coronavirus protein is an amino acid sequence as set forth in SEQ ID NO: 101, or a fragment, variant, or derivative thereof.

In some embodiments, an immunogenic amino acid sequence derived from, or corresponding to, a coronavirus protein comprises, consists of, consists essentially of, or is, an amino acid sequence as set forth in any one of SEQ ID NO:56, SEQ ID NO:57, SEQ ID NO:58, SEQ ID NO:64, SEQ ID NO:101, SEQ ID NO: 102, and/or SEQ ID NO:103 (or a fragment, variant, or derivative thereof), and any combination thereof.

In some embodiments, a coronavirus immunogenic amino acid sequence may be derived from, or correspond to, any one of the amino acid sequences as set forth in Examples 9 and/or 10 (and any associated Figures and/or Tables), or a fragment, variant, or derivative thereof.

In the preferred embodiments that contemplate a virus and/or an immunogenic amino acid sequence or immunogen derived from, or corresponding to, a virus, a viral protein and/or a viral protein sequence, the invention contemplates any member of the (−) negative sense RNA Virus group including (and without limitation thereto) any member of the family Filoviridae inclusive of a filovirus (e.g., a Marburg virus, an Ebola virus); any member of the family Paramyxoviridae inclusive of a paramyxovirus (e.g., a human parainfluenza virus 1), a morbillivirus (e.g., a measles virus), a rubulavirus (a mumps virus), a Hendra virus, and a Nipah virus; any member of the family Pneumovirinae inclusive of a pneumovirus (e.g., a human respiratory syncytial virus); any member of the family Rhabdoviridae inclusive of a vesiculovirus (e.g., a vesicular stomatitis virus, Indiana virus), a lyssavirus (e.g., a rabies virus) and an ephemerovirus (e.g., a bovine ephemeral fever virus); any member of the ambisense RNA Virus group inclusive of any member of the family Arenaviridae such as an arenavirus (e.g., lymphocytic choriomeningitis virus); any member of the family Bunyaviridae inclusive of a bunyavirus (e.g., a Bunyamwera virus) and a hantavirus (e.g., a Hantaan virus); any member of the family Orthomyxoviridae inclusive of an influenzavirus A (such as an influenza A virus, an avian influenza A virus), an influenzavirus B (such as an influenza B virus), an influenzavirus C (such as an influenza C virus) and a “Thogoto-like viruses” (e.g., a Thogoto virus).

In the preferred embodiments that contemplate a virus and/or an immunogenic amino acid sequence or immunogen derived from, or corresponding to, a virus, a viral protein and/or a viral protein sequence, the invention contemplates any member of the RNA Reverse Transcribing Viruses group including any member of the family Retroviridae inclusive of a mammalian type B retrovirus (e.g., a mouse mammary tumor virus), a mammalian type C retrovirus (e.g., a murine leukemia virus), an avian type C retrovirus (e.g., an avian leukosis virus), a type D retrovirus (e.g., a Mason-Pfizer monkey virus), a BLV-HTLV retrovirus (e.g., a bovine leukemia virus), a lentivirus (e.g., a human immunodeficiency virus 1) and a spumavirus (e.g., a human spumavirus).

In the preferred embodiments that contemplate a virus and/or an immunogenic amino acid sequence or immunogen derived from, or corresponding to, a virus, a viral protein and/or a viral protein sequence, the invention contemplates any member of the DNA Reverse Transcribing Viruses group including any member of the family Hepadnaviridae inclusive of an orthohepadnavirus (e.g., a hepatitis B virus) and an avihepadnavirus (e.g., a duck hepatitis B virus), although without limitation thereto.

In the preferred embodiments that contemplate a virus and/or an immunogenic amino acid sequence or immunogen derived from, or corresponding to, a virus, a viral protein and/or a viral protein sequence, the invention contemplates any member of the ssDNA Virus group including any member of the family Circoviridae; any member of the family Parvoviridae, although without limitation thereto.

In the preferred embodiments that contemplate a virus and/or an immunogenic amino acid sequence or immunogen derived from, or corresponding to, a virus, a viral protein and/or a viral protein sequence, the invention contemplates any member of the un-classified group of subviral agents such as satellites (e.g., tobacco necrosis virus), Viroids (hepatitis delta virus) and Agents of Spongiform Encephalopathies (e.g., prions, scrapie agent).

The present invention may be particularly useful in relation to a member of the family Flaviviridae. In preferred embodiments, the member of the Flaviviridae is a hepatitis C virus. By way of example, the HCV genome encodes several viral proteins, including core antigen, E1 (also known as E) and E2 (also known as E2/NSI), NS3, NS4, NS5, and the like, which will find use with the present invention (see, Houghton et al. Hepatology (1991) 14:381-388, for a discussion of HCV proteins, including E1 and E2). In particularly preferred embodiments, an HCV viral protein according to present invention may be selected from the group consisting of an E1 protein, an E2 protein, a NS3 protein, and a core antigen protein, or a fragment, variant, or derivative thereof, and any combination thereof. It will be appreciated that HCV proteins are expressed as a polyprotein that may be cleaved post-translation. An exemplary amino acid sequence of a polyprotein derived from a HCV genome is set forth in SEQ ID NO:44. In some embodiments, a HCV protein or immunogenic acid sequence thereof, may be derived from, or correspond to, an amino acid sequence as set forth in SEQ ID NO:44.

In some embodiments, a HCV core protein as used herein may comprise an amino acid sequence as set forth in SEQ ID NO:43. In some embodiments, a HCV core protein immunogenic amino acid sequence may be derived from a fragment, peptide, epitope, or portion of, or be, a HCV core protein comprising an amino acid sequence as set forth in SEQ ID NO:43. In some embodiments, an HCV core protein immunogenic amino acid sequence may comprise, consist essentially of, consist of, or is, an amino acid sequence as set forth in SEQ ID NO:28 and/or SEQ ID NO:43, or a fragment, variant, or derivative thereof.

In some embodiments, a HCV NS3 protein as used herein may comprise an amino acid sequence as set forth in SEQ ID NO:69. In some embodiments, a HCV NS3 protein immunogenic amino acid sequence may be derived from a fragment, peptide, epitope, or portion of, or is, a HCV NS3 protein having an amino acid sequence as set forth in SEQ ID NO:69. In some embodiments, an HCV NS3 protein immunogenic amino acid sequence may comprise, consist essentially of, consist of, or is, an amino acid sequence as set forth in SEQ ID NO:29, or a fragment, variant, or derivative thereof. In some embodiments, a HCV E1 protein may comprise, consist of, consist essentially of, or is, an amino acid sequence as set forth in SEQ ID NO:45. In some embodiments, a HCV E1 protein immunogenic amino acid sequence may be derived from a fragment, peptide, epitope, or portion of, or is, a HCV E1 protein having an amino acid sequence as set forth in SEQ ID NO:45. In certain embodiments, an HCV E1 protein immunogenic amino acid sequence may comprise, consist essentially of, consist of, or is, an amino acid sequence as set forth in SEQ ID NO:30 and/or SEQ ID NO:70, or a fragment, variant, or derivative thereof. In some embodiments, a HCV E2 protein may comprise an amino acid sequence as set forth in SEQ ID NO:46. In some embodiments, a HCV E2 protein immunogenic amino acid sequence may be derived from a fragment, peptide, epitope, or portion of, or is, a HCV E2 protein having an amino acid sequence as set forth in SEQ ID NO:46. In some embodiments, an HCV E2 protein immunogenic amino acid sequence may comprise, consist essentially of, consist of, or is, an amino acid sequence selected from the group consisting of an amino acid as set forth in SEQ ID NO:31, SEQ ID NO:71, and SEQ ID NO: 104, or a fragment, variant, or derivative thereof, and any combination thereof.

In some embodiments, a HCV immunogenic amino acid sequence may be derived from, or correspond to, any one of the amino acid sequences as set forth in Examples 5 and/or 6 (and any associated Figures and/or Tables), or a fragment, variant, or derivative thereof.

In some embodiments, an immunogenic amino acid sequence may be derived from, or corresponding to, an HCV protein which comprises, consists of, consists essentially of, or is, an amino acid sequence selected from the group consisting of an amino acid sequence as set forth in SEQ ID NO:28, SEQ ID NO:29, SEQ ID NO:30, SEQ ID NO:31, SEQ ID NO:43, SEQ ID NO:44, SEQ ID NO:45, SEQ ID NO:46, SEQ ID NO:69, SEQ ID NO:70, SEQ ID NO:71, and SEQ ID NO: 104, or a fragment, variant, or derivative of any one of the aforementioned sequences, and any combination thereof.

In some embodiments that relate to HCV, an immunogenic amino acid sequence may comprise, consist of, or consist essentially of an amino acid sequence as set forth in any one of SEQ ID NOs:28, 29, 30, 31, 70, and/or 104, or a fragment, variant, or derivative of any one of the aforementioned sequences, and any combination thereof.

In some embodiments that relate to HCV, an immunogenic amino acid sequence may comprise, consist of, or consist essentially of an amino acid sequence as set forth in any one of, or in, SEQ ID NOs:28, 29, 70, and/or 104, or a fragment, variant, or derivative of any one of the aforementioned sequences, and any combination thereof.

In some embodiments that relate to HCV, an immunogenic amino acid sequence may comprise, consist of, or consist essentially of an amino acid sequence as set forth in any one of, or in, SEQ ID NOs:29-31, or a fragment, variant, or derivative of any one of the aforementioned sequences, and any combination thereof.

In some embodiments that relate to HCV, an immunogenic amino acid sequence may comprise, consist of, or consist essentially of an amino acid sequence as set forth in SEQ ID NO:28, or a fragment, variant, or derivative thereof.

In some other embodiments, the member of the Flaviviridae is a Dengue virus. According to some embodiments, the amino acid sequence may be derived from or correspond to, a Dengue virus protein in the form of an envelope protein and/or a capsid protein, or a fragment, variant, or derivative thereof. In some embodiments, a Dengue virus envelope protein immunogenic amino acid sequence may be derived from a fragment, peptide, epitope, or portion of, or is, a Dengue envelope protein having an amino acid sequence as set forth in SEQ ID NO:47. In some embodiments, a Dengue virus envelope protein may comprise, consist essentially of, consist of, or is, an amino acid sequence as set forth in SEQ ID NO:41, or fragments, variants, or derivatives thereof. In some embodiments, a Dengue virus capsid protein immunogenic amino acid sequence may be derived from a fragment, peptide, epitope, or portion of, or is, a Dengue capsid protein having an amino acid sequence as set forth in SEQ ID NO:48. In other embodiments, the Dengue virus capsid protein may comprise, consist essentially of, consist of, or is, an amino acid sequence as set forth in SEQ ID NO:42, or a fragment, variant, or derivative thereof. According to these embodiments, the Dengue virus may be selected from the group consisting of a Dengue Type 1, a Dengue Type 2, a Dengue Type 3, and a Dengue Type 4, and any combination thereof.

In some embodiments, a Dengue virus immunogenic amino acid sequence may be derived from, or correspond to, any one of the amino acid sequences as set forth in Example 7 (and any associated Figures and/or Tables), or a fragment, variant, or derivative thereof.

An influenza virus is a further example of a virus for which the present invention will be particularly useful. In preferable embodiments, the influenza virus protein is selected from the group consisting of hemagglutinin (HA), neuraminidase (NA), nuclear protein (NP), matrix protein M1, and matrix protein M2, and combinations thereof. The envelope glycoproteins HA and NA of influenza A may be of particular interest for generating an immune response. In some embodiments, the immunogen derived from an influenza virus corresponds to a hypervariable region of HA. In other embodiments, the immunogen derived from an influenza virus is a domain of M2 and more preferably, M2e. Typically, although not exclusively, the domain of M2 is an ectodomain.

Other immunogens of particular interest to be used in the subject protein particle compositions include immunogens and polypeptides derived therefrom from human papillomavirus (HPV), such as one or more of the various early proteins including E6 and E7, tick-borne encephalitis viruses, HIV-1 (also known as HTLV-III, LAV, ARV, hTLR, etc.), including but not limited to immunogens from the isolates HIV_{IIIb}, HIV_{SF2}, HIV_{LAV}, HIV_{LAI}, HIV_{MN}) such as gp120, gp41, gp160, gag and pol. Reference is made to Mann and Ndungu (2015) Virol J., 12: 3, which describes non-limiting examples of HIV proteins and/or immunogens that may be suitable, and is incorporated herein by reference.

Non-viral pathogens and immunogens are contemplated including fungi, parasites, including apicomplexa, or uni cellular parasites, nematodes, trematodes, cestodes and plant pathogen or bacteria.

Non-limiting examples of fungi include primary systemic fungal pathogens such as Coccidioides immitis, Histoplasma capsulatum, Chlamydia trachomatis, Blastomyces dermatitidis, and Paracoccidioides brasiliensis. Opportunistic fungal pathogens which tend to rely upon an immunocompromised host include Cryptococcus neoformans, Pneumocystis jirovecii, a Candida sp., an Aspergillus sp., Penicillium marneffei, and Zygomycetes, Trichosporon beigelii, a Coccidioides species, and a Fusarium sp, and without limitation thereto. A range of pathogenic fungi are associated with immunocompromised subjects including those with AIDS, with chemotherapy induced neutropenia or patients undergoing haematopoietic stem cell transplantation, among others.

A non-limiting example of a parasite includes a protozoa such as a malaria parasites inclusive of a Plasmodium sp such as P. falciparum, P. ovale, P. knowlesii, P. malariae and P. vivax, although without limitation thereto. Some preferred embodiments related to P. falciparum. Other parasites include a Schistosoma sp, an Amebiasis sp, a Babesia sp, a Cryptosporidium sp, a Cyclosporia sp, a Giardia sp, a Microsporidia sp, a Toxoplasma sp, and Trypanosomes inclusive of a Leishmania sp, although without limitation thereto. A roundworm is inclusive of a filarial sp, a strongyloidial sp, a trichinellosis sp and a toxocariasis sp. A fluke is inclusive of a Paragonimus sp and a Schistosoma sp, although without limitation thereto. A tapeworm is inclusive of a Cysticercosis sp and an Echinococcosis sp although without limitation thereto.

A causative agent of schistosomiasis is also contemplated, inclusive of a one or more of Schistosoma mansoni, Schistosoma japonicum, and Schistosoma haematobium. Non-limiting examples of immunogens against Schistosoma species may be found in International Publication No. WO/2016/172762, which is incorporated herein by reference.

The invention also encompasses use of an immunogen derived from a bacterium, and in particular, Gram-positive and Gram-negative bacteria inclusive of a bacterial pathogen may be of genera such as Neisseria, Bordatella, Pseudomonas, Corynebacterium, Salmonella, Streptococcus, Shigella, Mycobacterium, Mycoplasma, Clostridium, Helicobacter, Borrelia, Yersinia, Legionella, Hemophilus, Rickettsia, Burkholderia, Listeria, Brucella, Coxiella, Chlamydophila, Vibrio, and Treponema, including species such as Staphylococcus aureus, Staphylococcus epidermidis, Helicobacter pylori, Bacillus anthracis, Bordatella pertussis, Corynebacterium diptheriae, Corynebacterium pseudotuberculosis, Clostridium tetani, Clostridium botulinum, a group A or group B Streptococcus, Streptococcus pneumoniae, Streptococcus agalactiae, Streptococcus mutans, Streptococcus oralis, Streptococcus parasanguis, Streptococcus pyogenes, Streptococcus viridans, Listeria monocytogenes, Hemophilus influenzae, Hemophilus influenzae type B, Pasteurella multocida, Shigella dysenteriae, Mycobacterium tuberculosis, Mycobacterium bovis, Mycobacterium avium, Mycobacterium avium subsp. paratuberculosis, Mycobacterium leprae, Mycobacterium asiaticum, Mycobacterium intracellulare, Mycoplasma pneumoniae, Mycoplasma hominis, Neisseria meningitidis, Neisseria gonorrhoeae, Rickettsia rickettsii, Brucella abortus, Brucella canis, Brucella suis, Legionella pneuophila, Klebsiella pneumoniae, Pseudomonas aeruginosa, Treponema pallidum, Treponema pertanue, Chlamydia trachomatis, Vibrio cholerae, Treponema carateum, Salmonella typhimurium, Salmonella typhi, Borrelia burgdorferi, Burkholderia pseudomallei, Burkholderia mallei, Coxiella burnetii, Chlamydophila pneumoniae, and Yersinia pestis, although without limitation thereto.

In embodiments relating to a group A streptococcus (GAS) bacteria, an exemplary immunogenic fragment/peptide that may be utilised to elicit an immune response against a group A streptococcus bacteria (e.g., Streptococcus pyogenes), and more particularly for use in immunising against group A streptococcus bacteria may derived from, or correspond to, a virulence factor, and in some embodiments, may be an M-protein, or a fragment, variant, or derivative thereof. M protein is a virulence factor which is strongly antiphagocytic and binds to serum factor H, destroying C3-converstase and preventing opsonization by C3b. In certain embodiments, the M-protein derived immunogenic fragment comprises, consists of, consists essentially of, or is the amino acid sequence LRRDLDASREAKNQVERALE (SEQ ID NO:17). A GAS immunogenic fragment may be derived from, or correspond to, a neutrophil inhibitor protein, or a fragment thereof. The neutrophil inhibitor may be a protease, or a fragment, variant, or derivative thereof. The protease may be an IL-8 protease, or a fragment thereof. The protease/IL-8 protease may be a SpyCEP protein, or a fragment, variant, or derivative thereof. In some embodiments, the SpyCEP protein fragment may be a linear B-cell epitope. In certain embodiments, the SpyCEP protein fragment comprises, consists of, consists essentially of, or is the amino acid sequence NSDNIKENQFEDFDEDWENF (SEQ ID NO:18). In some embodiments, a GAS immunogenic fragment may be derived from, or correspond to, a peptidase, or a fragment, variant, or derivative thereof. The peptidase may be a C5a peptidase (ScpA). A GAS immunogenic fragment may be a fibronectin-binding protein, or a fragment, variant, or derivative thereof. In some embodiments, a plurality of GAS-derived immunogenic fragments derived from the same or different GAS proteins, may be used. In some embodiments, the M-protein derived immunogenic fragment is co-administered with an amino acid sequence derived from or corresponding to a SpyCEP protein, or a fragment thereof. In other certain embodiments, the GAS immunogenic fragment comprises an amino acid sequence as set forth in SEQ ID NO:17 and/or 18. Other exemplary GAS immunogenic fragments may be found in International Publication Number WO/2015/157820 or International Publication Number WO/2019/036761, each of which is incorporated herein by reference.

In some embodiments, a Streptococcus immunogenic amino acid sequence may be derived from, or correspond to, any one of the amino acid sequences as set forth in Example 4 and/or Table 6, (and any associated Figures), or a fragment, variant, or derivative thereof.

In some embodiments, the bacterium is a Coxiella species. In further embodiments, the Coxiella species is Coxiella burnetti. Coxiella burnetti (C. burnetti) is an etiological agent of the infectious zoonotic disease Q (“query”) fever. It is a gram-negative intracellular bacterium that manifests as an incapacitating influenza-like illness. Acute Q fever often presents as a self-limiting febrile illness or pneumonia whereas chronic Q fever can be complicated by endocarditis and chronic hepatitis which are sometimes incurable. Identification of candidate immunodominant antigens of C. burnetti and the specific CD4+ and CD8+ epitopes of these antigens were mapped through bioinformatic analysis as described herein. Epitopes of these antigens, named COX, have a collective size of 101.1 kDa, which may find a use for potential Q fever therapeutic development. An amino acid sequence of a COX protein is set forth in SEQ ID NO:59.

In some embodiments relating to a relating to a Coxiella species, an immunogenic amino acid sequence may comprise, consist essentially of, consist of, or is, an amino acid sequence selected from the group consisting of an amino acid sequence as set forth in SEQ ID NO:59, SEQ ID NO:60, SEQ ID NO:61, SEQ ID NO:62, SEQ ID NO:63, SEQ ID NO:72, SEQ ID NO:73, and any one of SEQ ID NOs:74-100 (as set out in Table 7), or a fragment, variant, or derivative thereof, and any combination thereof. In some embodiments relating to a Coxiella species, an immunogenic amino acid sequence may comprise, consist essentially of, consist of, or is, an amino acid sequence as set forth in SEQ ID NO:59, or a fragment, variant, or derivative thereof.

Clinical diagnosis of Q fever is challenging as the signs are not pathognomonic and can easily be confused with other diseases such as leptospirosis and dengue. Several full length sequences of Coxiella burnetti antigens, including Com1, OmpH, YbgF, and GroEL as described in the Examples, can be used as diagnostic markers for Q fever, and may comprise, consist essentially of, consist of, or is, an amino acid sequence selected from the group consisting of an amino acid sequence as set forth in SEQ ID NO:59, SEQ ID NO:60, SEQ ID NO:61, SEQ ID NO:62, SEQ ID NO:63, SEQ ID NO:72, SEQ ID NO: 73, and any of one of SEQ ID NOs:74-100 (as set out in Table 7), or a fragment, variant, or derivative thereof, and any combination thereof.

In some embodiments, a Coxiella amino acid sequence or immunogenic amino acid sequence may be derived from, or correspond to, any one of the amino acid sequences as set forth in Examples 11 and/or 12, and Table 7 (and any associated Figures), or a fragment, variant, or derivative thereof. or a fragment, variant, or derivative thereof.

In other general embodiments relating to tuberculosis, preferably a Mycobacterium species is Mycobacterium tuberculosis and/or Mycobacterium bovis. In some embodiments, a Mycobacterium species immunogen may be an immunogenic amino acid sequence derived from, or corresponding to, an early stage antigen and/or a latency-associated antigen. In some embodiments, the early stage antigen is selected from an Ag85B antigen and/or an TB10.4 antigen. In some embodiments, the latency-associated antigen may be a Rv2660c protein. In some embodiments, the immunogenic amino acid sequence may comprise, consist essentially of, or consist of an amino acid sequence derived from, or corresponding to, an Ag85B antigen and a TB10.4 antigen, or a fragment, variant, or derivative thereof. Such a combination may be known in the art as H4 antigen. In some embodiments, the immunogenic amino acid sequence may comprise an amino acid sequence derived from, or corresponding to, an Ag85B antigen, a TB10.4 antigen, and a Rv2660c protein, inclusive of fragments, variants, and derivatives thereof. Such a combination may be known in the art as H28 antigen. Exemplary amino acid sequences for H4 and H28 antigens are set forth in SEQ ID NOS:6 and 7, respectively.

In some embodiments, the immunogenic amino acid sequence derived from, or corresponding to, a Mycobacterium tuberculosis and/or a Mycobacterium bovis comprises, consists of, consists essentially of, or is, an amino acid sequence selected from the group consisting of an amino acid sequence as set forth in SEQ ID NO:6, SEQ ID NO:7, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40, or a fragment, variant, or derivative of any one of the aforementioned sequences, and any combination thereof.

In some embodiments, one or more immunogenic amino acid sequences derived from, or corresponding to, a Mycobacterium protein comprises, consists essentially of, or consists of an amino acid sequence as set forth SEQ ID NO:6 and/or SEQ ID NO:7, or a fragment, variant, or derivative thereof. In some embodiments, a protein particle relating to a Mycobacterium as described herein may comprise, consist of, consist essentially of, or is, an amino acid sequence, and suitably an immunogenic amino acid sequence, derived from, or corresponding to, an amino acid sequence as set forth in any one of SEQ ID NOS:19, 20, and/or SEQ ID NOS:32 to 40, or a fragment, variant, or derivative thereof, and any combination thereof. An amino acid sequence in any one of SEQ ID NOS:32 to 40 may be particularly suitable in some embodiments that may relate to methods of detection relating to a Mycobacterium.

In some embodiments, a Mycobacterium immunogenic amino acid sequence may be derived from, or correspond to, any one of the amino acid sequences as set forth in any one of Example 1, Table 3 and/or Table 4, or a fragment, variant, or derivative thereof.

In certain broad aspects, the invention provides an isolated protein comprising a diphtheria toxin CRM amino acid sequence and an immunogenic amino acid sequence derived from a Mycobacterium species. In other broad aspects, the invention encompasses an isolated nucleic acid encoding the isolated protein or amino acid sequence comprising a CRM amino acid sequence and an immunogenic amino acid sequence derived from a Mycobacterium species. The isolated protein may be a chimera. In further broad aspects, the invention provides a genetic construct comprising the isolated nucleic acid an isolated nucleic acid encoding the isolated protein or amino acid sequence comprising a CRM amino acid sequence and an immunogenic amino acid sequence derived from a Mycobacterium species. In other broad aspects, the invention provides a host cell comprising the genetic construct. The host cell may be selected from a prokaryotic cell and a eukaryotic cell. The host cell may a prokaryotic cell. The prokaryotic cell may be a E. coli. In yet further broad aspects, the invention provides a protein particle comprising one or more isolated proteins comprising a CRM amino acid sequence and an immunogenic amino acid sequence derived from a Mycobacterium species. In broad aspects, the invention provides a method of producing a protein particle including the steps of introducing the isolated nucleic acid or the genetic construct into a host cell, culturing the host cell under conditions which facilitate production of the isolated protein encoded by the isolated nucleic acid, and forming the protein particle from the isolated protein. The method of production may optionally include purifying the isolated protein. Other broad aspects of the present invention include a protein particle comprising a comprising a CRM amino acid sequence and an immunogenic amino acid sequence derived from a Mycobacterium species produced by this method. The isolated protein or protein particle may be produced by recombinant technology. The protein particle may be derived from a cell. The protein particle may be a substantially insoluble protein particle, particularly when formed or expressed in a cell. The protein particle and/or substantially insoluble protein particle may be derived from an insoluble component of a cell. The insoluble component may be an inclusion body. In certain embodiments, the CRM amino acid sequence is not derived from a CRM protein, or a fragment thereof, that has been subjected to a protein refolding treatment. In some embodiments, the protein particle is not subjected to a protein refolding treatment. In broad aspects, the invention provides a composition, suitable a pharmaceutical composition, comprising an isolated protein comprising a CRM amino acid sequence and an immunogenic amino acid sequence derived from a Mycobacterium species and/or a protein particle comprising a CRM amino acid sequence and an immunogenic amino acid sequence derived from a Mycobacterium species, together with a pharmaceutically acceptable diluent, carrier, or excipient. The pharmaceutical composition may be an immunogenic composition, the immunogenic composition may be an immunotherapeutic composition, the immunotherapeutic composition may be a vaccine. In yet further broad aspects, the invention provides a method of eliciting an immune response to a Mycobacterium species in a subject, a method of immunising a subject against a Mycobacterium species, and a method of treating or preventing a Mycobacterium species infection in a subject, such methods including administering comprising an isolated protein comprising a CRM amino acid sequence and an immunogenic amino acid sequence derived from a Mycobacterium species and/or a protein particle comprising a CRM amino acid sequence and an immunogenic amino acid sequence derived from a Mycobacterium species, or a composition (e.g., pharmaceutical composition) comprising the same. Suitably, the immune response is, comprises, or elicits a protective immune response. In some embodiments, the subject may be a mammal. Suitably, the mammal may be a human. In certain embodiments, the Mycobacterium species is Mycobacterium tuberculosis and/or Mycobacterium bovis. In some embodiments, the immunogenic amino acid sequence may be derived from, or corresponds to, a Mycobacterium early stage antigen. In some embodiments, the Mycobacterium early stage antigen may be selected from an Ag85B antigen and/or an TB10.4 antigen. In some embodiments, the immunogenic amino acid sequence may be derived from, or correspond to, a Mycobacterium latency-associated antigen. In some embodiments, the latency associated antigen is a Rv2660c protein. In some embodiments, the immunogenic amino acid sequence may comprise an amino acid sequence derived from, or corresponding to, an Ag85B antigen, a TB10.4 antigen, and a Rv2660c protein, inclusive of fragments, variants, and derivatives thereof. Such a combination may be known in the art as H28 antigen. Amino acid sequences for exemplary H4 and H28 antigens are set forth in SEQ ID NOS:6 and 7. More preferably, one or more immunogenic amino acid sequences derived from, or corresponding to, a Mycobacterium protein comprises, consists essentially of, or consists of an amino acid sequence as set forth in SEQ ID NO:6 and/or SEQ ID NO:7, or a fragment, variant, or derivative thereof. In some embodiments, a protein particle as described herein may further comprise an amino acid sequence derived from, or corresponding to, an amino acid sequence as set forth in any one of SEQ ID NOS:32 to 40, wherein in some embodiments, the amino acid sequence may be an immunogenic amino acid sequence. According to these aspects, methods and other characteristics related to the isolated protein comprising a CRM amino acid sequence and an immunogenic amino acid sequence derived from a Mycobacterium species, and the isolated nucleic acid, host cell, genetic constructs, protein particles, methods of production of the same, recombinant expression, compositions (e.g., pharmaceutical compositions), and therapeutic methods etc related to the same, are generally described above and may be applied accordingly. According to these particular aspects and embodiments relating to a Mycobacterium species, the CRM amino acid sequence is derived from, or corresponds to, a CRM197 protein, or a fragment, variant, or derivative thereof. Preferably, the CRM amino acid sequence is derived from, or corresponds to, a CRM197 protein comprising, consisting of, or consisting essentially of, an amino acid sequence as set forth in SEQ ID NO:2, SEQ ID NO:49 and/or SEQ ID NO:50.

Other medically relevant microorganisms have been described extensively in the literature, e.g., see C. G. A Thomas, Medical Microbiology, Bailliere Tindall, Great Britain 1983, the entire contents of which is hereby incorporated by reference.

The invention also contemplates use of one or more immunogens derived from, or corresponding to, a protein associated with or causative of a cancer, a neurological disease, (and more preferably a degenerative neurological disease), an allergy and an autoimmune disease. Such proteins may be self-antigens.

In another broad embodiment, the disease, disorder or condition is a cancer. As generally used herein, the terms “cancer”, “tumour”, “malignant”, and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression. Non-limiting examples of cancers and tumours include sarcomas, carcinomas, adenomas, leukaemias and lymphomas, lung cancer, colon cancer, liver cancer, oesophageal cancer, stomach cancer, pancreatic cancer, neuroblastomas, glioblastomas and other neural cancers, brain, breast cancer, cervical cancer, uterine cancer, head and neck cancers, kidney cancer, prostate cancer and melanoma.

In some embodiments, the cancer may be amenable to treatment of, or responsive to, immunotherapy. Accordingly, in some embodiments, an immunogen against which an immune response is sought may be an antigen associated with or causative of a cancer, and in particular a condition such as a tumour i.e., a tumour antigen.

Therefore, the invention contemplates tumour antigens and tumour associated antigens (may collectively be referred to as “cancer antigens”) found in or associated with a germ cell tumour, a bowel cancer, a breast cancer, an ovarian cancer, a genitourinary cancer such as a prostate cancer and a testicular cancer, a brain cancer, a liver cancer, a pancreatic cancer, an oesophageal cancer, B cell lymphoma, T cell lymphoma, myeloma, leukemia, hematopoietic neoplasias, thymoma, lymphoma, sarcoma, lung cancer, non-Hodgkins lymphoma, Hodgkins lymphoma, uterine cancer, adenocarcinoma, pancreatic cancer, colon cancer, lung cancer, renal cancer, bladder cancer, a primary or metastatic melanoma, squamous cell carcinoma, basal cell carcinoma, angiosarcoma, hemangiosarcoma, head and neck carcinoma, thyroid carcinoma, soft tissue sarcoma, bone sarcoma, uterine cancer, cervical cancer, gastrointestinal cancer, biliary tract cancer, choriocarcinoma, colon cancer, endometrial cancer, esophageal cancer, gastric cancer, intraepithelial neoplasms, lymphomas, lung cancer (e.g., small cell and non-small cell), neuroblastomas, oral cancer, rectal cancer; skin cancer, as well as other carcinomas and sarcomas, although without limitation thereto and any other cancer now known or later identified (see, e.g., Rosenberg (1996) Ann. Rev. Med. 47:481-491, the entire contents of which are incorporated by reference herein). It will be appreciated that the cancer may be a malignant or non-malignant cancer.

Non-limiting examples of tumour and/or tumour-associated antigens are alphafetoprotein, carcinoembryonic antigen (CEA), CA-125, MUC-1, ras, p53, epithelial tumor antigen (ETA), tyrosinase, HER2/neu and BRCA1 antigens for breast cancer, MART-1/MelanA, gplOO, TRP-1, TRP-2, NY-ESO-1, CDK-4, l3-catenin, MUM-1, Caspase-8, KIAA0205, HPV E7, SART-1, PRAME, and p15 antigens, members of the Melanoma-associated antigen (MAGE) family, the BAGE family (such as BAGE-1), the DAGE/PRAME family (such as DAGE-1), the GAGE family, the RAGE family (such as RAGE-1), the SMAGE family, NAG, TAG-72, CA125, mutated proto-oncogenes such as p21 ras, mutated tumor suppressor genes such as p53, tumor associated viral antigens (e.g., HPV16 E7), the SSX family, HOM-MEL-55, NY-COL-2, HOM-HD-397, HOM-RCC1.14, HOM-HD-21, HOM-NSCLC-11, HOM-MEL-2.4, HOM-TES-11, RCC-3.1.3, NY-ESO-1, and the SCP family. Members of the MAGE family include, but are not limited to, MAGE-1, MAGE-2, MAGE-3, MAGE4 and MAGE-11. Members of the GAGE family include, but are not limited to, GAGE-1, GAGE-6. See, e.g., review by Van den Eynde and van der Bruggen (1997) in Curr. Opin. Immunol. 9: 684-693, Sahin et al. (1997) in Cum Opin. Immunol. 9: 709-716, and Shawler et al. (1997), the entire contents of which are incorporated by reference herein for their teachings of cancer antigens.

A cancer antigen can also be, but is not limited to, human epithelial cell mucin (Muc-1; a 20 amino acid core repeat for Muc-1 glycoprotein, present on breast cancer cells and pancreatic cancer cells), MUC-2, MUC-3, MUC-18, the Ha-ras oncogene product, carcino-embryonic antigen (CEA), ovarian carcinoma antigen (CA125), the raf oncogene product, CA-125, GD2, GD3, GM2, TF, sTn, gp75, EBV-LMP 1 & 2, HPV-F4, 6, 7, prostatic serum antigen (PSA), prostate-specific membrane antigen (PSMA), C017-1A, GA733, gp72, p53, the ras oncogene product, I3-HCG, gp43, HSP-70, pi 7 mel, HSP70, gp43, HMW, HOJ-1, melanoma gangliosides, TAG-72, HER2 antigen, mutated proto-oncogenes such as p21 ras, mutated tumor suppressor genes such as p53, estrogen receptor, milk fat globulin, telomerases, nuclear matrix proteins, prostatic acid phosphatase, protein MZ2-E, polymorphic epithelial mucin (PEM), folate-binding-protein LK26, truncated epidermal growth factor receptor (EGFR), Thomsen-Friedenreich (T) antigen, GM-2 and GD-2 gangliosides, polymorphic epithelial mucin, folate-binding protein LK26, human chorionic gonadotropin (HCG), pancreatic oncofetal antigen, cancer antigens 15-3,19-9, 549,195, squamous cell carcinoma antigen (SCCA), ovarian cancer antigen (OCA), pancreas cancer associated antigen (PaA), mutant K-ras proteins, mutant p53, and chimeric protein p210BCR_ABL and tumor associated viral antigens (e.g., HPV16 E7).

A cancer antigen can also be an antibody produced by a B cell tumor (e.g., B cell lymphoma; B cell leukemia; myeloma; hairy cell leukemia), a fragment of such an antibody, which contains an epitope of the idiotype of the antibody, a malignant B cell antigen receptor, a malignant B cell immunoglobulin idiotype, a variable region of an immunoglobulin, a hypervariable region or complementarity determining region (CDR) of a variable region of an immunoglobulin, a malignant T cell receptor (TCR), a variable region of a TCR and/or a hypervariable region of a TCR. In one embodiment, a cancer antigen of this invention can be a single chain antibody (scFv), comprising linked VH, and VL domains, which retains the conformation and specific binding activity of the native idiotype of the antibody. The cancer antigens that can be used in accordance with the present invention are in no way limited to the cancer antigens listed herein. Other cancer antigens can be identified, isolated and cloned by methods known in the art such as those disclosed in U.S. Pat. No. 4,514,506, the entire contents of which are incorporated by reference herein.

In other aspects, the disease, disorder, or condition is associated with a transplantation antigen. A non-limiting example of transplantation antigen-associated disease, disorder, or condition is a graft versus host disease. A wide variety of transplantation antigens have been described, including the MHC molecules, minor histocompatibility antigens, ABO blood group antigens, and monocytes/endothelial cell antigens, and may find use with the present invention.

An immunogen of interest may be one that is associated with or causative of autoimmune diseases such as rheumatoid arthritis and diabetes are particularly amenable for use in the present invention. In suitable embodiments that relate to rheumatoid arthritis, the antigen may be derived from arteriogenic auto-antigen.

An immunogen of interest can further be an autoantigen (for example, to enhance self-tolerance to an autoantigen in a subject, e.g., a subject in whom self-tolerance is impaired). Exemplary autoantigens include, but are not limited to, myelin basic protein, islet cell antigens, insulin, collagen and human collagen glycoprotein, muscle acetylcholine receptor and its separate polypeptide chains and peptide epitopes, glutamic acid decarboxylase and muscle-specific receptor tyrosine kinase.

The present invention also relates to use of immunogen that are allergens. An “allergen” refers to a substance that can induce an allergic or asthmatic response in a susceptible subject. An “allergy” refers to acquired hypersensitivity to a substance (allergen). Allergic conditions include but are not limited to eczema, allergic rhinitis or coryza, conjunctivitis, hay fever, bronchial asthma, urticaria (hives) and food allergies, and other atopic conditions. Allergies are generally caused by IgE antibody generation against harmless allergens. The list of allergens is enormous and can include pollens, insect venoms, plant proteins, animal dander dust, fungal spores and drugs (e.g., penicillin). Examples of natural, animal and plant allergens include but are not limited to proteins specific to the following genuses: Canine (e.g., Canis familiaris); Dermatophagoides (e.g., Dermatophagoides farinae); Felis (e.g., Felis domesticus); Ambrosia (e.g., Ambrosia artemisiifolin); Lolium (e.g., Lolium perenne); Cryptomeria (e.g., Cryptomeria japonica); Alternaria (e.g., Alternaria alternata); Alder, Alnus (e.g., Alnus glutinosa); Betula (e.g., Betula verrucosa); Quercus (e.g., Quercus alba); Olea (e.g., Olea europa); Artemisia (e.g., Artemisia vulgaris); Plantago (e.g. Plantago lanceolate); Panetaria (e.g., Parietaria officinalis or Panetaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis and Juniperus ashei); Thuya (e.g., Thuya orientalis); Chamaecyparis (e.g., Chamaecyparis obtusa); Penplaneta (e.g., Pehplaneta amehcana); Agropyron (e.g., Agropyron repens); Secale (e.g., Secale cereale); Triticum (e.g., Triticum aestivum); Dactylis (e.g., Dactylis glomerata); Festuca (e.g. Festuca elatior); Poa (e.g. Poapratensis or Poa compressa); Avena (e.g., Avena sativa); Holcus (e.g., Holcus lanatus); Anthoxanthum (e.g., Anthoxanthum odoratum); Arrhenatherum (e.g., Arrhenatherum elatius); Agrostis (e.g. Agrostis alba); Phleum (e.g., Phleum pratense); Phalaris (e.g., Phalaris arundinacea); Paspalum (e.g., Paspalum notatum); Sorghum (e.g., Sorghum halepensis); Ricinus (e.g., Ricinus communis and more preferably, ricin protein) and Bromus (e.g., Bromus inermis).

In relation to degenerative neurological diseases associated with dementia, the invention contemplates Alzheimer's disease and Lewy body dementia, although without limitation thereto. An immunogenic protein for Alzheimer's disease includes (and without limitation thereto) Amyloid beta (Aβ or Abeta), which is a peptide of 36-43 amino acids that appears to be the main constituent of amyloid plaques, which are deposits found in the brains of patients with Alzheimer's disease).

In broad aspects, the invention relates to compositions, including pharmaceutical compositions, for use in methods as described herein. In certain broad aspects, the invention provides a composition comprising a protein particle comprising a diphtheria toxin CRM amino acid sequence and optionally one or more immunogens other than a diphtheria toxin CRM amino acid sequence as herein described, wherein the protein particle is derived from a cell, and a pharmaceutically-acceptable diluent, carrier, or excipient. In some embodiments, the invention may provide a pharmaceutical composition comprising a protein particle comprising a diphtheria toxin CRM amino acid sequence and optionally one or more immunogens other than a diphtheria toxin CRM amino acid sequence as herein described, wherein the protein particle is derived from a cell, and a pharmaceutically-acceptable diluent, carrier, or excipient.

In some embodiments, combinations of immunogens derived from the organisms, proteins, and/or agents above can be conveniently used to elicit immunity or an immune response to multiple pathogens, proteins, and/or agents in a single composition, preferably a pharmaceutical composition, more preferably an immunogenic composition, and even more preferably an immunotherapeutic composition, and yet even more preferably, a vaccine.

In some embodiments where the intended use is to induce an immune response, the composition (including pharmaceutical composition) may be referred to as an immunogenic composition.

In some embodiments, an immunogenic composition may be an immunotherapeutic composition. In particularly preferred embodiments, the immunotherapeutic composition may be a vaccine. It will be appreciated that immunotherapeutic compositions of the invention may be used to prophylactically or therapeutically.

It will be appreciated that the compositions described herein include preventative compositions (i.e., compositions administered for the purpose of preventing a condition such as an infection or a cancer) and therapeutic compositions (i.e., compositions administered for the purpose of treating conditions such as an infection or a cancer). Therefore, a composition may therefore be administered to a recipient for prophylactic, ameliorative, palliative, or therapeutic purposes.

Any suitable procedure is contemplated for producing compositions as described herein, such as vaccine compositions. Exemplary procedures include, for example, those described in New Generation Vaccines (1997, Levine et al., Marcel Dekker, Inc. New York, Basel, Hong Kong), which is incorporated herein by reference.

The composition (e.g., pharmaceutical composition as described herein) may further comprise a pharmaceutically-acceptable carrier, diluent or excipient.

By “pharmaceutically-acceptable carrier, diluent or excipient” is generally meant a solid or liquid filler, diluent, solvent, vehicle or encapsulating substance that may be safely used in administration to a subject. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful general reference describing carriers, diluents and excipients is Remington's Pharmaceutical Sciences (Mack Publishing Co. N.J. USA, 1991) which is incorporated herein by reference.

In particular embodiments, the carrier, diluent, or excipient may include carriers, diluents and/or excipients that have immunological activity, or facilitate immunological activity. For example, these may include: thyroglobulin; albumins such as human serum albumin; toxins, toxoids or any mutant crossreactive material of the toxin from tetanus, diphtheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a T cell epitope of a bacterial toxin, or toxoid or the like may be used. In this regard, reference may be made to U.S. Pat. No. 5,785,973 which is incorporated herein by reference.

The composition may further comprise an adjuvant as is well known in the art. As described herein, a protein particle of the invention may be co-administered with an adjuvant.

As will be understood in the art, an “adjuvant” is, or comprises, one or more substances that enhance the immunogenicity and efficacy of a composition, such as a vaccine. Non-limiting examples of suitable adjuvants include squalane and squalene (or other oils of plant or animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Bordetella pertussis antigens; tetanus toxoid; diphtheria toxoid; surface active substances such as hexadecylamine, octadecylamine, octadecyl amino acid esters, lysolecithin, dimethyldioctadecylammonium bromide, N,N-dioctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol' EMA; acrylic copolymer emulsions such as Neocryl A640 (e.g. U.S. Pat. No. 5,047,238); water in oil emulsifiers such as Montanide ISA 720; poliovirus, vaccinia or animal poxvirus proteins; or mixtures thereof and immuno stimulatory DNA such as CpG oligonucleotides and Toll receptor agonists. In some embodiments, the adjuvant may be, or comprise, dimethyl dioctadecyl ammonium bromide. It will be appreciated that in some embodiments, an adjuvant may be used that facilitates, enhances, or supports one or more characteristics of the protein particle to be formulated. The choice of an adjuvant may aid in formulation or an activity of the protein particle (e.g., immunogenicity), although without limitation thereto. In some embodiments, an adjuvant may modulate surface charge of a protein particle as described herein. Modulation of surface charge may be required to facilitate or enhance formulation of a composition. In some other embodiments, an adjuvant may impact a size, and/or size distribution of a protein particle or composition containing said particles, or the heterogeneity of particle size in a sample, as described herein. By way of example only, an adjuvant may be useful to convert a protein particle sample or population that is heterogeneous with respect to particle size to a homogenous sample.

In some embodiments, the adjuvant may be an alum and/or dimethyl dioctadecyl ammonium bromide.

It will be appreciated that a composition as described herein in some embodiments may include one or more ancillary agents to assist with formulation or preparation of the composition or to assist with achieving a desired outcome for the protein particle as described herein (e.g. immunogenicity, minimise side effects, particle size distribution, particle charge).

As hereinbefore described, the immunogenic composition and/or vaccine of the invention may include an “immunologically-acceptable carrier, diluent or excipient”. An “immunologically-acceptable carrier, diluent or excipient” includes within its scope water, bicarbonate buffer, phosphate buffered saline or saline and/or an adjuvant as is well known in the art.

Any safe route of administration may be employed for providing an animal with the composition of the invention. For example, oral, rectal, parenteral, sublingual, buccal, intravenous, intranasal, intra-articular, intra-muscular, intradermal, subcutaneous, inhalational, intraocular, intraperitoneal, intracerebroventricular and transdermal administration may be employed. Intra-muscular and subcutaneous injection may be particularly appropriate, for example, for administration of immunogenic compositions and vaccines.

Dosage forms include tablets, dispersions, suspensions, injections, solutions, syrups, troches, capsules, nasal sprays, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of suitable for administration may be presented as discrete units such as capsules, caplets, sachets, functional foods/feeds or tablets, or as a powder or granules or as a solution or a suspension in an aqueous liquid, a non-aqueous liquid, an oil-in-water emulsion or a water-in-oil liquid emulsion. Such compositions may be particularly suitable for oral or parenteral administration. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The composition may be administered in a manner compatible with the dosage formulation, and in such amount as is immunologically effective. The dose administered to an animal should be sufficient to effect a beneficial response in an animal over an appropriate period of time. The quantity of agent(s) to be administered may depend on the animal to be treated inclusive of the age, sex, weight and general health condition thereof, factors that will depend on the judgement of the practitioner.

As will be appreciated, the protein particles and/or the compositions as described herein are administrable to a subject as described herein, inclusive of a vertebrate host, and more preferably a mammalian subject inclusive of livestock (e.g., cattle, sheep, pigs), performance animals (e.g., racehorses, greyhounds), companion animals (e.g., dogs, cats), laboratory animals (e.g., mice, rats), and humans, without limitation thereto and described herein. Accordingly, it will be appreciated that the protein particles and compositions comprising the protein particles as described herein are administrable to human and nonhuman vertebrates, inclusive of veterinary applications.

In some embodiments, a composition as described herein may comprise a protein particle as described herein that may be a substantially insoluble protein particle derived from, purified from, produced, isolated from, or obtained from, an insoluble component of a cell. In some embodiments, an insoluble component may be obtained, purified, or produced from a cell. In certain embodiments, the insoluble component may be an inclusion body.

According to some embodiments, a composition as described herein may comprise a protein particle as described herein in the form of an insoluble component obtained, isolated, derived, purified, or produced from a cell. In certain embodiments, the insoluble component may be an inclusion body as described herein.

It will be appreciated that a protein particle or a composition as described herein may be used in a method to detect a target in a sample. It is contemplated that in some embodiments, the protein particle as described herein may be an immunodiagnostic reagent. It is envisaged that that protein particles as described herein may be used for analytical, screening, and/or diagnostic applications. It will be appreciated that such methods may be methods of analysing, detecting, and/or quantifying molecules or biological structures of interests. Therefore, the target may be a ligand, protein, a peptide, a polypeptide, an immunoglobulin, biotin, an inhibitor, a co-factor, an enzyme, a receptor, a monosaccharide, an oligosaccharide, a polysaccharide, a glycoprotein, a lipid, a nucleic acid, a hormone, a toxin or any other molecule, a cell or fragment thereof, an organelle, a virus, a bacterium, a fungus, a protist, a parasite, an animal, a plant or any substructure, fragments or combinations thereof.

It is envisaged that in some embodiments, the compositions of the present invention may be suitable for used in a detection method as described herein. Such compositions may be suitable for diagnostics or may be suitable for immunodiagnostics as described herein.

In some embodiments, the detection methods as described herein may detect whether a subject has been exposed to an agent or a pathogen. According to such embodiments, the detection method may detect an immune response, or one or more elements of an immune response. In some embodiments, the detection methods as described herein may detect whether a subject is suffering from a disease, condition and/or disorder (e.g., caused by a pathogen or an agent). The, or each, elements of an immune response may be any suitable element e.g., an antibody, an immune cell, a T-cell, and or a B-cell, In some embodiments, the detection methods as described herein may detect whether a subject has been immunised against a pathogen or agent. It will be appreciated that for the purpose of a detection method of the present invention, the protein particles comprising a CRM amino acid sequence may further comprise one or more amino acid sequences, wherein the one or more amino acid sequences may or may not be an immunogenic amino acid sequence. In those embodiments which encompass protein particles further comprising one or more amino acid sequences which are not immunogenic per se, said one or more amino acid sequence may be suitable for use in detection methods by recognising a target molecule.

As will be appreciated in light of the foregoing, a protein particle comprising a CRM amino acid sequence in detection methods or methods of determining may be used as a carrier system to display diagnostic antigens, or fragments, variants, and derivatives thereof. In some embodiments, one or more diagnostic antigens comprise, consist of, or consist essentially of, an amino acid sequence suitable for use as a diagnostic antigen and/or in the methods of the present invention. Such amino acid sequences suitable for use as a diagnostic antigen may, or may not be, capable of eliciting an immune response, and in some embodiments, may be capable of recognising an immune response, or one or more elements of an immune response (e.g. an antibody). A protein particle comprising a CRM amino acid sequence as described may optionally include such one or more “diagnostic” amino acid sequences. It will be appreciated that in some embodiments, such a protein particle that includes a one or more “diagnostic” amino acid sequences may further comprise one or more immunogens (e.g. an immunogen may include an immunogenic amino acid sequence) as herein described.

In some embodiments that relate to detection or diagnostic methods, a protein particle comprising a diphtheria toxin CRM amino acid sequence as described herein may optionally further comprise an amino acid sequence as set forth in any one of SEQ ID NOS:17-22, 28-48, and 56-100, or a fragment, variant, or derivative thereof, and any combination thereof.

It is contemplated that when a protein particle according to the present invention is contacted with a sample containing a mixture of components, the particle selectively binds to a target molecule or biological entity. Thus, binding of the protein particle to the target molecule or biological entity, followed by removal of unbound particles, may allow for detection (and possibly quantification) of the target molecule or biological entity.

A sample, a test sample, is or comprises blood, serum, cells, tissues, plasma, organs, cultures (e.g., human, animal, plant), biological fluid, respiratory wash sample, lavage sample, mucus sample, plasma sample, cerebrospinal fluid, urine, skin or other tissues, or fractions thereof. Combinations of samples are contemplated. In some embodiments, the sample is a biological sample. In yet other embodiments, the sample, the test sample, and/or biological sample is obtained from a subject.

In some embodiments, the sample may comprise or is an intranasal tissue or cell, an oropharyngeal tissue or cell. It will be appreciated that use of such samples may be useful for detection of a coronavirus, preferably a SARS coronavirus, and more preferably a SARS-CoV-1 and/or a SARS-CoV-2. In some embodiments that relate to coronavirus, non-limiting examples of suitable specimens from a sample may be colled include a nasal mid-turbinate swab, an anterior nares (nasal swab) specimen, a nasopharyngeal wash/aspirate and/or nasal wash/aspirate specimen.

In many analyses or detection procedures on liquid samples it may be advantageous to adsorb the sample to a solid surface. This may be done in a number of ways including adding the sample to a well in a microtitre plate, depositing the sample onto a membrane made from materials such as nitrocellulose (NC), nylon, PVDF or any other suitable material, either by direct application or electroblotting following electrophoretic gel fractionation of the sample (e.g., Western blotting).

Non-limiting examples of suitable detection methods include colourimetric based methods including an ELISA, microfluidic-based methods, a receptor-binding assay, protein quantification-based methods, serological methods, and others that will be apparent to a person of skill the art.

In some embodiments, the detection method may be an immunodiagnostic method. In some embodiments, the immunodiagnostics method may detect an immune response.

Accordingly, the protein particles as described herein may be used in lieu of, or in addition to, normally used or typically antigens or other analytes in conventional detection methods. By way of example, the protein particles as described herein may be used in any and all formats of ELISA (e.g, direct, indirect, capture, sandwich), particularly if an immune response is being detected.

In some embodiments that relate to tuberculosis and/or a Mycobacterium-related infection, a detection method may include contacting a protein particle described herein with a sample wherein the sample is a skin portion. In such embodiments, the method may include contacting a skin portion of a test sample with a protein particle as described herein comprising a CRM amino acid sequence and one or more immunogenic amino acid sequence derived from, or corresponding to, one or more Mycobacterium immunogens. It will be understood that the tuberculosis skin test may be capable of discriminating between a subject exposed to Mycobacterium tuberculosis and/or Mycobacterium bovis, or suffering from tuberculosis, and/or a subject immunised against tuberculosis, for example with BCG. Although not wishing to be bound by any particular theory, it is proposed that the CRM protein particles comprising one or more Mycobacterium immunogenic amino acid sequences as described herein may be more efficient (antigen sparing) in inducing specific and sensitive skin responses for detection of tuberculosis than conventional methods. In some embodiments, the protein particle may be injected into a skin portion of a subject. Suitably, a skin-based test method for detecting a tuberculosis may measure or detect a delayed-type hypersensitivity response. An example of a suitable skin test is the Mantoux test or a tuberculin skin test as would be known by the skilled addressee. A non-limiting example of a positive skin test measuring a delayed type hypersensitivity response is as follows: (i) SICCT (single intradermal comparative cervical test) response considered positive if the change in (Δ) skin thickness for purified protein derivative B (PPD-B)—purified protein derivative A (PPD-A) may be >4 mm; or (ii) >2 mm (e.g., United Kingdom test); (iii) A single intradermal test (SIT) response is considered positive if Δ skin thickness for PPD-B may be ≥4 mm; and (iv) responses for CRM particles of the invention are considered positive if Δ skin thickness may be ≥1 mm. For example, a positive skin test with a diagnostic reagent of the present invention or in a method of the invention, including, for example, a method comprising the administration to a skin portion of a diagnostic reagent comprising less than 0.5 μg of each antigen present per dose may be a Δ skin thickness of ≥1 mm.

In other embodiments relating to tuberculosis, a blood sample, and preferably a whole blood sample, may be tested. In some embodiments that contemplate a blood sample, the method may be a method where a protein particle comprising a CRM amino acid sequence and one or more immunogenic amino acid sequence derived from, or corresponding to, one or more Mycobacterium tuberculosis immunogens as described herein are contacted with a blood-derived test sample, preferably white blood cells, to detect an interferon-gamma (IFN-γ) release, which is indicative of a positive results for Mycobacterium tuberculosis. Interferon-Gamma Release Assays (IGRAs) for Mycobacterium tuberculosis will be known to the skilled addressee. IGRA is a blood test that may be used to ascertain whether a subject has been infected with Mycobacterium tuberculosis/bovis. The IGRA test works by measuring the body's immune response to the Mycobacterium tuberculosis/bovis. White blood cells from most subjects that have been infected with Mycobacterium tuberculosis will release interferon-gamma (IFN-g) when mixed with antigens (substances that can produce an immune response) derived from Mycobacterium tuberculosis. An IGRA test may be used to diagnose latent tuberculosis infection. The method may be capable of discriminating between a subject exposed to Mycobacterium tuberculosis or Mycobacterium bovis or suffering from tuberculosis, and a subject immunised against tuberculosis, for example with BCG. The invention comprises a diagnostically or therapeutically effective amount is an amount effective to elicit an immunological response, such as, for example, a concentration of IFN-gamma in the blood of between about 0.5 ng/mL and about 20 ng/mL, between about 0.5 ng/mL and about 15 ng/mL, between about 0.5 ng/mL and about 10 ng/mL, between about 0.5 ng/mL and about 9 ng/mL, between about 1 ng/mL and about 8 ng/mL, between about 2 ng/mL and about 7 ng/mL, or between about 3 ng/mL and about 6 ng/mL. In some circumstances, including post infection or during prolonged infection, elevated IFN-gamma blood concentrations are observed, and such elevated concentrations should be accounted for in assessing a baseline against which elicitation of an effective immunological response by the protein particles of the invention is to be assessed. Although not wishing to be bound by any particular theory, it is proposed that CRM particles comprising one or more immunogenic amino acid sequences derived from, or corresponding to, a Mycobacterium are more efficiently taken up by APC, which may result in more sensitive and specific IGRAs.

As will be appreciated in light of the foregoing, a protein particle comprising a CRM amino acid sequence as described herein may be used as a carrier system to display one or more mycobacterial diagnostic antigens for the development of tuberculosis skin test and/or blood test reagents (e.g., IGRA). Tables 3 and 4 provides non-limiting examples of one or more amino acid sequences derived from, or corresponding to, a Mycobacterium protein that may be used in a protein particle in a diagnostic or detection method or kit of the present invention, inclusive of fragments, variants, or derivatives thereof. In some embodiments, the one or more amino acid sequences, diagnostic amino acid sequences, or one or more immunogenic amino acid sequences may comprise, consist essentially of, or consist of an amino acid sequence (or a fragment of said amino acid sequence) as set forth in any one of SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40, or fragments, variants, or derivatives, and any combination thereof. SEQ ID NOS:32 to 40 may be present any other myobacterial amino acid sequence as described herein and in some embodiments, an amino acid sequence as set forth in any one of SEQ ID NOS:6, 7, 19 and/or 20, or a fragment, variant, or derivative thereof.

According to some embodiments relating to detection of a Coxiella species, and preferably Coxiella burnetti, the one or more amino acid sequences, diagnostic amino acid sequences, or one or more immunogenic amino acid sequences may comprise, consist essentially of, or consist of an amino acid sequence (or a fragment of said amino acid sequence) as set forth in any one of SEQ ID NOS:59 to 63, or a fragment, variant, or derivative thereof, and any combination thereof. In some further embodiments, the sequences is an amino acid sequence as set forth in any one of SEQ ID NOS:60 to 63, or a fragment, variant, or derivative thereof, and any combination thereof.

According to some embodiments that may relate to a Coronaviridae virus, the one or more amino acid sequences, diagnostic amino acid sequences, or one or more immunogenic amino acid sequences may comprise, consist essentially of, or consist of an amino acid sequence (or a fragment of said amino acid sequence) as set forth in any one of SEQ ID NOS:56 to 58, or a fragment, variant, or derivative thereof, and any combination thereof. In some embodiments, the Coronaviridae virus may be a coronavirus. In some further embodiments, the coronavirus is a SARS coronavirus. In yet further embodiments, the SARS coronavirus may be a SARS CoV-1 and/or a SARS CoV-2.

The present invention also provides kits for detecting or quantifying a target from a sample, wherein the kits facilitate the employment of the protein particles and methods of the invention as described herein. Typically, kits for carrying out an analysis or diagnostic test contain at least a number of the reagents required to carry out the method. Typically, the kits of the invention will comprise one or more containers, containing for example, particles and wash reagents.

In the context of the present invention, a compartmentalised kit includes any kit in which particles and/or reagents are contained in separate containers, and may include small glass containers, plastic containers or strips of plastic or paper. Such containers may allow the efficient transfer of reagents from one compartment to another compartment whilst avoiding cross-contamination of. the samples and reagents, and the addition of agents or solutions of each container from one compartment to another in a quantitative fashion. Such kits may also include a container which will accept a test sample, a container which contains the particles used in the assay and containers which contain wash reagents (such as phosphate buffered saline, Tris-buffers, and like).

Typically, a kit of the present invention will also include instructions for using the kit components to conduct the appropriate methods.

Methods and kits of the present invention find application in any circumstance in which it is desirable to detect and/or quantify a component from a sample.

So that the invention may be fully understood and put into practical effect, reference is made to the following non-limiting Examples.

### EXAMPLES

### Example 1

### Immunogenicity Study of CRM197-Mycobacterium tuberculosis (TB) Particles

### Materials and Methods

### Strains, Plasmids, and Primers

All the bacterial strains, plasmids, and primers used in this study are listed in Table 1. XL1-Blue was used for plasmid construction and grown in Luria broth (LB; Thermo Fisher Scientific, USA), supplemented with ampicillin (100 μg/ml) at 37° C. ClearColi BL21 (DE3) was used for CRM197 inclusion body (particle) production.

### Plasmid Construction for Formation of CRM197 Particle and the CRM197 Particle Displaying a H4 or H28 Antigen

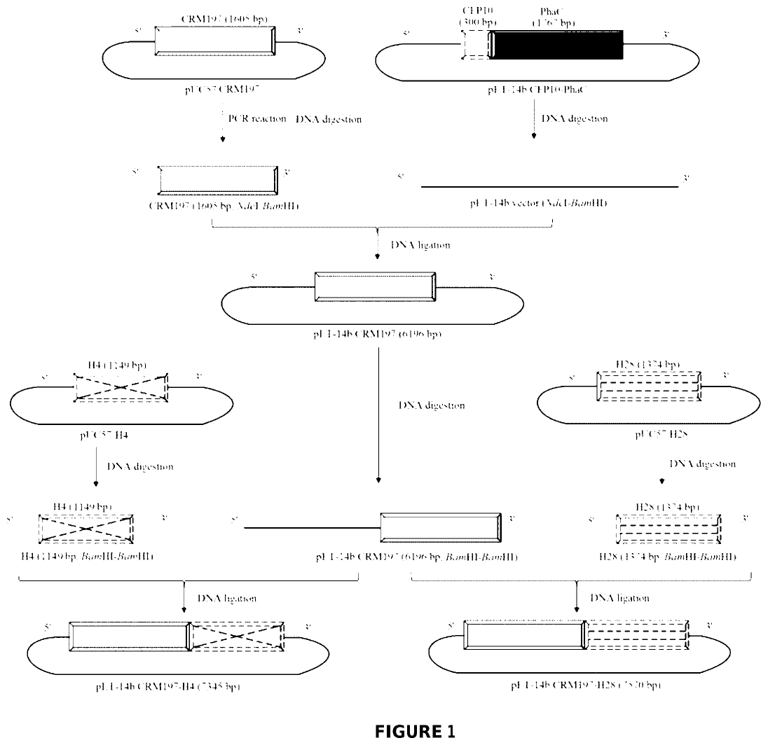

The gene fragment ATGGGTGCAGATGACGTGGTTGACAGCTCTAAATCTTTTGTGATGGAAAACTTCAGTTCC TATCATGGCACCAAACCGGGCTACGTTGATAGCATTCAGAAAGGCATCCAAAAACCGAA ATCTGGCACGCAGGGTAACTACGATGACGATTGGAAAGAATTTTACTCTACCGACAACAA ATACGATGCGGCCGGTTACTCAGTCGACAACGAAAATCCGCTGAGCGGTAAAGCGGGCG GTGTCGTGAAAGTGACGTATCCGGGTCTGACCAAAGTTCTGGCCCTGAAAGTCGATAATG CAGAAACCATCAAAAAAGAACTGGGTCTGAGTCTGACGGAACCGCTGATGGAACAGGTT GGCACCGAAGAATTTATCAAACGCTTCGGCGATGGTGCCAGTCGTGTTGTCCTGTCCCTG CCGTTCGCAGAAGGCTCATCGAGCGTTGAATATATTAACAATTGGGAACAAGCGAAAGC CCTGAGCGTCGAACTGGAAATCAACTTTGAAACCCGCGGCAAACGTGGTCAGGATGCCAT GTATGAATACATGGCACAGGCGTGCGCCGGTAATCGTGTGCGTCGCAGCGTTGGCTCTAG TCTGTCTTGTATCAACCTGGACTGGGATGTTATCCGTGATAAAACCAAAACGAAAATCGA AAGTCTGAAAGAACACGGTCCGATCAAAAACAAAATGTCAGAATCGCCGAATAAAACGG TGTCCGAAGAAAAAGCTAAACAGTATCTGGAAGAATTTCACCAAACCGCACTGGAACAT CCGGAACTGTCAGAACTGAAAACCGTTACGGGCACCAACCCGGTCTTTGCCGGCGCAAAT TACGCAGCTTGGGCTGTCAACGTGGCGCAAGTGATTGACTCGGAAACGGCGGATAATCTG GAAAAAACCACGGCGGCCCTGAGTATTCTGCCGGGCATCGGTTCCGTCATGGGTATTGCC GATGGCGCAGTGCATCACAACACCGAAGAAATTGTTGCCCAGAGTATCGCACTGTCCTCA CTGATGGTTGCTCAAGCGATTCCGCTGGTCGGTGAACTGGTGGATATTGGCTTTGCAGCTT ATAATTTCGTGGAATCCATTATCAACCTGTTTCAGGTGGTTCATAACTCATATAATCGCCC GGCGTACTCGCCGGGTCACAAAACCCAACCGTTCCTGCATGACGGCTACGCCGTGAGCTG GAATACGGTTGAAGATTCTATTATCCGTACCGGCTTTCAGGGTGAAAGCGGCCACGACAT TAAAATCACGGCTGAAAACACCCCGCTGCCGATTGCGGGTGTTCTGCTGCCGACCATCCC GGGTAAACTGGATGTCAATAAATCAAAAACCCATATCTCGGTGAACGGTCGCAAAATTCG TATGCGCTGCCGTGCCATCGACGGCGATGTGACCTTCTGTCGTCCGAAAAGCCCGGTTTA TGTCGGCAACGGTGTGCATGCTAATCTGCACGTTGCGTTTCATCGTAGCAGCAGCGAAAA AATTCACAGTAATGAAATCAGTTCCGACTCCATTGGTGTGCTGGGCTACCAGAAAACGGT CGATCATACCAAAGTGAACAGCAAACTGTCTCTGTTTTTCGAAATTAAATCTTAA (SEQ ID NO:1) encoding CRM197 protein sequence MGADDVVDSSKSFVMENFSSYHGTKPGYVDSIQKGIQKPKSGTQGNYDDDWKEFYSTDNKY DAAGYSVDNENPLSGKAGGVVKVTYPGLTKVLALKVDNAETIKKELGLSLTEPLMEQVGTE EFIKRFGDGASRVVLSLPFAEGSSSVEYINNWEQAKALSVELEINFETRGKRGQDAMYEYMA QACAGNRVRRSVGSSLSCINLDWDVIRDKTKTKIESLKEHGPIKNKMSESPNKTVSEEKAKQY LEEFHQTALEHPELSELKTVTGTNPVFAGANYAAWAVNVAQVIDSETADNLEKTTAALSILP GIGSVMGIADGAVHHNTEEIVAQSIALSSLMVAQAIPLVGELVDIGFAAYNFVESIINLFQVVH NSYNRPAYSPGHKTQPFLHDGYAVSWNTVEDSIIRTGFQGESGHDIKITAENTPLPIAGVLLPTI PGKLDVNKSKTHISVNGRKIRMRCRAIDGDVTFCRPKSPVYVGNGVHANLHVAFHRSSSEKI HSNEISSDSIGVLGYQKTVDHTKVNSKLSLFFEIKS (SEQ ID NO:2) was codon-optimized for E. coli cell (GenScript, USA). CRM197, an enzymatically inactive and nontoxic form of DTx produced by C. diphtheriae [1, 2] and the amino acid sequence used in this study did not contain the signal peptide and the encoded DNA sequence was codon-optimized by GenScript for E. coli using GeneArt™ GeneOptimizer™ software. The CRM197 gene sequence was excised from pUC57 vector (GenScript, USA) by restriction enzyme digestion with NdeI (BioLabs, USA), followed by DNA fragment separation using agarose gel electrophoresis with SYBR safe stain (Invitrogen, USA) and fragment extraction using DNA recovery kit (Zymo Research, USA). Polymerase chain reaction (PCR) was used to introduce BamHI restriction site to the 3′ end of the purified CRM197 gene fragment. The linear pET-14b vector, prepared by digesting the plasmid pET-14b CFP10-PhaC with NdeI and BamHI, was ligated to NdeI-CRM197-BamHI fragment to generate the final plasmid, pET-14b CRM197. In addition, H4 is recombinant mycobacterial fusion peptide, containing early stage antigens Ag85B and TB10.4 secreted during the acute phase of infection [3-5]. H4 demonstrated protective immunity in mice [3, 6, 7] and was a safe and immunogenic vaccine in South African adults.[8] H28 contains the H4 antigen backbone and a latency-associated antigen Rv2660c. Strong cellular and humoral immune responses was induced by Rv2660c in a Chinese latent TB infection population [9]. H28 was capable of protecting mice against M. tuberculosis challenge [7]. The gene fragments h4 (h4: TTCAGCCGTCCGGGTCTGCCGGTCGAATACCTGCAAGTTCCGTCGCCGAGCATGGGTCGTG ACATTAAAGTTCAGTTCCAAAGCGGTGGTAACAATAGCCCGGCCGTCTATCTGCTGGACGG TCTGCGTGCACAGGATGACTACAATGGCTGGGATATTAACACCCCGGCGTTTGAATGGTATT ACCAGTCAGGCCTGTCGATCGTGATGCCGGTTGGCGGTCAAAGCTCTTTCTATAGCGATTG GTACTCTCCGGCGTGCGGTAAAGCCGGCTGTCAGACCTATAAATGGGAAACCTTTCTGACG AGTGAACTGCCGCAGTGGCTGTCCGCAAATCGTGCAGTTAAACCGACGGGTTCAGCGGCC ATTGGCCTGTCGATGGCAGGTAGTTCCGCTATGATTCTGGCAGCTTATCATCCGCAGCAATT CATCTACGCAGGTAGTCTGTCCGCTCTGCTGGACCCGAGCCAGGGTATGGGTCCGTCTCTG ATCGGTCTGGCAATGGGTGATGCCGGCGGTTATAAAGCGGCCGATATGTGGGGTCCGTCAT CGGACCCGGCATGGGAACGTAACGATCCGACCCAGCAAATTCCGAAACTGGTCGCCAACA ATACCCGCCTGTGGGTGTACTGCGGCAACGGTACGCCGAATGAACTGGGCGGTGCAAATAT CCCGGCTGAATTTCTGGAAAATTTCGTTCGTAGCTCTAACCTGAAATTTCAGGATGCGTATA ACGCAGCTGGCGGTCATAACGCCGTCTTTAATTTCCCGCCGAACGGCACCCACAGTTGGGA ATACTGGGGTGCGCAACTGAATGCCATGAAAGGTGACCTGCAGAGTTCCCTGGGTGCAGG CATGTCTCAAATTATGTATAACTACCCGGCAATGCTGGGTCACGCAGGTGATATGGCAGGTT ATGCTGGCACGCTGCAGAGCCTGGGTGCGGAAATTGCCGTGGAACAGGCGGCCCTGCAAT CTGCGTGGCAGGGTGACACCGGCATCACGTATCAAGCATGGCAGGCTCAATGGAATCAGG CCATGGAAGATCTGGTTCGTGCGTACCATGCCATGTCATCGACCCACGAAGCAAACACGAT GGCAATGATGGCTCGCGACACCGCCGAAGCAGCTAAATGGGGCGGT (SEQ ID NO:15)) and h28 (h28: TTCAGCCGTCCGGGTCTGCCGGTCGAATACCTGCAAGTTCCGTCGCCGAGCATGGGTCGTG ACATTAAAGTTCAGTTCCAAAGCGGTGGTAACAATAGCCCGGCCGTCTATCTGCTGGACGG TCTGCGTGCACAGGATGACTACAATGGCTGGGATATTAACACCCCGGCGTTTGAATGGTATT ACCAGTCAGGCCTGTCGATCGTGATGCCGGTTGGCGGTCAAAGCTCTTTCTATAGCGATTG GTACTCTCCGGCGTGCGGTAAAGCCGGCTGTCAGACCTATAAATGGGAAACCTTTCTGACG AGTGAACTGCCGCAGTGGCTGTCCGCAAATCGTGCAGTTAAACCGACGGGTTCAGCGGCC ATTGGCCTGTCGATGGCAGGTAGTTCCGCTATGATTCTGGCAGCTTATCATCCGCAGCAATT CATCTACGCAGGTAGTCTGTCCGCTCTGCTGGACCCGAGCCAGGGTATGGGTCCGTCTCTG ATCGGTCTGGCAATGGGTGATGCCGGCGGTTATAAAGCGGCCGATATGTGGGGTCCGTCAT CGGACCCGGCATGGGAACGTAACGATCCGACCCAGCAAATTCCGAAACTGGTCGCCAACA ATACCCGCCTGTGGGTGTACTGCGGCAACGGTACGCCGAATGAACTGGGCGGTGCAAATAT CCCGGCTGAATTTCTGGAAAATTTCGTTCGTAGCTCTAACCTGAAATTTCAGGATGCGTATA ACGCAGCTGGCGGTCATAACGCCGTCTTTAATTTCCCGCCGAACGGCACCCACAGTTGGGA ATACTGGGGTGCGCAACTGAATGCCATGAAAGGTGACCTGCAGAGTTCCCTGGGTGCAGG CATGTCTCAAATTATGTATAACTACCCGGCAATGCTGGGTCACGCAGGTGATATGGCAGGTT ATGCTGGCACGCTGCAGAGCCTGGGTGCGGAAATTGCCGTGGAACAGGCGGCCCTGCAAT CTGCGTGGCAGGGTGACACCGGCATCACGTATCAAGCATGGCAGGCTCAATGGAATCAGG CCATGGAAGATCTGGTTCGTGCGTACCATGCCATGTCATCGACCCACGAAGCAAACACGAT GGCAATGATGGCTCGCGACACCGCCGAAGCAGCTAAATGGGGCGGTATGATCGCAGGCGT GGATCAGGCTCTGGCAGCAACGGGTCAGGCATCACAACGTGCAGCTGGTGCATCGGGCGG TGTCACCGTGGGCGTTGGTGTCGGCACGGAACAGCGTAATCTGAGTGTGGTTGCGCCGTC CCAATTTACCTTCAGCTCTCGCAGCCCGGATTTCGTTGACGAAACGGCGGGCCAAAGCTG GTGTGCCATTCTGGGTCTGAACCAATTCCAC (SEQ ID NO:16)) encoding the mycobacterial fusion peptides H4 with amino acid sequence (H4: FSRPGLPVEYLQVPSPSMGRDIKVQFQSGGNNSPAVYLLDGLRAQDDYNGWDINTPAFEWYY QSGLSIVMPVGGQSSFYSDWYSPACGKAGCQTYKWETFLTSELPQWLSANRAVKPTGSAAIG LSMAGSSAMILAAYHPQQFIYAGSLSALLDPSQGMGPSLIGLAMGDAGGYKAADMWGPSSD PAWERNDPTQQIPKLVANNTRLWVYCGNGTPNELGGANIPAEFLENFVRSSNLKFQDAYNAA GGHNAVFNFPPNGTHSWEYWGAQLNAMKGDLQSSLGAGMSQIMYNYPAMLGHAGDMAG YAGTLQSLGAEIAVEQAALQSAWQGDTGITYQAWQAQWNQAMEDLVRAYHAMSSTHEAN TMAMMARDTAEAAKWGG (SEQ ID NO:6)) and H28 with amino acid sequence (H28: FSRPGLPVEYLQVPSPSMGRDIKVQFQSGGNNSPAVYLLDGLRAQDDYNGWDINTPAFEWYY QSGLSIVMPVGGQSSFYSDWYSPACGKAGCQTYKWETFLTSELPQWLSANRAVKPTGSAAIG LSMAGSSAMILAAYHPQQFIYAGSLSALLDPSQGMGPSLIGLAMGDAGGYKAADMWGPSSD PAWERNDPTQQIPKLVANNTRLWVYCGNGTPNELGGANIPAEFLENFVRSSNLKFQDAYNAA GGHNAVFNFPPNGTHSWEYWGAQLNAMKGDLQSSLGAGMSQIMYNYPAMLGHAGDMAG YAGTLQSLGAEIAVEQAALQSAWQGDTGITYQAWQAQWNQAMEDLVRAYHAMSSTHEAN TMAMMARDTAEAAKWGGMIAGVDQALAATGQASQRAAGASGGVTVGVGVGTEQRNLSV VAPSQFTFSSRSPDFVDETAGQSWCAILGLNQFH (SEQ ID NO:7)) were codon optimized for E. coli strains and synthesized by GenScript (USA). Tuberculosis (TB) antigens H4 and H28 were also cloned to the 3′ end of CRM197. Briefly, pUC57 H4 or pUC57 H28 were digested with BamHI to prepare the H4 or H28 gene fragment. The purified H4 or H28 insert was ligated with BamHI-linearized vector, pET-14b CRM197, to generate final plasmids, pET-14b CRM197-H4 and pET-14b CRM197-H28. The cloning strategy was illustrated in FIG. 1. Molecular cloning of pET-14b plasmids for preparation of free soluble His6-H4 and His6-H28 proteins is described elsewhere [10].
Plasmid Transformation into E. coli

A 1.7 ml Eppendorf tube containing 200 W of frozen competent cells was thawed on ice for approximately 40 minutes. Subsequently, they were mixed thoroughly with 3 μl of purified plasmid DNA or 10 μl of a ligation mix and then incubated on ice for 20 minutes to allow plasmids to be adsorbed at the cell surface. To promote the uptake of the adsorbed plasmid DNA, the competent cells were gently mixed and heat-shocked at 42° C. for 90 seconds and then immediately incubated on ice for a further five minutes. Cells were regenerated by the addition of 800 μl liquid LB medium and incubated at 37° C. for one hour. To select and isolate recombinant clones, 100 μl of the cells was spread-plated on solid LB agar containing appropriate antibiotics.

### The Growth Condition Required for Overproduction and Self-Assembly of CRM197 Only and CRM197:Antigen Chimeric Particles

Genes encoding CRM197 alone and CRM197:chimeric antigens were regulated under a strong promoter, T7. Briefly, these genes were genetically manipulated and cloned into pET-14b expression vector containing the strong T7 promoter. The recombinant pET plasmid containing CRM197 gene was transformed into an endotoxin-free mutant of E. coli. An overnight cell culture at a volume of 10-20 ml was prepared and used to inoculate 1 litre of Luria broth supplemented with 0.5% (wt/vol) NaCl, 1% (wt/vol) glucose, and ampicillin at the final concentration of 100 μg/ml. The culture was incubated at 37° C. for approximately 3 hours at 200 rpm and induced by IPTG at the final concentration of 0.001 M when the OD600 reached about 0.5. The incubation was continued for 48 hours at 37° C. at 200 rpm.

### Particle Isolation and Purification

After growth at 37° C. for 48 h, cells were harvested by centrifugation at 6000×g for 20 min. Cells were re-suspended in 100 ml of 0.5× lysis buffer (25 mM Tris, 5 mM EDTA, and 0.04% w/v SDS at pH11) and then mechanically disrupted 5 times using a M-110P microfluidiser (Microfluidics, USA) at 20,000 psi. Cell lysate was centrifuged at 8000×g for 20 min at 4° C. to pellet protein particles, which were then sequentially washed three times by 0.5× lysis buffer, wash buffer (10 mM Tris, 5 mM EDTA, 2 M urea, 5% v/v Triton X-100, pH7.5), and Tris buffer (10 mM Tris, pH7.5). An efficient homogenization step assists to obtain a pure particle suspension. Thus, particles were re-suspended and homogenized for more than a minute prior to each washing step. Purified protein particles were stored in 10 mM Tris buffer pH7.5, with 20% ethanol at 4° C. for further analysis.

### Analysis of Particles Comprising CRM197

Purified protein particles were separated on a 10% Bis-Tris gel. Densitometry was used to determine a fusion protein percentage/purity of the total protein in particle fractions using Image Lab Software (Bio-Rad Laboratories, USA). The amount of a fusion protein was calculated using different amounts (50 ng, 100 ng, 300 ng, and 500 ng) of BSA as a standard curve. The molecular morphology and size of protein particles were visualized by TEM by the Manawatu Microscopy & Imaging Centre (MMIC) (Massey University, Palmerston North, New Zealand). Aggregation of protein particles in the ultimate storage solution, 10 mM Tris buffer pH7.5 with 20% ethanol, was measured by Mastersizer 3000 (Malvern, UK). Zeta potential of protein particles and their soluble forms was also analyzed by Zetasizer Nano ZS (Malvern, UK). The measurement of particle size and charge was performed at the Riddet Institute (Massey University, Palmerston North, New Zealand). Target protein bands on Bis-Tris gel were excised and protein sequence was identified using MALDI-TOF/MS. Protein sample preparation and identification using MALDI-TOF/MS were carried out by The Centre for Protein Research (Otago University, Dunedin, New Zealand).

### Formulation and Administration

Formulated compositions for the immunogenicity study contained 5 μg of TB antigens/dose, emulsified in DDA (dimethyl dioctadecyl ammonium bromide); 250 μg per dose; Sigma-Aldrich, USA) in a volume of 200 μL Tris buffer (10 mM Tris.HCl, pH7.5). TB antigen CRM197 particles tested are CRM197 particles displaying H4, CRM197 particles displaying H28, soluble His6-H4, and soluble His6-H28. All these samples were produced in endotoxin free host ClearColi BL21 (DE3). DDA (250 g per dose) alone was the negative control. The adjuvant, DDA, was prepared at a concentration of 10 mg/mL in sterile Tris buffer. DDA powder was added into the sterile Tris buffer and heated in an 80° C. water bath with stirring until dissolved. The homogeneous white DDA solution was cooled at room temperature (25° C.). Samples were mixed with the DDA solution freshly before use.

All animal experiments were approved by Otago University Animal Ethics Committee (Dunedin, New Zealand). This animal study was performed using 6- to 8-week-old female C57BL/6 mice, originally purchased from Jackson Laboratories (Bar Harbor, Me., USA) and bred within the Otago University animal unit. There were six mice per group. Formulated samples were injected into mice subcutaneously on the flank in a volume of 200 μL. Mice were immunized three times, 9 days apart. The immunisation animal trial was performed at the University of Otago in Dunedin (New Zealand).

### Enzyme-Linked Immunosorbent Assay (ELISA) Analysis

ELISA was used to analyse serum antibody responses. High-binding plates (Greiner Bio-One, Germany) were coated overnight at 4° C. with 100 μL of 5 μg/mL purified soluble TB antigents, His6-H4, and/or His6-H28, diluted in phosphate-buffered saline containing 0.05% (v/v) Tween 20, pH 7.5 (PBST). As controls, plates were also coated overnight at 4° C. with 100 μL of PBST. Plates were washed three times with PBST and blocked with 3% (wt/vol) BSA for 1 h at 25° C. Plates were washed with PBST and incubated with primary polyclonal antibodies, sera taken from individual mice diluted with PBST at concentrations ranging from 1/400 to 1/409600, at 25° C. for 1 h. After three times wash with PBST, plates were incubated with secondary HRP-conjugated antibodies, anti-mouse IgG1- or IgG2c-HRP (Abcam, UK) diluted with PBST at a concentration of 1/20 000, for 1 h at 25° C. After washing, o-phenylenediamine substrate (Abbott Diagnostics, IL, USA) was added on plates and incubated for 15 min at 25° C. The reaction was stopped by adding 50 μL of 0.5N H₂SO₄, and the results were measured at 490 nm on an ELx808iu ultramicrotitre plate reader (Bio-Tek Instruments Inc., USA). The ELISA was performed at the Institute of Fundamental Science (Massey University, Palmerston North, New Zealand).

### Western Blot Assay

To investigate the specificity of the IgG response, pooled sera from mice immunized with different test samples (CRM197 particles, CRM197 particles displaying H4, CRM197 particles displaying H28, soluble His6-H4, and soluble His6-H28) were diluted 2000-fold and used for immunoblotting against whole cell lysate containing various test particles and purified test particles after they were transferred from Bis-Tris gel to nitrocellulose membranes (Life Technology, USA). An antimouse IgG HRP-conjugate (Abcam, United Kingdom) was diluted 20 000-fold and used for detection of bound IgG antibodies. Signal was developed by incubating the membrane with SuperSignal West Pico Stable Peroxide Solution, and SuperSignal West Pico Luminol/Enhancer Solution (Thermo Scientific, USA). Film was developed with an X-ray film developer. The western blot was performed at the Institute of Fundamental Science (Massey University, Palmerston North, New Zealand).

### Preparation of Single Spleen Cell Suspension

Single cell suspensions were prepared from spleens by teasing the tissue through a 70×10⁻⁶ m cell strainer (Corning, USA). Cells were then washed twice with incomplete RPMI medium (Life Technologies, USA) supplemented with penicillin (100 U mL-1; Life Technologies, U.S.□) and streptomycin (100 U mL-1; Life Technologies, USA). Red blood cells were lysed using red blood cell lysis buffer (Sigma-Aldrich, USA). Cells were washed and resuspended in complete RPMI (Life Technologies, USA) supplemented with penicillin (100 U/mL), streptomycin (100 U/mL), and 5% (wt/vol) fetal calf serum (Life Technologies, USA). Cells were stained with Trypan blue (1:100) and counted using a haemocytometer. These assays were performed at the University of Otago in Dunedin (New Zealand).

### Splenocyte Stimulation and Measurement of Cytokines in Supernatants

Single spleen cell suspensions were prepared in complete RPMI media at a cell concentration of 5×10⁶ per mL, 100 μL of which was added to U-bottomed 96-well plates (Life Technologies, USA). Cells were stimulated with 100 μL of complete RPMI media alone or 40 μg/mL of soluble His6-H4 or soluble His6-H28 antigen. The culture was incubated at 37° C. in 5% CO₂ for 24 h or 60 h. Cytokine release in supernatant was measured using BD CBA Mouse Th1/Th2/Th17 cytokine kit (BD Biosciences, USA) with Falcon V bottom plates (Corning, USA) according to manufacturer's instructions. Data were obtained using a FACS Canto with BD FACSDiva software (BD Biosciences, USA). These assays were performed at the University of Otago in Dunedin (New Zealand).

### Statistical Analysis

The cytokine and antibody responses were analyzed by using the one-way ANOVA. Each data point stands for results from six mice ± the standard error of the mean. Statistical significance is determined when p<0.05. Statistical analysis was carried out using Minitab 17.

### Results and Discussion

### Engineering of CRM197, CRM197-H4, and CRM197-H28 Towards the Formation of Self-Assembly of Protein Particle In Vivo

In general, the immunogenicity of a CRM197 inclusion body and the potential application of CRM197 inclusion body toward the development of antigen carrier system have not been studied. The focus for CRM197 manufacture has been on the production and purification of soluble and bioactive CRM197 for vaccine conjugation applications, and thus solubilization and refolding of this protein was applied [6-8].

The aim of the present study was to investigate the potential of a CRM197 inclusion body/particle as an efficient antigen/immunogen carrier platform, particularly for various immunogenic preparations and diagnostic reagent development.

In previous studies, a CRM197 inclusion body was formed in bacterial hosts in the process of preparing soluble CRM197. However, in previous studies, the CRM197 inclusion body was treated as biological waste. Although the CRM197 inclusion body was formed when preparing the soluble version of CRM197, the cloning and growth condition of forming inclusion body is different to ours. For example, it was shown an affinity or purification tag is required to stimulate the overproduction of CRM197 protein and ultimately the inclusion body formation. Alessandra et al (2011) showed that the expression of CRM197 always failed in the absence of a histidine tag in E. coli. However, the addition of the histidine tag at the N-terminus of CRM197 dramatically stimulated protein production towards inclusion body formation [8]. A histidine tagged CRM197 inclusion body was also produced by Park et al (2018) [7]. In our study, no modification was made to the CRM197 protein sequence and the protein was successfully overproduced towards inclusion body/particle formation. It is very likely that the growth conditions and codon usage that were designed and optimized for CRM197 in the present study are more suitable for strong gene expression in one or more recombinant protein expression systems, particularly E. coli cells. Moreover, the bacterial strain and growth conditions for CRM197 inclusion body formation in the literature is different to the present study. For instance, the synthetic gene encoding recombinant CRM197 (His6-enterokinase cleavage site-CRM197) in Park et al (2018) were cloned into pET28a+. Moreover, this study did not prepare an overnight culture as the inoculum for CRM197 production [7]. Other growth condition parameters in Park et al (2018), including incubation time, growth media and supplements, are different to the present study.

Generally, intracellular self-assembly of protein particles (inclusion bodies) was often observed when recombinant fusion proteins were overproduced under a strong promoter and overwhelm bacterial cell repair system [11, 12]. In this study, the gene encoding immunogenic carrier CRM197 was codon-optimized for E. coli strains and genetically cloned into the pET-14b expression vector containing the strong T7 promoter. In addition, mycobacterial fusion peptides H4 or H28 were bioengineered to the C-terminus of CRM197. The modular compositions and molecular cloning strategies were elaborated in FIG. 1. These recombinant genes were transformed and expressed in ClearColi BL21(DE3).

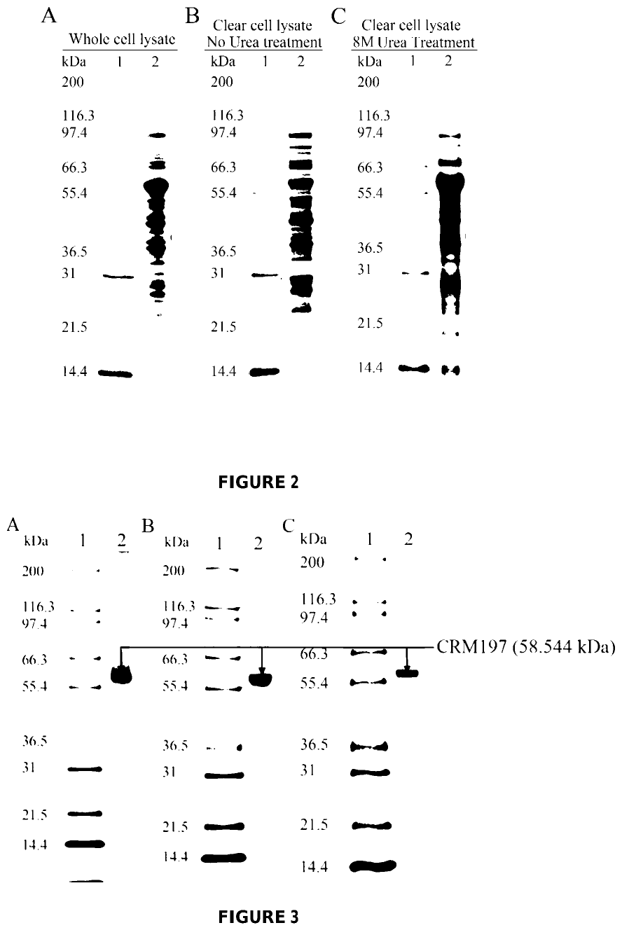

The solubility of the CRM197 was firstly determined by analyzing the protein profile of the cell producing CRM197, treated with or without 8 M urea (FIG. 2). The Bis-Tris gel showed that a dominant protein band corresponding to protein with theoretical molecular weight (MW) of CRM197 (58.544 kDa) was observed in the whole cell lysate (FIG. 2A), indicating CRM197 is tremendously overproduced. Furthermore, cell producing CRM197 was treated without (FIG. 2B) or with 8 M urea (FIG. 2C). The supernatant fraction of the crude cell lysate was analyzed using Bis-Tris gel after sonication and centrifugation. Dominant protein band with MW of CRM197 (58.544 kDa) was absent in the supernatant fraction without 8 M urea treatment and found only in the supernatant fraction treated with 8 M urea (FIGS. 2B and 2C), suggesting that CRM197 was produced as insoluble protein.

CRM197 particle isolation and purification condition was optimized and the result illustrated in FIG. 3 to demonstrate successful extraction of highly pure CRM197 particles from the complex E. coli cell mixture. Indeed, E. coli cells producing CRM197 particles were re-suspended in 0.5× lysis buffer and mechanically disrupted using a M-110P microfluidizer (Microfluidics, USA). After cell disruption, CRM197 particles in crude cell lysate were purified individually through two proposed washing procedures: a. 0.5× lysis buffer (FIG. 3B) and b. 0.5× lysis buffer and 10 mM Tris buffer containing 2 M urea and 5% Triton X-100 (FIG. 3C). In contrast to 0.5× lysis buffer wash after cell disruption, CRM197 particles showed relatively high purity when the particles were washed with both 0.5× lysis buffer and 10 mM Tris buffer containing 2 M urea and 5% Triton X-100 (FIGS. 3B and 3C). Particularly, the CRM197 protein purity is 95.6% of the total protein in the CRM197 particle fraction, analyzed by densitometry analysis using 10% Bis-Tris gel and Image Lab Software (Bio-Rad Laboratories, USA). The optimized CRM197 particle isolation and purification condition were elaborated in the Material and Method.

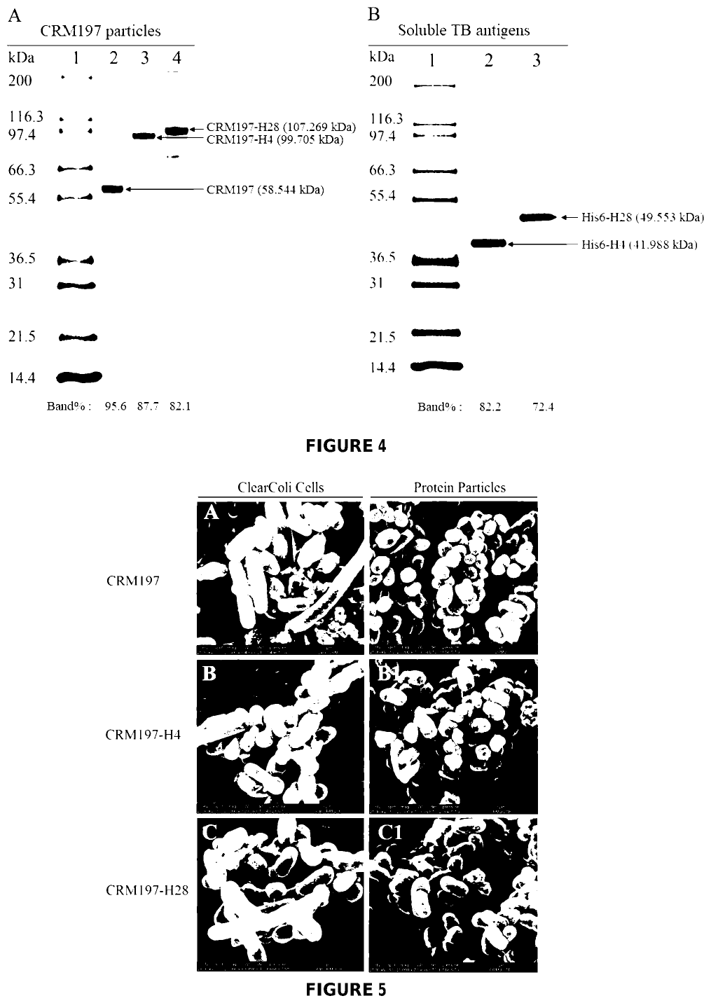

The protein profile of purified CRM197 particles and the particles carrying mycobacterial fusion peptide H4 or H28 were analyzed by Bis-Tris gel electrophoresis in FIG. 4. Densitometry analysis using 10% Bis-Tris gel and Image Lab Software exhibited that CRM197 particles and the particles displaying H4 or H28 accounted for 95.6%, 87.7%, and 82.1% of total proteins in their corresponding particle fractions (FIG. 4A). Moreover, Chen et al. (2018) demonstrated that His6-tagged H4 and H28 can be overproduced and form inclusion bodies in ClearColi BL21 (DE3), and free soluble H4 and H28 peptides can be prepared by solubilizing H4 and H28 antigen particles [13]. FIG. 4B illustrated the protein profile of purified soluble His6-H4 and His6-H28, which accounted for 82.2% and 72.4% in their soluble protein fractions. The target protein sequences of CRM197 particle samples and soluble mycobacterial antigens H4 and H28 were identified by MALDI-TOF MS (TABLES 2 and 3).

Characterization of Purified CRM197 Particles and the Particles Coated with H4 or H28 Mycobacterial Antigens

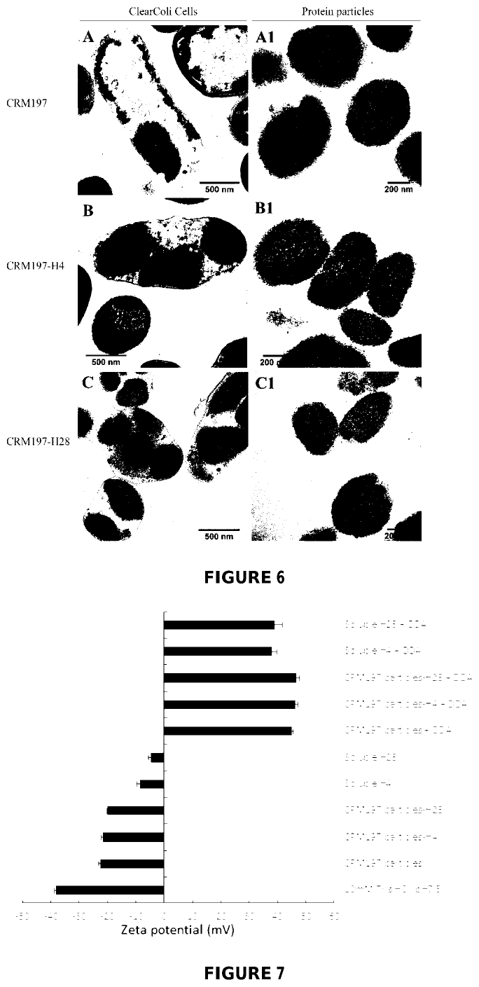

The presence of these intracellular CRM197 particles and the particles displaying H4 or H28 peptides were observed by SEM (FIG. 5) and TEM (FIG. 6). The CRM197 particle size varies and ranges between 200 nm to 800 nm in diameter (FIG. 6). The particles exhibited oval shape with surface cotton-like amorphous structures (FIG. 6). This amorphous structure is a loose protein network, containing unfolded and/or misfolded proteins interconnected by hydrophobic interaction and correctly folded proteins are arrested inside the network [14, 15]. Furthermore, the result demonstrated that E. coli cells can produce more than one protein particles (FIG. 6). However, during cell division, all particles will remain in one cell and protein production and self-assembly of new particles will restart in the other cell [16].

Zeta potential of CRM197 particle samples were analysed before and after emulsification in DDA. All the CRM197 particle samples and soluble His6-tagged antigens (H4 or H28) were stored in a formulation buffer, 10 mM Tris-HCl buffer pH7.5, which was negatively charged (FIG. 7). These test samples had negatively charged surface in Tris-HCL buffer; however, they became strongly positively charged after emulsification in DDA solution (FIG. 7). Surface charge of particles can affect cellular uptake by antigen presenting cells (APCs). It is known that uptake of particles by dendritic cells may be promoted when the particle possesses a positively charged surface [17]. However, a number of studies have demonstrated that negatively charged particles can be efficiently taken up by APCs, which can be possibly caused by opsonization [18-20] or adsorption of negatively charged particles at cationic sites in the cell membrane [19, 21, 22].

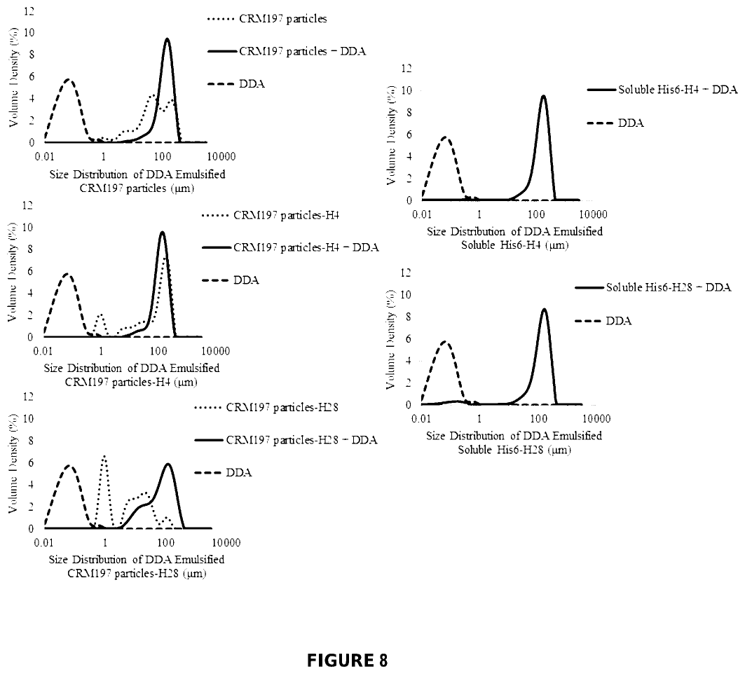

Size distribution of purified protein particles was also analyzed before and after emulsification in DDA. CRM197 particles and the particles displaying H4 or H28 were not monodispersed and their size ranges between 0.5 μm and 400 μm, suggesting particle aggregation occurs (FIG. 8). However, after formulation with DDA, all particles become monodispersed and the size was around 100 μm (FIG. 8), suggesting DDA influenced the physicochemical properties of the plain CRM197 particles and the particles displaying TB antigens. DDA possessed a small size range between 0.01 μm and 0.8 m. However, emulsification of soluble His6-H4 or His6-H28 in DDA shifted the size distribution to 10 μm-400 μm. Generally, particles with a size range between 0.5 μm and 10 μm are preferably taken up by APCs via phagocytosis [20, 23]. Nevertheless, smaller particles as well as soluble antigens are often taken up by endocytosis. Cellular uptake of particulate antigens via phagocytosis into phagosomes leads to antigen cross-presentation and ultimately may elicit both humoral and cell-mediated immune responses [24, 25].

### CRM197 Particle Formulation and Mice Immunization

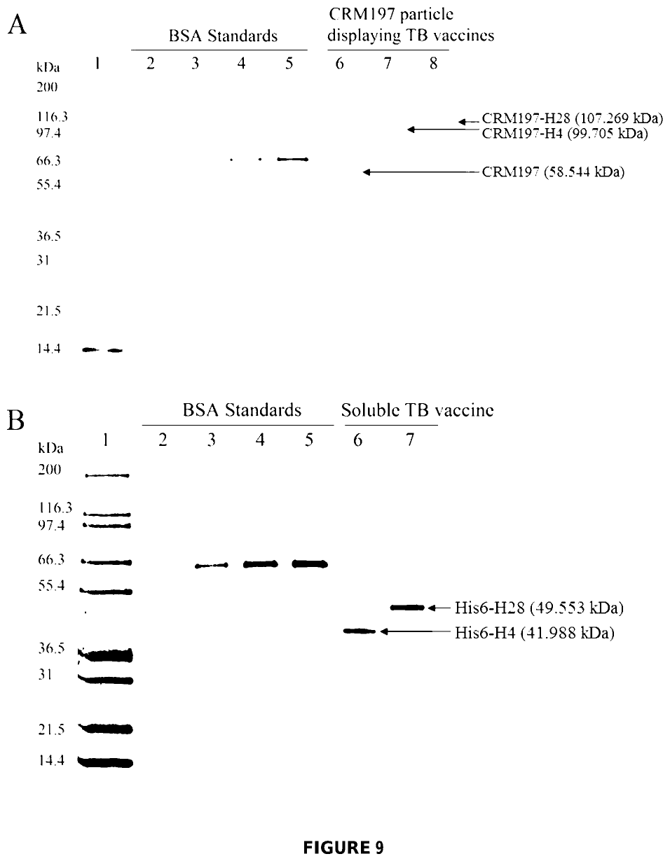

Mycobacterial antigen concentration was firstly determined for formulation by using different amounts of BSA standards (50 ng, 100 ng, 300 ng, and 500 ng) (FIG. 9) and analyzed by densitometry using Image Lab software. All test samples were produced from a LPS free E. coli strain, which only produces a genetically mutated non-toxic LPS and cannot trigger an endotoxic response in human cells [26]. Mice were administered subcutaneously with 5 μg of mycobacterial antigens per dose, emulsified in DDA (250 μg/dose) in a volume of 200 μl. All mice exhibited healthy and no adverse effects and abnormal behaviors were observed. They all gained weight and remained alive throughout the experiment (data not shown).

### Immunogenicity Analysis

### Antibody Responses

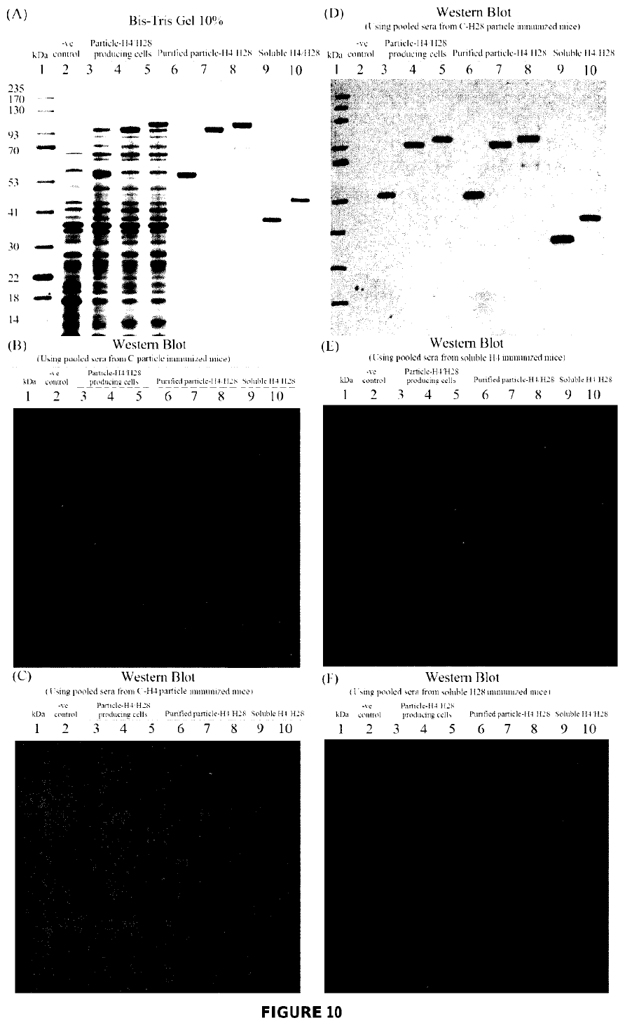

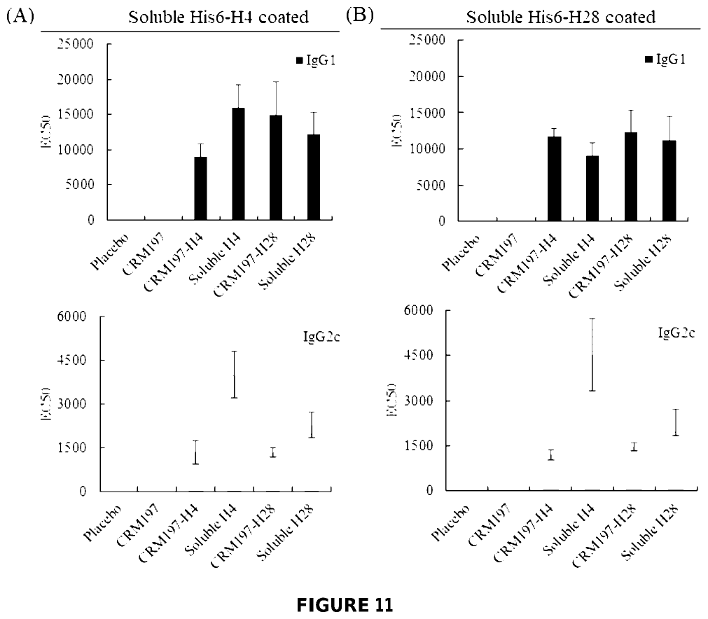

Both humoral and cellular immune responses play a role against bacterial pathogens. Nevertheless, cell-mediated immune response is considered more important for control of intracellular pathogens as cellular immunity was correlated with prevention of intracellular pathogen infection [27-29]. However, mycobacterial pathogen has a transient extracellular phase, and thus the pathogen can be susceptible to the antimicrobial effect of antibodies [27]. The immunoblot results in FIG. 10 demonstrated that pooled sera from mice immunized with various test samples only specifically recognized the corresponding target protein bands and did not non-specifically interact with the background proteins from the production host E. coli strain, suggesting that serum antibodies from mice immunized with different test samples were very specific (FIG. 10). There was no significant difference of IgG1 response to soluble His6-H4 or His6-H28 in mice immunized with CRM197 particles displaying H4 or H28 and soluble His6-H4 or His6-H28 (p>0.05) (FIG. 11). However, IgG2c response to soluble His6-H4 was significantly greater in mice immunized with soluble His-H4 than in mice immunised with CRM197 particle-H4 (p=0.049) (FIG. 11). This significant difference was also observed in IgG2c response to soluble His6-H28 between mice immunized with soluble His6-H28 and CRM197 particle-H28 (p=0.021) (FIG. 11).

### Cytokine Responses

Polyfunctional CD4+ T cells producing a variety of pro-inflammatory cytokines often correlated with protective immune responses against intracellular mycobacterial pathogens [30-33]. IL17A and IFNγ are biomarkers for development of cell-mediated immune response [34-37]. Particularly, the development of cell-mediated immunity was determined by measuring the release of cytokines from splenocytes, which were restimulated in vitro with soluble His6-H4 and soluble His6-H28 mycobacterial antigens. In this patent, cytokine release was analyzed at the early (24 hours) and late (60 hours) time points in order to detect cytokine release and consumption during culture.

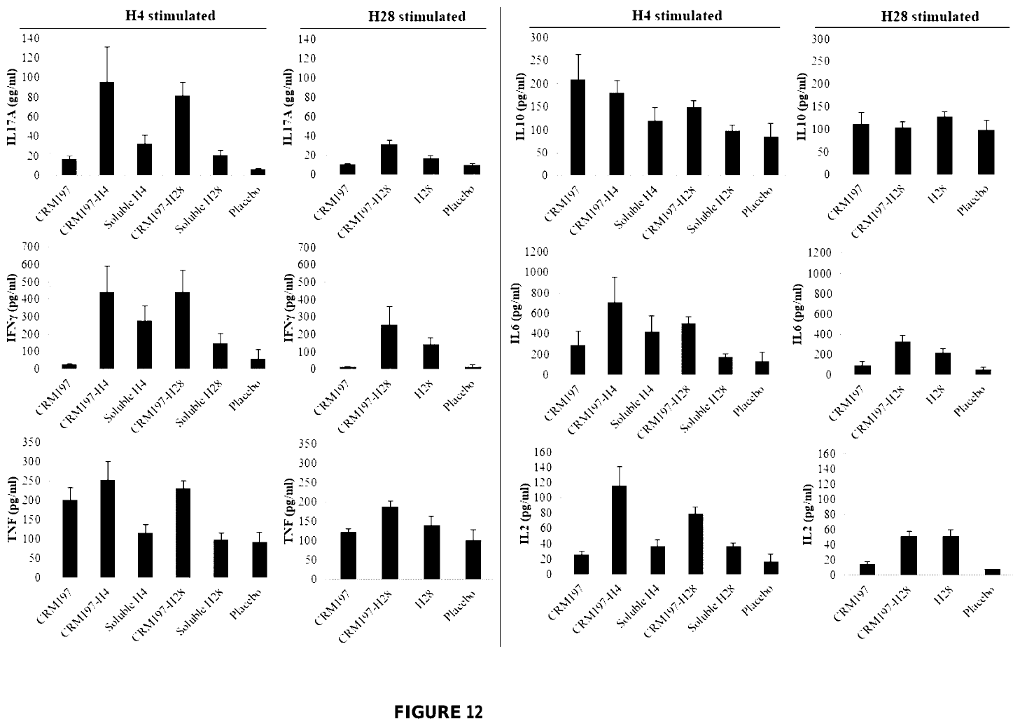

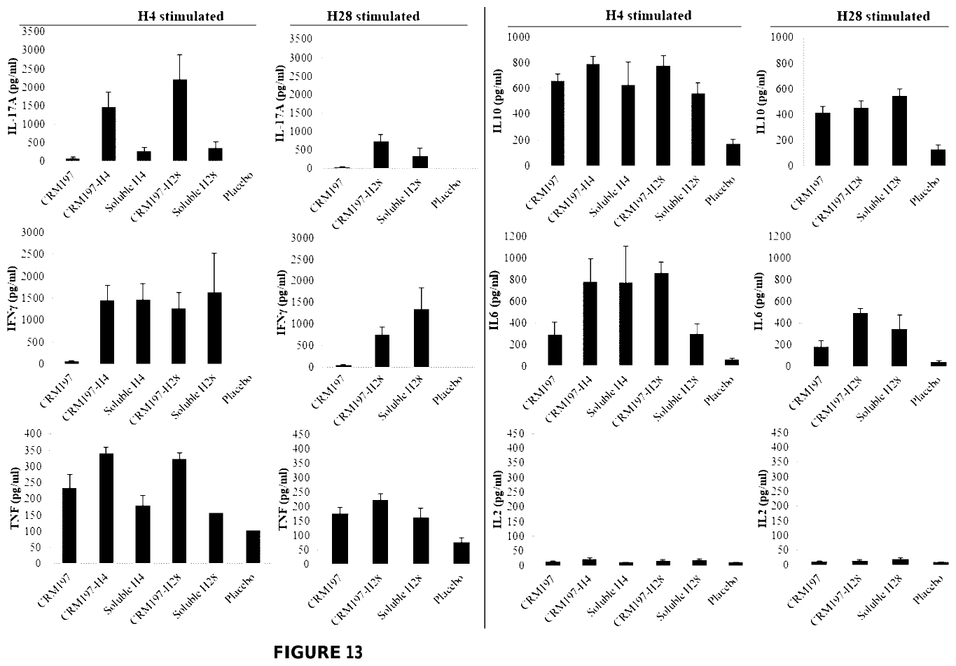

Upon the re-stimulation in vitro with soluble His6-H4 for 24 hours, splenocytes from mice tested with CRM197 particle displaying H28 showed significantly high IL17A secretion when compared to mice tested with soluble His6-H28 (p=0.008) (FIG. 12). Following 60 h re-stimulation with soluble His6-H4 or His6-H28, the amount of IL17A produced by splenocyte from mice immunized with CRM197 particle displaying H4 or H28 was significantly higher than their corresponding soluble counterparts His6-H4 (p=0.037) or His6-H28 (p=0.013) (FIG. 13). There was no statistical difference of IFNγ secretion between splenocytes from mice immunized with CRM197 particles displaying H4 or H28 and their soluble antigen versions (p>0.05) (FIGS. 12 and 13). Although IL17A and IFNγ are biomarkers for the development of cell-mediated immune response, there is no correlation between IL17A and IFNγ secretion and enhanced protective immunity [32, 38-40].

### Example 2

### Production of CRM197 Particles in Various E. coli Strains

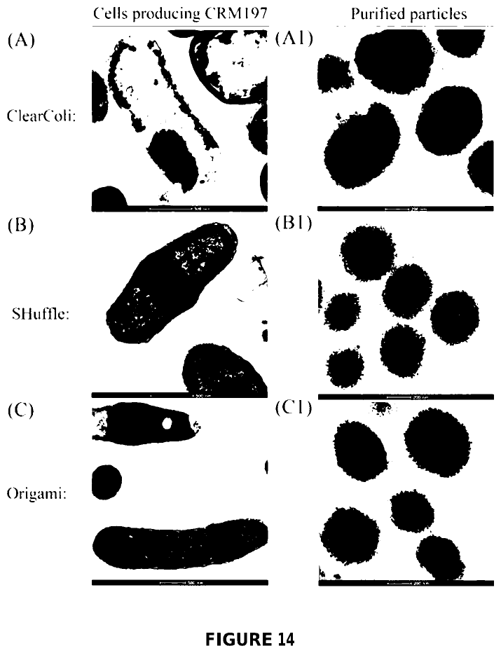

This example demonstrates that CRM197 particle can be formed in Shuffle and Origami E. coli strains. The TEM images of CRM197 particle produced in ClearColi, SHuffle T7, and Origami is shown in FIG. 14. These TEM images were performed by the MMIC (Massey University, Palmerston North, New Zealand). The growth condition required for overproduction and self-assembly of CRM197 particle in Shuffle and Origami strains is similar to the methods used for particle formation in ClearColi strain. Briefly, the pET plasmid containing CRM197 gene was transformed into E. coli SHuffle T7 and Origami strain respectively. An overnight cell culture at a volume of 10 ml grown at 37° C. was prepared and used to inoculate 1 litre of Luria broth supplemented with 1% (wt/vol) glucose, and ampicillin at the final concentration of 100 μg/ml. The culture was grown at 37° C. for about 3 h at 200 rpm and induced by IPTG at the final concentration of 0.001 M when the OD600 achieved 0.5. The incubation for 48 hours at 37° C. at 200 rpm can be used for CRM197 particle formation.

CRM197 produced in SHuffle T7 or Origami strains are likely properly folded and/or biologically active as these strains are able to facilitate proteins to properly form disulphide bonds.

### Example 3

### Evaluation of Self-Adjuvanting Property of CRM197-TB Particles with/without DDA Adjuvant and Evaluation of Immune Responses Thereof

The CRM-TB particles made in Example 1 were tested in mice to assess the self-adjuvanting property of the CRM particles as well as their ability to induce protective immunity. Briefly, there were 5 test samples (CRM197 particles, CRM197 particles displaying H4, soluble H4, BCG, and placebo), and 10 mice per group. Mice immunised three times subcutaneously at 2-wk intervals on the flank with the test samples containing 10 μg of TB antigens/dose, emulsified in DDA (250 μg/dose) in a volume of 200 μl. At the time of the first immunisation, one group of mice were treated with a single dose of BCG (5×10⁵ CFU), injected subcutaneously. Three weeks after the final injection, 4 mice were killed. The remaining mice received M. tuberculosis challenge six weeks after the final vaccination. Six weeks after M. tuberculosis challenge, mice will be killed. Immunogenicity analysis was performed by measuring IgG1 and IgG2c responses from serum, ELISpot to measure antigen specific INFγ secreting cells, and intracellular cytokine (IL2, IFNγ, TNF, and IL17) staining of the lung tissue. In addition, bacterial numbers were measured in the lung and spleen to determine protection.

### Materials and Methods

### Immunisation and Infection of Mice

Female C57BL/6 (6-8 weeks of age) were purchased from the Animal Resources Centre (Perth, Australia) and maintained under specific pathogen-free conditions. All experiments were performed with the approval of the Sydney Local Health District Animal Welfare Committee (approval number 2016-044D) in accordance with relevant guidelines and regulations. For protection experiments, mice were injected subcutaneously (s.c.) either once with 5×10⁵ CFU of BCG Pasteur (200 μl in PBS), or three times at two-week intervals with 10 μg/mL of H4, H28, C—H4, C—H28 or CFP formulated in 10 mM Tris buffer (pH 7.5) with 10 mg/mL DDA. Mice administered with vehicle only were used as negative controls. For challenge experiments, mice were infected with M. tuberculosis H37Rv six weeks after the final vaccination via the aerosol route, using a Middlebrook airborne infection apparatus (Glas-Col) with an infective dose of approximately 100 viable bacilli. The immunization animal trials were performed at the Centenary Institute at the University of Sydney (Australia).

IFNγ ELISpot assay Splenocytes were prepared from test mice by passage through a 70 μm cell strainer (BD). Cells were resuspended in buffered ammonium sulfate (ACK buffer; 0.1 mM EDTA (Sigma), 10 mM KHCO₃ (Sigma), 150 mM NH4Cl (Sigma) to lyse erythrocytes and then washed and resuspended in RPMI 1640 (Life Technologies) supplemented with 10% heat-inactivated fetal bovine serum (Scientifix, Cheltenham, Australia), 50 μM 2-mercaptoethanol (Sigma), and 100 U ml⁻¹ Penicillin/Streptomycin (Sigma).

Cells were counted, then cultured at a density of 2×10⁶ cells/mL in an ELISpot plate precoated with 15 μg/mL of anti-mouse IFN-γ monoclonal antibody (clone AN18) in the presence of H4 or H28 at a final concentration of 10 μg/mL. As controls, cells were incubated with media alone or ConA at 3 μg/mL. After 18 h of incubation, plates were thoroughly washed with PBS/0.01% Tween 20 and incubated with biotinylated anti-mouse IFN-γ monoclonal antibody (clone XMG1.2) at final concentration 2.5 μg/mL for at least 2 h at 37° C. Development was achieved by incubation with avidin-conjugated alkaline phosphatase (Sigma) followed by addition of AP conjugate substrate (Biorad). The numbers of spots in the wells were determined using an AID ELISpot Reader. These assays were performed at the Centenary Institute at the University of Sydney (Australia).

### Intracellular Cytokine Staining and Flow Cytometry

For intracellular cytokine staining, splenocytes were stimulated for 3-4 hours in the presence of the H4, H28, TB10.4 or CFP (10 μg/mL), then incubated with brefeldin A (10 μg/mL) for up to 12 hours. Two million cells were incubated with 1.25 μg/mL anti-CD32/CD16 (eBioscience, San Diego, Calif.) in FACS wash buffer (PBS/2% FCS/0.1%) for 30 min to block Fc receptors, then washed and incubated for 30 min with anti-CD3-Alexafluor 700 (clone 17A2, Biolegend), anti-CD4-PerCP (clone RM4-5, Biolegend), anti-CD8a-allophycocyanin (APC)-Cy7 (clone 53-6.7, Biolegend), or anti-CD44-fluorescein isothiocyanate (FITC) (clone IM7, BD). Fixable Blue Dead Cell Stain (Life Technologies) was added to allow dead cell discrimination. Cells were then fixed and permeabilized using the BD Cytofix/Cytoperm™ kit according to the manufacturer's protocol. Intracellular staining was performed using the following antibodies: anti-IFN-γ-phycoerythrin (PE)-Cy7 (clone XMG1.2), anti-TNF-APC (clone MP6-XT22, Biolegend, San Diego, Calif.), anti-IL-2-PE (clone JES6-5H4) (BD) or anti-IL-17A-Pacific Blue (clone TC11-18H10, Biolegend). All samples were acquired on a BD LSR-Fortessa flow cytometer (BD), and analyzed using FlowJo™ analysis software (Treestar, Macintosh Version 9.8, Ashland, Oreg.). These assays were performed at the Centenary Institute at the University of Sydney (Australia).

### T Cell Proliferation Assay

Bone marrow cells from female C57BL/6 mice (6-8 weeks of age) were induced to differentiate into CD11c+ bone marrow derived dendritic cells via incubation with 10 ng/mL recombinant mouse GM-CSF (ProSpec, Israel) in RPMI 1640 (Life Technologies) supplemented with 10% heat-inactivated fetal bovine serum (Scientifix, Cheltenham, Australia), 50 μM 2-mercaptoethanol (Sigma), and 100 U ml-1 Penicillin/Streptomycin (Sigma). After five days of differentiation and splitting in fresh media, non-adherent bone marrow derived dendritic cells (BMDCs) were collected, counted and seeded at 1×106 cells/mL, then pulsed with H4 or C—H4 at concentrations ranging from 0.1-100 μg/mL for 4 h at 37° C. Meanwhile, spleen cells from a female RAG−/− p25 epitope-specific mouse were processed to single cell suspension as described above and CD4+ T cells isolated at a purity of 90-95% using an EasySep™ Mouse CD4+ T Cell Isolation Kit (Stemcell Technologies, VN, Canada) as per manufacturer's directions. CD4+ T cells were then stained with Cell-Trace Violet (CTV) as per manufacturer's directions using the CellTrace™ Violet Cell Proliferation Kit for flow cytometry (Invitrogen, ThermoFisher Scientific, MA, USA). CD4+ T cells were then added to pulsed BMDCs at 4×107 cells/mL and co-cultured for 3.5 days before flow cytometric analysis to determine percentage T cell proliferation. Cells were incubated with 1.25 μg/mL anti-CD32/CD16 (eBioscience, San Diego, Calif.) in FACS wash buffer (PBS/2% FCS/0.1%) for 30 min to block Fc receptors, then washed and incubated for 30 min with anti-CD3-Alexafluor 700 (clone 17A2, Biolegend), anti-CD4-PerCP (clone RM4-5, Biolegend), anti-CD8a-allophycocyanin (APC)-Cy7 (clone 53-6.7, Biolegend), or anti-CD44-fluorescein isothiocyanate (FITC) (clone IM7, BD). Fixable Blue Dead Cell Stain (Life Technologies) was added to allow dead cell discrimination. Cells were then fixed and permeabilized using the BD Cytofix/Cytoperm™ kit according to the manufacturer's protocol. Percentage proliferation was determined by identifying cells as CTV low. These assays were performed at the Centenary Institute at the University of Sydney (Australia).

### Bacterial Quantification

Four weeks after aerosol M. tuberculosis infection, the lung and spleen were harvested, homogenised and plated after serial dilution on Middlebrook 7H10 agar plates supplemented with 10% oleic acid-albumin-dextrose-catalase. Plates were incubated at 37° C. and colony forming units (CFU) were determined approximately 3 weeks later. These assays were performed at the Centenary Institute at the University of Sydney (Australia).

### Statistical Analysis

The significance of differences between experimental groups was evaluated by one- or two-way analysis of variance (ANOVA), with pairwise comparison of multi-grouped data sets achieved using Tukey's or Dunnet's post hoc test (Prism). This analysis was performed at their facility at the Centenary Institute at the University of Sydney (Australia).

### Results and Discussion

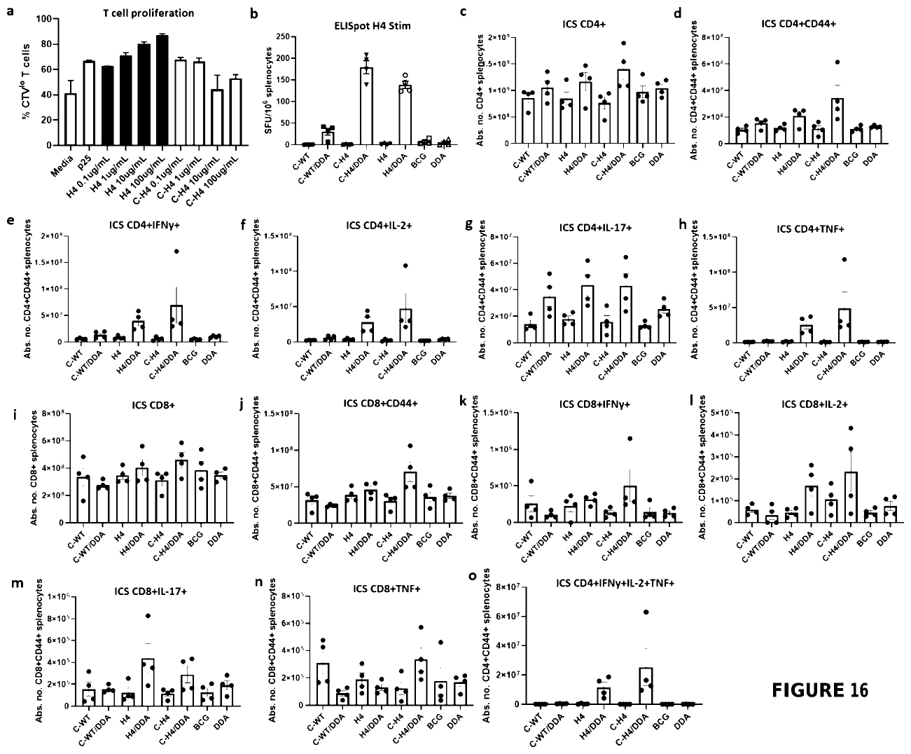

Self-adjuvanting property of CRM197 particles was first investigated by immunizing the mice with CRM197 particle or CRM197-H4 particle test samples in the absence or presence of DDA adjuvant. ELISpot assay (FIG. 16b) shows there was no IFNγ secretion from the mice immunized with CRM197-TB particles in the absence of DDA. However, the INFγ secretion was high in those splenocytes from mice immunized with particulate TB samples in the presence of DDA. Particularly, mice immunized with CRM197-H4/DDA showed relatively more INFγ secretion when compared to the mice tested with soluble H4/DDA. The ELISpot result suggested that CRM197 particle may have no or low self-adjuvanting property and DDA adjuvant may be required for induction of an immune response. In addition, different concentrations of soluble H4 and CRM197-H4 particles, ranging between 0.1-100 μg/mL, were applied in T cell proliferation assay (FIG. 16a). Soluble H4 gradually stimulated cell growth as the concentration increased. However, high concentration of CRM197-H4 particle inhibited cell proliferation. This may indicate CRM197-H4 could show adverse effect at a high dose.

Cytokine production of CD4+ (FIG. 16c-h) and CD8+ T cells (FIG. 16i-n) from mice immunised with different test samples was analysed using intracellularly cytokine staining (ICS). Both CD4+ and CD8+ T cells from mice injected with non-adjuvanted TB test samples did not show cytokine (IFNγ, IL-2, IL-17, and TNF) production in response to soluble H4 stimulation when compared to the negative control, non-adjuvanted CRM197 particle. This result is consistent with the ELISpot assay. However, CD4+ and CD8+ T cells from mice immunized with DDA adjuvanted TB test samples, soluble H4 and CRM197-H4 particles, demonstrated strong cytokine (IFNγ, IL-2, IL-17, and TNF) production when stimulated with soluble H4. This result may suggest that a CRM197 particle may not possess adjuvant property. However, there is a trend that the cytokine production of the CD4+ and CD8+ T cells from mice immunized with CRM197-H4 particles/DDA is higher than the one from mice administered with soluble H4/DDA in response to soluble H4. Thus, we can still benefit from particulate CRM197-TB in regard to immunogenicity and in particular, manufacturing of compositions.

### Challenge Study Assessing Induction of Protective Immunity by DDA Adjuvanted CRM197-TB Particles

CRM197-H4 particles and CRM197-H28 particles were formulated with DDA adjuvants. The formulated test samples were used to immunise female BALB/c mice to study the immunogenicity and protectivity of adjuvanted CRM197-TB particles.

The CRM-TB particles made in Example 1 were tested in challenge experiments. All the material and methods are as described in Examples 1 and 3. Briefly, there were 8 test samples (CRM197 particles, CRM197 particles displaying H4, CRM197 particles displaying H28, soluble H4, soluble H28, BCG, CFP, and placebo), and 12 mice per group. Mice were immunised three times subcutaneously at 2-wk intervals on the flank with test samples containing 10 μg of TB antigens/dose, emulsified in DDA (250 μg/dose) in a volume of 200 μl. Serum from all the mice were collected two weeks later after each injection for antibody (IgG1 and IgG2c) response analysis. At the time of the first vaccination, one group of mice were treated with a single dose of BCG (5×105 CFU), injected subcutaneously. Three weeks after the final vaccination, 4 mice were killed. The remaining mice received M. tuberculosis challenge six weeks after the final vaccination. Six weeks after M. tuberculosis challenge, mice were killed. The following experiments were performed to analyse immunogenicity: IgG1 and IgG2c responses from serum, ELISpot to measure antigen specific INFγ secreting cells, and intracellular cytokine (IL2, IFNγ, TNF, and IL17) staining of the lung tissue. In addition, bacterial numbers were measured in the lung and spleen to determine protection.

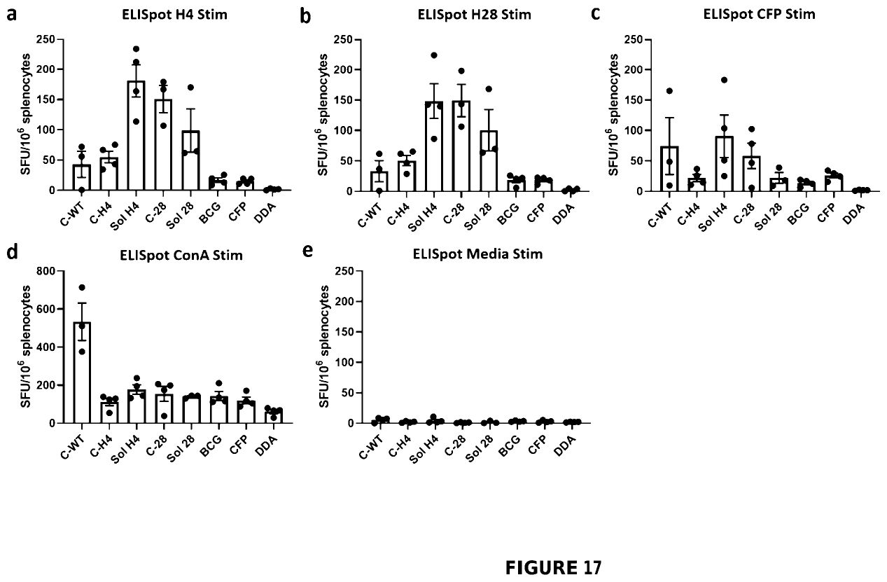

ELISpot assay showed splenocytes from soluble H4-immunized mice have higher IFNγ production in response to soluble H4 or soluble H28 stimulation when compared to the splenocytes from CRM197-H4 immunised mice (FIG. 17ab). However, in response to soluble H4 or H28 stimulation, splenocytes from CRM197-H28 particle immunized mice have a tendency of a relatively high IFNγ production in contrast to the splenocytes from soluble H28 immunized mice (FIG. 17ab).

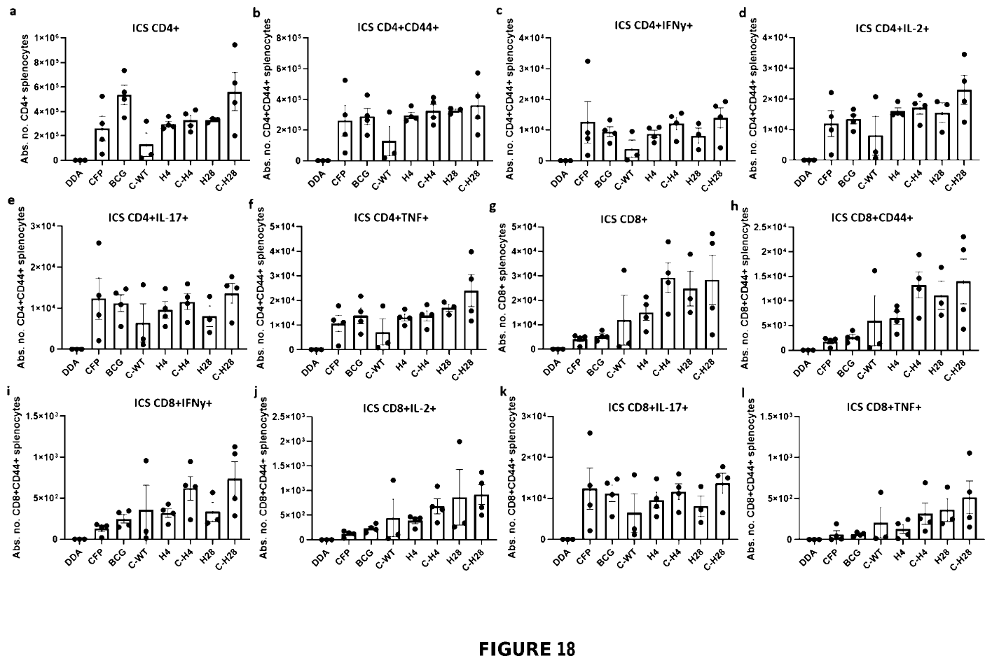

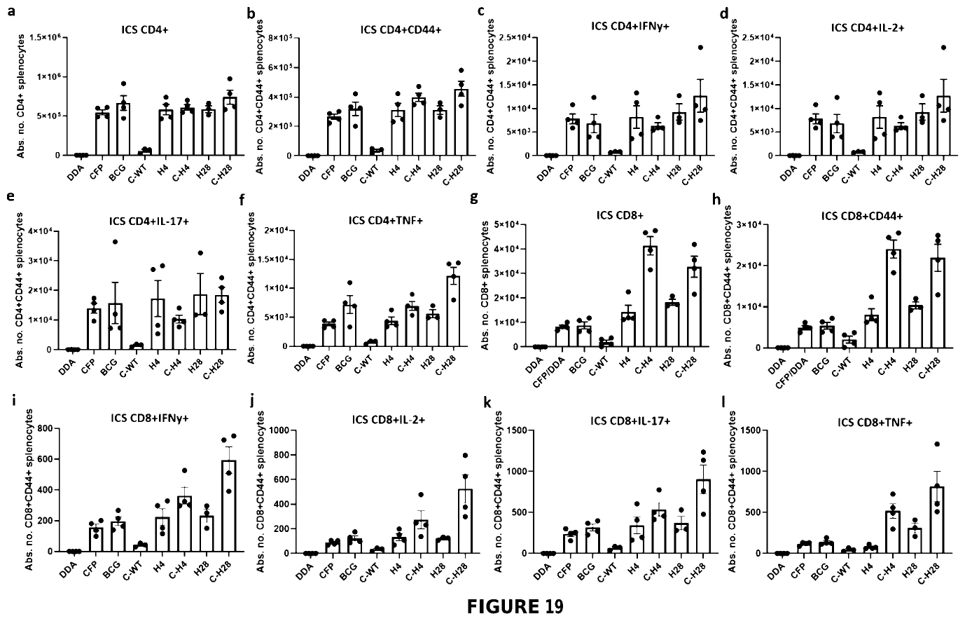

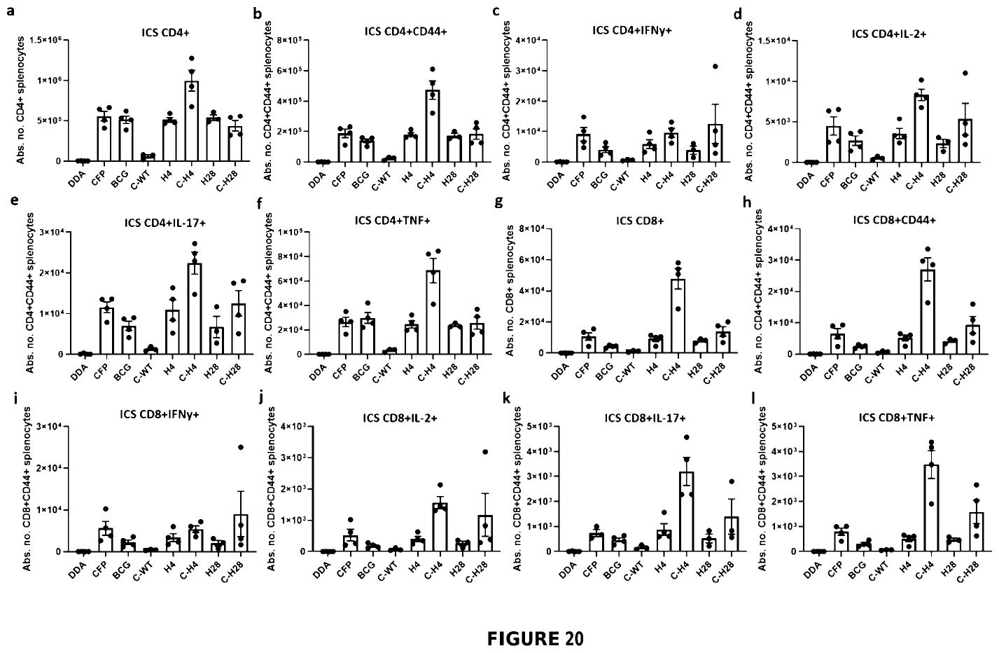

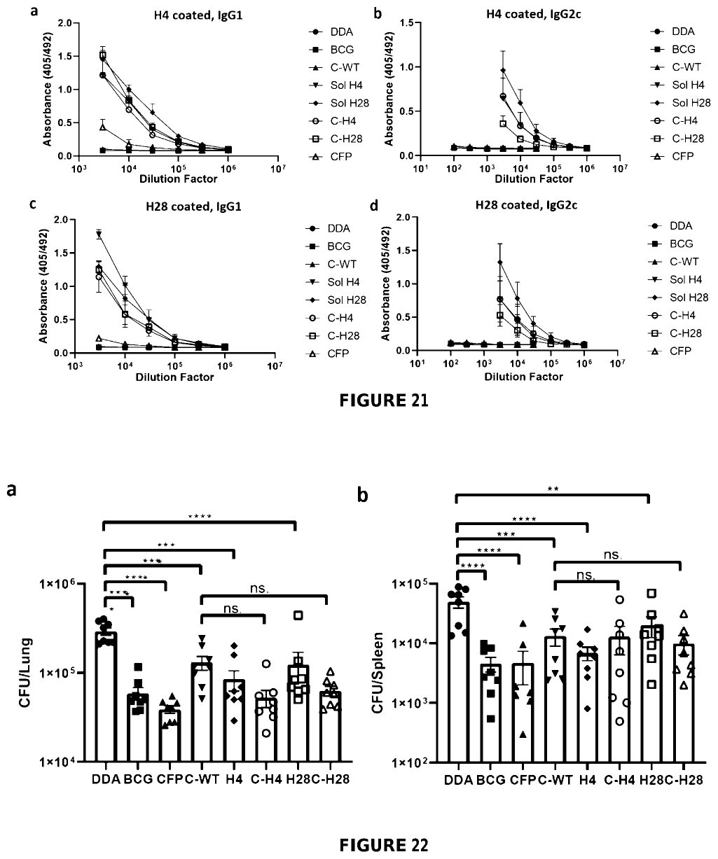

There is no significant difference of cytokine production of CD4+ and CD8+ T cells from mice immunised with various TB immunogens in response to soluble H4 stimulation (FIG. 18). In response to H28 stimulation, CD8+ T cells from mice immunised with CRM197 particles have high IFNγ, IL-2, IL-17 and TNF production in contrast to the CD8+ T cells from soluble H28 immunized mice (FIG. 19). Generally, the CD4+ and CD8+ T cells from mice immunized with CRM197-H4 particles or CRM197-H28 particles produce high levels of cytokines when compared with the cells from soluble H4 or H28 immunised mice in response to TB7.7 stimulation (FIG. 20).

### Lung and Spleen CFU of Mice Immunized by CRM197 TB Particles

Lung and spleen CFU counts from mice immunized with DDA adjuvant only are significantly higher than the lung and spleen CFU counts from mice immunized with BCG and CRM197 TB particles (FIG. 22). This suggests the controls performed as expected. FIG. 22 showed that the lung and spleen from mice immunized with soluble antigens (H4 and H28) have less CFU than the lung and spleen from BCG immunised mice. This suggested that soluble H4 and H28 antigens are able to induce protective immunity and this protectivity is stronger than the one BCG generated. This finding is consistent with previous studies [5, 6, 8, 41]. Furthermore, the lung and spleen from mice immunized with the particle test samples (CRM197-H4 and CRM197-H28) showed similar or less CFU than the one from mice immunised with soluble antigens (H4 or H28). This may indicate that particulate CRM197-H4 and/or CRM197-H28 may be able to provide equivalent or better protective immunity against M. tuberculosis when compared to the protective immunity generated by soluble TB antigens, H4 or H28.

### Example 4

### Immunogenicity Study of CRM197-Group a Streptococcal Peptide Particles

The ability of CRM197 containing protein particles designed to elicit an immune response to a group A streptococcal (GAS) bacteria will be investigated. A P*17 peptide (LRRDLDASREAKNQVERALE; SEQ ID NO:17) peptide and/or a S2 peptide (NSDNIKENQFEDFDEDWENF; SEQ ID NO:18) derived from a Streptococcus pyogenes have been utilised. Three genetic constructs for recombinant expression were constructed using routine techniques. The plasmids (1) CRM-P*17; (2) CRM-S2; and (3) CRM-P*17-S2. E. coli ClearColi strain were used for recombinant expression under suitable conditions. The proteins are chimeric proteins. The CRM197-peptide particles were prepared from the recombinant culture. The CRM197 particles with the P*17 and/or S2 peptides were used for administration to an animal to test immunogenicity. The CRM197 particles with the P*17 and/or S2 peptides were tested for their ability to elicit a specific immune response.

### Materials and Methods

### Bacterial Strains and Growth Conditions

Bacterial strains, plasmids and primers used in this study are listed in Table 5. Escherichia coli were grown in Luria Broth (LB) medium (Difco, Detroit, Mich.) at 37° C. with the appropriate antibiotic (ampicillin (Amp), 100 μg/mL). For the growth of the osmosensitive E. coli strain ClearColi™ BL21 (DE3) (Lucigen, USA), LB medium was supplemented with 1% NaCl. Primers were synthesized by Integrated DNA Technologies (IDT).

### Plasmid Construction for Production of CRM Particles

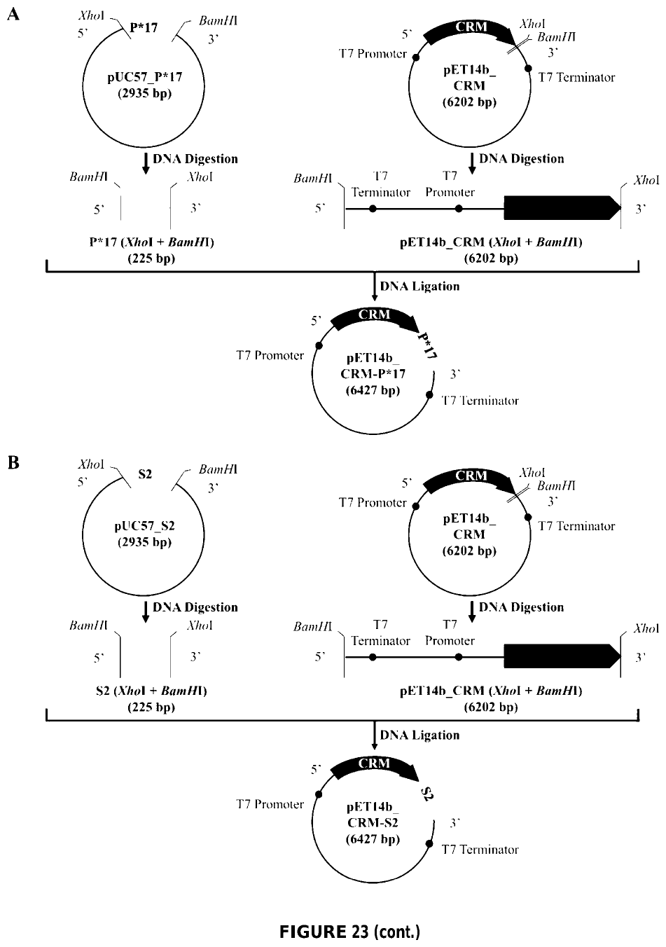

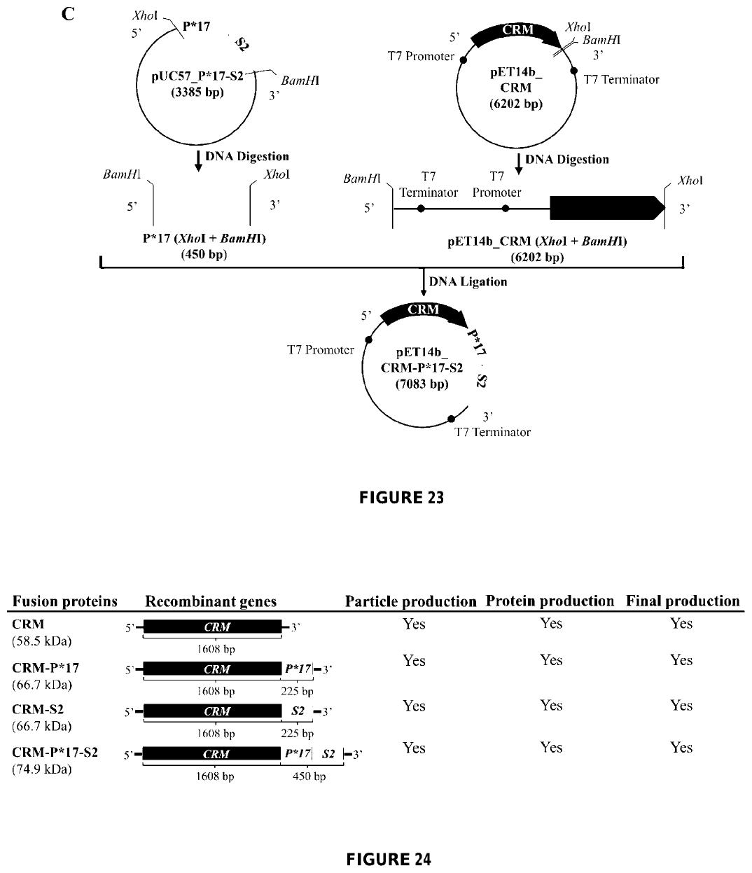

Cloning techniques were performed as described previously (Sambrook et al. 1989). DNA fragments were purchased from Biomatik (Canada). The cloning strategies for pET14b_CRM-P*17, pET14b_CRM-S2 and pET14b_CRM-P*17-S2 are demonstrated in FIG. 23. The fragments were excised from pUC57 vector by enzyme digestion with XhoI and BamHI, followed by fragment separation using agarose gel electrophoresis with SYBR safe stain (Invitrogen, USA) and gel purification (Qiagen, Thermo Fisher Scientific). The final plasmids were sequenced confirmed by Griffith University DNA Sequencing Facility (Griffith University, Nathan Campus, Australia) and transformed into the endotoxin free production host, E. coli strain ClearColi™ BL21 (DE3) (Lucigen, USA).

### CRM197 Particle Isolation and Purification

An overnight culture of the ClearColi™ BL21 (DE3) production host containing the respective plasmids was inoculated at 20 mL volume. The overnight culture was used for the large culture 4 L of LB medium supplemented with 1% (w/v) glucose and Amp, and was incubated at 37° C. at 200 rpm for approximately 3 h. The cell cultures were induced by IPTG at a final concentration of 1 mM when OD600 reached 0.5 and incubated further for 48 h at 37° C.

The cells were harvested by centrifugation at 8000×g for 20 min at 4° C. and resuspended in 0.5× lysis buffer (25 mM Tris, 5 mM EDTA and 0.04% (w/v) SDS, pH 7). The whole cell lysate was mechanically disrupted using Microfluidizer M-110P (Microfluidics, USA). The cell lysate was centrifuged at 8000×g for 20 min at 4° C. to pellet CRM particles. The isolated CRM particles were washed and purified three times by 0.5× lysis buffer. The purified CRM protein particles were sterilized with 1 mg/mL Ciprofloxacin and washed three times with Tris buffered saline (TBS) (50 mM Tris, 150 mM NaCl, pH 7.5). The sterile CRM particulate test samples were stored in TBS.

### Proteins and Particles Analysis

The purified CRM particles were separated on a 10% Bis-Tris polyacrylamide gel to visualize and quantify the fusion protein percentage using densitometry with BSA standards ranging from 62.5 ng to 500 ng. The target protein bands were excised and subjected protein identification using Q-TOF/MS. All the target protein sequences were identified and shown in Table 6. The images were captured using Image Lab Software (Bio-Rad Laboratories, USA). Particle size and zeta-potential were measured using Zetasizer Nano ZS (Malvern, UK) at Queensland Micro Nanotechnology Centre (Griffith University, Queensland, Australia). The target protein bands on Bis-Tris gel were excised for protein identification and confirmation using Mass Spectrometry (MS) in Clinical Research Center (The University of Queensland, Brisbane, Australia).

### Formulation and Immunization

Formulated test preparations for immunogenicity studies contained 5 μg of StrepA antigens/dose, mixed with Alhydrogel 2% (Alum) (25 μL/dose; InvivoGen, USA) in 100 μL volume for 1 h at room temperature rotating. Alum in TBS was used as negative control. Soluble P*17-DT+K4S2-DT (DT-Diphtheria toxoid, the toxic version of CRM) was used as positive control. Prepared formulations were mixed with alum freshly before injection. Animal experiments were approved by Griffith University Animal Ethics Committee (Gold Coast, Australia). The animal trial was performed using 6-week old female BALB/c mice. There were 5 mice per group. Formulated test preparations were injected into mice intramuscularly, 50 μL in each thigh, a total of 100 μL per mice. Mice were immunized three times (Day 0, 21 and 28). A challenge experiment will be conducted. The animal trials were performed at the Institute of Glycomics at Griffith University (Australia).

### Sera Collection

Blood was collected via submandibular bleeding at 20, 27 and 35 days and cardiac puncture at day 42. The blood was allowed to clot at room temperature and subsequently the blood clot was removed. The murine sera were separated by centrifugation at 664×g for 10 min and stored at −80° C. until analysis.

### ELISA

Serum antibody responses were analyzed by enzyme-linked immunosorbent assay (ELISA). High-binding plates (Greiner Bio-One, Germany) were coated overnight at 4° C. with 100 μL of 5 μg/mL of soluble proteins P*17 and K4S2 diluted in carbonate coating buffer (Na₂CO₃, NaHCO₃, pH 9.6). The next day, the plates were blocked with 200 μL of 5% skim milk in PBST for 60 min at 37° C. Plates were washed three times with PBST and incubated with primary polyclonal antibodies, murine sera taken from individual mice diluted with 0.5% skim milk in PBST for 60 min at 37° C. with concentration ranging from 1/100 to 1/3276800. Plates were washed three times before incubation with the secondary HRP-conjugated antibodies anti-mouse IgG or IgG1 or IgG2a or IgG2b or IgG3 (Abcam, UK) diluted with PBST at a concentration of 1/5000, at 37° C. for 60 min. After washing three times, o-phenylenediamine (OPD) was added on plates and incubated for 20-25 min at room temperature and measured at 450 nm on a plate reader. The ELISA was done at the Griffith Institute for Drug Discovery (GRIDD) (Griffith University, Queensland, Australia).

### Western Blot

The specificity of the IgG responses was investigated using western blot analysis. Pooled sera from the immunized mice diluted 2000-fold were used against CRM particles after subjected to SDS-PAGE and transfer to nitrocellulose membranes (Life Technology, USA). Antimouse IgG HRP-conjugate (Abcam, UK) diluted 20000-fold and used for bound IgG antibodies detection. SuperSignal West Pico Stable Peroxide Solution and SuperSignal West Pico Luminol/Enhancer Solution (Thermo Scientific, USA) were used to develop the signal. The blots were imaged using the Odyssey® Fc Imaging System (LI-COR®). The Western Blot was performed at the GRIDD (Griffith University, Queensland, Australia).

### Statistical Analysis

Antibody responses were analyzed using one-way ANOVA with statistical significance (p<0.05) indicated by letter-based representation of pairwise comparison between groups using Tukey's post-hoc test. Each data point represents results from five mice ± the standard error of the mean.

### Results and Discussion

Streptococcus pyogenes (group A Streptococcus; GAS) infections continue to be a primary problem causing high mortality in humans ranging 10 to 30%, resulting to 600,000 deaths per year globally, especially occurring in resource limiting areas [42, 43]. GAS is a versatile Gram-positive bacterium causing spectrum of human diseases ranging from mild infections to life-threatening diseases such as toxic shock syndrome and necrotising fasciitis, as well as post infection immune related diseases. An effective StrepA vaccine is desirable to prevent infections and decrease mortality and morbidity, particularly because treatments are expensive.

In this study, two target proteins/antigens that were previously demonstrated to induce immunity and give protection against the invasive streptococcal disease. P*17 is the first antigen, a p145 variant (conserved carboxyl terminal region of M protein) [44] developed using amino acid substitution strategy to enhance immunogenicity [45]. P*17 showed superior levels of antibodies in just single immunization, greater stability, and 10,000 fold enhanced protection from the streptococcal disease [45]. S2 is the second target antigen, non-M protein and highly conserved antigen from streptococcal IL-8 protease, SpyCEP showed significant reduction in systemic and local GAS burden in combination with J8 antigen (a 12 aa epitope existing within p145) demonstrated both in conjugation with DT and CRM [46]. Current preparations of these vaccines for human clinical trials are conjugated with CRM (enzymatically inactive and nontoxic form of diphtheria toxin (DT)) [47]. However, vaccine conjugated with CRM is expensive; hence in this study we used the cost-effective way to genetically modify E. coli to produce CRM particles displaying our target antigens, P*17 and S2.

### Bioengineering Towards In Vivo Self-Assembly of CRM Particles Displaying P*17 and S2 Antigens

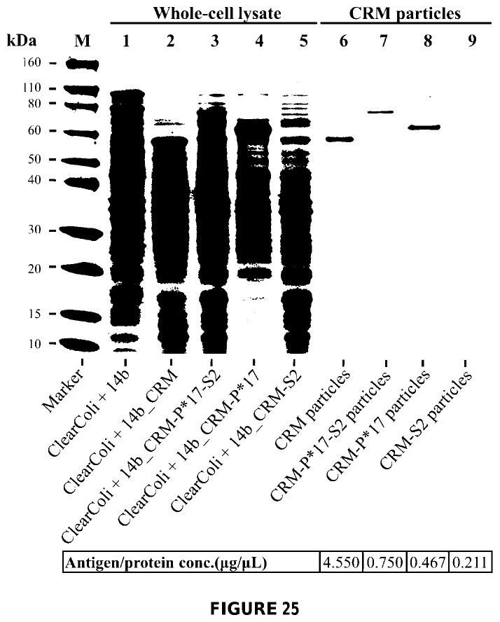

The modular composition of the hybrid genes and the respective encoded fusion proteins is shown in FIG. 24. E. coli ClearColi BL21™ (DE3) production strain harbouring the various plasmids encoding the StrepA antigens, P*17 and S2 were cultivated under conditions to produce the CRM particles displaying the recombinant proteins. To construct the hybrid genes, the CRM together with the P*17 and S2 genes were genetically manipulated and cloned into pET14b expression vector containing the T7 strong promoter [48, 49]. Four plasmid constructs were used in this study including the pET14b_CRM for only CRM particle production and three plasmids were constructed containing the StrepA antigens fused to the C-terminal of CRM: pET14b_CRM-P*17, pET14b_CRM-S2 and pET14b_CRM-P*17-S2 to produce CRM-P*17, CRM-S2 and CRM-P*17-S2 particles, respectively. When hybrid genes were overexpressed under the T7 strong promoter, in vivo self-assembly of CRM particles and CRM+ antigens particles were observed. In addition, it also led to the production of the recombinant proteins, CRM, CRM-P*17, CRM-S2 and CRM-P*17-S2 (FIG. 25), which were used for animal trials.

### Characterization of Purified CRM—StrepA Particles

CRM particles displaying the StrepA antigens were isolated and released from cells using mechanical disruption with a Microfluidizer. The protein profile of the whole cell lysate (before cell disruption) and the purified CRM particles diluted 500 times from 20% (w/v) suspension were analysed by SDS-PAGE (FIG. 25). Densitometry analysis using BSA standards (62.5 to 500 ng) showed the quantity of the CRM (4.550 μg/uL) and the antigens P*17-S2 (0.750 μg/uL), P*17 (0.467 μg/uL) and S2 (0.211 μg/uL). The dominant protein bands corresponding to the theoretical MW of CRM (58.5 kDa), CRM-P*17-S2 (74.9 kDa), CRM-P*17 (66.7 kDa) and CRM-S2 (66.7 kDa), were excised and protein sequences were confirmed by mass spectrometry (Table 6).

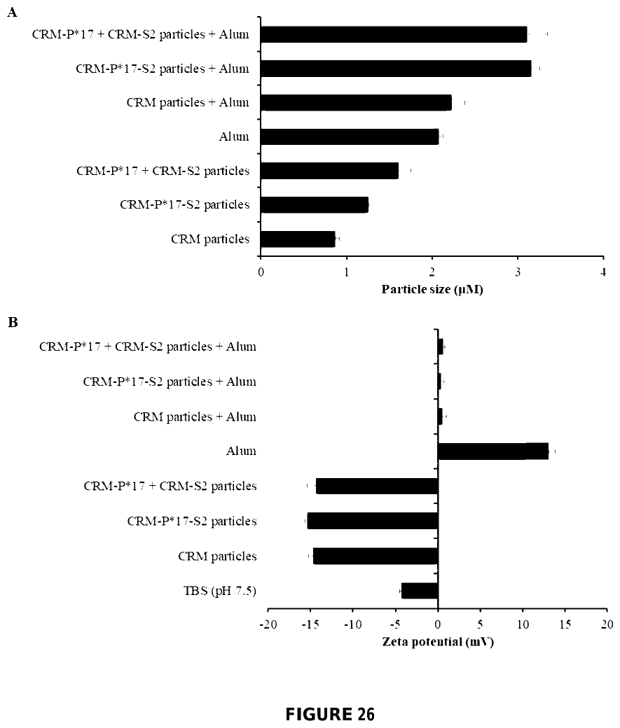

In order to examine the impact of Alum adjuvant to the size and surface charges of various CRM particle preparations in TBS buffer, the particle size and zeta-potential of particles were analysed before and after mixture with Alum (FIG. 26). In contrast to soluble antigens which are usually taken up by the antigen presenting cells (APCs) via endocytosis, particles with size ranging from 0.5 μm to 10 μm are taken up by phagocytosis [23, 50]. The uptake of particulate antigens by phagocytosis can results to antigen cross-presentation eventually inducing both humoral and cell-mediated immune responses [24]. The size of Alum colloids is larger (2.1 μm) compared to the various CRM particles (0.9 μm-1.6 μm) (FIG. 26A). Addition of Alum to various CRM particles caused an increase sizes to 2.2 μm to 3.1 μm. These aggregates were possibly due to electrostatic interactions within and between particles and Alum. Furthermore, similar to the particle size, Alum also influenced shift of the surface charge of the CRM particles from negative to positive (FIG. 26B). Nevertheless, the surface charge of the particles upon injection into mice in vivo is unknown. The surface charge of particles may affect the cellular uptake as positively charged particles are known to be increasingly taken up by dendritic cells [17]. Furthermore, cell membranes are dominated by negative surface charges that might repel negatively charged particles [51, 52]. However, numerous studies had demonstrated that negatively charged particles were efficiently taken up by APCs probably due to the cell membrane's cationic sites facilitating adsorption of negatively charged particles [19, 51, 52], or mediating opsonization [19, 50].

### Mice Immunisation and Immunological Evaluation

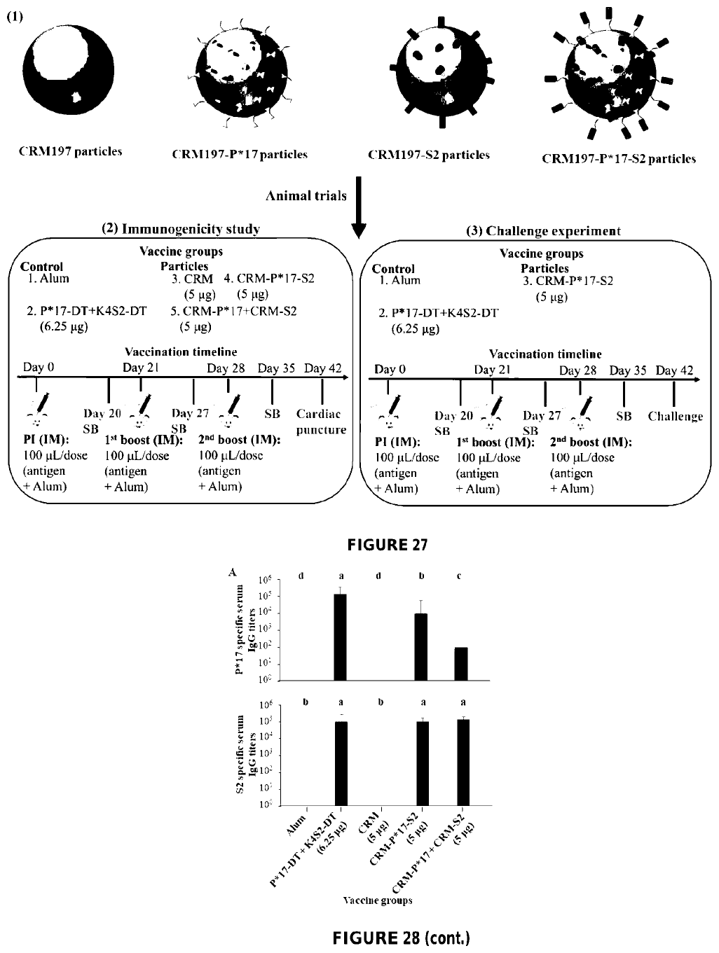

A schematic diagram of the CRM197 particles and experimental plan of animal study is illustrated in FIG. 27. (1) To avoid the presence of lipopolysaccharide (LPS) endotoxins, which can co-purify with various biological products [53] and can cause wide range of pathophysiological effects in both animals and humans [53, 54]; E. coli ClearColi BL21 (DE3) strain [55] was used as production host to produce an endotoxin free product. The plasmids encoding pET14b_CRM, pET14b_CRM-P*17-S2, pET14b_CRM-P*17 and pET14b_CRM-S2 were used to transform ClearColi™ BL21 (DE3) production strain. (2) Strains harbouring various plasmids were grown under optimum conditions at 37° C. for 48 h to mediate the overproduction of fusion proteins/antigens and (3) subsequent CRM particle in vivo self-assembly. (4) CRM particles were isolated from the E. coli cells via mechanical disruption and purified three times using lysis buffer wash. (5) For immunogenicity study, mice were injected intramuscularly with 5 μg of CRM and StrepA antigens per dose mixed with 25 μL of Alum in a total volume of 100 μL. Alum served as negative control and P*17-DT+K4S2-DT (6.25 μg) as positive control. CRM-P*17-S2 has the advantage of the two antigens being produced in a one-way process while the physical combination of CRM-P*17 and CRM-S2 are two different particles that needed to be separately produced; hence more time and cost. Following immunisation, all mice looked healthy, gained weight and remained alive throughout the trial. Submandibular bleeding (SB) was done to collect sera in mice at 20, 27 and 35 days after primary immunization (PI). Cardiac puncture was done at day 42 to cull the mice and collect sera. No noticeable abnormal behaviours and no adverse effects were observed (data not shown). (6) Mice challenge with the bacterial pathogen is ongoing.

Mice Vaccination with CRM-Containing Particles Induce Antigen Specific Immune Responses

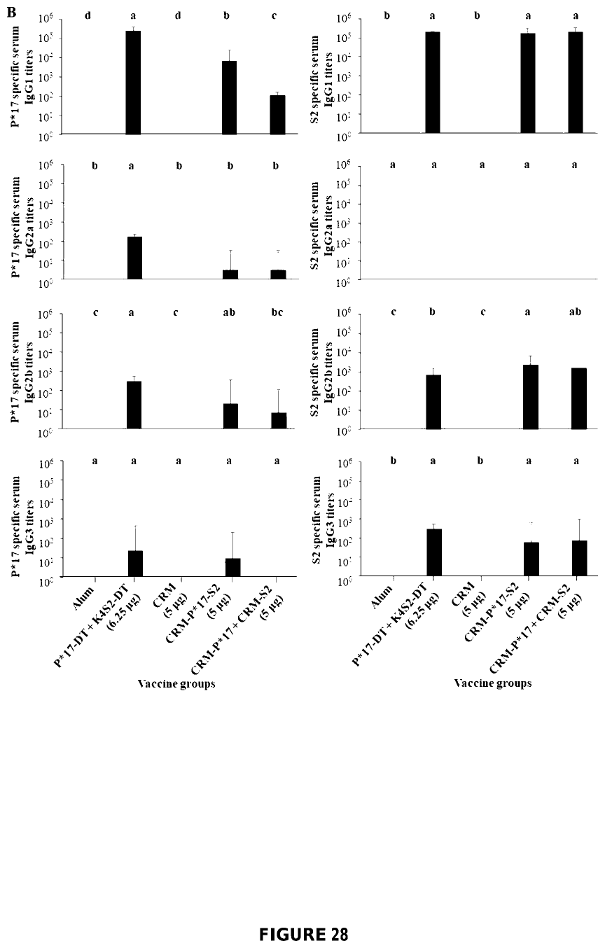

Immunogenicity of CRM, CRM-P*17-S2 and CRM-P*17+CRM-S2 particles together with the positive control conjugates P*17-DT+K4S2-DT and negative control Alum were assessed in a murine model. Serum samples were collected at defined time-points and ELISAs were performed to determine antibody titers. Specifically, total IgG and IgG subtypes (IgG1, IgG2a, IgG2b and IgG3) were measured in this study to characterize antigen associated humoral immune responses (FIG. 28). In P*17 specific total IgG response (FIG. 28A), the positive control P*17-DT+K4S2-DT was significantly higher compared to the CRM-P*17-S2 and the physical combination of CRM-P*17+CRM-S2. Although, P*17-DT+K4S2-DT had higher dose of 6.25 μg compared to the 5 μg of CRM-P*17-S2 and CRM-P*17+CRM-S2 particles. The reduced IgG response might also be due to the P*17 being embedded within the particle and not surface exposed. On the other hand, in S2 specific total IgG response (FIG. 28A), there was no significant difference between the P*17-DT+K4S2-DT conjugates and CRM-P*17-S2 and CRM-P*17+CRM-S2 particles. Hence, despite the lower dosage of CRM-P*17-S2 and CRM-P*17+CRM-S2, they performed similar in terms of IgG response compared to the positive control. There were no P*17 and S2 specific antibody titers detected in the negative control Alum and CRM. Previous study had demonstrated P*17 specific IgG titers slightly higher than the positive control P*17-DT+K4S2-DT and used 30 μg of the antigen [45]. While S2 specific IgG titers of the P*17-DT+K4S2-DT, CRM-P*17-S2 and CRM-P*17+CRM-S2 particles were comparable from previous study that injected 30 μg of S2 antigen [46]. There are room for the CRM-P*17-S2 and CRM-P*17+CRM-S2 to increase dosage to more than 100 μg with the potential to increase antigen specific antibody titers. Similar pattern and levels were observed in P*17- and S2-specific IgG1 antibody titers (FIG. 28B). The less abundant subtypes, IgG2a, IgG2b and IgG3 varied especially noticeable that there was no detectable S2 specific IgG2a titers. These results suggested the induction of a strong Th2 immunity characterized by the high IgG1 antibody titers. Some Th1 immune response was also stimulated characterized by the presence of IgG2a and IgG2b.

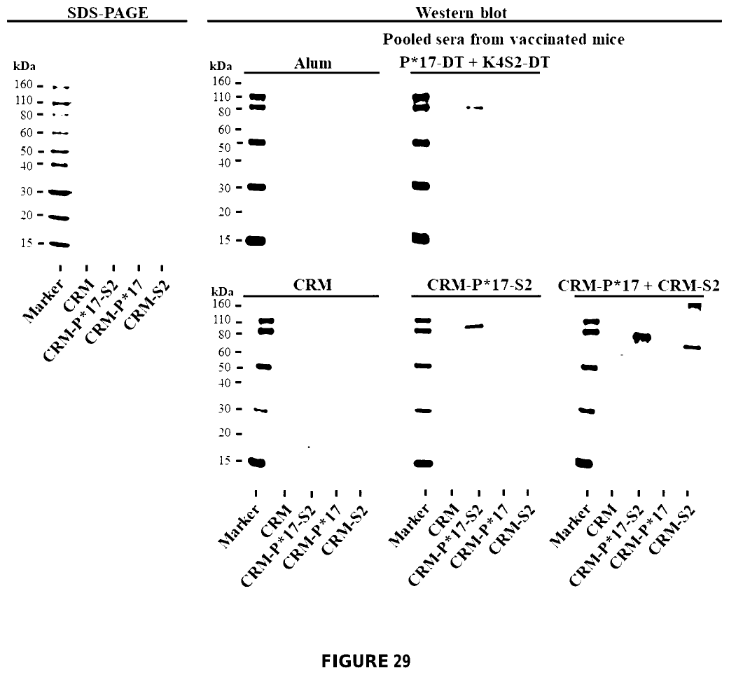

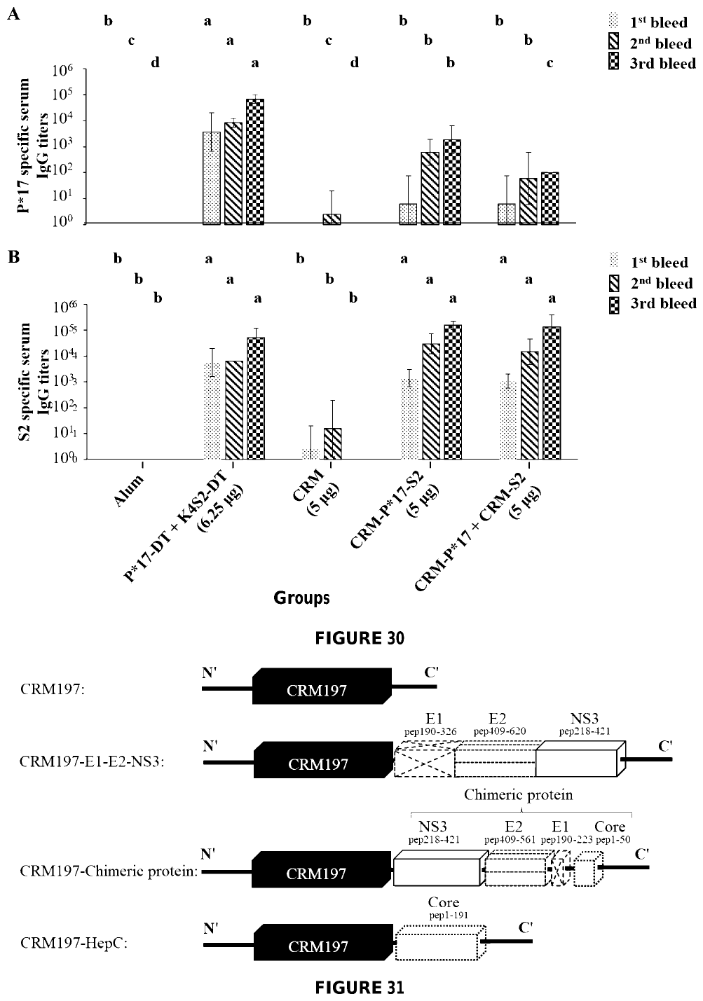

To test the specificity of antibody response, pooled sera from the immunised mice were used in western blot analysis against components of the various test formulations (FIG. 29). Pooled sera from mice injected with CRM-P*17-S2 and CRM-P*17+CRM-S2 specifically recognized protein bands corresponding to the theoretical MW of CRM (58.5 kDa), CRM-P*17-S2 (74.9 kDa), CRM-P*17 (66.7 kDa) and CRM-S2 (66.7 kDa). Furthermore, pooled sera from P*17-DT+K4S2-DT immunised mice had specifically recognized all protein bands except CRM. No bands were detected in the pooled sera from mice injected with Alum and CRM.

### Example 5

### CRM197-HCV Particles Produced in ClearColi BL21(DE3)

A CRM197 protein particle comprising one or more HCV immunogenic sequences were prepared as chimeric proteins, and the immunogenicity thereof will be tested. The one or more HCV immunogenic sequences that will be used will be an immunogenic amino acid sequence derived from, or corresponding to, an HCV viral protein selected from the group consisting of an E1 protein, an E2 protein, an NS3 protein and a core protein, and combinations thereof. The immunogenic amino acid sequences of HCV as described below and in FIG. 31 were translationally fused to the C-terminus of CRM197. Schematic design of hybrid genes encoding fusion proteins for production of CRM197 particles coated with HCV-containing particles (CRM197-E1-E2-NS3, CRM197-Chimeric protein, and CRM197-HepC) is illustrated in FIG. 31. In particular, each recombinant HCV antigen, including E1-E2-NS3, chimeric protein, and HepC antigen, was translationally fused to the C terminus of CRM197. The resulting recombinant proteins, including CRM197-E1-E2-NS3, CRM197-Chimeric protein, and CRM197-HepC, were respectively overproduced and self-assembled into CRM197 protein particles carrying designated HCV fusion (E1-E2-NS3, chimeric protein, or HepC) in the endotoxin free E. coli strain, ClearColi BL21(DE3). Hence, each recombinant HCV fusion was displayed on the surface of and/or incorporated into the CRM197 particles.

A core protein amino acid sequence in the form of peptide 1-50, also referred to as “pep1-50” (see FIG. 31) was tested in this study. An amino acid sequence of, or from, a wild-type HCV core protein amino acid sequence from which peptide 1-50 is derived is as follows:

MSTNPKPQRKTKRNTNRRPQDVKFPGGGQIVGGVYLLPRRGPRLGVRATR
KTSERSQPRGRRQPILRDRRSTGKSWGKPGYPWPLYGNEGCGWAGWLLSP
RGSRPTWGPTDPRHRSRNLGKVIDTITCSLADLMGYIPVIGAPVGGVARA
LAHGVRVLEDGVNYATGNLPGCSFSIFLLALLSCITVPVSA (GenBank
Accession Number BAC20466.1; SEQ ID NO: 43;
referred to as “pep1-191”).

Pep 1-191 is a 191 amino acid sequence derived from an HCV core protein.

An amino acid sequence of a HCV core protein fragment in the form of a 50 amino acid residue peptide (pep1-50; SEQ ID NO:28) corresponding to amino acid residues 1 to 50 of a wild-type HCV core protein peptide recited above (pep1-191; SEQ ID NO:43) used in this study is as follows:

(SEQ ID NO: 28)
MSTNPKPQRKTKRNTNRRPQDVKFPGGGQIVGGVYLLPRRGPRLGVRATR

An amino acid sequence of an HCV polyprotein of an entire HCV genome is provided as follows:

MSTNPKPQRKTKRNTNRRPQDVKFPGGGQIVGGVYLLPRRGPRLGVRATRKTSERSQ
PRGRRQPIPKARRPEGRTWAQPGYPWPLYGNEGCGWAGWLLSPRGSRPSWGPTDPRRRSRN
LGKVIDTLTCGFADLMGYIPLVGAPLGGAARALAHGVRVLEDGVNYATGNLPGCSFSIFLLA
LLSCLTVPASAYQVRNSSGLYHVTNDCPNSSIVYEAADAILHTPGCVPCVREGNASRCWVAV
TPTVATRDGKLPTTQLRRHIDLLVGSATLCSALYVGDLCGSVFLVGQLFTFSPRRHWTTQDCN
CSIYPGHITGHRMAWDMMMNWSPTAALVVAQLLRIPQAIMDMIAGAHWGVLAGIAYFSMV
GNWAKVLVVLLLFAGVDAETHVTGGSAGRTTAGLVGLLTPGAKQNIQLINTNGSWHINSTA
LNCNESLNTGWLAGLFYQHKFNSSGCPERLASCRRLTDFAQGWGPISYANGSGLDERPYCW
HYPPRPCGIVPAKSVCGPVYCFTPSPVVVGTTDRSGAPTYSWGANDTDVFVLNNTRPPLGNW
FGCTWMNSTGFTKVCGAPPCVIGGVGNNTLLCPTDCFRKHPEATYSRCGSGPWITPRCMVDY
PYRLWHYPCTINYTIFKVRMYVGGVEHRLEAACNWTRGERCDLEDRDRSELSPLLLSTTQWQ
VLPCSFTTLPALSTGLIHLHQNIVDVQYLYGVGSSIASWAIKWEYVVLLFLLLADARVCSCLW
MMLLISQAEAALENLVILNAASLAGTHGLVSFLVFFCFAWYLKGRWVPGAVYAFYGMWPLL
LLLLALPQRAYALDTEVAASCGGVVLVGLMALTLSPYYKRYISWCMWWLQYFLTRVEAQL
HVWVPPLNVRGGRDAVILLMCVVHPTLVFDITKLLLAIFGPLWILQASLLKVPYFVRVQGLLR
ICALARKIAGGHYVQMAIIKLGALTGTYVYNHLTPLRDWAHNGLRDLAVAVEPVVFSRMET
KLITWGADTAACGDIINGLPVSARRGQEILLGPADGMVSKGWRLLAPITAYAQQTRGLLGCII
TSLTGRDKNQVEGEVQIVSTATQTFLATCINGVCWTVYHGAGTRTIASPKGPVIQMYTNVDQ
DLVGWPAPQGSRSLTPCTCGSSDLYLVTRHADVIPVRRRGDSRGSLLSPRPISYLKGSSGGPLL
CPAGHAVGLFRAAVCTRGVAKAVDFIPVENLETTMRSPVFTDNSSPPAVPQSFQVAHLHAPT
GSGKSTKVPAAYAAQGYKVLVLNPSVAATLGFGAYMSKAHGVDPNIRTGVRTITTGSPITYS
TYGKFLADGGCSGGAYDIIICDECHSTDATSILGIGTVLDQAETAGARLVVLATATPPGSVTVS
HPNIEEVALSTTGEIPFYGKAIPLEVIKGGRHLIFCHSKKKCDELAAKLVALGINAVAYYRGLD
VSVIPTSGDVVVVSTDALMTGFTGDFDSVIDCNTCVTQTVDFSLDPTFTIETTTLPQDAVSRTQ
RRGRTGRGKPGIYRFVAPGERPSGMFDSSVLCECYDAGCAWYELTPAETTVRLRAYMNTPGL
PVCQDHLEFWEGVFTGLTHIDAHFLSQTKQSGENFPYLVAYQATVCARAQAPPPSWDQMWK
CLIRLKPTLHGPTPLLYRLGAVQNEVTLTHPITKYIMTCMSADLEVVTSTWVLVGGVLAALA
AYCLSTGCVVIVGRIVLSGKPAIIPDREVLYQEFDEMEECSQHLPYIEQGMMLAEQFKQKALG
LLQTASRQAEVITPAVQTNWQKLEVFWAKHMWNFISGIQYLAGLSTLPGNPAIASLMAFTAA
VTSPLTTGQTLLFNILGGWVAAQLAAPGAATAFVGAGLAGAAIGSVGLGKVLVDILAGYGA
GVAGALVAFKIMSGEVPSTEDLVNLLPAILSPGALVVGVVCAAILRRHVGPGEGAVQWMNR
LIAFASRGNHVSPTHYVPESDAAARVTAILSSLTVTQLLRRLHQWISSECTTPCSGSWLRDIWD
WICEVLSDFKTWLKAKLMPQLPGIPFVSCQRGYRGVWRGDGIMHTRCHCGAEITGHVKNGT
MRIVGPRTCRNMWSGTFPINAYTTGPCTPLPAPNYKFALWRVSAEEYVEIRRVGDFHYVSGM
TTDNLKCPCQIPSPEFFTELDGVRLHRFAPPCKPLLREEVSFRVGLHEYPVGSQLPCEPEPDVA
VLTSMLTDPSHITAEAAGRRLARGSPPSMASSSASQLSAPSLKATCTANHDSPDAELIEANLL
WRQEMGGNITRVESENKVVILDSFDPLVAEEDEREVSVPAEILRKSRRFARALPVWARPDYNP
PLVETWKKPDYEPPVVHGCPLPPPRSPPVPPPRKKRTVVLTESTLSTALAELATKSFGSSSTSGI
TGDNTTTSSEPAPSGCPPDSDVESYSSMPPLEGEPGDPDLSDGSWSTVSSGADTEDVVCCSMS
YSWTGALVTPCAAEEQKLPINALSNSLLRHHNLVYSTTSRSACQRQKKVTFDRLQVLDSHYQ
DVLKEVKAAASKVKANLLSVEEACSLTPPHSAKSKFGYGAKDVRCHARKAVAHINSVWKDL
LEDSVTPIDTTIMAKNEVFCVQPEKGGRKPARLIVFPDLGVRVCEKMALYDVVSKLPLAVMG
SSYGFQYSPGQRVEFLVQAWKSKKTPMGFSYDTRCFDSTVTESDIRTEEAIYQCCDLDPQARV
AIKSLTERLYVGGPLTNSRGENCGYRRCRASGVLTTSCGNTLTCYIKARAACRAAGLQDCTM
LVCGDDLVVICESAGVQEDAASLRAFTEAMTRYSAPPGDPPQPEYDLELITSCSSNVSVAHDG
AGKRVYYLTRDPTTPLARAAWETARHTPVNSWLGNIIMFAPTLWARMILMTHFFSVLIARDQ
LEQALNCEIYGACYSIEPLDLPPIIQRLHGLSAFSLHSYSPGEINRVAACLRKLGVPPLRAWRHR
ARSVRARLLSRGGRAAICGKYLFNWAVRTKLKLTPIAAAGRLDLSGWFTAGYSGGDIYHSVS
HARPRWFWFCLLLLAAGVGIYLLPNR (GenBank Accession Number AF009606.1; SEQ ID
NO: 44).

An amino acid sequence of an HCV NS3 protein is as follows:

GSVVIVGRIILSGSGSITAYSQQTRGLLGCIITSLTGRDKNQVEGEVQVV
STATQSFLATCVNGVCWTVYHGAGSKTLAGPKGPITQMYTNVDQDLVGWQ
APPGARSLTPCTCGSSDLYLVTRHADVIPVRRRGDSRGSLLSPRPVSYLK
GSSGGPLLCPSGHAVGIFRAAVCTRGVAKAVDFVPVESMETTMRSPVFTD
NSSPPAVPQSFQVAHLHAPTGSGKSTKVPAAYAAQGYKVLVLNPSVAATL
GFGAYMSKAHGIDPNIRTGVRTITTGAPVTYSTYGKFLADGGCSGGAYDI
IICDECHSTDSTTILGIGTVLDQAETAGARLVVLATATPPGSVTVPHPNI
EEVALSNTGEIPFYGKAIPIEAIRGGRHLIFCHSKKKCDELAAKLSGLGI
NAVAYYRGLDVSVIPTIGDVVVVATDALMTGYTGDFDSVIDCNTCVTQTV
DFSLDPTFTIETTTVPQDAVSRSQRRGRTGRGRRGIYRFVTPGERPSGMF
DSSVLCECYDAGCAWYELTPAETSVRLRAYLNTPGLPVCQDHLEFWESVF
TGLTHIDAHFLSQTKQAGDNFPYLVAYQATVCARAQAPPPSWDQMWKCLI
TRLKPTLHGPTPLLYRLGAVQNEVLTHPITKYIMACMSADLEVVT (PDB
Accession Number 1CU1_A; SEQ ID NO: 69).

An amino acid sequence of an HCV NS3 protein fragment spanning residues 218 to 421 of SEQ ID NO:69 used in this study is as follows:

(SEQ ID NO: 29)
APTGSGKSTKVPAAYAAQGYKVLVLNPSVAATLGFGAYMSKAHGIDPNIR
TGVRTITTGAPVTYSTYGKFLADGGCSGGAYDIIICDECHSTDSTTILGI
GTVLDQAETAGARLVVLATATPPGSVTVPHPNIEEVALSNTGEIPFYGKA
IPIEAIRGGRHLIFCHSKKKCDELAAKLSGLGINAVAYYRGLDVSVIPTI
GDVV.

This peptide is referred to “pep218-421” in FIG. 31.

E1 peptides used in this study are derived from a wild-type HCV E1 protein amino acid sequence as follows:

MSTNPKPQRKTKRNTNRRPQDVKFPGGGQIVGGVYLLPRRGPRLGVRATR
KTSERSQPRGRRQPIPKARRPEGRTWAQPGYPWPLYGNEGMGWAGWLLSP
RGSRPSWGPTDPRRRSRNLGKVIDTLTCGFADLMGHIPLVGAPLGGAARA
LAHGVRVLEDGVNYATGNLPGCSFSIFLLALLSCLTIPASAYEVRNASGV
YHVTNDCSNSSIVYETADMIMHTPGCVPCVREDNSSRCWVALTPTLAARN
ASIPTTTIRRHVDLLVGAAAFCSAMYVGDLCGSVFLVSQLFTFSPRRHET
VQDCNCSIYPGHVSGHRMAWDMMMNWSPTAALMVSQLLRIPQAVVDMVAG
AHWGVLAGLAYYSMAGNWAKVLIVMLLFAGVDGQTTVMGGVAGRTTFGFA
ALFNPGPSQKIQ (GenBank Accession Number ADV92203.1;
SEQ ID NO: 45).

An amino acid sequence of an E1 protein fragment spanning residues 190-326 of a wild-type HCV E1 protein amino acid sequence as set out in SEQ ID NO:45 and used in this study is as follows:

(SEQ ID NO: 30)
SAYEVRNASGVYHVTNDCSNSSIVYEADDMIMHTPGCVPCVREDNTSRCW
VALTPTLAARNASVPTTTIRRHVDLLVGAAALCSAMYVGDLCGSVFLVSQ
LFTFSPRRHETAQDCNCSIYPGHVSGHRMAWDMMMNW.

An amino acid sequence of an HCV E1 protein peptide spanning residues 190 to 223 (referred to as “pep190-223” in FIG. 31) of a wild-type HCV E1 protein amino acid sequence (SEQ ID NO:45) used in this study is as follows: SAYEVRNASGVYHVTNDCSNSSIVYEADDMIM (pep190-223; SEQ ID NO:70). This peptide is referred to as “pep190-326” in FIG. 31.

Peptides from E2 protein of HCV were tested in this study. HCV E1 and E2 proteins are initially produced as one polyprotein that is post-translationally cleaved into E1 and E2. An amino acid sequence of an HCV E1/E2 polyprotein is set forth in SEQ ID NO:46:

MSTNPKPQRKTKRNTNRRPQDVKFPGGGQIVGGVYLLPRRGPRLGVRATR
KTSGRSQPRGRRQPIPKARRPEGRSWAQPGYPWPLYGNEGMGWAGWLLSP
RGSRPSWGPTDPRRRSRNLGKVIDTLTCGFADLMGYIPLVGAPLGGAARA
LAHGVRVLEDGVNYATGNLPGCSFSLFLLALLSCLTIPVSAYEVRNASGV
YHVTNDCSNSSIVYEADDMIMHTPGCVPCVREDNTSRCWVALTPTLAARN
ASVPTTTIRRHVDLLVGAAALCSAMYVGDLCGSVFLVSQLFTFSPRRHET
VQDCNCSIYPGHVSGHRMAWDMMMNWSPSTALVVSQLLRIPQAVVDMVAG
AHWGVLAGLAYYSMVGNWAKVLIVMLLFAGVDGTGTYVTGGTAARGVSQF
TGLFTSGPSQKIQLVNTNGSWHINRTALNCNDSLQTGFLAALFYVHRFNS
SGCSDRMASCRPIDTFDQGWGPITYAEPRSLDQRPYCWHYAPQPCGIVPA
AEVCGPVYCFTPSPVVVGTTDRSGVPTYNWGENETDVLLLNNTRPPLGNW
FGCTWMNSTGFTKTCGGPPYNIGGVGNNTLTCPTDCFRKHPEATYTKCGL
GPWLTPRCLVDYPYRLWHYPCTVNFTIFKVRMYVGGVEHRLTAACNWTRG
(GenBank Accession Number ABX54697.1; SEQ ID NO:
46).

An amino acid sequence of an HCV E2 protein fragment used in this study which spans residues 409-620 of a wild-type HCV E2 protein amino acid sequence as set out in SEQ ID NO:46 is as follows:

(SEQ ID NO: 31)
SQKIQLVNTNGSWHINRTALNCNDSLQTGFLAALFYVHRFNSSGCSDRMA
SCRPIDTFDQGWGPITYAEPRSLDQRPYCWHYAPQPCGIVPAAEVCGPVY
CFTPSPVVVGTTDRSGVPTYNWGENETDVLLLNNTRPPLGNWFGCTWMNS
TGFTKTCGGPPYNIGGVGNNTLTCPTDCFRKHPEATYTKCGLGPWLTPRC
LVDYPYRLWHYP.

An HCV E2 amino acid sequence (pep409-561; SEQ ID NO:104) of the wild-type HCV E2 protein amino acid sequence (SEQ ID NO:46) used for the HCV chimeric protein development is shown below:

(SEQ ID NO: 104)
SQKIQLVNTNGSWHINRTALNCNDSLQTGFLAALFYVHRFNSSGCSDRMA
SCRPIDTFDQGWGPITYAEPRSLDQRPYCWHYAPQPCGIVPAAEVCGPVY
CFTPSPVVVGTTDRSGVPTYNWGENETDVLLLNNTRPPLGNWFGCTWMNS
TGF.

It is also envisaged that an amino acid sequence of an HCV E2 protein peptide which may be used in a protein particle spans residues 108 to 559 of a wild-type HCV E2 protein amino acid sequence as set forth in SEQ ID NO:46 is as follows:

(SEQ ID NO: 71)
GPTDPRRRSRNLGKVIDTLTCGFADLMGYIPLVGAPLGGAARALAHGVRV
LEDGVNYATGNLPGCSFSLFLLALLSCLTIPVSAYEVRNASGVYHVTNDC
SNSSIVYEADDMIMHTPGCVPCVREDNTSRCWVALTPTLAARNASVPTTT
IRRHVDLLVGAAALCSAMYVGDLCGSVFLVSQLFTFSPRRHETVQDCNCS
IYPGHVSGHRMAWDMMMNWSPSTALVVSQLLRIPQAVVDMVAGAHWGVLA
GLAYYSMVGNWAKVLIVMLLFAGVDGTGTYVTGGTAARGVSQFTGLFTSG
PSQKIQLVNTNGSWHINRTALNCNDSLQTGFLAALFYVHRFNSSGCSDRM
ASCRPIDTFDQGWGPITYAEPRSLDQRPYCWHYAPQPCGIVPAAEVCGPV
YCFTPSPVVVGTTDRSGVPTYNWGENETDVLLLNNTRPPLGNWFGCTWMN
ST.

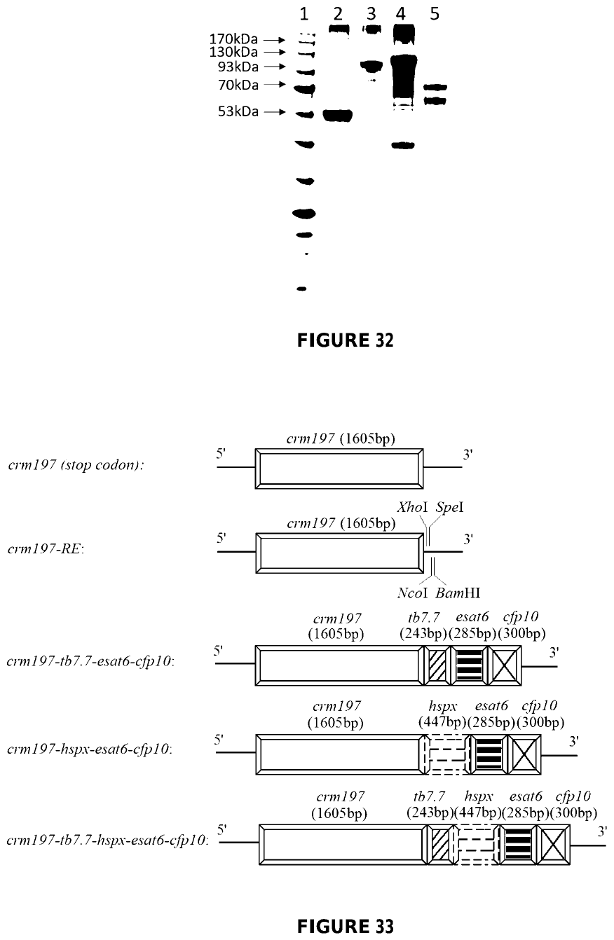

The CRM197-HCV particles as set in FIG. 31 were overproduced in ClearColi BL21(DE3) and purified particles were analysed on 10% Bis-Tris gel (FIG. 32) demonstrating that it possible to produce and isolate CRM197-HCV particles. At least one particle will be tested for immunogenicity.

### Material and Methods

E. coli Top10 and ClearColi BL21(DE3) were used for molecular cloning and CRM particle production respectively. The detail description of bacterial growth condition, plasmid transformation, CRM particle production, and CRM particle isolation and purification were described in Example 1.

### Plasmid Construction for the Formation of CRM197 Displaying HCV Antigens

The recombinant gene fragments encoding E1/E2/NS3, NS3/E2/E1/Core chimeric protein, and core antigen (HepC) encompassing the non-CRM197 regions as set out in FIG. 31 were codon-optimized for E. coli cells and excised from pUC57 vector (Biomatik, Canada) by enzyme digestion with XhoI and BamHI (BioLabs, USA) followed by DNA fragment separation using agarose gel electrophoresis with GelRed solution (Biotium, USA) and gel purification (BioLabs, USA). Meanwhile, the vector plasmid pET14b CRM197 was digested with XhoI and BamHI. The subsequent linearized pET14b CRM197 vector was ligated to the HCV DNA fragments encoding E1E2NS3, chimeric proteins, or hepC, generating the final plasmids, pET14b CRM197-E1E2NS3, pET14b CRM197-chimeric protein, and pET14b CRM197-hepC. The final plasmid DNA sequences were all confirmed by Griffith University genome sequencing centre (Griffith University, Australia).

### Results and Discussion

The immunogenic amino acid sequences of HCV as described herein were translationally fused to the C-terminus of CRM197. Schematic design of hybrid genes encoding fusion proteins for production of CRM197 particles coated with HCV antigens (CRM197-E1-E2-NS3, CRM197-Chimeric protein, and CRM197-HepC) is illustrated in FIG. 31. The CRM197-HCV particles were overproduced in ClearColi BL21(DE3) and purified particles were analysed on 10% Bis-Tris gel (FIG. 32) demonstrating that it possible to produce and isolate CRM197 HCV particles.

### Example 6

### CRM197 Particle Displaying Conformationally Folded and Glycosylated HCV Antigens Produced in Pichia pastoris Using spyCatcher/spyTag Chemistry

An immunogenic amino acid sequence derived from or corresponding to an HCV core protein, HCV E1 protein and/or a HCV E2 protein will be incorporated into a recombinantly expressed CRM197 protein particle/s as glycosylated proteins after production of the HCV proteins in Pichia pastoris or another system that is conducive to post-translation modifications.

The E1 and/or E2 glycosylated immunogenic sequences will be chemically conjugated to a CRM197 protein particle derived from a cell.

In addition, E1 and/or E2 glycosylated immunogenic amino acid sequences proteins produced will be ligated to a CRM197 protein particles using the spyCatcher/spyTag chemistry. The spyCatcher protein will be fused to CRM197 as a chimera by recombinant expression. The CRM197 protein particles will display spyCatcher. spyTagged HCV immunogens as described above will be produced using secretion and glycosylation by glycoengineered strains of Pichia pastoris. The CRM197-spyCatcher particles will be incubated with spytagged glycosylated HCV proteins for spontaneous ligation. The immunogenicity of these particles will be tested as described in Martinez-Donato et al., (2016) Clin. Vaccine Immunol. 23 (4): 370-378 [41], which is incorporated herein by reference. CRM197 particle comprising HCV antigens will be injected in mice in order to development specific antibody response. Mice sera will be collected, and an antibody neutralisation assay will be performed to evaluate the immunogenicity and efficacy of CRM197 particle-based HCV immunogenic compositions.

### Example 7

### CRM197-Dengue Protein Particles as Immunogens

An CRM197 particle as described herein will be tested as a carrier system to display immunogenic dengue antigen for the development of desirable particulate immunogenic compositions, and particularly vaccines, against dengue virus. The immunogenic sequences of dengue antigen to be displayed on or incorporated into CRM197 particles, individually or in combinations, are set out below:

An amino acid sequence of envelope protein in the form of peptide 286-426 (pep286-426; SEQ ID NO:41) of a wild-type dengue virus envelope protein amino acid sequence (SEQ ID NO:47; GenBank Accession Number AAA78919.1) is as follows:

(SEQ ID NO: 41)
RLRMDKLQLKGMSYSMCTGKFKIVKEIAETQHGTIVIRVQYEGDGSPCKI
PFEIMDLEKRHVLGRLITVNPIVTEKDSPVNIEAEPPFGDSYIIIGVEPG
QLKLNWFKKGSSIGQMFETTMRGAKRMAILGDTAWDFGSLG.

An amino acid sequence of a wild-type dengue virus envelope protein amino acid sequence is as follows:

MRCIGISNRDFVEGVSGGSWVDIVLEHGSCVTTMAKNKPTLDFELIKTEA
KQPATLRKYCIEARLTNTTTESRCPTQGEPSLKEEQDKRFVCKHSIVDRG
WGNGCGLFGKGGIVTCAMFTCKKNMEGKIVQPENLEYTIVITPHSGEEHA
SVGNDTGKHGKEIKITPQSSITEAELTGYGTITMECPRTGLDFNEMVLLQ
MEDKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKIVKEIAETQHGTIVIRVQYEGDGSPCKIPFEIMDLEKRHVLGR
LITVNPIVTEKDSPVNIEAEPPFGDSYIIIGVEPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVSLVLVGVVTLYLGAMVQA
(GenBank Accession Number AAA78919.1; SEQ ID NO:
47)

An amino acid sequence of capsid protein in the form of peptide 1-99 (pep1-99; SEQ ID NO:42) of a wild-type dengue virus capsid protein amino acid sequence (SEQ ID NO:48; GenBank Accession Number ABD15310.1) is as follows:

(SEQ ID NO: 42)
NNQRKKARSTPFNMLKRERNRVSTVQQLTKRFSLGMLQGRGPLKLFMALV
AFLRFLTIPPTAGILKRWGTIKKSKAINVLRGFRKEIGRMLNILNRRRR.

An amino acid sequence of a wild-type dengue virus capsid protein amino acid sequence is shown below:

NNQRKKARSTPFNMLKRERNRVSTVQQLTKRFSLGMLQGRGPLKLFMALV
AFLRFLTIPPTAGILKRWGTIKKSKAINVLRGFRKEIGRMLNILNRRRRT
AGVIVMLIPTAMAFHLTTRNGEPHMIVGRQEKGKSLLFKTEDGVNMCTLM
AIDLGELCEDTITYKCPLLRQNEPEDIDCWCNSTSTWVTYGTCTTTGEHR
REKRSVALVPHVGMGLETRTETWMSSEGAWKHVQRIETWILRHPGFTIMA
AILAYTIGTTHFQRALIFILLTAVAPSMTMRCIGISNRDFVEGVSGGSWV
DIVLEHGSCVTTMAKNKPTLDFELIKTEAKQPATLRKYCIEAKLTNTTTE
SRCPTQGEPSLNEEQDKRFICKHSMVDRGWGNGCGLFGKGGIVTCAMFTC
KKNMEGKVVQPENLEYTIVITPHSGEEHAVGNDTGKHGKEIKITPQSSIT
EAELTGYGTVTMECSPRTGLDFNEMVLLQMEDKAWLVHRQWFLDLPLPWL
PGADTQGSNWIQKETLVTFKNPHAKKQDVVVLGSQEGAMHTALTGATEIQ
MSSGNLLFTGHLKCRLRMDKLQLKGMSYSMCTGKFKIVKEIAETQHGTIV
IRVQYEGDGSPCKIPFEITDLEKRHVLGRLITVNPIITEKDSPVNIEAEP
PFGDSYIIIGVEPGQLKLNWFKKGSSIGQMFETTMRGAKRMAILGDTAWD
FGSLGGVFTSIGKALHQVFGAIYGAAFSGVSWTMKILIGVIITWIGMNSR
STSLSVSLVLVGVVTLYLGAMVQA
(GenBank Accession Number ABD15310.1;
SEQ ID NO: 48).

One or more dengue antigen sequence will be incorporated into CRM197 particles using one or more methods as described herein. The ability of the resulting particles to elicit an immune response will be tested.

### Example 8

### Particulate CRM197-TB as Diagnostic Reagents

CRM197 particle has been tested as a carrier system to display immunogenic TB diagnostic antigens (TB7.7, SEQ ID NO:38; HspX, SEQ ID NO:32; ESAT6, SEQ ID NO:33; CFP10, SEQ ID NO:34; see Table 4) for the development of specific and sensitive particulate CRM197 particle-based TB diagnostic reagents. The immunogenic sequences of the diagnostic antigens are displayed on or incorporated into CRM197 particles.

### Material and Methods

E. coli Top10 and ClearColi BL21(DE3) were used for molecular cloning and CRM particle production respectively. The detail description of bacterial growth condition, plasmid transformation, CRM particle production, and CRM particle isolation and purification were described in Example 1.

### Plasmid Construction for the Formation of CRM197 Displaying TB Diagnostic Antigens

All the DNA fragments encoding the TB diagnostic antigens were cloned to the 3′ end of CRM197. Hybrid genes encoding fusion proteins for production of CRM197 particles displaying TB diagnostic antigens (FIG. 33) are shown below:
TB7.7-ESAT6-CFP10 (see Table 4)
HspX-ESAT6-CFP10 (see Table 4)
TB7.7-HspX-ESAT6-CFP10 (see Table 4)

Particularly, the recombinant gene fragments encoding TB7.7-ESAT6-CFP10, HspX-ESAT6-CFP10, and TB7.7-HspX-ESAT6-CFP10 were codon-optimized for E. coli cells and excised from pUC57 vector (Biomatik, Canada) by enzyme digestion with XhoI and BamHI (BioLabs, USA) followed by DNA fragment separation using agarose gel electrophoresis with GelRed solution (Biotium, USA) and gel purification (BioLabs, USA). Meanwhile, the vector plasmid pET14b CRM197 was digested with XhoI and BamHI. The subsequent linearized pET14b CRM197 vector was ligated to the DNA fragments encoding TB diagnostic antigens, TB7.7-ESAT6-CFP10, HspX-ESAT6-CFP10, or TB7.7-HspX-ESAT6-CFP10, generating the final plasmids, pET14b CRM197-TB7.7-ESAT6-CFP10, pET14b CRM197-HspX-ESAT6-CFP10, and pET14b CRM197-TB7.7-HspX-ESAT6-CFP10. The final plasmid DNA sequences were all confirmed by Griffith University genome sequencing centre (Griffith University, Australia).

### TB Blood Assay

Blood was mix gently by inversion 10 times, and then 1 ml of the properly mixed fresh blood (<6 h post collection) was added to sterile 48 well plate. Subsequently, 50 μl of TB diagnostic reagent containing different amount TB antigens, 2 ng, 10 ng, and 50 ng, were added to the plates. The PBS buffer, dH2O, PPDA, and PPDB are the controls. The plates were incubated at 37° C. for 20 h in a humidified environment. The plasma was collected by centrifugation after incubation. ELISA was carried out to measure the levels of secreted IFNγ [56].

### Results and Discussion

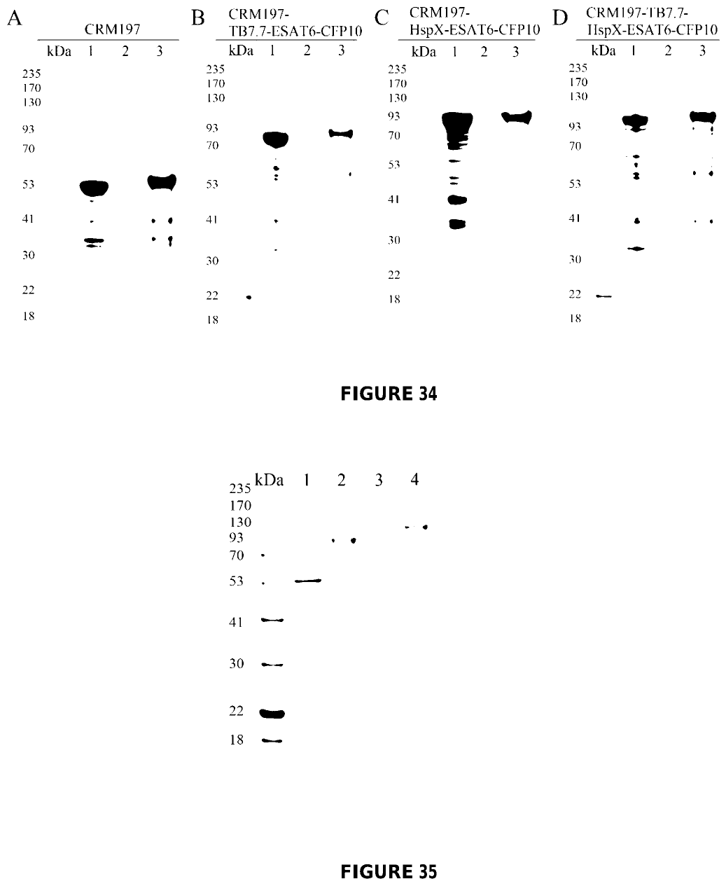

The above DNA constructs, pET14b CRM197-TB7.7-ESAT6-CFP10, pET14b CRM197-HspX-ESAT6-CFP10, and pET14b CRM197-TB7.7-HspX-ESAT6-CFP10, were overexpressed in ClearColi BL21(DE3) cells. The whole cells producing TB diagnostic reagents treated with and/or without 8 M urea were analysed on 10% Bis-Tris gel (FIG. 34). The protein profile showed that CRM197-TB diagnostic antigens are the dominant bands. After centrifugation, these dominant bands were not found in the clear cell lysate without 8 M urea treatment suggesting insoluble CRM particles were formed. However, heavy protein bands were observed in the supernatant fraction of crude cell lysate after 8 M urea treatment suggesting 8 M urea can solublise the CRM particles ie dissemble them into its constituents. These results suggest that CRM197-TB diagnostic antigens can be overproduced as protein particles. In addition, the purified CRM197-TB diagnostic reagents were also analysed on Bis-Tris gel and demonstrated high purity (FIG. 35).

### Example 9

CRM197-SARS-CoV-2 particles produced in ClearColi BL21(DE3) Severe acute respiratory syndrome coronavirus 2 (SARS-CoV-2) causes severe respiratory disease in humans and may result in death of the patient. This strain of coronavirus causes coronavirus disease 2019 (COVID-19), leading to COVID19 pandemic. Despite measures to control the outbreak, the WHO has declared SARS-CoV-2 a global health emergency. Vaccination may be able to prevent further spread and prevent COVID19 from becoming a severe pandemic crisis.

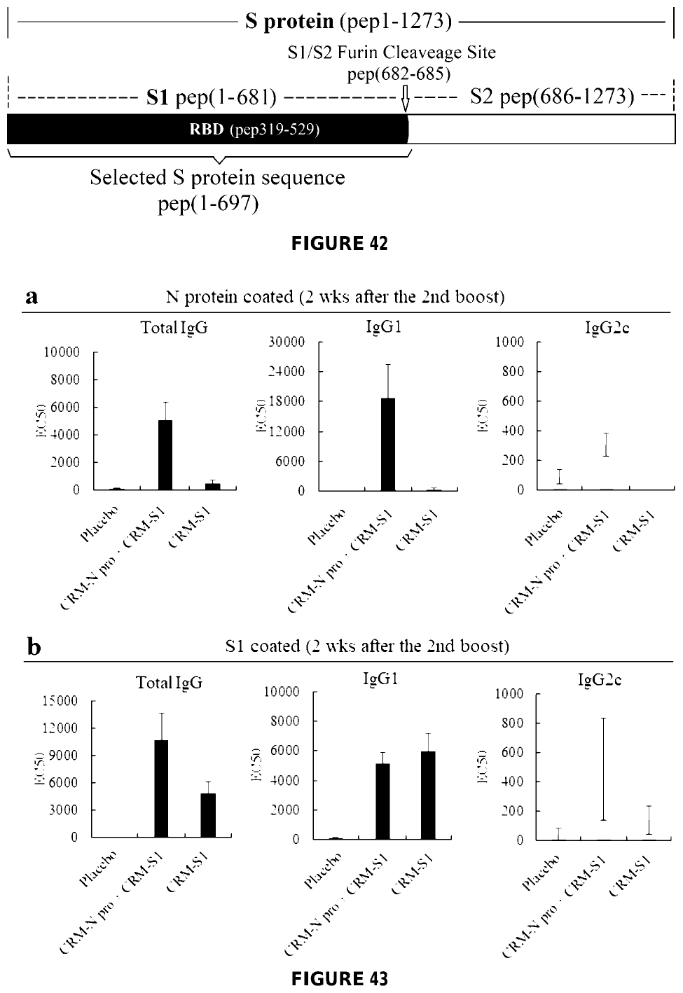

SARS-CoV-2 is a member of the family Coronaviridae. The coronavirus spike glycoproteins (S protein) form a trimeric structure on the viral envelope and facilitate binding and viral entry (see FIG. 42). The S protein includes the S1 domain, which contains a receptor binding domain (RBD, sequence set out below) and binds to the receptor on the cell surface. S1 protein amino acid sequence is set out below. The second antigen that was tested is the N protein (sequence set out below). Although this protein does may not lead to induction of strong antibody responses, the SARS-CoV N protein contains several conserved T cell epitopes.

### Materials and Methods

CRM197 particle has been tested as a carrier system to display SARS-CoV-2 antigens from the N protein and RBD of the S protein of SARS-CoV-2.

### Plasmid Construction for the Formation of CRM197 Displaying SARS-CoV-2 Antigens

E. coli Top10 and ClearColi BL21(DE3) were used for molecular cloning and CRM particle production respectively. The detail description of bacterial growth condition, plasmid transformation, CRM particle production, and CRM particle isolation and purification were described in Example 1.

An N protein amino acid sequence incorporated as a C-terminal fusion of CRM197 tested in this study as described below is as follows:

SDNGPQNQRNAPRITFGGPSDSTGSNQNGERSGARSKQRRPQGLPNNTAS
WFTALTQHGKEDLKFPRGQGVPINTNSSPDDQIGYYRRATRRIRGGDGKM
KDLSPRWYFYYLGTGPEAGLPYGANKDGIIWVATEGALNTPKDHIGTRNP
ANNAAIVLQLPQGTTLPKGFYAEGSRGGSQASSRSSSRSRNSSRNSTPGS
SRGTSPARMAGNGGDAALALLLLDRLNQLESKMSGKGQQQQGQTVTKKSA
AEASKKPRQKRTATKAYNVTQAFGRRGPEQTQGNFGDQELIRQGTDYKHW
PQIAQFAPSASAFFGMSRIGMEVTPSGTWLTYTGAIKLDDKDPNFKDQVI
LLNKHIDAYKTFPPTEPKKDKKKKADETQALPQRQKKQQTVTLLPAADLD
DFSKQLQQSMSSADSTQA (SEQ ID NO: 56; derived from
NCBI Reference Sequence YP_009724397.2).

An RBD amino acid sequence (SEQ ID NO:57) of a wild-type (full-length) SARS-CoV-2 S protein amino acid sequence (SEQ ID NO:64; GenBank Accession Number QHD43416.1) incorporated a C-terminal fusion of CRM197 in this study as described below is as follows: RVQPTESIVRFPNITNLCPFGEVFNATRFASVYAWNRKRISNCVADYSVLYNSASFSTFKCYG VSPTKLNDLCFTNVYADSFVIRGDEVRQIAPGQTGKIADYNYKLPDDFTGCVIAWNSNNLDS KVGGNYNYLYRLFRKSNLKPFERDISTEIYQAGSTPCNGVEGFNCYFPLQSYGFQPTNGVGYQ PYRVVVLSFELLHAPATVCGPKK (SEQ ID NO:57). This RBD sequence (pep319-529) is derived from the full-length S protein (see below; also referred to as SEQ ID NO:64) and spans amino acid residues 319 to 529 of the S protein. In particular, the schematic diagram of S protein encompassing RBD domain is illustrated in FIG. 42.

A full-length SARS-CoV-2 S protein has the following amino acid sequence:

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT
(GenBank Accession Number QHD43416.1;
SEQ ID NO: 64).

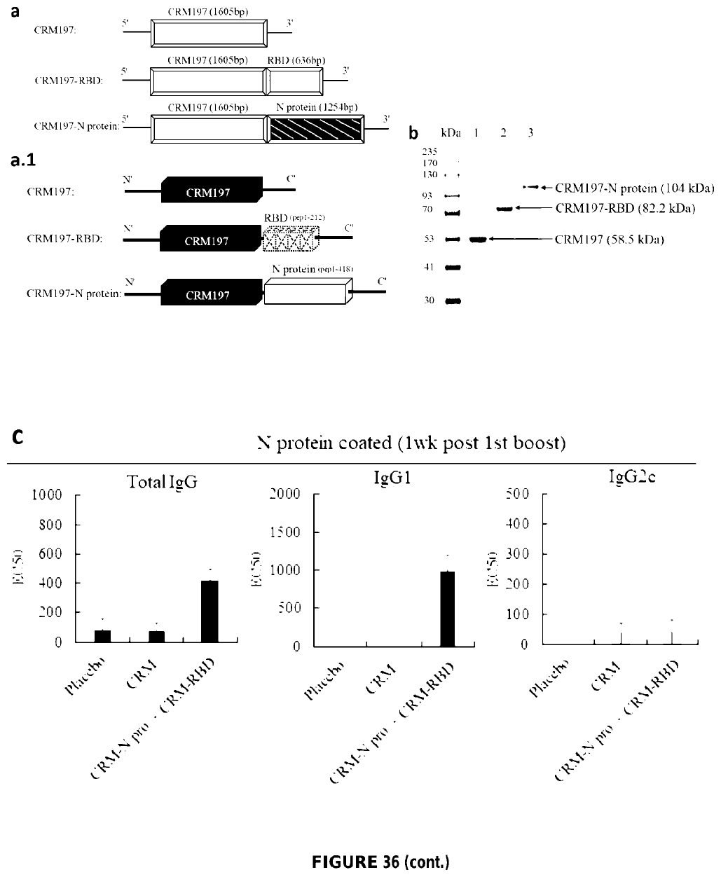

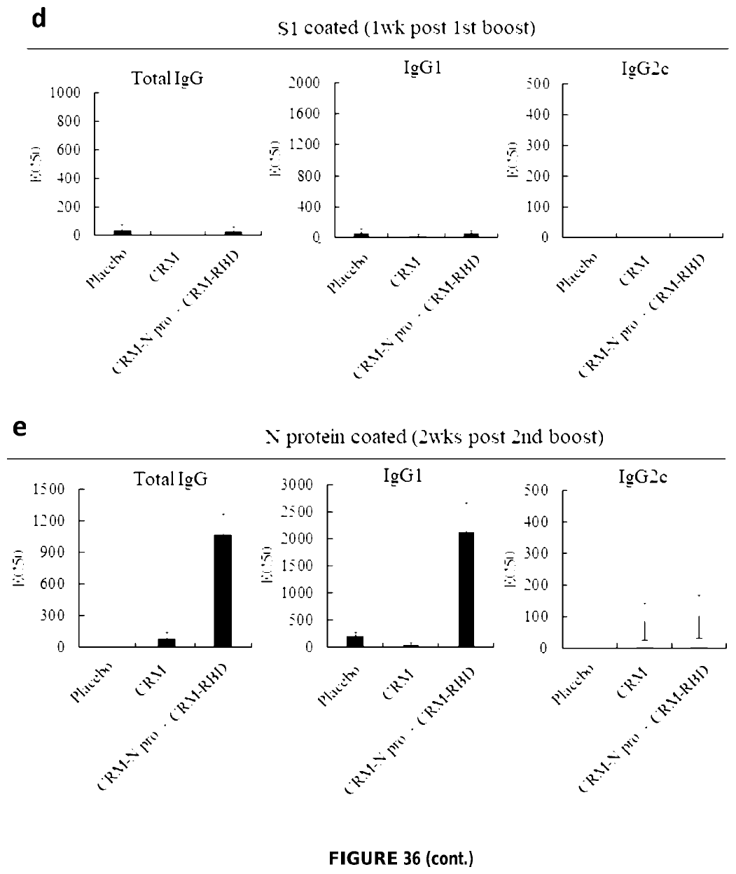

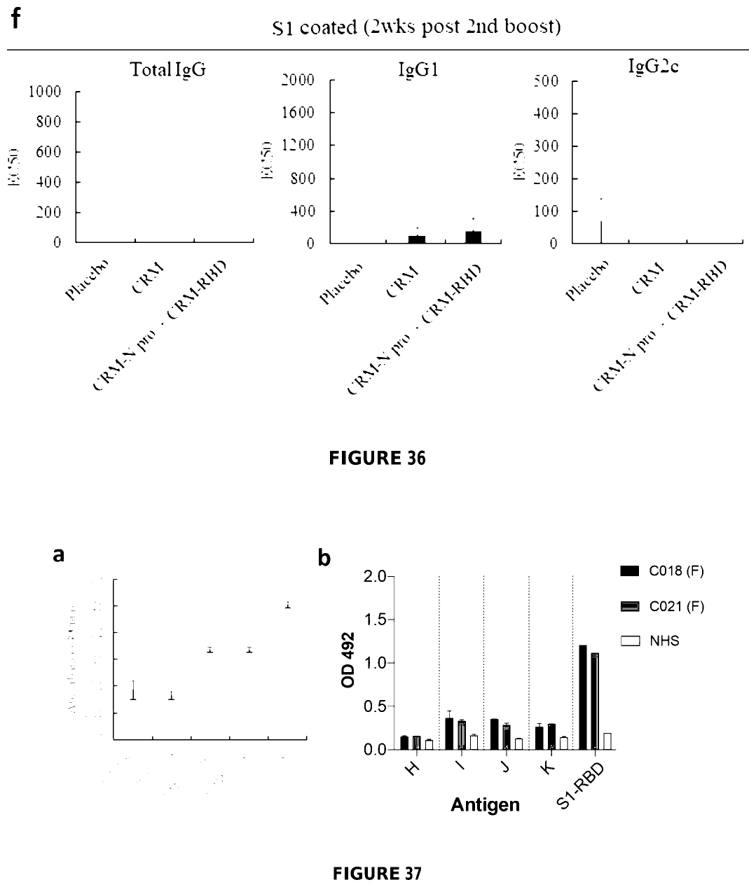

The N protein and RBD region of the spike protein having the above sequences were recombinantly fused to the C-terminus of CRM197, respectively. Hybrid genes encoding fusion proteins for production of CRM197-RBD particles and particulate CRM197-N protein particles is illustrated in FIG. 36a. Briefly, the recombinant gene fragments encoding RBD and N protein as set out above were codon-optimized for E. coli cells and excised from pUC57 vector (Biomatik, Canada) by enzyme digestion with XhoI and BamHI (BioLabs, USA) followed by DNA fragment separation using agarose gel electrophoresis with GelRed solution (Biotium, USA) and gel purification (BioLabs, USA). Meanwhile, the vector plasmid pET14b CRM197 was digested with XhoI and BamHI. The subsequent linearized pET14b CRM197 vector was ligated to the DNA fragments encoding RBD and N protein, generating the final plasmids, pET14b CRM197-RBD and pET14b CRM197-N protein. The final plasmid DNA sequences were all confirmed by Griffith University genome sequencing centre (Griffith University, Australia).

These plasmids encoding CRM197-SARS-CoV-2 antigens were transformed into an endotoxin free host, ClearColi BL21(DE3), for particle production as described above. The protein profile of purified CRM197-SARS-CoV-2 particles is shown in FIG. 36b. A dominant protein band with a high purity corresponding to proteins with the theoretical molecular weight of CRM197 (58.5 kDa), CRM197-RBD (82.2 kDa), and CRM197-N protein (104 kDa), suggesting CRM197, CRM197-RBD, and CRM197-N proteins are heavily produced. The CRM197-SARS-CoV2 particles were formulated with Alum adjuvant in the following formulations:
Placebo: Alum alone
CRM197 particles with alum (This formulation contains CRM197 particle alone without antigens)
CRM197-N protein particles+CRM197-RBD particles+alum (This formulation contains two separate CRM197 particles carrying each antigen)

### Mice Immunization

Animal experiments were approved by Griffith University Animal Ethics Committee (Australia). The animal trial was performed using 6-week old female C57BL/6 mice. There were 10 mice per group. The CRM197-COVID19 particles were formulated with Alhydrogel 2% (Alum) (InvivoGen, USA) in the following formulations:
Placebo: Alum alone
CRM197 particles with alum (as described above)
CRM197-N protein particles+CRM197-RBD particles+alum (as described above)

Formulated test samples containing 20 μg/dose of SARS-CoV-2 antigens and 25 μl/dose of alum in 100 μl. All formulated test samples were injected into mice intramuscularly, 50 μL in each thigh, a total of 100 μL per mice. Mice were immunized three times (day 0, 14, and 28). Prebleed, mid and final serum samples were collected (day 0, 21, 42). The mice immunization was carried out at the GRIDD (Griffith University, Queensland, Australia).

### Antibody Response Analysis Using ELISA

ELISA was used to analyse the antibody response of mice induced by CRM197 particles carrying SARS-CoV-2 antigens. The experiment procedure was described above in the Example 1. The ELISA was done at the GRIDD (Griffith University, Queensland, Australia).

### SARS-CoV-2 Plaque Reduction Assay

Sera were heat inactivated for 30 min at 56° C. (day prior to assay) and stored at −20° C. until day of processing and 96-well plates containing Vero cells is cultured to ensure ˜95 monolayer confluence.

Serial dilutions of the sera (1:20-1:10,240 in MEM) was prepared in 96 well plates. Indeed, 96 well plates of ˜95% confluent Vero cell monolayers was verified, and growth media was removed. Plates were washed with Infection Medium (MEM+Antibiotics, no FBS) and then 150 μl of Infection Medium containing 1 μg/ml TPCK Trypsin was added on plates. All the above experimental procedures were performed in PC2 lab.

The following lab work was performed in PC3 lab. Firstly, 100 TCID50 per 50 μl=103.3 TCID50/ml of SARS-CoV-2 was prepared in Infection Medium containing 0.5% BSA. The virus was then added 1:1 to each dilution of the pre-prepared sera dilutions, and incubated at room temperature for 1 h, with occasional rocking. The SARS-CoV-2/Sera samples was added to Vero cells in quadruplicate. Each plate includes a row of virus only and cell only (i.e. no sera or virus) as controls. The amount of virus present in the original inoculum was evaluated and verified by performing a back titration. Plates were then incubated at 37° C. with 5% CO₂ then microscopically monitored daily for cytopathic effect (CPE), for up to 4 days post-procedure. The serum dilution where still a significant reduction in plaques can be observed was reported as neutralizing antibody titer. This assay was conducted at the Peter Doherty Institute for Infection and Immunity at the University of Melbourne (Australia).

### Results and Discussion

Hybrid genes encoding fusion proteins for production of particulate CRM197-RBD and particulate CRM197-N protein is illustrated in FIG. 36a. These plasmids encoding CRM197-SARS-CoV-2 antigens are transformed into an endotoxin free host, ClearColi BL21(DE3), for particle production. The protein profile of purified CRM197-SARS-CoV-2 particles is shown in FIG. 36b. A dominant protein band with a high purity corresponding to proteins with the theoretical molecular weight of CRM197 (58.5 kDa), CRM197-RBD (82.2 kDa), and CRM197-N protein (104 kDa), suggesting CRM197, CRM197-RBD, and CRM197-N proteins are heavily produced.

The immunogenicity of formulated particle samples were tested in female C57BL/6. FIGS. 36c and 36e showed that adjuvanted serum samples from mice immunized with adjuvanted CRM197-N protein and CRM197-RBD have high total IgG and IgG1 titre on N protein coated plates, suggesting CRM197-N protein and CRM197-RBD induced strong total IgG and IgG1 response. An antibody response was observed on S1 coated plates (FIGS. 36d and 36f), suggesting the CRM197 particles containing RBD produced in ClearColi BL21(DE3) can elicit an immune response. In the SARS-CoV-2 plaque reduction assay induction of a neutralizing antibody titres was obtained for CRM197-N protein/CRM197-RBD formulation, while alum, CRM only and pre-vaccination sera showed no detectable neutralizing antibodies (FIG. 41).

### Example 10

### Particulate CRM197-SARS-Co-V-2 Particles Produced in ClearColi BL21(DE3) Harbouring pMCS69E

We used a different production strain background and redesigned the CRM197-SARS-CoV-2 containing particles for induction of an enhanced functional immune responses. This approach aimed at including extended and conformational antigens into CRM particles. Using the S1 subunit instead of only RBD offer an enhanced repertoire of epitopes for induction of neutralising antibodies and T cell responses. Retention of antigen structure enables induction of antibodies that recognise conformational epitopes considered to be important for virus neutralisation. This experiment is an extended study of Example 9. However, we have changed the production host from ClearColi BL21(DE3) to ClearColi BL21(DE3) harbouring pMCS69E, changed the SARS-CoV-2 antigen from RBD to S1 protein, and increased antigen dose from 20 μg/dose to 50-100 μg/dose.

The S1 subunit of the spike protein S comprises the RBD and additional multiple B and T-cell epitopes recognised by antibodies found in convalescent patients. Epitopes are also recognised by neutralising antibodies blocking virus entry into cells, hence eliciting such antibodies by considering S1 as vaccine antigen candidate could enhance the performance of a vaccine formulation. The SARS-CoV-2 S protein contains a unique S1/S2 furin cleavage site (RRAR, pep682-685). The furin cleavage site is absent in SARS-CoV and other SARS-related coronaviruses (SARSr-CoVs), which shows 96% identity of its genomic sequence to that of SARS-CoV-2. It has been speculated that the furin cleavage site makes SARS-CoV-2 easily enter into the host cells for infection, thus responsible for the high infectivity and transmissibility. As illustrated in FIG. 42, our selected S protein amino acid sequence (pep1-697; SEQ ID NO:101) contains full-length of S1 subunit (pep1-681; SEQ ID NO:58), furin cleavage site (pep682-685; SEQ ID NO: 102), and a short N terminus sequence of S2 (pep686-697; SEQ ID NO:103). The N terminus sequence of S2 (SEQ ID NO:103) is to ensure efficient S1/S2 furin cleavage.

The selected S protein amino acid sequence (pep1-697; SEQ ID NO:101) of a wild-type SARS-CoV-2 S protein amino acid sequence (SEQ ID NO:64) was used in this study as follows:

(SEQ ID NO: 101)
FVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSN
IIRGWIFGTTLDSKTQSLLIVNNATNVVIKVCEFQFCNDPFLGVYYHKN
NKSWMESEFRVYSSANNCTFEYVSQPFLMDLEGKQGNFKNLREFVFKNI
DGYFKIYSKHTPINLVRDLPQGFSALEPLVDLPIGINITRFQTLLALHR
SYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNA
TRFASVYAWNRKRISNCVADYSVLYNSASFSTFKCYGVSPTKLNDLCFT
NVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFP
LQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYST
GSNVFQTRAGCLIGAEHVNNSYECDIPIGAGICASYQTQTNSPRRARSV
ASQSIIAYTM.

A S1 subunit amino acid sequence (pep1-681; SEQ ID NO:58) of a wild-type SARS-CoV-2 S protein amino acid sequence (SEQ ID NO:64; GenBank Accession Number QHD43416.1) was used in this study as follows:

(SEQ ID NO: 58)
FVFLVLLPLVSSQCVNLTTRTQLPPAYTNSFTRGVYYPDKVFRSSVLHST
QDLFLPFFSNVTWFHAIHVSGTNGTKRFDNPVLPFNDGVYFASTEKSNII
RGWIFGTTLDSKTQSLLIVNNATNVVIKVCEFQFCNDPFLGVYYHKNNKS
WMESEFRVYSSANNCTFEYVSQPFLMDLEGKQGNFKNLREFVFKNIDGYF
KIYSKHTPINLVRDLPQGFSALEPLVDLPIGINITRFQTLLALHRSYLTP
GDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNY
LYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLI
GAEHVNNSYECDIPIGAGICASYQTQTNSP.

The furin cleavage site protein amino acid sequence (pep682-685; SEQ ID NO: 102) of a wild-type SARS-CoV-2 S protein amino acid sequence (SEQ ID NO:64) was used in this study as follows: RRAR (SEQ ID NO: 102).

The short N terminus protein amino acid sequence of S2 (pep686-697; SEQ ID NO:103) of a wild-type SARS-CoV-2 S protein amino acid sequence (SEQ ID NO:64) was used in this study as follows: SVASQSIIAYTM (SEQ ID NO:103).

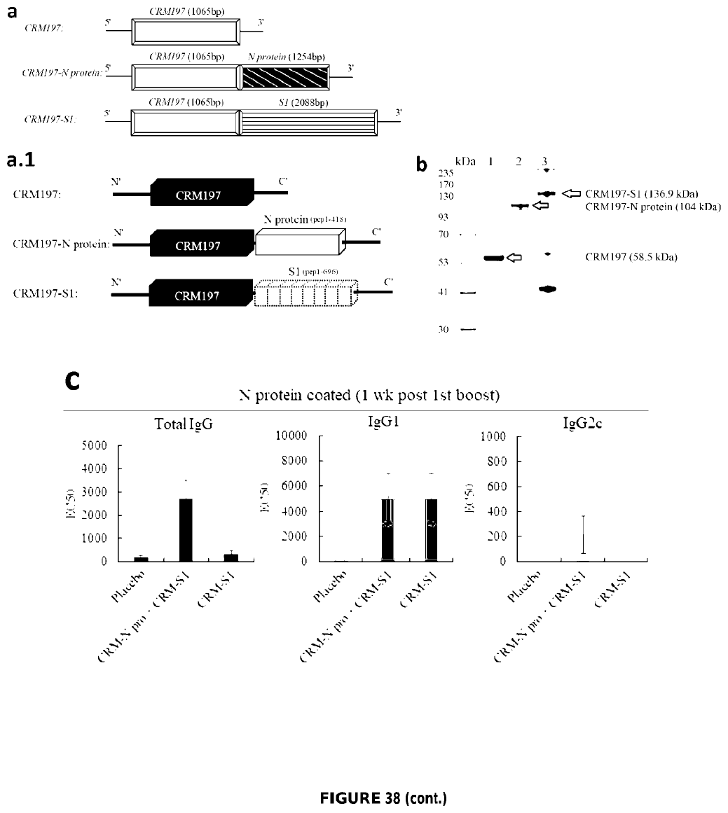

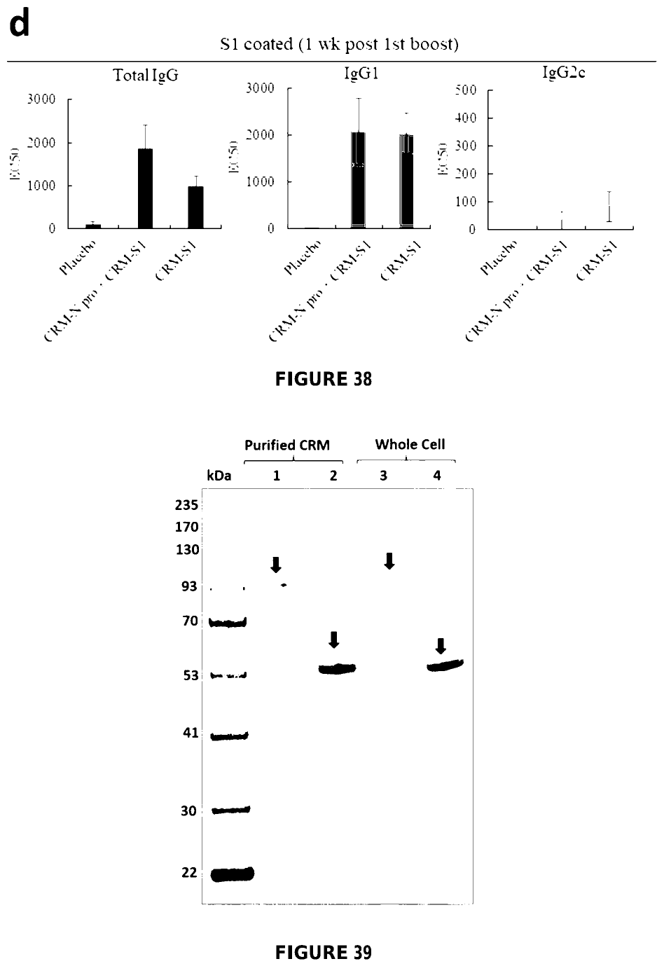

The selected S protein amino acid sequence (SEQ ID NO:101) was translationally fused to the C-terminus of CRM197 (FIG. 38a). Briefly, the recombinant gene fragment encoding S1 was codon-optimized for E. coli cells and excised from pUC57 vector (Biomatik, Canada) by enzyme digestion with XhoI and BamHI (BioLabs, USA) followed by DNA fragment separation using agarose gel electrophoresis with GelRed solution (Biotium, USA) and gel purification (BioLabs, USA). The vector plasmid pET14b CRM197 was digested with XhoI and BamHI. The subsequent linearized pET14b CRM197 vector was ligated to the DNA fragments encoding S1 protein, generating the final plasmids, pET14b CRM197-S1. The final plasmid DNA sequence was confirmed by Griffith University genome sequencing centre (Griffith University, Australia).

### CRM197-SARS-CoV-2 Antigen Particles Beads and ACE2 Binding Assay

High-binding plates (Greiner Bio-One, Germany) were coated overnight at 4° C. with 100 μL of 5 μg mL-1 purified CRM197-SARS-CoV-2 antigen particles diluted in phosphate-buffered saline containing 0.05% (v/v) Tween 20, pH7.5 (PBST). Positive and negative controls were also coated on plates overnight. Particularly, CRM197 particles and CRM197-N protein particles are negative controls. Glycosylated soluble S1 (University of Queensland, Australia) is used as a positive control. Plates were incubated with Angiotensin-Converting Enzyme (ACE2) (Human) Fc fusion (HEK293) (Aviscera Bioscience Inc, USA) diluted with PBST at the concentration of 1/1000 for 1 h at 25° C. After three times wash with PBST, plates were incubated with protein A-HRP for 1 h at 25° C. Plates were washed with PBST three times. o-phenylenediamine substrate (Abbott Diagnostics, IL, USA) was added on plate for signal development. The result was measured at 490 nm with on an ELx808iu ultramicrotiter plate reader (Bio-Tek Instruments Inc., USA). The ACE2 binding assay is performed at the GRIDD (Griffith University, Queensland, Australia).

### CRM197-SARS-CoV-2 Antigen Particles Beads ELISA Using Infected Human Serum Samples

This experiment was done as a single blind study. CRM197 particles, CRM197-RBD particles, CRM197-N protein particles, and CRM197-S1 particles are designated to samples H, I, J, and K, respectively. (CRM197 particles may refer to CRM197 only particles or CRM particles, and vice versa. RBD particles may refer to CRM197-RBD particles or CRM-RBD particles, and vice versa. N protein particles may refer to CRM197-N protein particles or CRM-N pro particles, and vice versa. S1 particles may refer to CRM197-S1 particles or CRM-S1 particles, and vice versa.) S1-RBD was used as a positive control. Briefly, high-binding plates were coated with 100 μL of 1 μg mL-1 of antigens in carbonate coating buffer pH9.6 at 4° C. overnight. Plates were blocked with 5% skim milk in PBST for 90 mins at 37° C. before adding the primary antibody (infected and noninfected human plasma samples) at the concentration of 1/2,000 for 90 mins at 37° C. After washings, plates were then incubated with the secondary IgG at the concentration of 1/3,000 and OPD was used as the substrate for signal development. The results were measured at 492 nm. The ELISA was done at the Institute of Glycomics (Griffith University, Queensland, Australia).

### Mice Immunization

The detail description of the animal experiments was described in the Example 9. The redesigned CRM197-SARS-CoV-2 antigen particles were formulated with Alhydrogel 2% (Alum) (InvivoGen, USA) in the following formulations:
Placebo: Alum alone
CRM197-N protein particles+CRM197-S1 particles+alum (This formulation contains separate CRM197 particles carrying each antigen)
CRM197-S1 particles+alum

Formulated test preparation containing 50-100 μg/dose of SARS-CoV-2 antigens and 25 μl/dose of alum in 100 μl. All samples were injected into mice intramuscularly, 50 μL in each thigh, a total of 100 μL per mice. Mice were immunized three times (day 0, 14, and 28). Prebleed, mid and final serum samples were collected (day 0, 21, 42). The mice immunization was conducted at the GRIDD (Griffith University, Queensland, Australia).

### Antibody Response

Antibody response of mice immunized with various CRM197-SARS-CoV-2 antigen particles was analyzed using ELISA. The experimental procedure was shown in the Example 1. The ELISA was done at the GRIDD (Griffith University, Queensland, Australia).

### Results and Discussion

An endotoxin free ClearColi BL21(DE3) does not have a proper environment for disulfide bond formation. Here we used ClearColi BL21(DE3) harbouring pMCS69E as the production host for CRM197-COVID19 vaccine production. Plasmid pMCS69E contains the yeast sulfhydryl oxidase Erv1p, which is able to improve the production of disulphide bonded proteins in the cytoplasm. The particulate CRM197-N protein and CRM197-S1 produced in ClearColi BL21(DE3) harbouring pMCS69E were isolated and the protein profile of purified particles were analysed on 10% Bis-Tris gel (FIG. 38b).

S1 contains the receptor binding domain (RBD), which can directly bind to the host receptor, peptidase domain (PD) of angiotensin-converting enzyme 2 (ACE2). Thus ACE2-S1 binding has been performed to analyse the functionality of S1 as part of the particulate CRM197-S1 prior to the animal trial. The binding results (FIG. 37) demonstrated that the particles containing the RBD domain show a higher ACE2 binding when compared to the negative controls, CRM197 particle and CRM197-N protein. This indicated that particulate CRM197 particles containing RBD domains produced in ClearColi BL21(DE3) harbouring pMCS69E is likely properly folded. S1 protein comprises the RBD domain.

In addition, we also evaluated whether the CRM197-SARS-CoV-2 antigen particle produced in ClearColi BL21(DE3) harbouring pMCS69E were able to perform as diagnostic reagent to accurately distinguish between infected and noninfected human serum samples. This experiment was done as a single blind study. H, I, J, and K refers to CRM197 particles, CRM197-RBD particles, CRM197-N protein particles, and CRM197-S1 particles, respectively. The result showed that the positive control, S1-RBD, can accurately discriminate infected from noninfected human serum samples, but the negative control, CRM197 particle, is not able to. This suggest all the controls worked properly. The CRM197-SARS-CoV-2 particles, containing selected SARS-CoV-2 antigens, were able to successfully identify the infected human serum samples. Overall, this ELISA result indicated that CRM197-SARS-CoV-2 antigen particles produced in ClearColi BL21(DE3) harbouring pMCS69E may be able to perform as COVID19 diagnostic reagents.

The immunogenicity of formulated particle preparation was tested in female C57BL/6. There were 10 mice per group. Formulated preparations containing 50-100 μg of SARS-CoV-2 antigens were injected into mice intramuscularly, 50 μL in each thigh, a total of 100 μL per mice. Such a high dose was chosen to ensure that a lack of immune response is not due to too low dose. This animal experiment is still ongoing. The mid serum samples of mice immunised with various CRM197-SARS-CoV-2 particles were analysed (FIGS. 38c and 38d). The results showed that serum samples from mice immunized with particulate CRM197-S1 alone and CRM197-N protein with CRM197-S1 showed high titers of total IgG and IgG1 against N protein or S1 in ELISA when compared to the placebo control or pre-vaccination sera.

The final serum samples of mice immunised with various CRM197-SARS-CoV-2 particles were analysed (FIGS. 43a and 43b). Generally, the total IgG and IgG1 levels in the final sera (after the third immunisation) were higher than the ones in the mid serum samples (after the second immunisation), while the IgG2c level remained similar between the final and mid serum samples (FIG. 43, FIG. 38c, and FIG. 38d). Regarding induction of anti-N protein antibodies, the total IgG and IgG1 level in the final serum samples from mice immunized with both CRM197-N protein and CRM197-S1 particles was approximately 3-fold higher than the total IgG and IgG1 in the med serum samples obtained from the mice immunized with the same vaccine formulation (FIG. 43a and FIG. 38c). Interestingly, the titre of IgG1 against N protein coated plates was decreased in the final serum samples from mice immunized with CRM197-S1 particles, when compared to the IgG1 level in mid serum samples from mice immunized with CRM197-S1 particles (FIG. 43a and FIG. 38c). This may be due to anti-S1 antibodies generated in mice immunized with CRM197-S1 particles after the first boost which were cross-reacting with N protein epitopes. The level of non-specific anti-S1 antibodies was decreased after the second boost due to the affinity maturation/seroconversion of specific anti-S1 antibodies. FIG. 43b and FIG. 38d show the total IgG and IgG1 levels in serum samples from mice immunized with the mixed CRM197-S1 and CRM197-N protein particles or the CRM197-S1 particle alone were about 3-5-fold higher than the total IgG and IgG1 level obtained from the mid serum samples.

### Example 11

### CRM197-Q Fever Particles

Coxiella burnetti (C. burnetti) is the etiological agent of the infectious zoonotic disease Q (“query”) fever. It is a gram-negative intracellular bacterium that manifests as an incapacitating influenza-like illness with two phases. Acute Q fever often presents as a self-limiting febrile illness or pneumonia whereas chronic Q fever can be complicated by endocarditis and chronic hepatitis which are sometimes incurable. Transmission is usually through contaminated aerosols generated by infected livestock. Due to its high stability and resistance to desiccation and environmental factors it remains infectious in the environment for a long period of time and is considered as a category B bioterrorism agent.

Prevention is with the current available vaccine “Q vax” a formalin inactivated whole cell vaccine that is licensed for use only in Australia. Though proven to be effective and immunogenic, it is associated with limitations and drawbacks. The vaccine cannot be administered to previously sensitized individuals as it induces severe local and systemic reactions at the site of injection, due to the LPS component in the whole cell preparation. As a result, individuals must be screened for Coxiella specific antibodies prior to administration. This emphasizes the need for a less reactogenic but equally efficacious vaccine that can be administered to individuals without the need for pre-vaccination screening.

Due to the intracellular nature of C. burnetti, T cell mediated immunity is predominantly required in eliminating the pathogen along with B cell humoral response amplified by the cognate T cells. The integral role of T cells in control of C. burnetti infection has been demonstrated in murine models, along with the role of APCs such as dendritic cells, that process and present the cognate antigens to initiate cell mediated responses. Hence an approach with immunodominant epitopes targeting T cell mediated response would serve as a potential immunogenic composition, and in particular a candidate vaccine, with the components of the pathogen used as triggers, instead of whole cell vaccine which contains the LPS phase variant that gives rise to adverse effects currently observed with Q vax.

Immunodominant antigens of C. burnetti were identified and the specific CD4+ and CD8+ epitopes of these antigens mapped through bioinformatic analysis (Tools for prediction of peptide binding NetMHCcons and NetMHCIIpan; (Andreatta and Nielsen, Bioinformatics Tools for the Prediction of T-Cell Epitopes, Epitope Mapping Protocols, 2018, Volume 1785, ISBN: 978-1-4939-7839-7)), were selected in designing a potential candidate. The recombinant peptide, named COX, having an amino acid sequence of MASFQNYLNDYGPGPGVTLVEFFDYGPGPGHYLVNHPEVLVEASQGPGPGDVNYGYNAATG EYGDGPGPGFDSSYKRGQPATFPLGPGPGGKLGVAYTYNRANAGGPGPGVAMIWSVAAVAQ TVGGPGPGPVSASITQFGPVGELGPGPGLLTKKQYDKAQASFQGPGPGTPTFVIGNKALTKFG FGPGPGKIGVIKAIRTITGLGLKEAGPGPGMMEHLQNITNLVSTGRQGAGPGPGKIPVKIIKPPF VRRGGPGPGRLGFMSFFTKAVVEALKRFGPGPGVAKLRGDLSSIIHKLGPGPGLSSIIHKLTSFS KTEAGPGPGSPAVLSAAKKIFGDGAGPGPGLRPVRYFTGVPSPVKTPEGPGPG (SEQ ID NO:59), containing the immunogenic epitopes has a collective size of 101.1 kDa and was displayed or incorporated into the CRM197 platform.

### Materials and Methods

E. coli Top10 and ClearColi BL21(DE3) were used for molecular cloning and CRM particle production respectively. The detail description of bacterial growth condition, plasmid transformation, CRM particle production, and CRM particle isolation and purification were described in the Example 1.

### Plasmid Construction for the Formation of CRM197 Displaying Q Fever Antigen COX

The recombinant gene fragment encoding COX was codon-optimized for E. coli cells and excised from pUC57 vector (Biomatik, Canada) by enzyme digestion with XhoI and BamHI (BioLabs, USA) followed by DNA fragment separation using agarose gel electrophoresis with GelRed solution (Biotium, USA) and gel purification (BioLabs, USA). The vector plasmid pET14b CRM197 was digested with XhoI and BamHI. The subsequent linearized pET14b CRM197 vector was ligated to the DNA fragments encoding COX protein, generating the final plasmids, pET14b CRM197-COX. The final plasmid DNA sequence was confirmed by Griffith University genome sequencing centre (Griffith University, Australia).

The immunogenicity of the Q fever particles as set out above will be tested in Guinea pigs in October 2019 at Griffith Institute for Drug Discovery.

### Results and Discussion

The CRM197 particles-based Q fever particles were purified and the protein profile was analysed on 10% Bis-Tris Gel shown in FIG. 39. There is a dominant protein band corresponding to protein with the theoretical molecular weight of CRM197-COX (101.1 kDa), suggesting CRM197-COX was heavily produced. This result demonstrated that CRM197 particles as the carrier platform to display Q fever antigen can be produced.

### Example 12

### Particulate CRM197-Q Fever Diagnostic Reagents

Clinical diagnosis of Q fever is challenging as the signs are not pathognomonic and can easily be confused with other diseases such as leptospirosis and dengue. Current diagnostic platforms include molecular detection for C. burnetti and serological detection of Coxiella specific antibodies with techniques such as Enzyme-linked immunosorbent assays (ELISAs) and Immuno Fluorescent assays (IFAs).

The antigens used for serological testing are often less specific as it contains antigens similar to the one in the environmental bacteria. Moreover, commercially available kits offer poor sensitivity, and consistency varies among laboratories. Molecular testing comes with the drawback of reagent contamination, false-positives and reliability with only acute stages of Q fever. Hence there is a need for a method which provide an alternative to one or more conventional Q Fever diagnostic methods.

Full length sequences of four immunodominant antigens, namely Com1, OmpH, YbgF, and GroEL having amino acid sequences set out below, were individually displayed or incorporated into CRM197 particle platform:

Com1: An amino acid sequence of wild-type Com1 used in this study is as follows:

MKNRLTALFLAGTLTAGVAIAAPSQFSFSPQQVKDIQSIVHHYLVNHPE
VLVEASQALQKKTEAQQEEHAQQAIKENAKKLFNDPASPVAGNPHGNVT
LVEFFDYQCGHCKAMNSVIQAIVKQNKNLRVVFKELPIFGGQSQYAAKV
SLAAAKQGKYYAFHDALLSVDGQLSEQITLQTAEKVGLNVAQLKKDMDN
PAIQKQLRDNFQLAQSLQLAGTPTFVIGNKALTKFGFIPGATSQQNLQK
EIDRVEK
(SEQ ID NO: 60; NCBI Reference Sequence Accession
Number WP_010958530.1);

OmpH: An amino acid sequence of the predicted immunodominant B and T cell epitopes derived from a wild-type OmpH protein amino acid sequence (SEQ ID NO:72) were rearranged and used in this study is as follows: APQIKDINTRLEKQFSGGGGGMSKVNGAVKRVAERENGGGGGLSAICLSVAMIWSVAAGGG GGLRKEIQNDESTLRQQQGGGGGTRLEKQFSPQREKMTKGGGGGRVAERENLDLVLPKDTG GGGGYAKNSKDITSNGGGGGQNKAMSDGGGGGLYAKNSKDITSNGGGGGGKKEAENLRKE IQNDEGGGGGTLRQQQQQFQQEGGGGGQELFVAQNKAMSDFM, (SEQ ID NO:61). There is a quintuple glycine linker (GGGGG) between each epitope in SEQ ID NO:61. The sequence of the individual epitopes are set out in Table 7.

An amino acid sequence of a wild-type OmpH having an amino acid sequence is as follows:

MIKRLLSAICLSVAMIWSVAAVAQTVGLVDMRQIFQTAPQIKDINTRLE
KQFSPQREKMTKLTQSLQQNLQKLKRDEAVMGKKEAENLRKEIQNDEST
LRQQQQQFQQELFVAQNKAMSDFMSKVNGAVKRVAERENLDLVLPKDTV
LYAKNSKDITSNVVSALK
(SEQ ID NO: 72;_NCBI Reference Sequence Accession
Number NP_819642.1).
YbgF: An amino acid sequence of wild-type YbgF
used in this study is as follows
MRLIKMKIKTLCVSSALAALMLSAPLTWADAPVEDISAQPQPTKTTVSP
SETPETAIPTAPVSLPTTQTDLTVTHRLARLEQQLNNIINMNLPQQISD
LQQRLAQVRGQLQVQERNLELLNNQQRSFYRDLDQRITQLKNLNSNNSD
SSNDNSASSSQKPSSGDTSNTNNIQLQDSNTYRQALDLLTKKQYDKAQA
SFQNYLNDYPNGSYVANAHYWLGEIYLQQKDRKNAAHEFQTVRDKFPKS
EKVLDAKLKLAIIDAEDGKIKQAKEELTEIKKQHPESTAAQLANIRLQQ
LEEVDSATTTP
(SEQ ID NO: 62; NCBI Reference Sequence
Accession Number WP_010957373.1)

GroEL: An amino acid sequence of the predicted immunodominant B and T cell epitopes derived from a wild-type GroEL protein amino acid sequence (SEQ ID NO:73) were rearranged and used in this study is as follows: TEVEMKEKKARVEDALGGGGGGYLSPYFIGGGGGVEEGVVPGGGVGGGGGKKISNIRGGGG GVTKDDTTIIDGSGDAGDIKNGGGGGIKNRVEQIRKEIENSSSDYDKEKLQERLGGGGGTEAP KKKEESMPGGGGGASRTSDDAGDGTTTATGGGGGSKPCKDQKAGGGGGSANSDKSIGDGG GGGEKVGKEGGGGGGNNQQNMSGGGGGDSVEVENEDQRVGGGGGGATGEYGDGGGGGS HEVLHAMSRGVEVLA (SEQ ID NO:63). There is a quintuple glycine linker (GGGGG) between each epitope in SEQ ID NO:63. The sequence of the individual epitopes are set out in Table 7.

An amino acid sequence of a wild-type GroEL having an amino acid sequence is as follows:

MAAKVLKFSHEVLHAMSRGVEVLANAVKVTLGPKGRNVVLDKSFGAPTI
TKDGVSVAKEIELEDKFENMGAQMVKEVASRTSDDAGDGTTTATVLAQA
ILVEGIKAVIAGMNPMDLKRGIDKAVTAAVAELKKISKPCKDQKAIAQV
GTISANSDKSIGDIIAEAMEKVGKEGVITVEDGSGLENALEVVEGMQFD
RGYLSPYFINNQQNMSAELENPFILLVDKKISNIRELIPLLENVAKSGR
PLLVIAEDIEGEALATLVVNNIRGVVKVAAVKAPGFGDRRKAMLQDIAV
LTGGKVISEEVGLSLEAASLDDLGSAKRVVVTKDDTTIIDGSGDAGDIK
NRVEQIRKEIENSSSDYDKEKLQERLAKLAGGVAVIKVGAATEVEMKEK
KARVEDALHATRAAVEEGVVPGGGVALIRVLKSLDSVEVENEDQRVGVE
IARRAMAYPLSQIVKNTGVQAAVVADKVLNHKDVNYGYNAATGEYGDMI
EMGILDPTKVTRTALQNAASIAGLMITTECMVTEAPKKKEESMPGGGDM
GGMGGMGGMGGMM
(SEQ ID NO: 73; UniProtKB/Swiss-Prot Accession
Number P19421.1).

### Materials Methods

E. coli Top10 and ClearColi BL21(DE3) were used for molecular cloning and CRM particle production respectively. The detail description of bacterial growth condition, plasmid transformation, CRM particle production, and CRM particle isolation and purification were described in the Example 1.

### Plasmid Construction for the Formation of CRM197 Displaying Q Fever Diagnostic Antigens

The recombinant gene fragments encoding Com1, OmpH, YbgF, and GroEL was codon-optimized for E. coli cells and excised from pUC57 vector (Biomatik, Canada) by enzyme digestion with XhoI and BamHI (BioLabs, USA) followed by DNA fragment separation using agarose gel electrophoresis with GelRed solution (Biotium, USA) and gel purification (BioLabs, USA). The vector plasmid pET14b CRM197 was digested with XhoI and BamHI. The subsequent linearized pET14b CRM197 vector was ligated to the DNA fragments encoding Com1, OmpH, YbgF, and GroEL protein, generating the final plasmids, pET14b CRM197-Com1, pET14b CRM197-OmpH, pET14b CRM197-YbgF, and pET14b CRM197-GroEL. The final plasmid DNA sequences were confirmed by Griffith University genome sequencing centre (Griffith University, Australia).

### Results and Discussion

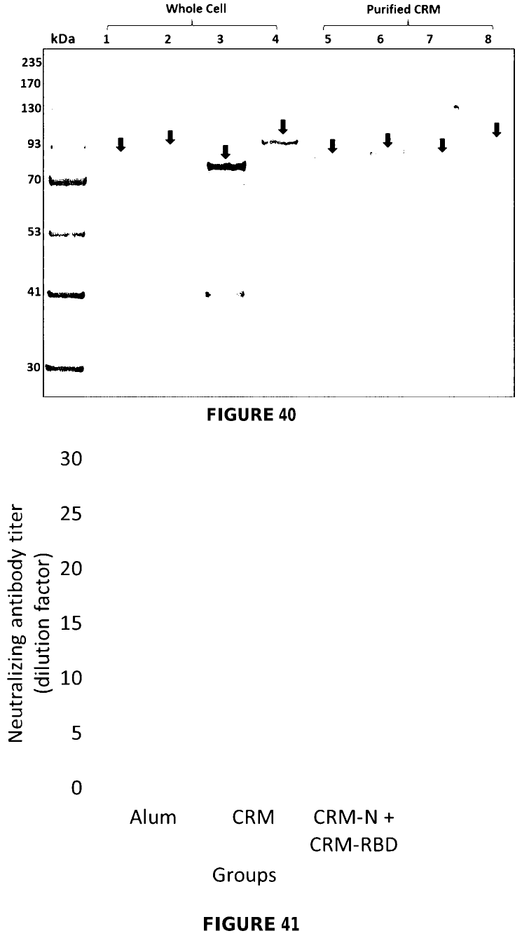

The CRM197 carrier Q fever diagnostic antigens were produced in ClearColi BL21(DE3). The protein profile of purified Q fever diagnostic reagents was analysed on 10% Bis-Tris Gel (FIG. 40). The SDS-PAGE showed that a dominant protein band with a high purity corresponding to the proteins with the theoretical MWs of CRM197-Com1 (86.4 kDa), CRM197-GroEL (83.1 kDa), CRM197-OmpH (81.5 kDa), and CRM197-YbgF (93.0 kDa). This result demonstrated that CRM197 particle-based Q fever diagnostic antigens can be successfully produced by ClearColi BL21(DE3).

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

All computer programs, algorithms, patent literature, scientific literature referred to herein is incorporated by reference in their entirety.

Each embodiment described herein is to be applied mutatis mutandis to each and every embodiment unless specifically stated otherwise.

When any number or range is described herein, unless clearly stated otherwise, that number or range is approximate. Recitation of ranges of values herein are intended to serve as a shorthand method of referring individually to each separate value falling within the range, unless otherwise indicated herein, and each separate value and each separate subrange defined by such separate values is incorporated into the specification as if it were individually recited herein.

Unless the meaning is clearly to the contrary, all ranges set forth herein are deemed inclusive of the endpoints.

Throughout this specification, reference to any advantages, promises, objects or the like should not be regarded as cumulative, composite and/or collective and should be regarded as preferable or desirable rather than stated as a warranty.

The appended claims are to be considered as incorporated into the above description.

### REFERENCES

1. Pappenhe.Am and D. M. Gill, Diphtheria. Science, 1973. 182 (4110): p. 353-358.
2. Miyaji, E. N., et al., Induction of neutralizing antibodies against diphtheria toxin by priming with recombinant Mycobacterium bovis BCG expressing CRM197, a mutant diphtheria toxin. Infection and Immunity, 2001. 69 (2): p. 869-874.
3. Dietrich, J., et al., Exchanging ESAT6 with TB10.4 in an Ag85B fusion molecule-based tuberculosis subunit vaccine: Efficient protection and ESAT6-based sensitive monitoring of vaccine efficacy. Journal of Immunology, 2005. 174 (10): p. 6332-6339.
4. Kaufmann, S. H. E., Tuberculosis vaccines-a new kid on the block. Nature Medicine, 2011. 17 (2): p. 159-160.
5. Lin, P. L., et al., The multistage vaccine H56 boosts the effects of BCG to protect cynomolgus macaques against active tuberculosis and reactivation of latent Mycobacterium tuberculosis infection. Journal of Clinical Investigation, 2012. 122 (1): p. 303-314.
6. Billeskov, R., et al., The HyVac4 subunit vaccine efficiently boosts BCG-primed anti-mycobacterial protective immunity. Plos One, 2012. 7 (6).
7. Truc, H., et al., ESAT-6 (EsxA) and TB10.4 (EsxH) based vaccinesfor pre-and post-exposure tuberculosis vaccination. Plos One, 2013. 8 (12).
8. Geldenhuys, H., et al., The tuberculosis vaccine H4:IC31 is safe and induces a persistent polyfunctional CD4 T cell response in South African adults: A randomized controlled trial. Vaccine, 2015. 33 (30): p. 3592-9.
9. He, H., H. Yang, and Y. Deng, Mycobacterium tuberculosis dormancy-associated antigen of Rv2660c induces stronger immune response in latent Mycobacterium tuberculosis infection than that in active tuberculosis in a Chinese population. European Journal of Clinical Microbiology & Infectious Diseases, 2015. 34 (6): p. 1103-1109.
10. Chen, S., et al., Innovative antigen carrier system for the development of tuberculosis vaccines. The FASEB Journal, 2019: p. fj. 201802501RR.
11. Peters, V. and B. H. A. Rehm, In vivo monitoring of PHA granule formation using GFP-labeled PHA synthases. Fems Microbiology Letters, 2005. 248 (1): p. 93-100.
12. Jahns, A. C., et al., In vivo self-assembly of fluorescent protein microparticles displaying specific binding domains. Bioconjugate Chemistry, 2013. 24 (8): p. 1314-1323.
13. Chen, S., et al., Design of Bacterial Inclusion Bodies as Antigen Carrier Systems. Advanced Biosystems, 2018. 2 (11): p. 1800118.
14. Morell, M., et al., Inclusion bodies: Specificity in their aggregation process and amyloid-like structure. Biochimica Et Biophysica Acta-Molecular Cell Research, 2008. 1783 (10): p. 1815-1825.
15. Peternel, S., et al., Inclusion bodies contraction with implications in biotechnology. Acta Chimica Slovenica, 2008. 55 (3): p. 608-612.
16. Peternel, S. and R. Komel, Active protein aggregates produced in Escherichia coli. International Journal of Molecular Sciences, 2011. 12 (11): p. 8275-8287.
17. Foged, C., et al., Particle size and surface charge affect particle uptake by human dendritic cells in an in vitro model. International Journal of Pharmaceutics, 2005. 298 (2): p. 315-322.
18. Bernfield, M., et al., Functions of cell surface heparan sulfate proteoglycans. Annual Review of Biochemistry, 1999. 68 (1): p. 729-777.
19. Limbach, L. K., et al., Oxide nanoparticle uptake in human lung fibroblasts: effects of particle size, agglomeration, and diffusion at low concentrations. Environmental Science & Technology, 2005. 39 (23): p. 9370-9376.
20. Rehm, B. H., Bioengineering towards self-assembly of particulate vaccines. Current opinion in biotechnology, 2017. 48: p. 42-53.
21. Honary, S. and F. Zahir, Effect of Zeta Potential on the Properties of Nano-Drug Delivery Systems—A Review (Part 1). Tropical Journal of Pharmaceutical Research, 2013. 12 (2): p. 255-264.
22. Honary, S. and F. Zahir, Effect of Zeta Potential on the Properties of Nano-Drug Delivery Systems—A Review (Part 2). Tropical Journal of Pharmaceutical Research, 2013. 12 (2): p. 265-273.
23. De Temmerman, M. L., et al., Particulate vaccines: on the quest for optimal delivery and immune response. Drug discovery today, 2011. 16 (13-14): p. 569-582.
24. Ackerman, A. L., et al., Early phagosomes in dendritic cells form a cellular compartment sufficient for cross presentation of exogenous antigens. Proceedings of the National Academy of Sciences, 2003. 100 (22): p. 12889-12894.
25. Gonzalez-Miro, M., et al., Polyester as antigen carrier toward particulate vaccines. Biomacromolecules, 2019.
26. Woodard, R. W., T. C. Meredith, and P. Aggarwal, Viable non-toxic gram-negative bacteria. 2012, Google Patents.
27. Chan, J., et al. The role of B cells and humoral immunity in Mycobacterium tuberculosis infection. in Seminars in immunology. 2014. Elsevier.
28. Maggioli, M. F., et al., Polyfunctional cytokine production by central memory T cells from cattle in response to Mycobacterium bovis infection and BCG vaccination. 2016, Am Assoc Immnol.
29. Orlando, V., et al., human cD4 T-cells With a naive Phenotype Produce Multiple cytokines During Mycobacterium Tuberculosis infection and correlate With active Disease. Frontiers in Immunology, 2018. 9.
30. Andersen, P. and K. B. Urdahl, TB vaccines; promoting rapid and durable protection in the lung. Current Opinion in Immunology, 2015. 35: p. 55-62.
31. Jasenosky, L. D., et al., T cells and adaptive immunity to Mycobacterium tuberculosis in humans. Immunological Reviews, 2015. 264 (1): p. 74-87.
32. Lewinsohn, D. A., D. M. Lewinsohn, and T. J. Scriba, Polyfunctional CD4+ T cells as targets for tuberculosis vaccination. Frontiers in Immunology, 2017. 8: p. 1262.
33. Lin, L. C. W., et al., Advances and Opportunities in Nanoparticle-and Nanomaterial-Based Vaccines against Bacterial Infections. Advanced Healthcare Materials, 2018: p. 1701395.
34. Fenton, M. J., et al., Induction of gamma interferon production in human alveolar macrophages by Mycobacterium tuberculosis. Infection and Immunity, 1997. 65 (12): p. 5149-5156.
35. Cooper, A. M., Cell-mediated immune responses in tuberculosis. Annual Review of Immunology, 2009. 27: p. 393-422.
36. Khader, S. A. and R. Gopal, IL-17 in protective immunity to intracellular pathogens. Virulence, 2010. 1 (5): p. 423-427.
37. Torrado, E. and A. M. Cooper, IL-17 and Th17 cells in tuberculosis. Cytokine & Growth Factor Reviews, 2010. 21 (6): p. 455-462.
38. Rubio Reyes, P., et al., Immunogencity of antigens from Mycobacterium tuberculosis self-assembled as particulate vaccines. International Journal of Medical Microbiology: IJMM, 2016. 306 (8): p. 624-632.
39. Bhatt, K., et al., Quest for correlates of protection against tuberculosis. Clinical and Vaccine Immunology, 2015. 22 (3): p. 258-266.
40. Rubio-Reyes, P., et al., Immunological properties and protective efficacy of a single mycobacterial antigen displayed on polyhydroxybutyrate beads. Microbial biotechnology, 2017. 10 (6): p. 1434-1440.
41. Billeskov, R., et al., Comparing Adjuvanted H28 and Modified Vaccinia Virus Ankara Expressing H28 in a Mouse and a Non-Human Primate Tuberculosis Model. PLoS One, 2013. 8 (8): p. e72185-Article No.: e72185.
42. Walker, M. J., et al., Disease manifestations and pathogenic mechanisms of group A Streptococcus. Clinical microbiology reviews, 2014. 27 (2): p. 264-301.
43. Laabei, M. and D. J. J.o.i.i. Ermert, Catch me if you can: Streptococcus pyogenes complement evasion strategies. 2019. 11 (1): p. 3-12.
44. Good, M. F., et al., Strategic development of the conserved region of the M protein and other candidates as vaccines to prevent infection with group A streptococci. 2015. 14 (11): p. 1459-1470.
45. Nordström, T., et al., Enhancing Vaccine Efficacy by Engineering a Complex Synthetic Peptide To Become a Super Immunogen. 2017. 199 (8): p. 2794-2802.
46. Pandey, M., et al., Physicochemical characterisation, immunogenicity and protective efficacy of a lead streptococcal vaccine: progress towards phase I trial. Scientific reports, 2017. 7 (1): p. 1-11.
47. Malito, E., et al., Structural basis for lack of toxicity of the diphtheria toxin mutant CRM197. Proceedings of the National Academy of Sciences, 2012. 109 (14): p. 5229-5234.
48. Peters, V. and B. H. A. Rehm, In vivo monitoring of PHA granule formation using GFP-labeled PHA synthases. FEMS Microbiol. Lett., 2005. 248 (1): p. 93-100.
49. Jahns, A. C., et al., In vivo self-assembly of fluorescent protein microparticles displaying specific binding domains. Bioconjugate chemistry, 2013. 24 (8): p. 1314-1323.
50. Rehm, B. H. A., Bioengineering towards self-assembly of particulate vaccines. Curr. Opin. Biotech., 2017. 48: p. 42-53.
51. Honary, S. and F. Zahir, Effect of zeta potential on the properties of nano-drug delivery systems—a review (Part 1). Tropical Journal of Pharmaceutical Research, 2013. 12 (2): p. 255-264.
52. Honary, S. and F. Zahir, Effect of zeta potential on the properties of nano-drug delivery systems—a review (Part 2). Tropical Journal of Pharmaceutical Research, 2013. 12 (2): p. 265-273.
53. Batista, P. d. O. M. D., et al., Methods of endotoxin removal from biological preparations: a review. 2007.
54. Yu, H. H., et al., Expressed protein ligation for the preparation of fusion proteins with cell penetrating peptides for endotoxin removal and intracellular delivery. Biochimica et Biophysica Acta (BBA)-Biomembranes, 2010. 1798 (12): p. 2249-2257.
55. Mamat, U., et al., Endotoxin-free protein production ClearColi™ technology. Nat. Methods, 2013. 10 (9).
56. Chen, S., et al., New skin test for detection of bovine tuberculosis on the basis of antigen-displaying polyester inclusions produced by recombinant Escherichia coli. Appl. Environ. Microbiol., 2014. 80 (8): p. 2526-2535.

### Tables

TABLE 1
Strains, plasmids, and oligouncleotides used in Example 1.

| Strains/plasmids/ | | Sources or |
|---|---|---|
| oligonucleotides | Characteristics^{a} | references |
| E. coli | | |
| XLI-Blue | recA1 endA1 gyrA96 thi-1 hsdR17 supE44 | Stratagene |
| | relA1 lac [F′ proAB lacl^{q} lacZΔM15 Tn10 | |
| | (Tet^{R})] | |
| ClearColi BL21 (DE3) | F⁻ ompT hsdS_{B}/r_{B′} m_{B′}) gal dcm ion λ(DE3 | Lucigen |
| | [lacI lacUV5-T7 gene 1 ind1 sam7 nin5]) | |
| | msbA148 ΔgutQ ΔkdsD ΔlpxL ΔlpxM ΔpagP | |
| | ΔlpxP ΔeptA | |
| Plasmids | | |
| pET-14b | Ap^{R}; T7 promoter | Novagen |
| pET-14b cfp10-phaC | pET-14b derivative containing NdeI fragment | [56] |
| | gene cfp10 fused to the 5′ end of phaC | |
| pUC57 CRM197 | pET-14b derivative containing NdeI fragment | GenScript |
| | gene CRM197 | |
| pUC57 H4 | Cloning vector, ColE1 origin, Ap^{R}; BamHI | [13] |
| | fragment gene H4 | |
| pUC57 H28 | Cloning vector, ColE1 origin, Ap^{R}; BamHI | [13] |
| | fragment gene H28 | |
| pET-14b His6-H4 | pET-14b derivative containing NdeI/BamHI | [10] |
| | fragment gene his6-h4 | |
| pET-14b His6-H28 | pET-14b derivative containing NdeI/BamHI | [10] |
| | fragment gene his6-h28 | |
| pET-14b CRM197 | pET-14b derivative containing NdeI/BamHI | This study |
| | fragment gene CRM197 | |
| pET-14b CRM197-H4 | pET-14b derivative containing BamHI | This study |
| | fragment gene H4 fused to the 3′ end of | |
| | CRM197 | |
| pET-14b CRM197-H28 | pET-14b derivative containing BamHI | This study |
| | fragment gene H28 fused to the 3′ end of | |
| | CRM197 | |
| Oligonucleotides | | |
| CRM197_NdeI_Fwd | CCCATATGGGTGCAGATGACGTGGTTGACAGCTC | This study |
| | (SEQ ID NO: 3) | |
| CRM197_BamHI_Rev | CCGGATCCAGATTTAATTTCGAAAAACAGAGACAGTTTG | This study |
| | SEQ ID NO: 4) | |
| CRM197_stop_BamHI_Rev | CCGGATCCTTAAGATTTAATTTCGAAAAACAGAGACAGTTTG | This study |
| | (SEQ ID NO: 5) | |

Tet^{R}, tetracycline resistance; Ap^{R}, ampicillin resistance; h4, ag85b-tb10.4; h28,
ag85b-tb10.4-rv2660c; underline, gene sequence of restriction enzyme.

TABLE 2
MALDI-TOF/MS analysis of CRM197-mycobacterial antigen fusion proteins

| | |
|---|---|
| | Peptide fragments assigned to |
| Protein/Protein sequence | the various protein regions |

CRM197 (MW: 58.544 kDa)

| | |
|---|---|
| MGADDWDSSKSFVMENFSSYHGTKPGYVDSIQKGIQKPKSGTQGN | CRM197: S12-K34, S41-K77, |
| YDDDWKEFYSTDNKYDAAGYSVDNENPLSGKAGGVVKVTYPGLTK | E106-R127, V135-R171, R174-R191, |
| VLALKVDNAETIKKELGLSLTEPLMEQVGTEEFIKRFGDGASRVV | S195-K213, T216-K228, Q246-K300, |
| LSLPFAEGSSSVEYINNWEQAKALSVELEINFETRGKRGQDAMYE | T387-K446, T499-R456, A464-R494, |
| YMAQACAGNRVRRSVGSSLSCINLDWDVIRDKTKTKIESLKEHGP | I500-K517, L528-S536 |
| IKNKMSESPNKTVSEEKAKQYLEEFHQTALEHPELSELKTVTGTN | |
| PVFAGANYAAWAVNVAQVIDSETADNLEKTTAALSILPGIGSVMG | |
| IADGAVHHNTEEIVAQSIALSSLMVAQAIPLVGELVDIGFAAYNF | |
| VESIINLFQVVHNSYNRPAYSPGHKTQPFLHDGYAVSWNTVEDSI | |
| IRTGFQGESGHDIKITAENTPLPIAGVLLPTIPGKLDVNKSKTHI | |
| SVNGRKIRMRCRAIDGDVTFCRPKSPVYVGNGVHANLHVAFHRSS | |
| SEKIHSNEISSDSIGVLGYQKTVDHTKVNSKLSLFFEIKS | |
| (SEQ ID NO: 2) | |

CRM197-Ag85B-TB10.4 (MW: 99.705 kDa; SEQ ID NO: 19)

| | |
|---|---|
| MGADDVVDSSKSFVMENFSSYHGTKPGYVDSIQKGIQKPKSGTQG | CRM197: S12-K34, S41-K77, |
| NYDDDWKEFYSTDNKYDAAGYSVDNENPLSGKAGGVVKVTYPGLT | E106-R127, V135-R171, G175-R191, |
| KVLALKVDNAETIKKELGLSLTEPLMEQVGTEEFIKRFGDGASRV | S195-K213, T266-K300, T387-R408, |
| VLSLPFAEGSSSVEYINNWEQAKALSVELEINFETRGKRGQDAMY | I421-K446, T449-R456, A464-R494, |
| EYMAQACAGNRVRRSVGSSLSCINLDWDVIRDKTKTKIESLKEHG | I500-K517, L528-S536 |
| PIKNKMSESPNKTVSEEKAKQYLEEFHQTALEHPELSELKTVTGT | Ag85B: F539-R558, V562-R581, |
| NPVFAGANYAAWAVNVAQVIDSETADNLEKTTAALSILPGIGSVM | A714-K737, L745-R772, F778-K813 |
| GIADGAVHHNTEEIVAQSIALSSLMVAQAIPLVGELVDIGFAAYN | TB10.4: No peptides are identified |
| FVESIINLFQVVHNSYNRPAYSPGHKTQPFLHDGYAVSWNTVEDS | |
| IIRTGFQGESGHDIKITAENTPLPIAGVLLPTIPGKLDVNKSKTH | |
| ISVNGRKIRMRCRAIDGDVTFCRPKSPVYVGNGVHANLHVAFHRS | |
| SSEKIHSNEISSDSIGVLGYQKTVDHTKVNSKLSLFFEIKSGSFS | |
| RPGLPVEYLQVPSPSMGRDIKVQFQSGGNNSPAVYLLDGLRAQDD | |
| YNGWDINTPAFEWYYQSGLSIVMPVGGQSSFYSDWYSPACGKAGC | |
| QTYKWETFLTSELPQWLSANRAVKPTGSAAIGLSMAGSSAMILAA | |
| YHPQQFIYAGSLSALLDPSQGMGPSLIGLAMGDAGGYKAADMWGP | |
| SSDPAWERNDPTQQIPKLVANNTRLWVYCGNGTPNELGGANIPAE | |
| FLENFVRSSNLKFQDAYNAAGGHNAVFNFPPNGTHSWEYWGAQLN | |
| AMKGDLQSSLGAGMSQIMYNYPAMLGHAGDMAGYAGTLQSLGAEI | |
| AVEQAALQSAWQGDTGITYQAWQAQWNQAMEDLVRAYHAMSSTHE | |
| ANTMAMMARDTAEAAKWGG | |
| (SEQ ID NO: 19) | |

CRM197-Ag85B-TB10.4-Rv2660c (107.269 kDa; SEQ ID NO: 20)

| | |
|---|---|
| MGADDVVDSSKSFVMENFSSYHGTKPGYVDSIQKGIQKPKSGTQG | CRM197: S12-K34, S41-K77, |
| NYDDDWKEFYSTDNKYDAAGYSVDNENPLSGKAGGVVKVTYPGLT | E106-R127, V135-R171, G175-R191, |
| KVLALKVDNAETIKKELGLSLTEPLMEQVGTEEFIKRFGDGASRV | S195-R211, T387-K446, T449-R456, |
| VLSLPFAEGSSSVEYINNWEQAKALSVELEINFETRGKRGQDAMY | A464-K475, I500-K517, L528-S536 |
| EYMAQACAGNRVRRSVGSSLSCINLDWDVIRDKTKTKIESLKEHG | Ag85B: F539-R558, V562-R581, |
| PIKNKMSESPNKTVSEEKAKQYLEEFHQTALEHPELSELKTVTGT | W635-R651, N729-K737, L745-R772, |
| NPVFAGANYAAWAVNVAQVIDSETADNLEKTTAALSILPGIGSVM | F778-K813 |
| GIADGAVHHNTEEIVAQSIALSSLMVAQAIPLVGELVDIGFAAYN | TB10.4: A891-R909 |
| FVESIINLFQVVHNSYNRPAYSPGHKTQPFLHDGYAVSWNTVEDS | Rv2660c: A938-H994 |
| IIRTGFQGESGHDIKITAENTPLPIAGVLLPTIPGKLDVNKSKTH | |
| ISVNGRKIRMRCRAIDGDVTFCRPKSPVYVGNGVHANLHVAFHRS | |
| SSEKIHSNEISSDSIGVLGYQKTVDHTKVNSKLSLFFEIKSGSFS | |
| RPGLPVEYLQVPSPSMGRDIKVQFQSGGNNSPAVYLLDGLRAQDD | |
| YNGWDINTPAFEWYYQSGLSIVMPVGGQSSFYSDWYSPACGKAGC | |
| QTYKWETFLTSELPQWLSANRAVKPTGSAAIGLSMAGSSAMILAA | |
| YHPQQFIYAGSLSALLDPSQGMGPSLIGLAMGDAGGYKAADMWGP | |
| SSDPAWERNDPTQQIPKLVANNTRLWVYCGNGTPNELGGANIPAE | |
| FLENFVRSSNLKFQDAYNAAGGHNAVFNFPPNGTHSWEYWGAQLN | |
| AMKGDLQSSLGAGMSQIMYNYPAMLGHAGDMAGYAGTLQSLGAEI | |
| AVEQAALQSAWQGDTGITYQAWQAQWNQAMEDLVRAYHAMSSTHE | |
| ANTMAMMARDTAEAAKWGGMIAGVDQALAATGQASQRAAGASGGV | |
| TVGVGVGTEQRNLSVVAPSQFTFSSRSPDFVDETAGQSWCAILGL | |
| NQFH | |
| (SEQ ID NO: 20) | |

TABLE 3
MALDI-TOF/MS analysis of His6-tagged H4 and H28 antigen fusion proteins

| | |
|---|---|
| | Peptide fragments assigned to |
| Protein/Protein sequence | the various protein regions |

His6-Ag85B-TB10.4 (MW: 106.698 kDa; SEQ ID NO: 21)

| | |
|---|---|
| MHHHHHHFSRPGLPVEYLQVPSPSMGRDIKVQFQSGGNNSPAVYLLDGLR | Ag85B: F8-F33, L45-Y68, S74-Y86, |
| AQDDYNGWDINTPAFEWYYQSGLSIVMPVGGQSSFYSDWYSPACGKAGCQ | S91-Y102, S117-L132, A143-F150, |
| TYKWETFLTSELPQWLSANRAVKPTGSAAIGLSMAGSSAMILAAYHPQQF | K182-W215, K246-Y251, N262-W271 |
| IYAGSLSALLDPSQGMGPSLIGLAMGDAGGYKAADMWGPSSDPAWERNDP | TB10.4: N299-Y313, N351-Y361, |
| TQQIPKLVANNTRLWVYCGNGTPNELGGANIPAEFLENFVRSSNLKFQDA | A374-G388 |
| YNAAGGHNAVFNFPPNGTHSWEYWGAQLNAMKGDLQSSLGAGMSQIMYNY | |
| PAMLGHAGDMAGYAGTLQSLGAEIAVEQAALQSAWQGDTGITYQAWQAQW | |
| NQAMEDLVRAYHAMSSTHEANTMAMMARDTAEAAKWGG | |
| (SEQ ID NO: 21) | |

His6-Ag85B-TB10.4-Rv2660c (114.263 kDa; SEQ ID NO: 22)

| | |
|---|---|
| MHHHHHHFSRPGLPVEYLQVPSPSMGRDIKVQFQSGGNNSPAVYLLDGLR | Ag85B: F8-R27, V31-R50, |
| AQDDYNGWDINTPAFEWYYQSGLSIVMPVGGQSSFYSDWYSPACGKAGCQ | A183-R241 |
| TYKWETFLTSELPQWLSANRAVKPTGSAAIGLSMAGSSAMILAAYHPQQF | TB10.4: A360-R378 |
| IYAGSLSALLDPSQGMGPSLIGLAMGDAGGYKAADMWGPSSDPAWERNDP | Rv2660c: M389-R440 |
| TQQIPKLVANNTRLWVYCGNGTPNELGGANIPAEFLENFVRSSNLKFQDA | |
| YNAAGGHNAVFNFPPNGTHSWEYWGAQLNAMKGDLQSSLGAGMSQIMYNY | |
| PAMLGHAGDMAGYAGTLQSLGAEIAVEQAALQSAWQGDTGITYQAWQAQW | |
| NQAMEDLVRAYHAMSSTHEANTMAMMARDTAEAAKWGGMIAGVDQALAAT | |
| GQASQRAAGASGGVTVGVGVGTEQRNLSVVAPSQFTFSSRSPDFVDETAG | |
| QSWCAILGLNQFH | |
| (SEQ ID NO: 22) | |

TABLE 4
Mycobacterium tuberculosis-specific diagnostic antigens

| Antigen Name/origin | Amino acid Sequence |
|---|---|
| α-crystallin (HspX) | MATTLPVQRHPRSLFPEFSELFAAFPSFAGLRPTFDTRLMRLEDEMKEGRYEVR |
| | AELPGVDPDKDVDIMVRDGQLTIKAERTEQKDFDGRSEFAYGSFVRTVSLPVGA |
| | DEDDIKATYDKGILTVSVAVSEGKPTEKHIQIRSTNGGGGG |
| | (SEQ ID NO: 32) |
| Early secreted | MTEQQWNFAGIEAAASAIQGNVTSIHSLLDEGKQSLTKLAAAWGGSGSEAYQGV |
| antigenic target 6 | QQKWDATATELNNALQNLARTISEAGQAMASTEGNVTGMFA |
| kDA (ESAT6) | (SEQ ID NO: 33) |
| Culture filtrate protein | MAEMKTDAATLAQEAGNFERISGDLKTQIDQVESTAGSLQGQWRGAAGTAAQAA |
| 10 kDa (CFP10) | VVRFQEAANKQKQELDEISTNIRQAGVQYSRADEEQQQALSSQMGF |
| | (SEQ ID NO: 34) |
| RV1509 | MVFALSNNLNRVNACMDGFLARIRSHVDAHAPELRSLFDTMAAEARFARDWLSE |
| | DLARLPVGAALLEVGGGVLLLSCQLAAEGFDITAIEPTGEGFGKFRQLGDIVLE |
| | LAAARPTIAPCKAEDFISEKRFDFAFSLNVMEHIDLPDEAVRRVSEVLKPGASY |
| | HFLCPNYVFPYEPHFNIPTFFTKELTCRVMRHRIEGNTGMDDPKGVWRSLNWIT |
| | VPKVKRFAAKDATLTLRFHRAMLVWMLERALTDKEFAGRRAQWMVAAIRSAVKL |
| | RVHHLAGYVPATLQPIMDVRLTKR |
| | (SEQ ID NO: 35) |
| Rv2658c | ADAVKYVVMCNCDDEPGALIIAWIDDERPAGGHIQMRSNTRFTETQWGRHIEWK |
| | LECRACRKYAPISEMTAAAILDGFGAKLHELRTSTIPDADDPSIAEARHVIPFS |
| | ALCLRLSQLGG |
| | (SEQ ID NO: 36) |
| Rv1508c | VIPVMSARFTGFPLLPVALRHGITSGRGCGFILDVGAQRPFGNDVLLSVATRKI |
| | RSRLPGDRVGNHGALLPFRAEPRRIQMKRPPEVLRGAVTASRERLWAIGSQSER |
| | TLMLGTILLASVISAATAYALSQWYAVDVFSTLLVVPGDCWLDWGMNIGRHCFS |
| | DYAMVAAAGIQPNPADYLISLPADYQPTAVAAWAPARIPYAIFGLPSHWLGAPR |
| | LGLICYLVALTMAVISPAIWAARGARGLERVVIFVTLGAAAIPAWGVIDRGNST |
| | GFVVPIALAYFVALSRQRWGLATITVILAVLVKPQFVVLGVVLLAARQWRWAGI |
| | GITGVVVSNIAAFLLWPRGFPGTIAQSIHGIIKFNSSFGGLRDPRNVSFGKALL |
| | LIPDSIKNYQSGKIPEGFLTGPRTQIGFAVLVIVVVAVLALGRRIPPVMVGIVL |
| | LATATFSPADVAFYYLVFVLPIAALVARDPNGPPGAGIFDQLAAHGDRRRAVGV |
| | VSLCAVALSIVNVAVPGQPFYVPLYGQLGAKGVVGTTPLVFTTVTWAPFLWLVT |
| | CVVIIVSYARKPARPHDSHNGPTRESDQDTAASTTSCLPNPVEESSPRGPGPIC |
| | QNYTP |
| | (SEQ ID NO: 37) |
| TB7.7 | MSGHALAARTLLAAADELVGGPPVEASAAALAGDAAGAWRTAAVELARALVRAV |
| | AESHGVAAVLFAATAAAAAAVDRGDPP(SEQ ID NO: 38) |
| Rv3615c | PMTENLTVQPERLGVLASHHDNAAVDASSGVEAAAGLGESVAITHGPYCSQFND |
| | TLNVYLTAHNALGSSLHTAGVDLAKSLRIAAKIYSEADEAWRKAIDGLFT |
| | (SEQ ID NO: 39) |
| Rv3020c | MSLLDAHIPQLIASHTAFAAKAGLMRHTIGQAEQQAMSAQAFHQGESAAAFQGA |
| | HARFVAAAAKVNTLLDIAQANLGEAAGTYVAADAAAASSYTGF |
| | (SEQ ID NO: 40) |

TABLE 5
Bacterial strains, plasmids, and primers used in Example 4 to generate GAS particles

| Strains, Plasmids | | |
|---|---|---|
| and Primers | Relevant characteristics | References |
| 1. Bacterial strains | | |
| E. coli | | |
| XL1-Blue | recA1 endA1 gyrA96 thi-1 hsdR17 supE44 relA1 lac | Stratagene |
| ClearColi™ BL21(DE3) | [F′ proAB lacI^{q} lacZ ΔM15 Tn10 (Tet¹)] | Lucigen |
| | F- ompT hsdSB (rB-mB-) gal dcm ion λ(DE3 [lacI | |
| | lacUV5-T7 gene 1 ind1 sam7 nin5]) msbA148 | |
| | ΔgutQΔkdsD ΔlpxLΔlpxMΔpagP ΔlpxP ΔeptA | |
| 2. Plasmids | Relevant characteristics | References |
| pET14b | Amp^{r}; T7 promoter | Novagen |
| pMCS69 | Cm^{r}; T7 promoter; pBBR1MCS derivative containing | Amara & Rehm, |
| pET14b_PhaC-RV1626 | codon optimised genes phaA and phaB from C. necator | 2003 |
| | pET-14b_PhaC derivative containing RV1626 | Rubio et al. 2016 |
| pET14b_CRM | pET-14b derivative containing np gene fragment | Shuxiong Chen |
| pUC57_P*17 | pUC57 derivative containing E. coli codon optimized | This study |
| pET14b_CRM-P*17 | P*17 fragment flanked by XhoI/BamHI sites | This study |
| | Codon optimized enol fragment from pUC57_P*17 | |
| | inserting into XhoI/BamHI sites of pET14b_NP | |
| pUC57_S2 | pUC57 derivative containing E. coli codon optimized | This study |
| | S2 fragment flanked by XhoI/BamHI sites | |
| pET14b_CRM-S2 | Codon optimized enol fragment from pUC57_S2 | This study |
| | inserting into XhoI/BamHI sites of pET14b_NP | |
| pUC57_P*17-S2 | pUC57 derivative containing E. coli codon optimized | This study |
| | P*17-S2 fragment flanked by XhoI/BamHI sites | |
| pET14b_NP-P*17-S2 | Codon optimized enol fragment from pUC57_P*17- | This study |
| | S2 inserting into XhoI/BamHI sites of pET14b_NP | |
| 3. Primers | 5′-3′ | |
| T7 promoter | TAATACGACTCACTATAGGG | GenScript, USA |
| T7 terminator | GCTAGTTATTGCTCAGCGG | GenScript, USA |

TABLE 6
Mass Spectrometry (MS) analysis of CRM197 and CRM197-StrepA antigens

| | Protein sequence coverage and |
|---|---|
| Protein Sequence | the confirmed fragments |
| CRM (MW: 58.5 kDa; SEQ ID NO: 2) | M1-N168, R175-R192, S196-N279, |
| 1 MGADDVVDSSKSFVMENFSSYHGTKPGYVDSIQKGIQKPKSGTQGNYDDD | A284-I326, I334-V352, S376-K458, |
| 51 WKEFYSTDNKYDAAGYSVDNENPLSGKAGGVVKVTYPGLTKVLALKVDNA | A465-K527 |
| 101 ETIKKELGLSLTEPLMEQVGTEEFIKRFGDGASRVVLSLPFAEGSSSVEY | |
| 151 INNWEQAKALSVELEINFETRGKRGQDAMYEYMAQACAGNRVRRSVGSSL | |
| 201 SCINLDWDVIRDKTKTKIESLKEHGPIKNKMSESPNKTVSEEKAKQYLEE | |
| 251 FHQTALEHPELSELKTVTGTNPVFAGANYAAWAVNVAQVIDSETADNLEK | |
| 301 TTAALSILPGIGSVMGIADGAVHHNTEEIVAQSIALSSLMVAQAIPLVGE | |
| 351 LVDIGFAAYNFVESIINLFQVVHNSYNRPAYSPGHKTQPFLHDGYAVSWN | |
| 401 TVEDSIIRTGFQGESGHDIKrrAENTPLPIAGVLLPTIPGKLDVNKSKTH | |
| 451 ISVNGRKIRMRCRAIDGDVTFCRPKSPVYVGNGVHANLHVAFHRSSSEKI | |
| 501 HSNEISSDSIGVLGYQKTVDHTKVNSKLSLFFEIKS | |
| CRM-P*17 (MW: 66.7 kDa) | M1-R192, R195-Q332, S376-K457, |
| 1 MGADDVVDSSKSFVMENFSSYHGTKPGYVDSIQKGIQKPKSGTQGNYDDD | C462-A609 |
| 51 WKEFYSTDNKYDAAGYSVDNENPLSGKAGGVVKVTYPGLTKVLALKVDNA | |
| 101 ETIKKELGLSLTEPLMEQVGTEEFIKRFGDGASRVVLSLPFAEGSSSVEY | |
| 151 INNWEQAKALSVELEINFETRGKRGQDAMYEYMAQACAGNRVRRSVGSSL | |
| 201 SCINLDWDVIRDKTKTKIESLKEHGPIKNKMSESPNKTVSEEKAKQYLEE | |
| 251 FHQTALEHPELSELKTVTGTNPVFAGANYAAWAVNVAQVIDSETADNLEK | |
| 301 TTAALSILPGIGSVMGIADGAVHHNTEEIVAQSIALSSLMVAQAIPLVGE | |
| 351 LVDIGFAAYNFVESIINLFQVVHNSYNRPAYSPGHKTQPFLHDGYAVSWN | |
| 401 TVEDSIIRTGFQGESGHDIK1TAENTPLPIAGVLLPTIPGKLDVNKSKTH | |
| 451 ISVNGRKIRMRCRAIDGDVTFCRPKSPVYVGNGVHANLHVAFHRSSSEKI | |
| 501 HSNEISSDSIGVLGYQKTVDHTKVNSKLSLFFEIKSGPGPGLRRDLDASR | |
| 551 EAKNQVERALEGPGPGLRRDLDASREAKNQVERALEGPGPGLRRDLDASR | |
| 601 EAKNQVERALE (SEQ ID NO: 65) | |
| CRM-S2 (MW: 66.7 kDa;) | M1-K158, R174-D319, S333-M340, |
| 1 MGADDVVDSSKSFVMENFSSYHGTKPGYVDSIQKGIQKPKSGTQGNYDDD | S375-H450, I452-K457, A464-F611 |
| 51 WKEFYSTDNKYDAAGYSVDNENPLSGKAGGVVKVTYPGLTKVLALKVDNA | |
| 101 ETIKKELGLSLTEPLMEQVGTEEFIKRFGDGASRVVLSLPFAEGSSSVEY | |
| 151 INNWEQAKALSVELEINFETRGKRGQDAMYEYMAQACAGNRVRRSVGSSL | |
| 201 SCINLDWDVIRDKTKTKIESLKEHGPIKNKMSESPNKTVSEEKAKQYLEE | |
| 251 FHQTALEHPELSELKTVTGTNPVFAGANYAAWAVNVAQVIDSETADNLEK | |
| 301 TTAALSILPGIGSVMGIADGAVHHNTEEIVAQSIALSSLMVAQAIPLVGE | |
| 351 LVDIGFAAYNFVESIINLFQVVHNSYNRPAYSPGHKTQPFLHDGYAVSWN | |
| 401 TVEDSIIRTGFQGESGHDIK1TAENTPLPIAGVLLPTIPGKLDVNKSKTH | |
| 451 ISVNGRKIRMRCRAIDGDVTFCRPKSPVYVGNGVHANLHVAFHRSSSEKI | |
| 501 HSNEISSDSIGVLGYQKTVDHTKVNSKLSLFFEIKSGPGPGNSDNIKENQ | |
| 551 FEDFDEDWENFGPGPGNSDNIKENQFEDFDEDWENFGPGPGNSDNIKENQ | |
| 601 FEDFDEDWENF (SEQ ID NO: 66) | |
| CRM-P*17-S2 (MW: 74.9 kDa) | M1-K158, R174-R211, K215-N278, |
| 1 MGADDVVDSSKSFVMENFSSYHGTKPGYVDSIQKGIQKPKSGTQGNYDDD | A283-M315, N377-K457, A464-K475, |
| 51 WKEFYSTDNKYDAAGYSVDNENPLSGKAGGVVKVTYPGLTKVLALKVDNA | S495-K527, K-535-F686 |
| 101 ETIKKELGLSLTEPLMEQVGTEEFIKRFGDGASRVVLSLPFAEGSSSVEY | |
| 151 INNWEQAKALSVELEINFETRGKRGQDAMYEYMAQACAGNRVRRSVGSSL | |
| 201 SCINLDWDVIRDKTKTKIESLKEHGPIKNKMSESPNKTVSEEKAKQYLEE | |
| 251 FHQTALEHPELSELKTVTGTNPVFAGANYAAWAVNVAQVIDSETADNLEK | |
| 301 TTAALSILPGIGSVMGIADGAVHHNTEEIVAQSIALSSLMVAQAIPLVGE | |
| 351 LVDIGFAAYNFVESIINLFQVVHNSYNRPAYSPGHKTQPFLHDGYAVSWN | |
| 401 TVEDSIIRTGFQGESGHDIK1TAENTPLPIAGVLLPTIPGKLDVNKSKTH | |
| 451 ISVNGRKIRMRCRAIDGDVTFCRPKSPVYVGNGVHANLHVAFHRSSSEKI | |
| 501 HSNEISSDSIGVLGYQKTVDHTKVNSKLSLFFEIKSGPGPGLRRDLDASR | |
| 551 EAKNQVERALEGPGPGLRRDLDASREAKNQVERALEGPGPGLRRDLDASR | |
| 601 EAKNQVERALEGPGPGNSDNIKENQFEDFDEDWENFGPGPGNSDNIKENQ | |
| 651 FEDFDEDWENFGPGPGNSDNIKENQFEDFDEDWENF (SEQ ID NO: 67) | |
| CRM-P*17-S2- 2^{nd} band | M1-K11, P26-K34, Y61-K77, |
| 1 MGADDVVDSSKSFVMENFSSYHGTKPGYVDSIQKGIQKPKSGTQGNYDDD | V84-K91, V97-K105; R127-R134, |
| 51 WKEFYSTDNKYDAAGYSVDNENPLSGKAGGVVKVTYPGLTKVLALKVDNA | I203-R211, T216-K229, P236-246, |
| 101 ETIKKELGLSLTEPLMEQVGTEEFIKRFGDGASRVVLSLPFAEGSSSVEY | T268-Y279, A304-G312, T387-Y395, |
| 151 INNWEQAKALSVELEINFETRGKRGQDAMYEYMAQACAGNRVRRSVGSSL | T401-K420, I438-K446, T449-K457, |
| 201 SCINLDWDVIRDKTKTKIESLKEHGPIKNKMSESPNKTVSEEKAKQYLEE | A464-K475, I500-K517, K535-K622 |
| 251 FHQTALEHPELSELKTVTGTNPVFAGANYAAWAVNVAQVIDSETADNLEK | |
| 301 TTAALSILPGIGSVMGIADGAVHHNTEEIVAQSIALSSLMVAQAIPLVGE | |
| 351 LVDIGFAAYNFVESIINLFQVVHNSYNRPAYSPGHKTQPFLHDGYAVSWN | |
| 401 TVEDSIIRTGFQGESGHDIK1TAENTPLPIAGVLLPTIPGKLDVNKSKTH | |
| 451 ISVNGRKIRMRCRAIDGDVTFCRPKSPVYVGNGVHANLHVAFHRSSSEKI | |
| 501 HSNEISSDSIGVLGYQKTVDHTKVNSKLSLFFEIKSGPGPGLRRDLDASR | |
| 551 EAKNQVERALEGPGPGLRRDLDASREAKNQVERALEGPGPGLRRDLDASR | |
| 601 EAKNQVERALEGPGPGNSDNIKENQFEDFDEDWENFGPGPGNSDNIKENQ | |
| 651 FEDFDEDWENFGPGPGNSDNIKENQFEDFDEDWENF (SEQ ID NO: 68) | |

TABLE 7
T and B Cell epitopes of Q fever antigens

| | | | Sequence |
|---|---|---|---|
| Epitopes of OmpH | B cell | T cell | Identifier |
| APQIKDINTRLEKQFS | ✓ | | SEQ ID NO: 74 |
| MSKVNGAVKRVAEREN | ✓ | | SEQ ID NO: 75 |
| LSAICLSVAMIWSVAA | ✓ | | SEQ ID NO: 76 |
| LRKEIQNDESTLRQQQ | ✓ | | SEQ ID NO: 77 |
| TRLEKQFSPQREKMTK | ✓ | | SEQ ID NO: 78 |
| RVAERENLDLVLPKDT | ✓ | | SEQ ID NO: 79 |
| YAKNSKDITSN | ✓ | | SEQ ID NO: 80 |
| QNKAMSD | ✓ | | SEQ ID NO: 81 |
| LYAKNSKDITSN | ✓ | | SEQ ID NO: 82 |
| GKKEAENLRKEIQNDE | ✓ | | SEQ ID NO: 83 |
| TLRQQQQQFQQE | ✓ | | SEQ ID NO: 84 |
| QELFVAQNKAMSDFM | | ✓ | SEQ ID NO: 85 |
| | | | Sequence |
| Epitopes of GroEL | B cell | T cell | Identifier |
| TEVEMKEKKARVEDAL | ✓ | | SEQ ID NO: 86 |
| GYLSPYFI | ✓ | | SEQ ID NO: 87 |
| VEEGVVPGGGV | ✓ | | SEQ ID NO: 88 |
| KKISNIR | ✓ | | SEQ ID NO: 89 |
| VTKDDTTIIDGSGDAGDIKN | ✓ | | SEQ ID NO: 90 |
| IKNRVEQIRKEIENSSSDYD | ✓ | | SEQ ID NO: 91 |
| KEKLQERL | ✓ | | |
| TEAPKKKEESMP | ✓ | | SEQ ID NO: 92 |
| ASRTSDDAGDGTTTAT | ✓ | | SEQ ID NO: 93 |
| SKPCKDQKA | ✓ | | SEQ ID NO: 94 |
| SANSDKSIGD | ✓ | | SEQ ID NO: 95 |
| EKVGKEG | ✓ | | SEQ ID NO: 96 |
| NNQQNMS | ✓ | | SEQ ID NO: 97 |
| DSVEVENEDQRVG | ✓ | | SEQ ID NO: 98 |
| ATGEYGD | ✓ | | SEQ ID NO: 99 |
| SHEVLHAMSRGVEVLA | | ✓ | SEQ ID NO: 100 |

1-78. (canceled)

79. A method of eliciting in a subject an immune response to an agent, the method including the step of administering to the subject an effective amount of a protein particle comprising a diphtheria toxin Cross Reacting Material (CRM) amino acid sequence, wherein the protein particle comprising the diphtheria toxin CRM amino acid sequence is derived from a cell, to thereby elicit in the subject the immune response against the agent.

80. The method of claim 79, wherein the protein particle comprising a diphtheria toxin CRM amino acid sequence is formed when the diphtheria toxin CRM amino acid sequence is expressed in the cell.

81. The method of claim 79, wherein the protein particle comprising a diphtheria toxin CRM amino acid sequence is derived from an insoluble component of the cell, wherein the insoluble component of the cell has not been subjected to a protein refolding treatment.

82. The method of claim 81, wherein the insoluble component is an inclusion body formed in the cell.

83. The method of claim 79, wherein the diphtheria toxin CRM amino acid sequence is derived from a CRM197 protein, or a fragment, variant, or derivative thereof.

84. A method of immunising a subject against a disease, disorder, or condition, the method including the step of administering to the subject an effective amount of a protein particle comprising a diphtheria toxin Cross Reacting Material (CRM) amino acid sequence, wherein the protein particle comprising the diphtheria toxin CRM amino acid sequence is derived from a cell, to thereby immunise the subject against the disease, disorder, or condition.

85. The method of claim 84, wherein the protein particle comprising a diphtheria toxin CRM amino acid sequence is formed when the diphtheria toxin CRM amino acid sequence is expressed in the cell.

86. The method of claim 84, wherein the protein particle comprising a diphtheria toxin CRM amino acid sequence is derived from an insoluble component of the cell, wherein the insoluble component of the cell has not been subjected to a protein refolding treatment.

87. The method of claim 86, wherein the insoluble component is an inclusion body formed in the cell.

88. The method of claim 84, wherein the diphtheria toxin CRM amino acid sequence is derived from a CRM197 protein, or a fragment, variant, or derivative thereof.

89. A method of treating or preventing a disease, disorder, or condition in a subject, the method including the step of administering to the subject an effective amount of a protein particle comprising a diphtheria toxin Cross Reacting Material (CRM) amino acid sequence, wherein the protein particle comprising the diphtheria toxin CRM amino acid sequence is derived from a cell, to thereby treat or prevent the disease, disorder, or condition, in the subject.

90. The method of claim 89, wherein the protein particle comprising a diphtheria toxin CRM amino acid sequence is formed when the diphtheria toxin CRM amino acid sequence is expressed in the cell.

91. The method of claim 89, wherein the protein particle comprising a diphtheria toxin CRM amino acid sequence is derived from an insoluble component of the cell, wherein the insoluble component of the cell has not been subjected to a protein refolding treatment.

92. The method of claim 91, wherein the insoluble component is an inclusion body formed in the cell.

93. The method of claim 89, wherein the diphtheria toxin CRM amino acid sequence is derived from a CRM197 protein, or a fragment, variant, or derivative thereof.

94. A composition, the composition comprising a protein particle comprising a diphtheria toxin Cross Reacting Material (CRM) amino acid sequence, wherein the protein particle comprising the diphtheria toxin CRM amino acid sequence is derived from a cell, and a pharmaceutically-acceptable diluent, carrier, or excipient.

95. The composition of claim 94, wherein the protein particle comprising a diphtheria toxin CRM amino acid sequence is formed when the diphtheria toxin CRM amino acid sequence is expressed in the cell.

96. The composition of claim 94, wherein the protein particle comprising a diphtheria toxin CRM amino acid sequence is derived from an insoluble component of the cell, wherein the insoluble component of the cell has not been subjected to a protein refolding treatment.

97. The composition of claim 95, wherein the insoluble component is an inclusion body formed in the cell.

98. The composition of claim 94, wherein the diphtheria toxin CRM amino acid sequence is derived from a CRM197 protein, or a fragment, variant, or derivative thereof.

99. The composition of claim 94, wherein the composition is a pharmaceutical composition.
